(19) United States
(12) Patent Application Publication
(10) Pub. No.: US 2022/0243225 A1
(43) Pub. Date: Aug. 4, 2022

(54) SYSTEMS AND METHODS FOR PRODUCING BACULOVIRAL INFECTED INSECT CELLS (BIICs) IN BIOREACTORS

(71) Applicant: Voyager Therapeutics, Inc., Cambridge, MA (US)

(72) Inventors: Krishanu MATHUR, Somerville, MA (US); Andrade HENDRICKS, Cambridge, MA (US); Christopher J. MORRISON, Arlington, MA (US); Taylor POLHEMUS, Cambridge, MA (US); Peter SLADE, Milton, MA (US); Anastasia NEUMAN, Philadelphia, PA (US)

(21) Appl. No.: 17/606,635

(22) PCT Filed: Apr. 29, 2020
(86) PCT No.: PCT/US2020/030355

§ 371 Date: Oct. 26, 2021

Related U.S. Application Data
(60) Provisional application No. 63/010,342, filed on Apr. 15, 2020.
(60) Provisional application No. 62/884,827, filed on Aug. 9, 2019.
(60) Provisional application No. 62/839,893, filed on Apr. 29, 2019.

(51) Int. Cl. C12N 15/86; C12N 5/07; C12N 7/00; C12M 1/00; A61K 35/76

(52) U.S. Cl. CPC C12N 15/86; C12N 5/0601; C12N 7/00; C12N 2750/14151; A61K 35/76; C12N 2710/14043; C12N 2750/14143; C12M 29/10

(57) ABSTRACT

The present disclosure presents methods for producing baculovirus infected insect cells (BIICs). The present disclosure describes methods and systems for use in the production of adeno-associated virus (AAV) particles, compositions and formulations, including recombinant adeno-associated viruses (rAAV). In certain embodiments, the production process and system use Baculoviral Expression Vectors (BEVs) and/or Baculoviral Infected Insect Cells (BIICs) in the production of rAAVs. In certain embodiments, the present disclosure presents methods and systems for designing, producing, clarifying, purifying, formulating, filtering and processing rAAVs and rAAV formulations. In certain embodiments, the production process and system use Spodoptera frugiperda insect cells (such as Sf9 or Sf21) as viral production cells (VPCs).

### CROSS REFERENCE TO RELATED APPLICATIONS

This application claims the benefit of: U.S. Provisional Patent Application No. 62/839,893, filed Apr. 29, 2019, entitled SYSTEMS AND METHODS FOR PRODUCING GENE THERAPY PRODUCTS IN BIOREACTORS; U.S. Provisional Patent Application No. 62/884,827, filed Aug. 9, 2019, entitled SYSTEMS AND METHODS FOR PRODUCING GENE THERAPY PRODUCTS IN BIOREACTORS; U.S. Provisional Patent Application No. 63/010,342, filed Apr. 15, 2020, entitled SYSTEMS AND METHODS FOR PRODUCING BACULOVIRAL INFECTED INSECT CELLS (BIICs) IN BIOREACTORS; the contents of which are each incorporated herein by reference in their entireties.

### FIELD OF THE DISCLOSURE

The present disclosure presents methods for producing baculovirus infected insect cells (BIICs). The present disclosure describes methods and systems for use in the production of adeno-associated virus (AAV) particles, compositions and formulations, including recombinant adeno-associated viruses (rAAV). In certain embodiments, the production process and system use Baculoviral Expression Vectors (BEVs) and/or Baculoviral Infected Insect Cells (BIICs) in the production of rAAVs. In certain embodiments, the present disclosure presents methods and systems for designing, producing, clarifying, purifying, formulating, filtering and processing rAAVs and rAAV formulations. In certain embodiments, the production process and system use Spodoptera frugiperda insect cells (such as Sf9 or Sf21) as viral production cells (VPCs).

### BACKGROUND

AAVs have emerged as one of the most widely studied and utilized viral vectors for gene transfer to mammalian cells. See, e.g., Tratschin et al., Mol. Cell Biol., 5(11):3251-3260 (1985) and Grimm et al., Hum. Gene Ther., 10(15):2445-2450 (1999), the contents of which are each incorporated herein by reference in their entireties insofar as they do not conflict with the present disclosure. Adeno-associated viral (AAV) vectors are promising candidates for therapeutic gene delivery and have proven safe and efficacious in clinical trials. The design and production of improved AAV particles for this purpose is an active field of study.

There remains a need for improved systems and methods for producing AAV capsids proteins, AAV capsids, and corresponding AAV vectors (such as AAV particles).

### SUMMARY

The details of various embodiments of the present disclosure are set forth in the description below.

The present disclosure presents methods for producing baculovirus infected insect cells (BIICs). In certain embodiments, the present disclosure presents methods for producing a baculovirus infected insect cell (BIIC) which includes one or more of the following steps: (a) introducing a volume of cell culture medium into a bioreactor; (b) introducing at least one viral production cell (VPC) into the bioreactor and expanding the number of VPCs in the bioreactor to a target VPC cell density; (c) introducing at least one Baculoviral Expression Vector (BEV) into the bioreactor, wherein the BEV comprises an AAV viral expression construct or an AAV payload construct; (d) incubating the mixture of VPCs and BEVs in the bioreactor under conditions which allow at least one BEV to infect at least one VPC to produce a baculovirus infected insect cell (BIIC); (e) incubating the bioreactor under conditions which allow the number of BIICs in the bioreactor to reach a target BIIC cell density; and (f) optionally harvesting the BIICs from the bioreactor. In certain embodiments, the bioreactor comprises a perfusion system for managing the cell culture medium within the bioreactor. In certain embodiments, the perfusion system is an alternating tangential flow (ATF) perfusion system.

The present disclosure presents methods for producing a baculovirus infected insect cell (BIIC) in which the bioreactor includes a perfusion system for managing the cell culture medium within the bioreactor. In certain embodiments, the present disclosure presents methods for producing a baculovirus infected insect cell (BIIC) which includes the following steps: (a) introducing a volume of cell culture medium into a bioreactor, wherein the bioreactor includes perfusion system for managing the cell culture medium within the bioreactor; (b) introducing at least one viral production cell (VPC) into the bioreactor and expanding the number of VPCs in the bioreactor to a target VPC cell density; (c) introduction at least one Baculoviral Expression Vector (BEV) into the bioreactor, wherein the BEV comprises an AAV viral expression construct or an AAV payload construct; (d) incubating the mixture of VPCs and BEVs in the bioreactor under conditions which allow at least one BEV to infect at least one VPC to produce a baculovirus infected insect cell (BIIC); (e) incubating the bioreactor under conditions which allow the number of BIICs in the bioreactor to reach a target BIIC cell density; and (f) harvesting the BIICs from the bioreactor. In certain embodiments, the perfusion system is an alternating tangential flow (ATF) perfusion system (e.g. XCell ATF system).

In certain embodiments, the perfusion system replaces at least a portion of the culture medium in the bioreactor while retaining at least 90% of the VPCs and BIICs within the bioreactor. In certain embodiments, the perfusion system removes cell waste products from the cell culture medium within the bioreactor. In certain embodiments, the perfusion system replaces cell culture media which has been depleted of nutrients by cellular metabolism. In certain embodiments, the perfusion system replaces the cell culture media with a cell culture media supplemented with growth or production boosting factors to increase the quality and quantity of the AAV product. In certain embodiments, the perfusion system replaces cell culture media with a cryopreservation media after the bioreactor reaches the target BIIC cell density, which allows for BIIC cells to be frozen and preserved before or after being harvested from the bioreactor.

In certain embodiments, the bioreactor has a volume of at least 5 L, 10 L, 20 L, 50 L, 100 L, or 200 L. In certain embodiments, the volume of cell culture medium (i.e. working volume) in the bioreactor is at least 5 L, 10 L, 20 L, 50 L, 100 L, or 200 L. In certain embodiments, the target VPC cell density at BEV infection is 1.5-4.0×10⁶ cells/mL, or more specifically 2.0-3.5×10⁶ cells/mL. In certain embodiments, the VPCs are insect cells. In certain embodiments, the VPCs are Sf9 cells.

In certain embodiments, the BEVs are introduced into the bioreactor at a target Multiplicity of Infection (MOI) of BEVs to VPCs. In certain embodiments, the BEV MOI is 0.0005-0.003, or more specifically 0.001-0.002.

In certain embodiments, the BIICs are harvested from the bioreactor at a specific BIIC cell density. In certain embodiments, the BIICs harvested from the bioreactor have a specific BIIC cell density. In certain embodiments, the BIIC cell density at harvesting is 6.0-18.0×10⁶ cells/mL, more specifically 8.0-16.5×10⁶ cells/mL or 10.0-16.5×10⁶ cells/mL.

The present disclosure presents baculovirus infected insect cells (BIIC) produced by the methods of the present disclosure.

The present disclosure presents methods for producing an adeno-associated virus (AAV) comprising a polynucleotide encoding a payload, wherein the method uses one or more BIICs of the present disclosure. In certain embodiments, the present disclosure presents methods for producing an adeno-associated virus (AAV) which includes one or more of the following steps: (a) culturing viral production cells (VPCs) in a bioreactor to a target cell density, wherein the bioreactor comprises a cell culture medium; (b) introducing into the bioreactor at least one baculovirus infected insect cell (BIIC), wherein the at least one BIIC introduced into the bioreactor comprises at least one expressionBIIC which comprises at least one expressionBac, wherein the at least one BIIC introduced into the bioreactor comprises at least one payloadBIIC which comprises at least one payloadBac, and wherein at least one of the BIICs introduced into the bioreactor is a BIIC of the present disclosure; (c) incubating the VPCs in the bioreactor under conditions that result in the production of one or more AAVs within one or more VPCs, wherein one or more of the AAVs comprise the polynucleotide encoding the payload; and (d) harvesting a viral production pool from the bioreactor, wherein the viral production pool comprises one or more VPCs comprising one or more AAVs.

In certain embodiments, the VPCs are insect cells. In certain embodiments, the VPCs are Sf9 cells. In certain embodiments, the target cell density of the VPCs of step (a) is 3.0×106-3.4×106 cells/mL (e.g., 3.2×106-3.4×106 cells/mL; e.g., 3.2×106 cells/mL).

The present disclosure presents an adeno-associated virus (AAV) produced by a method of the present disclosure. The present disclosure presents pharmaceutical compositions comprising an AAV produced by methods of the present disclosure. In certain embodiments, the pharmaceutical composition is for use in treating and/or preventing a disease. In certain embodiments the pharmaceutical composition can be used in a method of treating a disease, wherein the method comprises administering an effective amount of the pharmaceutical to a subject. In certain embodiments the pharmaceutical composition can be used in the manufacture of a medicament for treating and/or preventing a disease.

### BRIEF DESCRIPTION OF THE FIGURES

The foregoing and other objects, features and advantages will be apparent from the following description of particular embodiments of the present disclosure, as illustrated in the accompanying figures. The figures are not necessarily to scale or comprehensive, with emphasis instead being placed upon illustrating the principles of various embodiments of the present disclosure.

FIG. 1 shows a schematic for one embodiment of a system, and a flow diagram for one embodiment of a process, for producing baculovirus infected insect cells (BIICs) using Viral Production Cells (VPC) and plasmid constructs.

FIG. 2 shows a schematic for one embodiment of a system, and a flow diagram for one embodiment of a process, for producing AAV Particles using Viral Production Cells (VPC) and baculovirus infected insect cells (BIICs).

FIG. 3 shows schematic for one embodiment of a system, and a flow diagram for one embodiment of a process, for producing a Drug Substance by processing, clarifying and purifying a bulk harvest of AAV particles and Viral Production Cells.

FIG. 4A presents Viable Cell Density data (×10⁶ cells/mL) corresponding with certain embodiments of BIIC production systems of the present disclosure.

FIG. 4B presents Cell Viability data (%) corresponding with certain embodiments of BIIC production systems of the present disclosure.

FIG. 5 presents a graph showing the correlation between BIIC production processes and AAV Titer according to certain embodiments of AAV particle production of the present disclosure.

### DETAILED DESCRIPTION

### I. Adeno-Associated Viruses (AAVs)

### Overview

Adeno-associated viruses (AAV) are small non-enveloped icosahedral capsid viruses of the Parvoviridae family characterized by a single stranded DNA viral genome. Parvoviridae family viruses consist of two subfamilies: Parvovirinae, which infect vertebrates, and Densovirinae, which infect invertebrates. The Parvoviridae family includes the Dependovirus genus which includes AAV, capable of replication in vertebrate hosts including, but not limited to, human, primate, bovine, canine, equine, and ovine species.

The parvoviruses and other members of the Parvoviridae family are generally described in Kenneth I. Berns, “Parvoviridae: The Viruses and Their Replication,” Chapter 69 in Fields Virology (3d Ed. 1996), the contents of which are incorporated by reference in their entirety.

AAV have proven to be useful as a biological tool due to their relatively simple structure, their ability to infect a wide range of cells (including quiescent and dividing cells) without integration into the host genome and without replicating, and their relatively benign immunogenic profile. The genome of the virus may be manipulated to contain a minimum of components for the assembly of a functional recombinant virus, or viral particle, which is loaded with or engineered to target a particular tissue and express or deliver a desired payload.

### AAV Viral Genomes

The wild-type AAV viral genome is a linear, single-stranded DNA (ssDNA) molecule approximately 5,000 nucleotides (nt) in length. Inverted terminal repeats (ITRs) traditionally cap the viral genome at both the 5′ and the 3′ end, providing origins of replication for the viral genome. While not wishing to be bound by theory, an AAV viral genome typically comprises two ITR sequences. These ITRs have a characteristic T-shaped hairpin structure defined by a self-complementary region (145 nt in wild-type AAV) at the 5′ and 3′ ends of the ssDNA which form an energetically stable double stranded region. The double stranded hairpin structures comprise multiple functions comprising, but not limited to, acting as an origin for DNA replication by functioning as primers for the endogenous DNA polymerase complex of the host viral replication cell.

The wild-type AAV viral genome further comprises nucleotide sequences for two open reading frames, one for the four non-structural Rep proteins (Rep78, Rep68, Rep52, Rep40, encoded by Rep genes) and one for the three capsid, or structural, proteins (VP1, VP2, VP3, encoded by capsid genes or Cap genes). The Rep proteins are important for replication and packaging, while the capsid proteins are assembled to create the protein shell of the AAV, or AAV capsid. Alternative splicing and alternate initiation codons and promoters result in the generation of four different Rep proteins from a single open reading frame and the generation of three capsid proteins from a single open reading frame. Though it varies by AAV serotype, as a non-limiting example, for AAV9/hu.14 (SEQ ID NO: 123 of U.S. Pat. No. 7,906,111, the content of which is incorporated herein by reference in its entirety as related to AAV9/hu.14, insofar as it does not conflict with the present disclosure) VP1 refers to amino acids 1-736, VP2 refers to amino acids 138-736, and VP3 refers to amino acids 203-736. In other words, VP1 is the full-length capsid sequence, while VP2 and VP3 are shorter components of the whole. As a result, changes in the sequence in the VP3 region, are also changes to VP1 and VP2, however, the percent difference as compared to the parent sequence will be greatest for VP3 since it is the shortest sequence of the three. Though described here in relation to the amino acid sequence, the nucleic acid sequence encoding these proteins can be similarly described. Together, the three capsid proteins assemble to create the AAV capsid protein. While not wishing to be bound by theory, the AAV capsid protein typically comprises a molar ratio of 1:1:10 of VP1:VP2:VP3. As used herein, an “AAV serotype” is defined primarily by the AAV capsid. In some instances, the ITRs are also specifically described by the AAV serotype (e.g., AAV2/9).

For use as a biological tool, the wild-type AAV viral genome can be modified to replace the rep/cap sequences with a nucleic acid sequence comprising a payload region with at least one ITR region. Typically, in recombinant AAV viral genomes there are two ITR regions. The rep/cap sequences can be provided in trans during production to generate AAV particles.

In addition to the encoded heterologous payload, AAV vectors may comprise the viral genome, in whole or in part, of any naturally occurring and/or recombinant AAV serotype nucleotide sequence or variant. AAV variants may have sequences of significant homology at the nucleic acid (genome or capsid) and amino acid levels (capsids), to produce constructs which are generally physical and functional equivalents, replicate by similar mechanisms, and assemble by similar mechanisms. See Chiorini et al., J. Vir. 71: 6823-33(1997); Srivastava et al., J. Vir. 45:555-64 (1983); Chiorini et al., J. Vir. 73:1309-1319 (1999); Rutledge et al., J. Vir. 72:309-319 (1998); and Wu et al., J. Vir. 74: 8635-47 (2000), the contents of each of which are incorporated herein by reference in their entireties as related to AAV variants and equivalents, insofar as they do not conflict with the present disclosure.

In certain embodiments, AAV particles, viral genomes and/or payloads of the present disclosure, and the methods of their use, may be as described in WO2017189963, the content of which is incorporated herein by reference in its entirety as related to AAV particles, viral genomes and/or payloads, insofar as it does not conflict with the present disclosure.

AAV particles of the present disclosure may be formulated in any of the gene therapy formulations of the disclosure comprising any variations of such formulations apparent to those skilled in the art. The reference to “AAV particles”, “AAV particle formulations” and “formulated AAV particles” in the present application refers to the AAV particles which may be formulated and those which are formulated without limiting either.

In certain embodiments, AAV particles of the present disclosure are recombinant AAV (rAAV) viral particles which are replication defective, lacking sequences encoding functional Rep and Cap proteins within their viral genome. These defective AAV particles may lack most or all parental coding sequences and essentially carry only one or two AAV ITR sequences and the nucleic acid of interest (i.e. payload) for delivery to a cell, a tissue, an organ or an organism.

In certain embodiments, the viral genome of the AAV particles of the present disclosure comprises at least one control element which provides for the replication, transcription and translation of a coding sequence encoded therein. Not all of the control elements need always be present as long as the coding sequence is capable of being replicated, transcribed and/or translated in an appropriate host cell. Non-limiting examples of expression control elements comprise sequences for transcription initiation and/or termination, promoter and/or enhancer sequences, efficient RNA processing signals such as splicing and polyadenylation signals, sequences that stabilize cytoplasmic mRNA, sequences that enhance translation efficacy (e.g., Kozak consensus sequence), sequences that enhance protein stability, and/or sequences that enhance protein processing and/or secretion.

According to the present disclosure, AAV particles for use in therapeutics and/or diagnostics comprise a virus that has been distilled or reduced to the minimum components necessary for transduction of a nucleic acid payload or cargo of interest. In this manner, AAV particles are engineered as vehicles for specific delivery while lacking the deleterious replication and/or integration features found in wild-type viruses.

AAV particles of the present disclosure may be produced recombinantly and may be based on adeno-associated virus (AAV) parent or reference sequences. As used herein, a “vector” is any molecule or moiety which transports, transduces or otherwise acts as a carrier of a heterologous molecule such as the nucleic acids described herein.

In addition to single stranded AAV viral genomes (e.g., ssAAVs), the present disclosure also provides for self-complementary AAV (scAAVs) viral genomes. scAAV viral genomes contain DNA strands which anneal together to form double stranded DNA. By skipping second strand synthesis, scAAVs allow for rapid expression in the cell.

In certain embodiments, the AAV viral genome of the present disclosure is a scAAV. In certain embodiments, the AAV viral genome of the present disclosure is a ssAAV.

Methods for producing and/or modifying AAV particles are disclosed in the art, such as pseudotyped AAV particles (PCT Patent Publication Nos. WO200028004; WO200123001; WO2004112727; WO 2005005610 and WO 2005072364, the contents of each of which are incorporated herein by reference in their entireties as related to producing and/or modifying AAV particles, insofar as they do not conflict with the present disclosure).

AAV particles may be modified to enhance the efficiency of delivery. Such modified AAV particles can be packaged efficiently and be used to successfully infect the target cells at high frequency and with minimal toxicity. In certain embodiments the capsids of the AAV particles are engineered according to the methods described in US Publication Number US 20130195801, the content of which is incorporated herein by reference in its entirety as related to modifying AAV particles to enhance the efficiency of delivery, insofar as it does not conflict with the present disclosure.

In certain embodiments, the AAV particles comprise a payload construct and/or region encoding a polypeptide or protein of the present disclosure, and may be introduced into mammalian cells. In certain embodiments, the AAV particles comprise a payload construct and/or region encoding a polypeptide or protein of the present disclosure, and may be introduced into insect cells.

In certain embodiments, the AAV particles of the present disclosure comprise a viral genome with at least one ITR region and a payload region. In certain embodiments, the viral genome has two ITRs. These two ITRs flank the payload region at the 5′ and 3′ ends. The ITRs function as origins of replication comprising recognition sites for replication. ITRs comprise sequence regions which can be complementary and symmetrically arranged. ITRs incorporated into viral genomes of the present disclosure may be comprised of naturally occurring polynucleotide sequences or recombinantly derived polynucleotide sequences.

The ITRs may be derived from the same serotype as the capsid, or a derivative thereof. The ITR may be of a different serotype than the capsid. In certain embodiments, the AAV particle has more than one ITR. In a non-limiting example, the AAV particle has a viral genome comprising two ITRs. In certain embodiments, the ITRs are of the same serotype as one another. In another embodiment, the ITRs are of different serotypes. Non-limiting examples comprise zero, one or both of the ITRs having the same serotype as the capsid. In certain embodiments both ITRs of the viral genome of the AAV particle are AAV2 ITRs.

Independently, each ITR may be about 100 to about 150 nucleotides in length. An ITR may be about 100-105 nucleotides in length, 106-110 nucleotides in length, 111-115 nucleotides in length, 116-120 nucleotides in length, 121-125 nucleotides in length, 126-130 nucleotides in length, 131-135 nucleotides in length, 136-140 nucleotides in length, 141-145 nucleotides in length or 146-150 nucleotides in length. In certain embodiments, the ITRs are 140-142 nucleotides in length. Non-limiting examples of ITR length are 102, 130, 140, 141, 142, 145 nucleotides in length.

In certain embodiments, each ITR may be 141 nucleotides in length. In certain embodiments, each ITR may be 130 nucleotides in length. In certain embodiments, each ITR may be 119 nucleotides in length.

### Viral Genome Size

In certain embodiments, the AAV particle which includes a payload described herein may be single stranded or double stranded viral genome. The size of the viral genome may be small, medium, large or the maximum size. Additionally, the viral genome may include a promoter and a polyA tail.

In certain embodiments, the viral genome which includes a payload described herein may be a small single stranded viral genome. A small single stranded viral genome may be 2.1 to 3.5 kb in size such as about 2.1, 2.2, 2.3, 2.4, 2.5, 2.6, 2.7, 2.8, 2.9, 3.0, 3.1, 3.2, 3.3, 3.4, and 3.5 kb in size. As a non-limiting example, the small single stranded viral genome may be 3.2 kb in size. As another non-limiting example, the small single stranded viral genome may be 2.2 kb in size. Additionally, the viral genome may include a promoter and a polyA tail.

In certain embodiments, the viral genome which includes a payload described herein may be a small double stranded viral genome. A small double stranded viral genome may be 1.3 to 1.7 kb in size such as about 1.3, 1.4, 1.5, 1.6, and 1.7 kb in size. As a non-limiting example, the small double stranded viral genome may be 1.6 kb in size. Additionally, the viral genome may include a promoter and a polyA tail.

In certain embodiments, the viral genome which includes a payload described herein e.g., polynucleotide, siRNA or dsRNA, may be a medium single stranded viral genome. A medium single stranded viral genome may be 3.6 to 4.3 kb in size such as about 3.6, 3.7, 3.8, 3.9, 4.0, 4.1, 4.2 and 4.3 kb in size. As a non-limiting example, the medium single stranded viral genome may be 4.0 kb in size. Additionally, the viral genome may include a promoter and a polyA tail.

In certain embodiments, the viral genome which includes a payload described herein may be a medium double stranded viral genome. A medium double stranded viral genome may be 1.8 to 2.1 kb in size such as about 1.8, 1.9, 2.0, and 2.1 kb in size. As a non-limiting example, the medium double stranded viral genome may be 2.0 kb in size. Additionally, the viral genome may include a promoter and a polyA tail.

In certain embodiments, the viral genome which includes a payload described herein may be a large single stranded viral genome. A large single stranded viral genome may be 4.4 to 6.0 kb in size such as about 4.4, 4.5, 4.6, 4.7, 4.8, 4.9, 5.0, 5.1, 5.2, 5.3, 5.4, 5.5, 5.6, 5.7, 5.8, 5.9 and 6.0 kb in size. As a non-limiting example, the large single stranded viral genome may be 4.7 kb in size. As another non-limiting example, the large single stranded viral genome may be 4.8 kb in size. As yet another non-limiting example, the large single stranded viral genome may be 6.0 kb in size. Additionally, the viral genome may include a promoter and a polyA tail.

In certain embodiments, the viral genome which includes a payload described herein may be a large double stranded viral genome. A large double stranded viral genome may be 2.2 to 3.0 kb in size such as about 2.2, 2.3, 2.4, 2.5, 2.6, 2.7, 2.8, 2.9 and 3.0 kb in size. As a non-limiting example, the large double stranded viral genome may be 2.4 kb in size. Additionally, the viral genome may include a promoter and a polyA tail.

In certain embodiments, an viral genome of the present disclosure can include at least one filler region. In certain embodiments, an viral genome of the present disclosure can include at least one multiple cloning site (MCS) region. In certain embodiments, an viral genome of the present disclosure can include at least one promoter region. In certain embodiments, an viral genome of the present disclosure can include at least one exon region. In certain embodiments, an viral genome of the present disclosure can include at least one intron region.

### Viral Genome Regions: Inverted Terminal Repeats (ITRs)

The AAV particles of the present disclosure include a viral genome with at least one Inverted Terminal Repeat (ITR) region and a payload region. In certain embodiments, the viral genome has two ITRs. These two ITRs flank the payload region at the 5′ and 3′ ends. The ITRs function as origins of replication including recognition sites for replication. ITRs include sequence regions which can be complementary and symmetrically arranged. ITRs incorporated into viral genomes of the present disclosure may be included of naturally occurring polynucleotide sequences or recombinantly derived polynucleotide sequences.

The ITRs may be derived from the same serotype as the capsid, or a derivative thereof. The ITR may be of a different serotype than the capsid. In certain embodiments, the AAV particle has more than one ITR. In a non-limiting example, the AAV particle has a viral genome including two ITRs. In certain embodiments, the ITRs are of the same serotype as one another. In another embodiment, the ITRs are of different serotypes. Non-limiting examples include zero, one or both of the ITRs having the same serotype as the capsid. In certain embodiments both ITRs of the viral genome of the AAV particle are AAV2 ITRs.

Independently, each ITR may be about 100 to about 150 nucleotides in length. An ITR may be about 100-105 nucleotides in length, 106-110 nucleotides in length, 111-115 nucleotides in length, 116-120 nucleotides in length, 121-125 nucleotides in length, 126-130 nucleotides in length, 131-135 nucleotides in length, 136-140 nucleotides in length, 141-145 nucleotides in length or 146-150 nucleotides in length. In certain embodiments, the ITRs are 140-142 nucleotides in length. Non-limiting examples of ITR length are 102, 130, 140, 141, 142, 145 nucleotides in length, and those having at least 95% identity thereto.

In certain embodiments, each ITR may be 141 nucleotides in length. In certain embodiments, each ITR may be 130 nucleotides in length. In certain embodiments, each ITR may be 119 nucleotides in length.

In certain embodiments, the AAV particles include two ITRs and one ITR is 141 nucleotides in length and the other ITR is 130 nucleotides in length. In certain embodiments, the AAV particles include two ITRs and both ITR are 141 nucleotides in length.

Independently, each ITR may be about 75 to about 175 nucleotides in length. The ITR may, independently, have a length such as, but not limited to, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, and 175 nucleotides. The length of the ITR for the viral genome may be 75-80, 75-85, 75-100, 80-85, 80-90, 80-105, 85-90, 85-95, 85-110, 90-95, 90-100, 90-115, 95-100, 95-105, 95-120, 100-105, 100-110, 100-125, 105-110, 105-115, 105-130, 110-115, 110-120, 110-135, 115-120, 115-125, 115-140, 120-125, 120-130, 120-145, 125-130, 125-135, 125-150, 130-135, 130-140, 130-155, 135-140, 135-145, 135-160, 140-145, 140-150, 140-165, 145-150, 145-155, 145-170, 150-155, 150-160, 150-175, 155-160, 155-165, 160-165, 160-170, 165-170, 165-175, and 170-175 nucleotides. As a non-limiting example, the viral genome comprises an ITR that is about 105 nucleotides in length. As a non-limiting example, the viral genome comprises an ITR that is about 141 nucleotides in length. As a non-limiting example, the viral genome comprises an ITR that is about 130 nucleotides in length. As a non-limiting example, the viral genome comprises an ITR that is about 105 nucleotides in length and 141 nucleotides in length. As a non-limiting example, the viral genome comprises an ITR that is about 105 nucleotides in length and 130 nucleotides in length. As a non-limiting example, the viral genome comprises an ITR that is about 130 nucleotides in length and 141 nucleotides in length.

### AAV Serotypes

AAV particles of the present disclosure may include or be derived from any natural or recombinant AAV serotype. According to the present disclosure, the AAV particles may utilize or be based on a serotype or include a peptide selected from any of the following: VOY101, VOY201, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3 (G2A3), AAVG2B4 (G2B4), AAVG2B5 (G2B5), PHP.S, AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, AAVF9/HSC9, AAVrh20, AAVrh32/33, AAVrh39, AAVrh46, AAVrh73, AAVrh74, AAVhu.26, or variants or derivatives thereof.

The AAV-DJ sequence may include two mutations: (1) R587Q where arginine (R; Arg) at amino acid 587 is changed to glutamine (Q; Gln) and (2) R590T where arginine (R; Arg) at amino acid 590 is changed to threonine (T; Thr). As another non-limiting example, may include three mutations: (1) K406R where lysine (K; Lys) at amino acid 406 is changed to arginine (R; Arg), (2) R587Q where arginine (R; Arg) at amino acid 587 is changed to glutamine (Q; Gln) and (3) R590T where arginine (R; Arg) at amino acid 590 is changed to threonine (T; Thr).

In certain embodiments, the AAV may be a serotype generated by the AAV9 capsid library with mutations in amino acids 390-627 (VP1 numbering) The serotype and corresponding nucleotide and amino acid substitutions may be, but is not limited to, AAV9.1 (G1594C; D532H), AAV6.2 (T1418A and T1436X; V473D and 1479K), AAV9.3 (T1238A; F413Y), AAV9.4 (T1250C and A1617T; F417S), AAV9.5 (A1235G, A1314T, A1642G, C1760T; Q412R, T548A, A587V), AAV9.6 (T1231A; F411I), AAV9.9 (G1203A, G1785T; W595C), AAV9.10 (A1500G, T1676C; M559T), AAV9.11 (A1425T, A1702C, A1769T; T568P, Q590L), AAV9.13 (A1369C, A1720T; N457H, T574S), AAV9.14 (T1340A, T1362C, T1560C, G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V606I), AAV9.40 (A1694T, E565V), AAV9.41 (A 1348T, T1362C; T450S), AAV9.44 (A 1684C, A 1701T, A1737G; N562H, K567N), AAV9.45 (A1492T, C1804T; N498Y, L602F), AAV9.46 (G1441C, T1525C, T1549G; G481R, W509R, L517V), 9.47 (G1241A, G1358A, A1669G, C1745T; S414N, G453D, K557E, T5821), AAV9.48 (C1445T, A1736T; P482L, Q579L), AAV9.50 (A 1638T, C1683T, T1805A; Q546H, L602H), AAV9.53 (G1301A, A1405C, C1664T, G1811T; R134Q, S469R, A555V, G604V), AAV9.54 (C1531A, T1609A; L511I, L537M), AAV9.55 (T1605A; F535L), AAV9.58 (C1475T, C1579A; T492I, H527N), AAV.59 (T1336C; Y446H), AAV9.61 (A 1493T; N4981), AAV9.64 (C1531A, A1617T; L511I), AAV9.65 (C1335T, T1530C, C1568A; A523D), AAV9.68 (C1510A; P504T), AAV9.80 (G1441A; G481R), AAV9.83 (C1402A, A1500T; P468T, E500D), AAV9.87 (T1464C, T1468C; S490P), AAV9.90 (A1196T; Y399F), AAV9.91 (T1316G, A1583T, C1782G, T1806C; L439R, K5281), AAV9.93 (A 1273G, A1421G, A 1638C, C1712T, G1732A, A1744T, A1832T; S425G, Q474R, Q546H, P571L, G578R, T582S, D611V), AAV9.94 (A 1675T; M559L) and AAV9.95 (T1605A; F535L).

In any of the DNA and RNA sequences referenced and/or described herein, the single letter symbol has the following description: A for adenine; C for cytosine; G for guanine; T for thymine; U for Uracil; W for weak bases such as adenine or thymine; S for strong nucleotides such as cytosine and guanine; M for amino nucleotides such as adenine and cytosine; K for keto nucleotides such as guanine and thymine; R for purines adenine and guanine; Y for pyrimidine cytosine and thymine; B for any base that is not A (e.g., cytosine, guanine, and thymine); D for any base that is not C (e.g., adenine, guanine, and thymine); H for any base that is not G (e.g., adenine, cytosine, and thymine); V for any base that is not T (e.g., adenine, cytosine, and guanine); N for any nucleotide (which is not a gap); and Z is for zero.

In any of the amino acid sequences referenced and/or described herein, the single letter symbol has the following description: G (Gly) for Glycine; A (Ala) for Alanine; L (Leu) for Leucine; M (Met) for Methionine; F (Phe) for Phenylalanine; W (Trp) for Tryptophan; K (Lys) for Lysine; Q (Gln) for Glutamine; E (Glu) for Glutamic Acid; S (Ser) for Serine; P (Pro) for Proline; V (Val) for Valine; I (Ile) for Isoleucine; C (Cys) for Cysteine; Y (Tyr) for Tyrosine; H (His) for Histidine; R (Arg) for Arginine; N (Asn) for Asparagine; D (Asp) for Aspartic Acid; T (Thr) for Threonine; B (Asx) for Aspartic acid or Asparagine; J (Xle) for Leucine or Isoleucine; O (Pyl) for Pyrrolysine; U (Sec) for Selenocysteine; X (Xaa) for any amino acid; and Z (Glx) for Glutamine or Glutamic acid.

In certain embodiments, the AAV serotype may be, or may include a sequence, insert, modification or mutation as described in Patent Publications WO2015038958, WO2017100671, WO2016134375, WO2017083722, WO2017015102, WO2017058892, WO2017066764, U.S. Pat. Nos. 9,624,274, 9,475,845, US20160369298, US20170145405, the contents of which are herein incorporated by reference in their entirety.

In certain embodiments, the AAV may be a serotype generated by Cre-recombination-based AAV targeted evolution (CREATE) as described by Deverman et al., (Nature Biotechnology 34(2):204-209 (2016)), the contents of which are herein incorporated by reference in their entirety. In certain embodiments, the AAV serotype may be as described in Jackson et al (Frontiers in Molecular Neuroscience 9:154 (2016)), the contents of which are herein incorporated by reference in their entirety.

In certain embodiments, the AAV serotype is selected for use due to its tropism for cells of the central nervous system. In certain embodiments, the cells of the central nervous system are neurons. In another embodiment, the cells of the central nervous system are astrocytes.

In certain embodiments, the AAV serotype is selected for use due to its tropism for cells of the muscle(s).

In certain embodiments, the initiation codon for translation of the AAV VP1 capsid protein may be CTG, TUG, or GTG as described in U.S. Pat. No. 8,163,543, the contents of which are herein incorporated by reference in its entirety.

The present disclosure refers to structural capsid proteins (including VP1, VP2 and VP3) which are encoded by capsid (Cap) genes. These capsid proteins form an outer protein structural shell (i.e. capsid) of a viral vector such as AAV. VP capsid proteins synthesized from Cap polynucleotides generally include a methionine as the first amino acid in the peptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins.

Where the Met/AA-clipping is incomplete, a mixture of one or more (one, two or three) VP capsid proteins including the viral capsid may be produced, some of which may include a Met1/AA1 amino acid (Met+/AA+) and some of which may lack a Met1/AA1 amino acid as a result of Met/AA-clipping (Met−/AA−). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods. 2017 Oct. 28(5):255-267; Hwang, et al. N-Terminal Acetylation of Cellular Proteins Creates Specific Degradation Signals. Science. 2010 Feb. 19. 327(5968): 973-977; the contents of which are each incorporated herein by reference in their entirety.

According to the present disclosure, references to capsid proteins is not limited to either clipped (Met−/AA−) or unclipped (Met+/AA+) and may, in context, refer to independent capsid proteins, viral capsids included of a mixture of capsid proteins, and/or polynucleotide sequences (or fragments thereof) which encode, describe, produce or result in capsid proteins of the present disclosure. A direct reference to a “capsid protein” or “capsid polypeptide” (such as VP1, VP2 or VP2) may also include VP capsid proteins which include a Met1/AA1 amino acid (Met+/AA+) as well as corresponding VP capsid proteins which lack the Met1/AA1 amino acid as a result of Met/AA-clipping (Met−/AA−).

Further according to the present disclosure, a reference to a specific SEQ ID NO: (whether a protein or nucleic acid) which includes or encodes, respectively, one or more capsid proteins which include a Met1/AA1 amino acid (Met+/AA+) should be understood to teach the VP capsid proteins which lack the Met1/AA1 amino acid as upon review of the sequence, it is readily apparent any sequence which merely lacks the first listed amino acid (whether or not Met1/AA1).

As a non-limiting example, reference to a VP1 polypeptide sequence which is 736 amino acids in length and which includes a “Met1” amino acid (Met+) encoded by the AUG/ATG start codon may also be understood to teach a VP1 polypeptide sequence which is 735 amino acids in length and which does not include the “Met1” amino acid (Met−) of the 736 amino acid Met+ sequence. As a second non-limiting example, reference to a VP1 polypeptide sequence which is 736 amino acids in length and which includes an “AA1” amino acid (AA1+) encoded by any NNN initiator codon may also be understood to teach a VP1 polypeptide sequence which is 735 amino acids in length and which does not include the “AA1” amino acid (AA1−) of the 736 amino acid AA1+ sequence.

References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid as a result of Met/AA1-clipping (Met−/AA1−), and combinations thereof (Met+/AA1+ and Met−/AA1−).

As a non-limiting example, an AAV capsid serotype can include VP1 (Met+/AA1+), VP1 (Met−/AA1−), or a combination of VP1 (Met+/AA1+) and VP1 (Met−/AA1−). An AAV capsid serotype can also include VP3 (Met+/AA1+), VP3 (Met−/AA1−), or a combination of VP3 (Met+/AA1+) and VP3 (Met−/AA1−); and can also include similar optional combinations of VP2 (Met+/AA1) and VP2 (Met−/AA1−).

### Payloads

AAV particles of the present disclosure can comprise, or be produced using, at least one payload construct which comprises at least one payload region. In certain embodiments, the payload region may be located within a viral genome, such as the viral genome of a payload construct. At the 5′ and/or the 3′ end of the payload region there may be at least one inverted terminal repeat (ITR). Within the payload region, there may be a promoter region, an intron region and a coding region.

In certain embodiments, the payload region of the AAV particle comprises one or more nucleic acid sequences encoding one or more payload, such as a payload polypeptide or polynucleotide. In certain embodiments, the payload region of the AAV particle comprises one or more nucleic acid sequences encoding one or more polypeptides or proteins of interest. In certain embodiments, the payload region of the AAV particle comprises one or more nucleic acid sequences encoding one or more modulatory polynucleotides, e.g., RNA or DNA molecules as therapeutic agents. Accordingly, the present disclosure provides viral genomes which encode polynucleotides which are processed into small double stranded RNA (dsRNA) molecules (small interfering RNA, siRNA, miRNA, pre-miRNA) targeting a gene of interest. The present disclosure also provides methods of their use for inhibiting gene expression and protein production of an allele of the gene of interest, for treating diseases, disorders, and/or conditions.

In certain embodiments, the payload region can be included in a payload construct used for producing AAV particles. In certain embodiments, a payload construct of the present disclosure can be a bacmid, also known as a baculovirus plasmid or recombinant baculovirus genome. In certain embodiments, a payload construct of the present disclosure can be a baculovirus expression vector (BEV). In certain embodiments, a payload construct of the present disclosure can be a BIIC which includes a BEV. As used herein, the term “payloadBac” refers to a bacmid (such as a BEV) comprising a payload construct and/or payload region. Viral production cells (e.g., Sf9 cells) may be transfected with payloadBacs and/or with BIICs comprising payloadBacs.

In certain embodiments, the AAV particles of the present disclosure comprise one or more nucleic acid sequences encoding one or more payload, such as a payload polypeptide or polynucleotide, which are useful in the field of medicine for the treatment, prophylaxis, palliation or amelioration of diseases and/or disorders, including neurological diseases and/or disorders. In certain embodiments, the AAV particles of the present disclosure are useful in the field of medicine for the treatment, prophylaxis, palliation or amelioration of Friedreich's ataxia, or any disease stemming from a loss or partial loss of frataxin protein. In certain embodiments, the AAV particles of the present disclosure are useful in the field of medicine for the treatment, prophylaxis, palliation or amelioration of Parkinson's Disease. In certain embodiments, the AAV particles of the present disclosure are useful in the field of medicine for the treatment, prophylaxis, palliation or amelioration of Amyotrophic lateral sclerosis. In certain embodiments, the AAV particles of the present disclosure are useful in the field of medicine for the treatment, prophylaxis, palliation or amelioration of Huntington's Disease. In certain embodiments, the AAV particles of the present disclosure are useful in the field of medicine for the treatment, prophylaxis, palliation or amelioration of Alzheimer's Disease.

### Payloads: Polypeptides and Variants

In certain embodiments, the payload region of the AAV particle comprises one or more nucleic acid sequences encoding a polypeptide or protein of interest. In certain embodiments, the AAV particle comprises a viral genome with a payload region comprising nucleic acid sequences encoding more than one polypeptide of interest. In certain embodiments, a viral genome encoding one or more polypeptides may be replicated and packaged into a viral particle. A target cell transduced with a viral particle comprising the viral genome may express each of the one or more polypeptides in the single target cell.

Where the AAV particle payload region encodes a polypeptide, the polypeptide may be a peptide, polypeptide or protein. As a non-limiting example, the payload region may encode at least one therapeutic protein of interest. The AAV viral genomes encoding polypeptides described herein may be useful in the fields of human disease, viruses, infections veterinary applications and a variety of in vivo and in vitro settings.

In certain embodiments, administration of the formulated AAV particles (which comprise the viral genome) to a subject will increase the expression of a protein in a subject. In certain embodiments, the increase of the expression of the protein will reduce the effects and/or symptoms of a disease or ailment associated with the polypeptide encoded by the payload.

In certain embodiments, the AAV particle comprises a viral genome with a payload region comprising a nucleic acid sequence encoding a protein of interest (i.e. a payload protein, therapeutic protein).

Amino acid sequences encoded by payload regions of the viral genomes of the disclosure may be translated as a whole polypeptide, a plurality of polypeptides or fragments of polypeptides, which independently may be encoded by one or more nucleic acids, fragments of nucleic acids or variants of any of the aforementioned. As used herein, “polypeptide” means a polymer of amino acid residues (natural or unnatural) linked together most often by peptide bonds. The term, as used herein, refers to proteins, polypeptides, and peptides of any size, structure, or function. In some instances, the polypeptide encoded is smaller than about 50 amino acids and the polypeptide is then termed a peptide. If the polypeptide is a peptide, it will be at least about 2, 3, 4, or at least 5 amino acid residues long. Thus, polypeptides comprise gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The term polypeptide may also apply to amino acid polymers in which one or more amino acid residues are an artificial chemical analogue of a corresponding naturally occurring amino acid.

In certain embodiments a “polypeptide variant” is provided. The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants will possess at least about 50% identity (homology) to a native or reference sequence, and in certain embodiments, they will be at least about 80%, or at least about 90% identical (homologous) to a native or reference sequence.

In certain embodiments, the payload region of the AAV particle comprises one or more nucleic acid sequences encoding a polypeptide or protein of interest.

In certain embodiments, the AAV particle comprises a viral genome with a payload region comprising nucleic acid sequences encoding more than one polypeptide of interest. In certain embodiments, a viral genome encoding one or more polypeptides may be replicated and packaged into a viral particle. A target cell transduced with a viral particle comprising the viral genome may express each of the one or more polypeptides in the single target cell.

Where the AAV particle payload region encodes a polypeptide, the polypeptide may be a peptide, polypeptide or protein. As a non-limiting example, the payload region may encode at least one therapeutic protein of interest. The AAV viral genomes encoding polypeptides described herein may be useful in the fields of human disease, viruses, infections veterinary applications and a variety of in vivo and in vitro settings.

In certain embodiments, administration of the formulated AAV particles (which comprise the viral genome) to a subject will increase the expression of a protein in a subject. In certain embodiments, the increase of the expression of the protein will reduce the effects and/or symptoms of a disease or ailment associated with the polypeptide encoded by the payload.

In certain embodiments, the formulated AAV particles of the present disclosure may be used to reduce the decline of functional capacity and activities of daily living as measured by a standard evaluation system such as, but not limited to, the total functional capacity (TFC) scale.

In certain embodiments, the AAV particle comprises a viral genome with a payload region comprising a nucleic acid sequence encoding a protein of interest (i.e. a payload protein, therapeutic protein).

In certain embodiments, the payload region comprises a nucleic acid sequence encoding a protein including but not limited to an antibody, Aromatic L-Amino Acid Decarboxylase (AADC), ApoE2, Frataxin, survival motor neuron (SMN) protein, glucocerebrosidase, N-sulfoglucosamine sulfohydrolase, N-acetyl-alpha-glucosaminidase, iduronate 2-sulfatase, alpha-L-iduronidase, palmitoyl-protein thioesterase 1, tripeptidyl peptidase 1, battenin, CLN5, CLN6 (linclin), MFSD8, CLN8, aspartoacylase (ASPA), progranulin (GRN), McCP2, beta-galactosidase (GLB1) and/or gigaxonin (GAN).

In certain embodiments, the AAV particle includes a viral genome with a payload region comprising a nucleic acid sequence encoding AADC or any other payload known in the art for treating Parkinson's disease. As a non-limiting example, the payload may include a sequence such as NM_001082971.1 (GI: 132814447), NM_000790.3 (GI: 132814459), NM_001242886.1 (GI: 338968913), NM_001242887.1 (GI: 338968916), NM_001242888.1 (GI: 338968918), NM_001242889.1 (GI: 338968920), NM_001242890.1 (GI: 338968922) and fragment or variants thereof.

In certain embodiments, the AAV particle comprises a viral genome with a payload region comprising a nucleic acid sequence encoding frataxin or any other payload known in the art for treating Friedreich's Ataxia. As a non-limiting example, the payload may comprise a sequence such as NM_000144.4 (GI: 239787167), NM_181425.2 (GI: 239787185), NM_001161706.1 (GI: 239787197) and fragment or variants thereof.

In certain embodiments, the AAV particle comprises a viral genome with a payload region comprising a nucleic acid sequence encoding SMN or any other payload known in the art for treating spinal muscular atrophy (SMA). As a non-limiting example, the payload may comprise a sequence such as NM_001297715.1 (GI: 663070993), NM_000344.3 (GI: 196115055), NM_022874.2 (GI: 196115040) and fragment or variants thereof.

In certain embodiments, the AAV particle comprises a viral genome with a payload region comprising a nucleic acid sequence encoding any of the disease-associated proteins (and fragment or variants thereof) described in U. S. Patent publication No. 20180258424; the content of which is herein incorporated by reference in its entirety.

In certain embodiments, the AAV particle includes a viral genome with a payload region comprising a nucleic acid sequence encoding any of the disease-associated proteins (and fragment or variants thereof) described in any one of the following International Publications: WO2016073693, WO2017023724, WO2018232055, WO2016077687, WO2016077689, WO2018204786, WO2017201258, WO2017201248, WO2018204803, WO2018204797, WO2017189959, WO2017189963, WO2017189964, WO2015191508, WO2016094783, WO20160137949, WO2017075335; the contents of which are each herein incorporated by reference in their entirety

In certain embodiments, the formulated AAV particles of the present disclosure may be used to improve performance on any assessment used to measure symptoms of a neurodegenerative disorder/disease. Such assessments comprise, but are not limited to ADAS-cog (Alzheimer Disease Assessment Scale—cognitive), MMSE (Mini-Mental State Examination), GDS (Geriatric Depression Scale), FAQ (Functional Activities Questionnaire), ADL (Activities of Daily Living), GPCOG (General Practitioner Assessment of Cognition), Mini-Cog, AMTS (Abbreviated Mental Test Score), Clock-drawing test, 6-CIT (6-item Cognitive Impairment Test), TYM (Test Your Memory), MoCa (Montreal Cognitive Assessment), ACE-R (Addenbrookes Cognitive Assessment), MIS (Memory Impairment Screen), BADLS (Bristol Activities of Daily Living Scale), Barthel Index, Functional Independence Measure, Instrumental Activities of Daily Living, IQCODE (Informant Questionnaire on Cognitive Decline in the Elderly), Neuropsychiatric Inventory, The Cohen-Mansfield Agitation Inventory, BEHAVE-AD, EuroQol, Short Form-36 and/or MBR Caregiver Strain Instrument, or any of the other tests as described in Sheehan B Ther Adv Neurol Disord 5(6):349-358 (2012), the contents of which are herein incorporated by reference in their entirety.

In certain embodiments “variant mimics” are provided. As used herein, the term “variant mimic” is one which contains one or more amino acids which would mimic an activated sequence. For example, glutamate may serve as a mimic for phosphoro-threonine and/or phosphoro-serine. Alternatively, variant mimics may result in deactivation or in an inactivated product containing the mimic, e.g., phenylalanine may act as an inactivating substitution for tyrosine; or alanine may act as an inactivating substitution for serine.

In certain embodiments an “amino acid sequence variant” is provided. The term “amino acid sequence variant” refers to molecules with some differences in their amino acid sequences as compared to a native or starting sequence. The amino acid sequence variants may possess substitutions, deletions, and/or insertions at certain positions within the amino acid sequence. “Native” or “starting” sequence should not be confused with a wild type sequence. As used herein, a native or starting sequence is a relative term referring to an original molecule against which a comparison may be made. “Native” or “starting” sequences or molecules may represent the wild-type (that sequence found in nature) but do not have to be the wild-type sequence.

Ordinarily, variants will possess at least about 70% homology to a native sequence, and in certain embodiments, they will be at least about 80% or at least about 90% homologous to a native sequence. “Homology” as it applies to amino acid sequences is defined as the percentage of residues in the candidate amino acid sequence that are identical with the residues in the amino acid sequence of a second sequence after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation.

By “homologs” as it applies to amino acid sequences is meant the corresponding sequence of other species having substantial identity to a second sequence of a second species.

“Analogs” is meant to comprise polypeptide variants which differ by one or more amino acid alterations, e.g., substitutions, additions or deletions of amino acid residues that still maintain the properties of the parent polypeptide.

Sequence tags or amino acids, such as one or more lysines, can be added to the peptide sequences of the disclosure (e.g., at the N-terminal or C-terminal ends). Sequence tags can be used for peptide purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble or linked to a solid support.

In certain embodiments a “substitutional variant” is provided. “Substitutional variants” when referring to proteins are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions comprise the substitution of a non-polar (hydrophobic) residue such as isoleucine, valine and leucine for another non-polar residue. Likewise, examples of conservative substitutions comprise the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions comprise the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

In certain embodiments an “insertional variant” is provided. “Insertional variants” when referring to proteins are those with one or more amino acids inserted immediately adjacent to an amino acid at a particular position in a native or starting sequence. “Immediately adjacent” to an amino acid means connected to either the alpha-carboxy or alpha-amino functional group of the amino acid.

In certain embodiments a “deletional variant” is provided. “Deletional variants” when referring to proteins, are those with one or more amino acids in the native or starting amino acid sequence removed. Ordinarily, deletional variants will have one or more amino acids deleted in a particular region of the molecule.

As used herein, the term “derivative” is used synonymously with the term “variant” and refers to a molecule that has been modified or changed in any way relative to a reference molecule or starting molecule. In certain embodiments, derivatives comprise native or starting proteins that have been modified with an organic proteinaceous or non-proteinaceous derivatizing agent, and post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Either form of these residues may be present in the proteins used in accordance with the present disclosure.

Other post-translational modifications comprise hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)).

“Features” when referring to proteins are defined as distinct amino acid sequence-based components of a molecule. Features of the proteins of the present disclosure comprise surface manifestations, local conformational shape, folds, loops, half-loops, domains, half-domains, sites, termini or any combination thereof.

As used herein when referring to proteins the term “surface manifestation” refers to a polypeptide-based component of a protein appearing on an outermost surface.

As used herein when referring to proteins the term “local conformational shape” means a polypeptide based structural manifestation of a protein which is located within a definable space of the protein.

As used herein when referring to proteins the term “fold” means the resultant conformation of an amino acid sequence upon energy minimization. A fold may occur at the secondary or tertiary level of the folding process. Examples of secondary level folds comprise beta sheets and alpha helices. Examples of tertiary folds comprise domains and regions formed due to aggregation or separation of energetic forces. Regions formed in this way comprise hydrophobic and hydrophilic pockets, and the like.

As used herein the term “turn” as it relates to protein conformation means a bend which alters the direction of the backbone of a peptide or polypeptide and may involve one, two, three or more amino acid residues.

As used herein when referring to proteins the term “loop” refers to a structural feature of a peptide or polypeptide which reverses the direction of the backbone of a peptide or polypeptide and comprises four or more amino acid residues. Oliva et al. have identified at least 5 classes of protein loops (J. Mol Biol 266 (4): 814-830; 1997).

As used herein when referring to proteins the term “half-loop” refers to a portion of an identified loop having at least half the number of amino acid residues as the loop from which it is derived. It is understood that loops may not always contain an even number of amino acid residues. Therefore, in those cases where a loop contains or is identified to comprise an odd number of amino acids, a half-loop of the odd-numbered loop will comprise the whole number portion or next whole number portion of the loop (number of amino acids of the loop/2+/−0.5 amino acids). For example, a loop identified as a 7 amino acid loop could produce half-loops of 3 amino acids or 4 amino acids (7/2=3.5+/−0.5 being 3 or 4).

As used herein when referring to proteins the term “domain” refers to a motif of a polypeptide having one or more identifiable structural or functional characteristics or properties (e.g., binding capacity, serving as a site for protein-protein interactions).

As used herein when referring to proteins the term “half-domain” means portion of an identified domain having at least half the number of amino acid residues as the domain from which it is derived. It is understood that domains may not always contain an even number of amino acid residues. Therefore, in those cases where a domain contains or is identified to comprise an odd number of amino acids, a half-domain of the odd-numbered domain will comprise the whole number portion or next whole number portion of the domain (number of amino acids of the domain/2+/−0.5 amino acids). For example, a domain identified as a 7 amino acid domain could produce half-domains of 3 amino acids or 4 amino acids (7/2=3.5+/−0.5 being 3 or 4). It is also understood that sub-domains may be identified within domains or half-domains, these subdomains possessing less than all of the structural or functional properties identified in the domains or half domains from which they were derived. It is also understood that the amino acids that comprise any of the domain types herein need not be contiguous along the backbone of the polypeptide (i.e., nonadjacent amino acids may fold structurally to produce a domain, half-domain or subdomain).

As used herein when referring to proteins the terms “site” as it pertains to amino acid-based embodiments is used synonymous with “amino acid residue” and “amino acid side chain”. A site represents a position within a peptide or polypeptide that may be modified, manipulated, altered, derivatized or varied within the polypeptide-based molecules of the present disclosure.

As used herein the terms “termini or terminus” when referring to proteins refers to an extremity of a peptide or polypeptide. Such extremity is not limited only to the first or final site of the peptide or polypeptide but may comprise additional amino acids in the terminal regions. The polypeptide-based molecules of the present disclosure may be characterized as having both an N-terminus (terminated by an amino acid with a free amino group (NH2)) and a C-terminus (terminated by an amino acid with a free carboxyl group (COOH)). Proteins of the disclosure are in certain embodiments made up of multiple polypeptide chains brought together by disulfide bonds or by non-covalent forces (multimers, oligomers). These sorts of proteins will have multiple N- and C-termini. Alternatively, the termini of the polypeptides may be modified such that they begin or end, as the case may be, with a non-polypeptide-based moiety such as an organic conjugate.

Once any of the features have been identified or defined as a component of a molecule of the disclosure, any of several manipulations and/or modifications of these features may be performed by moving, swapping, inverting, deleting, randomizing or duplicating. Furthermore, it is understood that manipulation of features may result in the same outcome as a modification to the molecules of the disclosure. For example, a manipulation which involves deleting a domain would result in the alteration of the length of a molecule just as modification of a nucleic acid to encode less than a full-length molecule would.

Modifications and manipulations can be accomplished by methods known in the art such as site directed mutagenesis. The resulting modified molecules may then be tested for activity using in vitro or in vivo assays such as those described herein, or any other suitable screening assay known in the art.

### Payloads: Modulatory Polynucleotides Targeting a Gene of Interest

The present disclosure comprises the use of formulated AAV particles whose viral genomes encode modulatory polynucleotides, e.g., RNA or DNA molecules as therapeutic agents. Accordingly, the present disclosure provides viral genomes which encode polynucleotides which are processed into small double stranded RNA (dsRNA) molecules (small interfering RNA, siRNA, miRNA, pre-miRNA) targeting a gene of interest. The present disclosure also provides methods of their use for inhibiting gene expression and protein production of an allele of the gene of interest, for treating diseases, disorders, and/or conditions.

In certain embodiments, the AAV particle comprises a viral genome with a payload region comprising a nucleic acid sequence encoding or comprising one or more modulatory polynucleotides. In certain embodiments, the AAV particle comprises a viral genome with a payload region comprising a nucleic acid sequence encoding a modulatory polynucleotide of interest. In certain embodiments of the present disclosure, modulatory polynucleotides, e.g., RNA or DNA molecules, are presented as therapeutic agents. RNA interference mediated gene silencing can specifically inhibit targeted gene expression.

In certain embodiments, a nucleic acid sequence encoding such siRNA molecules, or a single strand of the siRNA molecules, is inserted into adeno-associated viral vectors and introduced into cells, specifically cells in the central nervous system.

AAV particles have been investigated for siRNA delivery because of several unique features. Non-limiting examples of the features comprise (i) the ability to infect both dividing and non-dividing cells; (ii) a broad host range for infectivity, comprising human cells; (iii) wild-type AAV has not been associated with any disease and has not been shown to replicate in infected cells; (iv) the lack of cell-mediated immune response against the vector and (v) the non-integrative nature in a host chromosome thereby reducing potential for long-term expression. Moreover, infection with AAV particles has minimal influence on changing the pattern of cellular gene expression (Stilwell and Samulski et al., Biotechniques, 2003, 34, 148).

In certain embodiments, the encoded siRNA duplex of the present disclosure contains an antisense strand and a sense strand hybridized together forming a duplex structure, wherein the antisense strand is complementary to the nucleic acid sequence of the targeted gene of interest, and wherein the sense strand is homologous to the nucleic acid sequence of the targeted gene of interest. In other aspects, there are 0, 1 or 2 nucleotide overhangs at the 3′end of each strand.

The payloads of the formulated AAV particles of the present disclosure may encode one or more agents which are subject to RNA interference (RNAi) induced inhibition of gene expression. Provided herein are encoded siRNA duplexes or encoded dsRNA that target a gene of interest (referred to herein collectively as “siRNA molecules”). Such siRNA molecules, e.g., encoded siRNA duplexes, encoded dsRNA or encoded siRNA or dsRNA precursors can reduce or silence gene expression in cells, for example, astrocytes or microglia, cortical, hippocampal, entorhinal, thalamic, sensory or motor neurons.

RNAi (also known as post-transcriptional gene silencing (PTGS), quelling, or co-suppression) is a post-transcriptional gene silencing process in which RNA molecules, in a sequence specific manner, inhibit gene expression, typically by causing the destruction of specific mRNA molecules. The active components of RNAi are short/small double stranded RNAs (dsRNAs), called small interfering RNAs (siRNAs), that typically contain 15-30 nucleotides (e.g., 19 to 25, 19 to 24 or 19-21 nucleotides) and 2-nucleotide 3′ overhangs and that match the nucleic acid sequence of the target gene. These short RNA species may be naturally produced in vivo by Dicer-mediated cleavage of larger dsRNAs and they are functional in mammalian cells.

Naturally expressed small RNA molecules, known as microRNAs (miRNAs), elicit gene silencing by regulating the expression of mRNAs. The miRNAs containing RNA Induced Silencing Complex (RISC) targets mRNAs presenting a perfect sequence complementarity with nucleotides 2-7 in the 5′ region of the miRNA which is called the seed region, and other base pairs with its 3′ region. miRNA mediated down regulation of gene expression may be caused by cleavage of the target mRNAs, translational inhibition of the target mRNAs, or mRNA decay. miRNA targeting sequences are usually located in the 3′ UTR of the target mRNAs. A single miRNA may target more than 100 transcripts from various genes, and one mRNA may be targeted by different miRNAs.

siRNA duplexes or dsRNA targeting a specific mRNA may be designed as a payload of an AAV particle and introduced into cells for activating RNAi processes. Elbashir et al. demonstrated that 21-nucleotide siRNA duplexes (termed small interfering RNAs) were capable of effecting potent and specific gene knockdown without inducing immune response in mammalian cells (Elbashir S M et al., Nature, 2001, 411, 494498). Since this initial report, post-transcriptional gene silencing by siRNAs quickly emerged as a powerful tool for genetic analysis in mammalian cells and has the potential to produce novel therapeutics.

The siRNA duplex comprised of a sense strand homologous to the target mRNA and an antisense strand that is complementary to the target mRNA offers much more advantage in terms of efficiency for target RNA destruction compared to the use of the single strand (ss)-siRNAs (e.g. antisense strand RNA or antisense oligonucleotides). In many cases it requires higher concentration of the ss-siRNA to achieve the effective gene silencing potency of the corresponding duplex.

In certain embodiments, the siRNA molecules may be encoded in a modulatory polynucleotide which also comprises a molecular scaffold. As used herein a “molecular scaffold” is a framework or starting molecule that forms the sequence or structural basis against which to design or make a subsequent molecule.

In certain embodiments, the modulatory polynucleotide which comprises the payload (e.g., siRNA, miRNA or other RNAi agent described herein) comprises molecular scaffold which comprises a leading 5′ flanking sequence which may be of any length and may be derived in whole or in part from wild type microRNA sequence or be completely artificial. A 3′ flanking sequence may mirror the 5′ flanking sequence in size and origin. In certain embodiments, one or both of the 5′ and 3′ flanking sequences are absent.

In certain embodiments, the molecular scaffold may comprise one or more linkers known in the art. The linkers may separate regions or one molecular scaffold from another. As a non-limiting example, the molecular scaffold may be polycistronic.

In certain embodiments, the modulatory polynucleotide is designed using at least one of the following properties: loop variant, seed mismatch/bulge/wobble variant, stem mismatch, loop variant and basal stem mismatch variant, seed mismatch and basal stem mismatch variant, stem mismatch and basal stem mismatch variant, seed wobble and basal stem wobble variant, or a stem sequence variant.

In certain embodiments, the present disclosure presents the use of formulated AAV particles whose viral genomes encode modulatory polynucleotides, e.g., RNA or DNA molecules as therapeutic agents. Accordingly, the present disclosure provides viral genomes which encode polynucleotides which are processed into small double stranded RNA (dsRNA) molecules (small interfering RNA, siRNA, miRNA, pre-miRNA) targeting a gene of interest. The present disclosure also provides methods of their use for inhibiting gene expression and protein production of an allele of the gene of interest, for treating diseases, disorders, and/or conditions.

In certain embodiments, the AAV particle comprises a viral genome with a payload region comprising a nucleic acid sequence encoding or comprising one or more modulatory polynucleotides. In certain embodiments, the AAV particle comprises a viral genome with a payload region comprising a nucleic acid sequence encoding a modulatory polynucleotide of interest. In certain embodiments of the present disclosure, modulatory polynucleotides, e.g., RNA or DNA molecules, are presented as therapeutic agents. RNA interference mediated gene silencing can specifically inhibit targeted gene expression.

In certain embodiments, the payload region comprises a nucleic acid sequence encoding a modulatory polynucleotide which interferes with a target gene expression and/or a target protein production. In certain embodiments, the gene expression or protein production to be inhibited/modified may comprise but are not limited to superoxide dismutase 1 (SOD1), chromosome 9 open reading frame 72 (C9ORF72), TAR DNA binding protein (TARDBP), ataxin-3 (ATXN3), huntingtin (HTT), amyloid precursor protein (APP), apolipoprotein E (ApoE), microtubule-associated protein tau (MAPT), alpha-synuclein (SNCA), voltage-gated sodium channel alpha subunit 9 (SCN9A), and/or voltage-gated sodium channel alpha subunit 10 (SCN10A).

The present disclosure provides small interfering RNA (siRNA) duplexes (and modulatory polynucleotides encoding them) that target SOD1 mRNA to interfere with the gene expression and/or protein production of SOD1. The present disclosure also provides methods of their use for inhibiting gene expression and protein production of an allele of SOD1, for treating amyotrophic lateral sclerosis (ALS). In certain embodiments, the siRNA duplexes of the present disclosure may target SOD1 along any segment of the respective nucleotide sequence. In certain embodiments, the siRNA duplexes of the present disclosure may target SOD1 at the location of a SNP or variant within the nucleotide sequence.

The present disclosure provides small interfering RNA (siRNA) duplexes (and modulatory polynucleotides encoding them) that target HTT mRNA to interfere with the gene expression and/or protein production of HTT. The present disclosure also provides methods of their use for inhibiting gene expression and protein production of an allele of HTT, for treating Huntington's disease (HD). In certain embodiments, the siRNA duplexes of the present disclosure may target HTT along any segment of the respective nucleotide sequence. In certain embodiments, the siRNA duplexes of the present disclosure may target HTT at the location of a SNP or variant within the nucleotide sequence.

In certain embodiments, the AAV particle comprises a viral genome with a payload region comprising a nucleic acid sequence encoding any of the modulatory polynucleotides, RNAi molecules, siRNA molecules, dsRNA molecules, and/or RNA duplexes described in any one of the following International Publications: WO2016077687, WO2016077689, WO2018204786, WO2017201258, WO2017201248, WO2018204803, WO2018204797, WO2017189959, WO2017189963, WO2017189964, WO2015191508, WO2016094783, WO20160137949, WO2017075335; the contents of which are each herein incorporated by reference in their entirety.

In certain embodiments, a nucleic acid sequence encoding such siRNA molecules, or a single strand of the siRNA molecules, is inserted into adeno-associated viral vectors and introduced into cells, specifically cells in the central nervous system.

AAV particles have been investigated for siRNA delivery because of several unique features. Non-limiting examples of the features comprise (i) the ability to infect both dividing and non-dividing cells; (ii) a broad host range for infectivity, comprising human cells; (iii) wild-type AAV has not been associated with any disease and has not been shown to replicate in infected cells; (iv) the lack of cell-mediated immune response against the vector and (v) the non-integrative nature in a host chromosome thereby reducing potential for long-term expression. Moreover, infection with AAV particles has minimal influence on changing the pattern of cellular gene expression (Stilwell and Samulski et al., Biotechniques, 2003, 34, 148).

In certain embodiments, the encoded siRNA duplex of the present disclosure contains an antisense strand and a sense strand hybridized together forming a duplex structure, wherein the antisense strand is complementary to the nucleic acid sequence of the targeted gene of interest, and wherein the sense strand is homologous to the nucleic acid sequence of the targeted gene of interest. In other aspects, there are 0, for 2 nucleotide overhangs at the 3′end of each strand.

According to the present disclosure, each strand of the siRNA duplex targeting the gene of interest can be about 19 to 25, 19 to 24 or 19 to 21 nucleotides in length, such as about 19 nucleotides, 20 nucleotides, 21 nucleotides, 22 nucleotides, 23 nucleotides, 24 nucleotides, or 25 nucleotides in length.

In certain embodiments, an siRNA or dsRNA comprises at least two sequences that are complementary to each other. The dsRNA comprises a sense strand having a first sequence and an antisense strand having a second sequence. The antisense strand comprises a nucleotide sequence that is substantially complementary to at least part of an mRNA encoding a gene of interest, and the region of complementarity is 30 nucleotides or less, and at least 15 nucleotides in length. Generally, the dsRNA is 19 to 25, 19 to 24 or 19 to 21 nucleotides in length. In certain embodiments, the dsRNA is from about 15 to about 25 nucleotides in length, and in certain embodiments the dsRNA is from about 25 to about 30 nucleotides in length.

The dsRNA encoded in an expression vector upon contacting with a cell expressing protein encoded by the gene of interest, inhibits the expression of protein encoded by the gene of interest by at least 10%, at least 20%, at least 25%, at least 30%, at least 35%, at least 40%, at least 50%, at least 60%, at least 70%, at least 80%, or more, when assayed by methods known in the art or a method as described herein.

According to the present disclosure, the siRNA molecules are designed and tested for their ability in reducing mRNA levels in cultured cells.

In certain embodiments, the siRNA molecules are designed and tested for their ability in reducing levels of the gene of interest in cultured cells.

The present disclosure also provides pharmaceutical compositions comprising at least one siRNA duplex targeting the gene of interest and a pharmaceutically acceptable carrier. In some aspects, the siRNA duplex is encoded by a viral genome in an AAV particle.

In certain embodiments, the present disclosure provides methods for inhibiting/silencing gene expression in a cell. In some aspects, the inhibition of gene expression refers to an inhibition by at least about 20%, such as by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 3040%, 3540%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

In certain embodiments, the encoded siRNA duplexes may be used to reduce the expression of protein or mRNA encoded by the gene of interest by at least about 20%, 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 35-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. As a non-limiting example, the expression of protein or mRNA may be reduced 50-90%. As a non-limiting example, the expression of protein or mRNA may be reduced 30-70%. As a non-limiting example, the expression of protein or mRNA may be reduced 40-70%.

In certain embodiments, the encoded siRNA duplexes may be used to reduce the expression of protein encoded by the gene of interest and/or transcribed mRNA in at least one region of the CNS. As a non-limiting example, the region is the neurons (e.g., cortical neurons).

In certain embodiments, the formulated AAV particles comprising such encoded siRNA molecules may be introduced directly into the central nervous system of the subject, for example, by infusion into the putamen.

In certain embodiments, the formulated AAV particles comprising such encoded siRNA molecules may be introduced directly into the central nervous system of the subject, for example, by infusion into the thalamus of a subject.

In certain embodiments, the formulated AAV particles comprising such encoded siRNA molecules may be introduced directly into the central nervous system of the subject, for example, by infusion into the white matter of a subject.

In certain embodiments, the formulated AAV particles comprising such encoded siRNA molecules may be introduced to the central nervous system of the subject, for example, by intravenous administration to a subject.

In certain embodiments, the pharmaceutical composition of the present disclosure is used as a solo therapy. In certain embodiments, the pharmaceutical composition of the present disclosure is used in combination therapy. The combination therapy may be in combination with one or more neuroprotective agents such as small molecule compounds, growth factors and hormones which have been tested for their neuroprotective effect on motor neuron degeneration.

The payloads of the formulated AAV particles of the present disclosure may encode one or more agents which are subject to RNA interference (RNAi) induced inhibition of gene expression. Provided herein are encoded siRNA duplexes or encoded dsRNA that target a gene of interest (referred to herein collectively as “siRNA molecules”). Such siRNA molecules, e.g., encoded siRNA duplexes, encoded dsRNA or encoded siRNA or dsRNA precursors can reduce or silence gene expression in cells, for example, astrocytes or microglia, cortical, hippocampal, entorhinal, thalamic, sensory or motor neurons.

RNAi (also known as post-transcriptional gene silencing (PTGS), quelling, or co-suppression) is a post-transcriptional gene silencing process in which RNA molecules, in a sequence specific manner, inhibit gene expression, typically by causing the destruction of specific mRNA molecules. The active components of RNAi are short/small double stranded RNAs (dsRNAs), called small interfering RNAs (siRNAs), that typically contain 15-30 nucleotides (e.g., 19 to 25, 19 to 24 or 19-21 nucleotides) and 2-nucleotide 3′ overhangs and that match the nucleic acid sequence of the target gene. These short RNA species may be naturally produced in vivo by Dicer-mediated cleavage of larger dsRNAs and they are functional in mammalian cells.

In some embodiments, the modulatory polynucleotides of the viral genome may comprise at least one nucleic acid sequence encoding at least one siRNA molecule. The nucleic acid sequence may, independently if there is more than one, encode 1, 2, 3, 4, 5, 6, 7, 8, 9, or more than 9 siRNA molecules.

Naturally expressed small RNA molecules, known as microRNAs (miRNAs), elicit gene silencing by regulating the expression of mRNAs. The miRNAs containing RNA Induced Silencing Complex (RISC) targets mRNAs presenting a perfect sequence complementarity with nucleotides 2-7 in the 5′ region of the miRNA which is called the seed region, and other base pairs with its 3′ region. miRNA mediated down regulation of gene expression may be caused by cleavage of the target mRNAs, translational inhibition of the target mRNAs, or mRNA decay. miRNA targeting sequences are usually located in the 3′ UTR of the target mRNAs. A single miRNA may target more than 100 transcripts from various genes, and one mRNA may be targeted by different miRNAs.

siRNA duplexes or dsRNA targeting a specific mRNA may be designed as a payload of an AAV particle and introduced into cells for activating RNAi processes. Elbashir et al. demonstrated that 21-nucleotide siRNA duplexes (termed small interfering RNAs) were capable of effecting potent and specific gene knockdown without inducing immune response in mammalian cells (Elbashir S M et al., Nature, 2001, 411, 494-498). Since this initial report, post-transcriptional gene silencing by siRNAs quickly emerged as a powerful tool for genetic analysis in mammalian cells and has the potential to produce novel therapeutics.

The siRNA duplex comprised of a sense strand homologous to the target mRNA and an antisense strand that is complementary to the target mRNA offers much more advantage in terms of efficiency for target RNA destruction compared to the use of the single strand (ss)-siRNAs (e.g. antisense strand RNA or antisense oligonucleotides). In many cases it requires higher concentration of the ss-siRNA to achieve the effective gene silencing potency of the corresponding duplex.

Any of the foregoing molecules may be encoded by an AAV particle or viral genome.

Introduction into Cells

The encoded payload of the present disclosure may be introduced into cells by being encoded by the viral genome of an AAV particle. These AAV particles can be engineered and optimized to facilitate the entry into cells that are not readily amendable to transfection/transduction. Also, some synthetic viral vectors possess an ability to integrate the payload into the cell genome, thereby leading to stable payload expression and long-term therapeutic effect. In this manner, viral vectors are engineered as vehicles for specific delivery while lacking the deleterious replication and/or integration features found in wild-type virus.

In certain embodiments, the encoded payload is introduced into a cell by transfecting, infecting or transducing the cell with an AAV particle comprising nucleic acid sequences capable of producing the payload when processed in the cell. In certain embodiments, the payload is introduced into a cell by injecting into the cell or tissue an AAV particle comprising a nucleic acid sequence capable of producing the payload when processed in the cell.

Other methods for introducing AAV particles comprising the nucleic acid sequence for the payloads described herein may comprise photochemical internalization as described in U. S. Patent publication No. 20120264807, the content of which is incorporated herein by reference in its entirety as related to photochemical internalizations, insofar as it does not conflict with the present disclosure.

In certain embodiments, the formulations described herein may contain at least one AAV particle comprising the nucleic acid sequence encoding the payloads described herein. In certain embodiments, the payloads may target the gene of interest at one target site. In another embodiment, the formulation comprises a plurality of AAV particles, each AAV particle comprising a nucleic acid sequence encoding a payload targeting a gene of interest at a different target site. The gene of interest may be targeted at 2, 3, 4, 5 or more than 5 sites.

In certain embodiments, the AAV particles from any relevant species, such as, but not limited to, human, pig, dog, mouse, rat or monkey may be introduced into cells.

In certain embodiments, the formulated AAV particles may be introduced into cells or tissues which are relevant to the disease to be treated. In certain embodiments, the formulated AAV particles may be introduced into cells which have a high level of endogenous expression of the target gene. In another embodiment, the formulated AAV particles may be introduced into cells which have a low level of endogenous expression of the target gene. In certain embodiments, the cells may be those which have a high efficiency of AAV transduction.

In certain embodiments, formulated AAV particles comprising a nucleic acid sequence encoding a payload of the present disclosure may be used to deliver the payload to the central nervous system (e.g., U.S. Pat. No. 6,180,613; the content of which is incorporated herein by reference in its entirety as related to the delivery and therapeutic use of siRNA molecules and AAV particles, insofar as it does not conflict with the present disclosure).

In certain embodiments, the formulated AAV particles comprising a nucleic acid sequence encoding a payload of the present disclosure may further comprise a modified capsid comprising peptides from non-viral origin. In other aspects, the AAV particle may contain a CNS specific chimeric capsid to facilitate the delivery of encoded siRNA duplexes into the brain and the spinal cord. For example, an alignment of cap nucleotide sequences from AAV variants exhibiting CNS tropism may be constructed to identify variable region (VR) sequence and structure.

In certain embodiments, AAV particle comprising the nucleic acid sequence for the siRNA molecules of the present disclosure may be formulated for CNS delivery. Agents that cross the brain blood barrier may be used. For example, some cell penetrating peptides that can target siRNA molecules to the brain blood barrier endothelium may be used to formulate the siRNA duplexes targeting the gene of interest.

In certain embodiments, the formulated AAV particle comprising a nucleic acid sequence encoding a payload of the present disclosure may be administered directly to the CNS. As a non-limiting example, the vector comprises a nucleic acid sequence encoding an siRNA molecule targeting the gene of interest. As a non-limiting example, the vector comprises a nucleic acid sequence encoding an polypeptide targeting a gene of interest.

In certain embodiments, the formulated AAV particle may be administered to a subject (e.g., to the CNS of a subject) in a therapeutically effective amount.

### II. AAV Production

### General Viral Production Process

Mammalian cells and/or insect cells are often used as viral production cells for the production of rAAV particles. In various embodiments, the methods and systems disclosed herein employ insect cells, e.g., Sf9 cells.

Viral production cells for the production of rAAV particles generally comprise mammalian cell types. However, mammalian cells present several complications to the large-scale production of rAAV particles, comprising general low yield of viral-particles-per-replication-cell as well as high risks for undesirable contamination from other mammalian biomaterials in the viral production cell. As a result, insect cells have become an alternative vehicle for large-scale production of rAAV particles.

AAV production systems using insect cells also present a range of complications. For example, high-yield production of rAAV particles often requires a lower expression of Rep78 compared to Rep52. Controlling the relative expression of Rep78 and Rep52 in insect cells thus requires carefully designed control mechanisms within the Rep operon. These control mechanisms can comprise individually engineered insect cell promoters, such as ΔIE1 promoters for Rep78 and PolH promoters for Rep52, or the division of the Rep-encoding nucleotide sequences onto independently engineered sequences or constructs. However, implementation of these control mechanisms often leads to reduced rAAV particle yield or to structurally unstable virions.

In another example, production of rAAV particles requires VP1, VP2 and VP3 proteins which assemble to form the AAV capsid. High-yield production of rAAV particles requires adjusted ratios of VP1, VP2 and VP3, which should generally be around 1:1:10, respectively, but can vary from 1-2 for VP1 and/or 1-2 for VP2, relative to 10 VP3 copies. This ratio is important for the quality of the capsid, as too much VP1 destabilizes the capsid and too little VP1 will decrease the infectivity of the virus.

Wild type AAV use a deficient splicing method to control VP1 expression; a weak start codon (ACG) with special surrounding (“Kozak” sequence) to control VP2; and a standard start codon (ATG) for VP3 expression. However, in some baculovirus systems, the mammalian splicing sequences are not always recognized and unable to properly control the production of VP1, VP2 and VP3. Consequently, neighboring nucleotides and the ACG start sequence from VP2 can be used to drive capsid protein production. Unfortunately, for most of the AAV serotypes, this method creates a capsid with a lower ratio of VP1 compared to VP2 (<1 relative to 10 VP3 copies). To more effectively control the production of VP proteins, non-canonical or start codons have been used, like TTG, GTG or CTG. However, these start codons can be considered suboptimal by those in the art relative to the wild type ATG or ACG start codons (See, WO2007046703 and WO2007148971, the content of which is incorporated herein by reference in its entirety as related to production of AAV capsid proteins, insofar as it does not conflict with the present disclosure).

In another example, production of rAAV particles using a baculovirus/Sf9 system generally requires the widely used bacmid-based Baculovirus Expression Vector System (BEVs), which are not optimized for large-scale AAV production. Aberrant proteolytic degradation of viral proteins in the bacmid-based BEVs is an unexpected issue, precluding the reliable large-scale production of AAV capsid proteins using the baculovirus/Sf9 system.

There is continued need for methods and systems which allow for effective and efficient large scale (commercial) production of rAAV particles in mammalian and insect cells.

The details of one or more embodiments of the present disclosure are set forth in the accompanying description below. Other features, objects, and advantages of the present disclosure will be apparent from the description, drawings, and the claims. In the description, the singular forms also include the plural unless the context clearly dictates otherwise. Unless defined otherwise, all technical and scientific terms used herein have the same meaning as commonly understood by one of ordinary skill in the art to which this present disclosure belongs. In the case of conflict with disclosures incorporated by reference, the present express description will control.

In certain embodiments, the constructs, polynucleotides, polypeptides, vectors, serotypes, capsids formulations, or particles of the present disclosure may be, may comprise, may be modified by, may be used by, may be used for, may be used with, or may be produced with any sequence, element, construct, system, target or process described in one of the following International Publications: WO2016073693, WO2017023724, WO2018232055, WO2016077687, WO2016077689, WO2018204786, WO2017201258, WO2017201248, WO2018204803, WO2018204797, WO2017189959, WO2017189963, WO2017189964, WO2015191508, WO2016094783, WO20160137949, WO2017075335; the contents of which are each incorporated herein by reference in their entireties, insofar as they do not conflict with the present disclosure.

AAV production of the present disclosure comprises processes and methods for producing AAV particles and viral vectors which can contact a target cell to deliver a payload, e.g. a recombinant viral construct, which comprises a nucleotide encoding a payload molecule. In certain embodiments, the viral vectors are adeno-associated viral (AAV) vectors such as recombinant adeno-associated viral (rAAV) vectors. In certain embodiments, the AAV particles are adeno-associated viral (AAV) particles such as recombinant adeno-associated viral (rAAV) particles.

The present disclosure provides methods of producing AAV particles or viral vectors by (a) contacting a viral production cell with one or more viral expression constructs encoding at least one AAV capsid protein and/or at least one AAV replication protein, and one or more payload construct vectors, wherein said payload construct vector comprises a payload construct encoding a payload molecule selected from the group consisting of a transgene, a polynucleotide encoding protein, and a modulatory nucleic acid; (b) culturing said viral production cell under conditions such that at least one AAV particle or viral vector is produced, and (c) isolating said at least one AAV particle or viral vector.

In these methods a viral expression construct may encode at least one structural protein and/or at least one non-structural protein. The structural protein may comprise any of the native or wild type capsid proteins VP1, VP2 and/or VP3 or a chimeric protein. The non-structural protein may comprise any of the native or wild type Rep78, Rep68, Rep52 and/or Rep40 proteins or a chimeric protein.

In certain embodiments, an rAAV production method as disclosed herein comprises transient transfection, viral transduction and/or electroporation.

In certain embodiments, the viral production cell is selected from the group consisting of a mammalian cell and an insect cell. In certain embodiments, the insect cell comprises a Spodoptera frugiperda insect cell. In certain embodiments, the insect cell comprises a Sf9 insect cell. In certain embodiments, the insect cell comprises a Sf21 insect cell.

The payload construct vector of the present disclosure may comprise at least one inverted terminal repeat (ITR) and may comprise mammalian DNA.

Also provided are AAV particles and viral vectors produced according to the methods described herein.

The AAV particles of the present disclosure may be formulated as a pharmaceutical composition with one or more acceptable excipients.

In certain embodiments, an AAV particle or viral vector may be produced by a method described herein.

In certain embodiments, the AAV particles may be produced by contacting a viral production cell (e.g., an insect cell) with at least one viral expression construct encoding at least one capsid protein and at least one AAV replication protein, and at least one payload construct vector. In certain embodiments, separate viral expression constructs encoding the at least one capsid protein and the at least one AAV replication protein may be used. The viral production cell may be contacted by transient transfection, viral transduction and/or electroporation. The payload construct vector may comprise a payload construct encoding a payload molecule such as, but not limited to, a transgene, a polynucleotide encoding protein, and a modulatory nucleic acid. The viral production cell can be cultured under conditions such that at least one AAV particle or viral vector is produced, isolated (e.g., using temperature-induced lysis, mechanical lysis and/or chemical lysis) and/or purified (e.g., using filtration, chromatography and/or immunoaffinity purification). As a non-limiting example, the payload construct vector may comprise mammalian DNA.

In certain embodiments, the AAV particles are produced in an insect cell (e.g., Spodoptera frugiperda (Sf9) cell) using the method described herein. As a non-limiting example, the insect cell is contacted using viral transduction which may comprise baculoviral transduction.

In another embodiment, the AAV particles are produced in a mammalian cell using the method described herein. As a non-limiting example, the mammalian cell is contacted using transient transfection.

In certain embodiments, the viral expression construct may encode at least one structural protein and at least one non-structural protein. As a non-limiting example, the structural protein may comprise VP1, VP2 and/or VP3 capsid proteins. As another non-limiting example, the non-structural protein may comprise Rep78, Rep68, Rep52 and/or Rep40 replication proteins.

In certain embodiments, the AAV particle production method described herein produces greater than 10¹, greater than 10², greater than 10³, greater than 10⁴ or greater than 10⁵ AAV particles in a viral production cell.

In certain embodiments, a process of the present disclosure comprises production of viral particles in a viral production cell using a viral production system which comprises at least one viral expression construct and at least one payload construct. The at least one viral expression construct and at least one payload construct can be co-transfected (e.g. dual transfection, triple transfection) into a viral production cell. The transfection is completed using standard molecular biology techniques known and routinely performed by a person skilled in the art. The viral production cell provides the cellular machinery necessary for expression of the proteins and other biomaterials necessary for producing the AAV particles, comprising Rep proteins which replicate the payload construct and Cap proteins which assemble to form a capsid that encloses the replicated payload constructs. The resulting AAV particle is extracted from the viral production cells and processed into a pharmaceutical preparation for administration.

In certain embodiments, the process for production of viral particles utilizes seed cultures of viral production cells that comprise one or more baculoviruses (e.g., a Baculoviral Expression Vector (BEV) or baculovirus infected insect cells (BIICs) that have been transfected with a viral expression construct (e.g., comprised in an expressionBac) and a payload construct (e.g., comprised in a payloadBac)). In certain embodiments, the seed cultures are harvested, divided into aliquots and frozen, and may be used at a later time point to initiate an infection of a naïve population of production cells.

Large scale production of AAV particles may utilize a bioreactor. The use of a bioreactor allows for the precise measurement and/or control of variables that support the growth and activity of viral production cells such as mass, temperature, mixing conditions (impellor RPM or wave oscillation), CO₂ concentration, O₂ concentration, gas sparge rates and volumes, gas overlay rates and volumes, pH, Viable Cell Density (VCD), cell viability, cell diameter, and/or optical density (OD). In certain embodiments, the bioreactor is used for batch production in which the entire culture is harvested at an experimentally determined time point and AAV particles are purified. In another embodiment, the bioreactor is used for continuous production in which a portion of the culture is harvested at an experimentally determined time point for purification of AAV particles, and the remaining culture in the bioreactor is refreshed with additional growth media components.

In certain embodiments, AAV viral particles can be extracted from viral production cells in a process which comprises cell lysis, clarification, sterilization and purification. Cell lysis comprises any process that disrupts the structure of the viral production cell, thereby releasing AAV particles. In certain embodiments cell lysis may comprise thermal shock, chemical, or mechanical lysis methods. In some embodiments, cell lysis is done chemically. Clarification of the lysed cells can comprise the gross purification of the mixture of lysed cells, media components, and AAV particles. In certain embodiments, clarification comprises centrifugation and/or filtration, comprising but not limited to depth end, tangential flow, and/or hollow fiber filtration.

The end result of viral production is a purified collection of AAV particles which comprise two components: (1) a payload construct (e.g. a recombinant viral genome construct) and (2) a viral capsid.

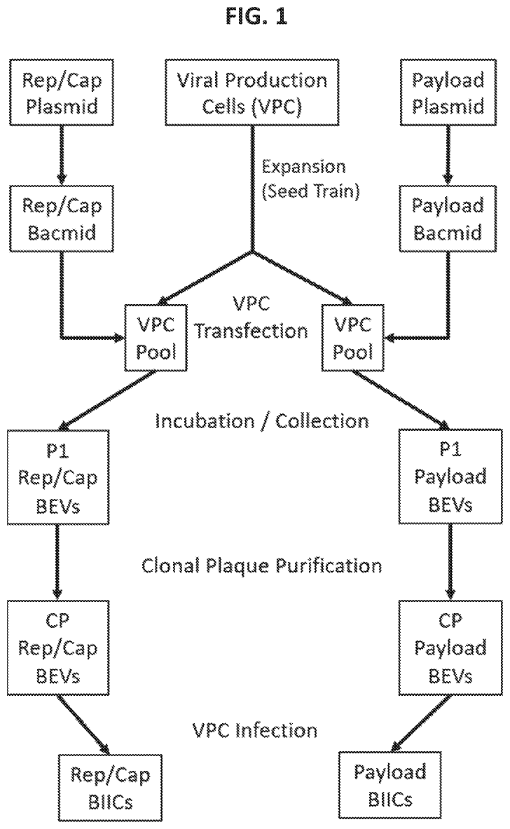

In certain embodiments, such as the embodiment presented in FIG. 1, a viral production system or process of the present disclosure comprises steps for producing baculovirus infected insect cells (BIICs) using Viral Production Cells (VPC) and plasmid constructs. Viral Production Cells (VPCs) from a Cell Bank (CB) are thawed and expanded to provide a target working volume and VPC concentration. The resulting pool of VPCs is split into a Rep/Cap VPC pool and a Payload VPC pool. One or more Rep/Cap plasmid constructs (viral expression constructs) are processed into Rep/Cap Bacmid polynucleotides and transfected into the Rep/Cap VPC pool. One or more Payload plasmid constructs (payload constructs) are processed into Payload Bacmid polynucleotides and transfected into the Payload VPC pool. The two VPC pools are incubated to produce P1 Rep/Cap Baculoviral Expression Vectors (BEVs) and P1 Payload BEVs. The two BEV pools are expanded into a collection of Plaques, with a single Plaque being selected for Clonal Plaque (CP) Purification (also referred to as Single Plaque Expansion). The process can comprise a single CP Purification step or can comprise multiple CP Purification steps either in series or separated by other processing steps. The one-or-more CP Purification steps provide a CP Rep/Cap BEV pool and a CP Payload BEV pool. These two BEV pools can then be stored and used for future production steps, or they can be then transfected into VPCs to produce a Rep/Cap BIIC pool and a Payload BIIC pool.

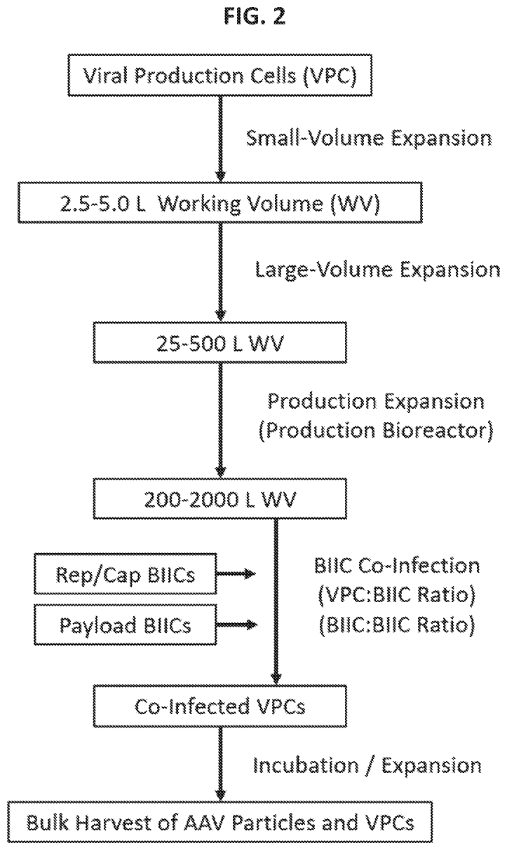

In certain embodiments, such as the embodiment presented in FIG. 2, a viral production system or process of the present disclosure comprises steps for producing AAV particles using Viral Production Cells (VPC) and baculovirus infected insect cells (BIICs). Viral Production Cells (VPCs) from a Cell Bank (CB) are thawed and expanded to provide a target working volume and VPC concentration. This expansion can include one or more small-volume expansion steps up to a working volume of 2000-5000 mL, followed by one or more large-volume expansion steps in large-scale bioreactors (e.g. Wave and/or N-1 bioreactors) up to a working volume of 25-500 L. The working volume of Viral Production Cells is seeded into a Production Bioreactor and can be further expanded to a working volume of 200-2500 L with a target VPC concentration for BIIC infection.

The working volume of VPCs in the Production Bioreactor is then co-infected with Rep/Cap BIICs and Payload BIICs, e.g., with a target VPC:BIIC ratio and a target BIIC:BIIC ratio. VCD infection can also utilize BEVs. The co-infected VPCs are incubated and expanded in the Production Bioreactor to produce a bulk harvest of AAV particles and VPCs.

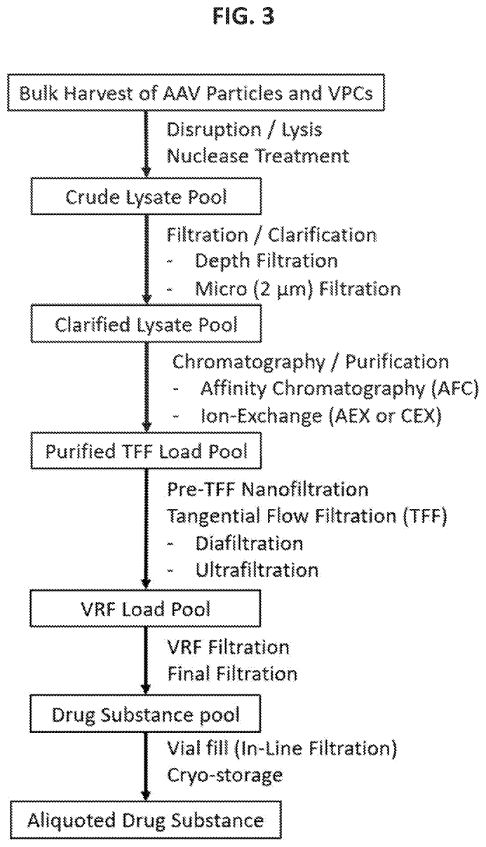

In certain embodiments, such as the embodiment presented in FIG. 3, a viral production system or process of the present disclosure comprises steps for producing a Drug Substance by processing, clarifying and purifying a bulk harvest of AAV particles and Viral Production Cells. A bulk harvest of AAV particles and VPCs (within a Production Bioreactor) are processed through cellular disruption and lysis (e.g. chemical lysis and/or mechanical lysis), followed by nuclease treatment of the lysis pool, thereby producing a crude lysate pool. The crude lysate pool is processed through one or more filtration and clarification steps, comprising depth filtration and microfiltration to provide a clarified lysate pool. The clarified lysate pool is processed through one or more chromatography and purification steps, comprising affinity chromatography (AFC) and ion-exchange chromatography (AEX or CEX) to provide a purified product pool. The purified product pool is then optionally processed through nanofiltration, and then through tangential flow filtration (TFF). The TFF process comprises one or more diafiltration (DF) steps and one or more ultrafiltration (UF) steps, either in series or alternating. The product pool is further processed through viral retention filtration (VRF) and another filtration step to provide a drug substance pool. The drug substance pool can be further filtered, then aliquoted into vials for storage and treatment.

### Viral Expression Constructs

The viral production system of the present disclosure comprises one or more viral expression constructs which can be transfected/transduced into a viral production cell (e.g., Sf9). In certain embodiments, a viral expression construct or a payload construct of the present disclosure can be a bacmid, also known as a baculovirus plasmid or recombinant baculovirus genome. In certain embodiments, a viral expression construct of the present disclosure can be a baculovirus expression vector (BEV). In certain embodiments, a viral expression construct of the present disclosure can be a BIIC which includes a BEV. As used herein, the term “expressionBac” or “Rep/Cap Bac” refers to a bacmid (such as a BEV) comprising a viral expression construct and/or viral expression region. Viral production cells (e.g., Sf9 cells) may be transfected with expressionBacs and/or with BIICs comprising expressionBacs.

In certain embodiments, the viral expression region comprises a protein-coding nucleotide sequence and at least one expression control sequence for expression in a viral production cell. In certain embodiments, the viral expression region comprises a protein-coding nucleotide sequence operably linked to least one expression control sequence for expression in a viral production cell. In certain embodiments, the viral expression construct contains parvoviral genes under control of one or more promoters. Parvoviral genes can comprise nucleotide sequences encoding non-structural AAV replication proteins, such as Rep genes which encode Rep52, Rep40, Rep68 or Rep78 proteins, e.g., a combination of Rep78 and Rep52. Parvoviral genes can comprise nucleotide sequences encoding structural AAV proteins, such as Cap genes which encode VP1, VP2 and VP3 proteins.

The viral production system of the present disclosure is not limited by the viral expression vector used to introduce the parvoviral functions into the virus replication cell. The presence of the viral expression construct in the virus replication cell need not be permanent. The viral expression constructs can be introduced by any means known, for example by chemical treatment of the cells, electroporation, or infection.

Viral expression constructs of the present disclosure may comprise any compound or formulation, biological or chemical, which facilitates transformation, transfection, or transduction of a cell with a nucleic acid. Exemplary biological viral expression constructs comprise plasmids, linear nucleic acid molecules, and recombinant viruses comprising baculovirus. Exemplary chemical vectors comprise lipid complexes. Viral expression constructs are used to incorporate nucleic acid sequences into virus replication cells in accordance with the present disclosure. (O'Reilly, David R., Lois K. Miller, and Verne A. Luckow. Baculovirus expression vectors: a laboratory manual. Oxford University Press, 1994.); Maniatis et al., eds. Molecular Cloning. CSH Laboratory, NY, N.Y. (1982); and, Philiport and Scluber, eds. Liposoes as tools in Basic Research and Industry. CRC Press, Ann Arbor, Mich. (1995), the contents of which are each incorporated herein by reference in their entireties as related to viral expression constructs and uses thereof, insofar as they do not conflict with the present disclosure.

In certain embodiments, the viral expression construct is an AAV expression construct which comprises one or more nucleotide sequences encoding non-structural AAV replication proteins, structural AAV capsid proteins, or a combination thereof. In certain embodiments, the viral expression region is an AAV expression region of an expression construct which comprises one or more nucleotide sequences encoding non-structural AAV replication proteins, structural AAV capsid proteins, or a combination thereof.

In certain embodiments, the viral expression construct of the present disclosure may be a plasmid vector. In certain embodiments, the viral expression construct of the present disclosure may be a baculoviral construct.

The present disclosure is not limited by the number of viral expression constructs employed to produce AAV particles or viral vectors. In certain embodiments, one, two, three, four, five, six, or more viral expression constructs can be employed to produce AAV particles in viral production cells in accordance with the present disclosure. In one non-limiting example, five expression constructs may individually encode AAV VP1, AAV VP2, AAV VP3, Rep52, Rep78, and with an accompanying payload construct comprising a payload polynucleotide and at least one AAV ITR. In another embodiment, expression constructs may be employed to express, for example, Rep52 and Rep40, or Rep78 and Rep 68. Expression constructs may comprise any combination of VP1, VP2, VP3, Rep52/Rep40, and Rep78/Rep68 coding sequences.

In certain embodiments of the present disclosure, a viral expression construct may be used for the production of an AAV particles in insect cells. In certain embodiments, modifications may be made to the wild type AAV sequences of the capsid and/or rep genes, for example to improve attributes of the viral particle, such as increased infectivity or specificity, or to enhance production yields.

In certain embodiments, the viral expression construct may encode the components of a Parvoviral capsid with incorporated Gly-Ala repeat region, which may function as an immune invasion sequence, as described in US Patent Application 20110171262, the content of which is incorporated herein by reference in its entirety as related to Parvoviral capsid proteins, insofar as it does not conflict with the present disclosure.

In certain embodiments of the present disclosure, a viral expression construct may be used for the production of AAV particles in insect cells. In certain embodiments, modifications may be made to the wild type AAV sequences of the capsid and/or rep genes, for example to improve attributes of the viral particle, such as increased infectivity or specificity, or to enhance production yields from insect cells.

### VP-Coding Region

In certain embodiments, a viral expression construct can comprise a VP-coding region; a VP-coding region is a nucleotide sequence which comprises a VP nucleotide sequence encoding VP1, VP2, VP3, or a combination thereof. In certain embodiments, a viral expression construct can comprise a VP1-coding region; a VP1-coding region is a nucleotide sequence which comprises a VP1 nucleotide sequence encoding a VP1 protein. In certain embodiments, a viral expression construct can comprise a VP2-coding region; a VP2-coding region is a nucleotide sequence which comprises a VP2 nucleotide sequence encoding a VP2 protein. In certain embodiments, a viral expression construct can comprise a VP3-coding region; a VP3-coding region is a nucleotide sequence which comprises a VP3 nucleotide sequence encoding a VP3 protein.

In certain embodiments, a VP-coding region encodes one or more AAV capsid proteins of a specific AAV serotype. The AAV serotypes for VP-coding regions can be the same or different. In certain embodiments, a VP-coding region can be codon optimized. In certain embodiments, a VP-coding region or nucleotide sequence can be codon optimized for a mammal cell. In certain embodiments, a VP-coding region or nucleotide sequence can be codon optimized for an insect cell. In certain embodiments, a VP-coding region or nucleotide sequence can be codon optimized for a Spodoptera frugiperda cell. In certain embodiments, a VP-coding region or nucleotide sequence can be codon optimized for Sf9 or Sf21 cell lines.

In certain embodiments, the viral expression construct comprises a first VP-coding region which comprises a nucleotide sequence encoding one or more AAV capsid proteins selected from VP1, VP2 and VP3. In certain embodiments, the first VP-coding region comprises a nucleotide sequence encoding one or more AAV capsid proteins selected from VP2 and VP3. In certain embodiments, the first VP-coding region comprises a nucleotide sequence encoding VP1, VP2 and VP3 AAV capsid proteins. In certain embodiments, the first VP-coding region comprises a nucleotide sequence encoding VP2 and VP3 AAV capsid proteins. In certain embodiments, the first VP-coding region comprises a nucleotide sequence encoding only VP2 and VP3 AAV capsid proteins. In certain embodiments, the first VP-coding region comprises a nucleotide sequence encoding VP2 and VP3 AAV capsid proteins, but not VP1.

In certain embodiments, the nucleic acid construct comprises a second VP-coding region which comprises a nucleotide sequence encoding one or more AAV capsid proteins selected from VP1, VP2 and VP3. In certain embodiments, the second VP-coding region comprises a nucleotide sequence encoding VP1 AAV capsid proteins. In certain embodiments, the second VP-coding region comprises a nucleotide sequence encoding only VP1 AAV capsid proteins. In certain embodiments, the second VP-coding region comprises a nucleotide sequence encoding VP1 AAV capsid proteins, but not VP2 or VP3.

In certain embodiments, the viral expression construct is an engineered nucleic acid construct. In certain embodiments, the viral expression construct comprises a first nucleotide sequence which comprises the first VP-coding region and the second VP-coding region. In certain embodiments, the first nucleotide sequence comprises a first open reading frame (ORF) which comprises the first VP-coding region, and a second open reading frame (ORF) which comprises the second VP-coding region.

In certain embodiments, the viral expression construct comprises a first nucleotide sequence which comprises the first VP-coding region and a second nucleotide sequence which comprises the second VP-coding region. In certain embodiments, the first nucleotide sequence comprises a first open reading frame (ORF) which comprises the first VP-coding region, and the second nucleotide sequence comprises a second open reading frame (ORF) which comprises the second VP-coding region. In certain embodiments, the first open reading frame is different from the second open reading frame.

In certain embodiments, the viral expression construct comprises a first VP-coding region which comprises a nucleotide sequence encoding one or more AAV capsid proteins selected from VP1, VP2 and VP3; and a second VP-coding region which comprises a nucleotide sequence encoding one or more AAV capsid proteins selected from VP1, VP2 and VP3. In certain embodiments, the first VP-coding region comprises a nucleotide sequence encoding VP1, VP2 and VP3 AAV capsid proteins; and the second VP-coding region comprises a nucleotide sequence encoding only VP1 AAV capsid proteins. In certain embodiments, the first VP-coding region comprises a nucleotide sequence encoding VP1, VP2 and VP3 AAV capsid proteins; and the second VP-coding region comprises a nucleotide sequence encoding VP1 AAV capsid proteins, but not VP2 or VP3. In certain embodiments, the first VP-coding region comprises a nucleotide sequence encoding only VP2 and VP3 AAV capsid proteins; and the second VP-coding region comprises a nucleotide sequence encoding only VP1 AAV capsid proteins. In certain embodiments, the first VP-coding region comprises a nucleotide sequence encoding VP2 and VP3 AAV capsid proteins, but not VP1; and the second VP-coding region which comprises a nucleotide sequence encoding VP1 AAV capsid proteins, but not VP2 or VP3.

In certain embodiments, the first VP-coding region encodes AAV capsid proteins of an AAV serotype, e.g., AAV2. In certain embodiments, the second VP-coding region encodes AAV capsid proteins of an AAV serotype, e.g., AAV2. In certain embodiments, the AAV serotype of the first VP-coding region is the same as the AAV serotype of the second VP-coding region. In certain embodiments, the AAV serotype of the first VP-coding region is different from the AAV serotype of the second VP-coding region. In certain embodiments, a VP-coding region can be codon optimized for an insect cell. In certain embodiments, a VP-coding region can be codon optimized for a Spodoptera frugiperda cell.

In certain embodiments, the viral expression construct comprises: (i) a first nucleotide sequence which comprises a first expression control region comprising a first promoter sequence, and a first VP-coding region which comprises a nucleotide sequence encoding one or more AAV capsid proteins selected from VP1, VP2 and VP3; and (ii) a second nucleotide sequence which comprises a second expression control region comprising a second promoter sequence, and a second VP-coding region which comprises a nucleotide sequence encoding VP1 AAV capsid proteins, but not VP2 or VP3. In certain embodiments, the viral expression construct comprises: (i) a first nucleotide sequence which comprises a first expression control region comprising a first promoter sequence, and a first VP-coding region which comprises a nucleotide sequence encoding VP2 and VP3 AAV capsid proteins, but not VP1; and (ii) a second nucleotide sequence which comprises a second expression control region comprising a second promoter sequence, and a second VP-coding region which comprises a nucleotide sequence encoding VP1 AAV capsid proteins, but not VP2 or VP3. In certain embodiments, the nucleotide sequence of the second VP-coding region is codon optimized. In certain embodiments, the nucleotide sequence of the second VP-coding region is codon optimized for an insect cell, or more specifically for a Spodoptera frugiperda cell. In certain embodiments, the nucleotide sequence of the second VP-coding region is codon optimized codon optimized to have a nucleotide homology with the reference nucleotide sequence of less than 100%, less than 90%, or less than 80%.

In certain embodiments, the viral expression construct comprises: (i) a first nucleotide sequence which comprises a first expression control region comprising a first promoter sequence, a first start codon region which comprises a first start codon, a first VP-coding region which comprises a nucleotide sequence encoding one or more AAV capsid proteins selected from VP1, VP2 and VP3, and a first stop codon region which comprises a first stop codon; and (ii) a second nucleotide sequence which comprises a second expression control region comprising a second promoter sequence, a second start codon region which comprises a second start codon, a second VP-coding region which comprises a nucleotide sequence encoding VP1 AAV capsid proteins, but not VP2 or VP3, and a second stop codon region which comprises a second stop codon. In certain embodiments, the nucleic acid construct comprises: (i) a first nucleotide sequence which comprises a first expression control region comprising a first promoter sequence, a first start codon region which comprises a first start codon, a first VP-coding region which comprises a nucleotide sequence encoding VP2 and VP3 AAV capsid proteins, but not VP1, and a first stop codon region which comprises a first stop codon; and (ii) a second nucleotide sequence which comprises a second expression control region comprising a second promoter sequence, a second start codon region which comprises a second start codon, a second VP-coding region which comprises a nucleotide sequence encoding VP1 AAV capsid proteins, but not VP2 or VP3, and a second stop codon region which comprises a second stop codon. In certain embodiments, the first start codon is ATG, the second start codon is ATG, or both the first and second start codons are ATG.

In certain embodiments, a nucleotide sequence encoding a VP1 capsid protein can be codon optimized. In certain embodiments, a nucleotide sequence encoding a VP1 capsid protein can be codon optimized for an insect cell. In certain embodiments, a nucleotide sequence encoding a VP2 capsid protein can be codon optimized. In certain embodiments, a nucleotide sequence encoding a VP2 capsid protein can be codon optimized for an insect cell. In certain embodiments, a nucleotide sequence encoding a VP3 capsid protein can be codon optimized. In certain embodiments, a nucleotide sequence encoding a VP3 capsid protein can be codon optimized for an insect cell.

In certain embodiments, a nucleotide sequence encoding a VP1 capsid protein can be codon optimized to have a nucleotide homology with the reference nucleotide sequence of less than 100%. In certain embodiments, the nucleotide homology between the codon-optimized VP1 nucleotide sequence and the reference VP1 nucleotide sequence is less than 100%, less than 99%, less than 98%, less than 97%, less than 96%, less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 78%, less than 76%, less than 74%, less than 72%, less than 70%, less than 68%, less than 66%, less than 64%, less than 62%, less than 60%, less than 55%, less than 50%, and less than 40%.

In certain embodiments, a nucleotide sequence encoding a VP2 capsid protein can be codon optimized to have a nucleotide homology with the reference nucleotide sequence of less than 100%. In certain embodiments, the nucleotide homology between the codon-optimized VP1 nucleotide sequence and the reference VP1 nucleotide sequence is less than 100%, less than 99%, less than 98%, less than 97%, less than 96%, less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 78%, less than 76%, less than 74%, less than 72%, less than 70%, less than 68%, less than 66%, less than 64%, less than 62%, less than 60%, less than 55%, less than 50%, and less than 40%.

In certain embodiments, a nucleotide sequence encoding a VP3 capsid protein can be codon optimized to have a nucleotide homology with the reference nucleotide sequence of less than 100%. In certain embodiments, the nucleotide homology between the codon-optimized VP1 nucleotide sequence and the reference VP1 nucleotide sequence is less than 100%, less than 99%, less than 98%, less than 97%, less than 96%, less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 78%, less than 76%, less than 74%, less than 72%, less than 700%, less than 68%, less than 66%, less than 64%, less than 62%, less than 60%, less than 55%, less than 50%, and less than 40%.

Structural VP proteins, VP1, VP2, and VP3 of a viral expression construct can be encoded in a single open reading frame regulated by utilization of both alternative splice acceptor and non-canonical translational initiation codons. VP1, VP2 and VP3 can be transcribed and translated from a single transcript in which both in-frame and/or out-of-frame start codons are engineered to control the VP1:VP2:VP3 ratio produced by the nucleotide transcript. In certain embodiments, VP1 can be produced from a sequence which encodes for VP1 only. As use herein, the terms “only for VP1” or “VP1 only” refers to a nucleotide sequence or transcript which encodes for a VP1 capsid protein and: (i) lacks the necessary start codons within the VP1 sequence (i.e. deleted or mutated) for full transcription or translation of VP2 and VP3 from the same sequence; (ii) comprises additional codons within the VP1 sequence which prevent transcription or translation of VP2 and VP3 from the same sequence; or (iii) comprises a start codon for VP1 (e.g. ATG), such that VP1 is the primary VP protein produced by the nucleotide transcript.

In certain embodiments, VP2 can be produced from a sequence which encodes for VP2 only. As use herein, the terms “only for VP2” or “VP2 only” refers to a nucleotide sequence or transcript which encodes for a VP2 capsid protein and: (i) the nucleotide transcript is a truncated variant of a full VP capsid sequence which encodes only VP2 and VP3 capsid proteins; and (ii) which comprise a start codon for VP2 (e.g. ATG), such that VP2 is the primary VP protein produced by the nucleotide transcript.

In certain embodiments, VP1 and VP2 can be produced from a sequence which encodes for VP1 and VP2 only. As use herein, the terms “only for VP1 and VP2” or “VP1 and VP2 only” refer to a nucleotide sequence or transcript which encodes for VP1 and VP2 capsid proteins and: (i) lacks the necessary start codons within the VP sequence (i.e. deleted or mutated) for full transcription or translation of VP3 from the same sequence; (ii) comprises additional codons within the VP sequence which prevent transcription or translation of VP3 from the same sequence; (iii) comprises a start codon for VP1 (e.g. ATG) and VP2 (e.g. ATG), such that VP1 and VP2 are the primary VP protein produced by the nucleotide transcript; or (iv) comprises VP1-only nucleotide transcript and a VP2-only nucleotide transcript connected by a linker, such as an IRES region.

In certain embodiments, the viral expression construct may contain a nucleotide sequence which comprises a start codon region, such as a sequence encoding AAV capsid proteins which comprise one or more start codon regions. In certain embodiments, the start codon region can be within an expression control sequence. The start codon can be ATG or a non-ATG codon (i.e., a suboptimal start codon where the start codon of the AAV VP1 capsid protein is a non-ATG). In certain embodiments, the viral expression construct used for AAV production may contain a nucleotide sequence encoding the AAV capsid proteins where the initiation codon of the AAV VP1 capsid protein is a non-ATG, i.e., a suboptimal initiation codon, allowing the expression of a modified ratio of the viral capsid proteins in the production system, to provide improved infectivity of the host cell. In a non-limiting example, a viral construct vector may contain a nucleic acid construct comprising a nucleotide sequence encoding AAV VP1, VP2, and VP3 capsid proteins, wherein the initiation codon for translation of the AAV VP1 capsid protein is CTG, TTG, or GTG, as described in U.S. Pat. No. 8,163,543, the content of which is incorporated herein by reference in its entirety as related to AAV capsid proteins and the production thereof, insofar as it does not conflict with the present disclosure.

### Rep-Coding Region

In certain embodiments, a viral expression construct can comprise a Rep52-coding region. A Rep52-coding region is a nucleotide sequence which comprises a Rep52 nucleotide sequence encoding a Rep52 protein. In certain embodiments, a viral expression construct can comprise a Rep78-coding region. A Rep78-coding region is a nucleotide sequence which comprises a Rep78 nucleotide sequence encoding a Rep78 protein. In certain embodiments, a viral expression construct can comprise a Rep40-coding region. A Rep40-coding region is a nucleotide sequence which comprises a Rep40 nucleotide sequence encoding a Rep40 protein. In certain embodiments, a viral expression construct can comprise a Rep68-coding region. A Rep68-coding region is a nucleotide sequence which comprises a Rep68 nucleotide sequence encoding a Rep68 protein.

In certain embodiments, the viral expression construct comprises a first nucleotide sequence which comprises: a Rep52-coding region which comprises a Rep52 sequence encoding a Rep52 protein, a Rep78-coding region which comprises a Rep78 sequence encoding a Rep78 protein, or a combination thereof. In certain embodiments, the first nucleotide sequence comprises both a Rep52-coding region and a Rep78-coding region. In certain embodiments, the first nucleotide sequence comprises a single open reading frame, consists essentially of a single open reading frame, or consists of a single open reading frame. In certain embodiments, the first nucleotide sequence comprises a first open reading frame which comprises a Rep52-coding region, and a second open reading frame which comprises a Rep78-coding region and which is different from the first open reading frame.

In certain embodiments, non-structural proteins, Rep52 and Rep78, of a viral expression construct can be encoded in a single open reading frame regulated by utilization of both alternative splice acceptor and non-canonical translational initiation codons.

Both Rep78 and Rep52 can be translated from a single transcript: Rep78 translation initiates at a first start codon (AUG or non-AUG) and Rep52 translation initiates from a Rep52 start codon (e.g. AUG) within the Rep78 sequence. Rep78 and Rep52 can also be translated from separate transcripts with independent start codons. The Rep52 initiation codons within the Rep78 sequence can be mutated, modified or removed, such that processing of the modified Rep78 sequence will not produce Rep52 proteins.

In certain embodiments, the viral expression construct of the present disclosure may be a plasmid vector or a baculoviral construct that encodes the parvoviral rep proteins for expression in insect cells. In certain embodiments, a single coding sequence is used for the Rep78 and Rep52 proteins, wherein start codon for translation of the Rep78 protein is a suboptimal start codon, selected from the group consisting of ACG, TTG, CTG and GTG, that effects partial exon skipping upon expression in insect cells, as described in U.S. Pat. No. 8,512,981, the content of which is incorporated herein by reference in its entirety as related to the promotion of less abundant expression of Rep78 as compared to Rep52 to promote high vector yields, insofar as it does not conflict with the present disclosure.

In certain embodiments, the viral expression construct may be a plasmid vector or a baculoviral construct for the expression in insect cells that contains repeating codons with differential codon biases, for example to achieve improved ratios of Rep proteins, e.g. Rep78 and Rep52 thereby improving large scale (commercial) production of viral expression construct and/or payload construct vectors in insect cells, as taught in U.S. Pat. No. 8,697,417, the content of which is incorporated herein by reference in its entirety as related to AAV replication proteins and the production thereof, insofar as it does not conflict with the present disclosure.

In certain embodiment, improved ratios of rep proteins may be achieved using the method and constructs described in U.S. Pat. No. 8,642,314, the content of which is incorporated herein by reference in its entirety as related to AAV replications proteins and the production thereof, insofar as it does not conflict with the present disclosure.

In certain embodiments, the viral expression construct may encode mutant parvoviral Rep polypeptides which have one or more improved properties as compared with their corresponding wild type Rep polypeptide, such as the preparation of higher virus titers for large scale production. Alternatively, they may be able to allow the production of better-quality viral particles or sustain more stable production of virus. In a non-limiting example, the viral expression construct may encode mutant Rep polypeptides with a mutated nuclear localization sequence or zinc finger domain, as described in Patent Application US 20130023034, the content of which is incorporated herein by reference in its entirety as related to AAV replications proteins and the production thereof, insofar as it does not conflict with the present disclosure.

In certain embodiments, the nucleic acid construct comprises a first nucleotide sequence, and a second nucleotide sequence which is separate from the first nucleotide sequence within the nucleic acid construct. In certain embodiments, the nucleic acid construct comprises a first nucleotide sequence which comprises a Rep52-coding region, and a separate second nucleotide sequence which comprises a Rep78-coding region. In certain embodiments, the nucleic acid construct comprises a first nucleotide sequence and a separate second nucleotide sequence; wherein the first nucleotide sequence comprises a Rep52-coding region and a 2A sequence region; and wherein the second nucleotide sequence comprises a Rep78-coding region and a 2A sequence region.

In certain embodiments, a first nucleotide sequence comprises a Rep52-coding region and 2A sequence region. In certain embodiments, a first nucleotide sequence comprises a Rep78-coding region and 2A sequence region. In certain embodiments, a first nucleotide sequence comprises a Rep52-coding region, a Rep78-coding region, and 2A sequence region. In certain embodiments, a first nucleotide sequence comprises a 2A sequence region located between a Rep52-coding region and a Rep78-coding region on the nucleotide sequence. In certain embodiments, a first nucleotide comprises, in order from the 5′-end to the 3′-end, a Rep52-coding region, a 2A sequence region, and a Rep78-coding region. In certain embodiments, a first nucleotide comprises, in order from the 5′-end to the 3′-end, a Rep78-coding region, a 2A sequence region, and a Rep52-coding region.

For example, in certain embodiments, a first nucleotide sequence comprises a start codon region, a Rep52-coding region, 2A sequence region, and a stop codon region. In certain embodiments, a first nucleotide sequence comprises a start codon region, a Rep78-coding region, 2A sequence region, and a stop codon region. In certain embodiments, a first nucleotide sequence comprises a start codon region, a Rep52-coding region, a 2A sequence region, a Rep78-coding region, and a stop codon region. In certain embodiments, a first nucleotide comprises, in order from the 5′-end to the 3′-end, a start codon region, a Rep52-coding region, a 2A sequence region, a Rep78-coding region, and a stop codon region. In certain embodiments, a first nucleotide comprises, in order from the 5′-end to the 3′-end, a start codon region, a Rep78-coding region, a 2A sequence region, a Rep52-coding region, and a stop codon region.

In certain embodiments, the viral expression construct comprises one or more essential-gene regions which comprises an essential-gene nucleotide sequence encoding an essential protein for the nucleic acid construct. In certain embodiments, the essential-gene nucleotide sequence is a baculoviral sequence encoding an essential baculoviral protein. In certain embodiments, the essential baculoviral protein is a baculoviral envelope protein or a baculoviral capsid protein. For example, in certain embodiments, the nucleic acid construct comprises a first nucleotide sequence and a separate second nucleotide sequence; wherein the first nucleotide sequence comprises a Rep52-coding region and a first essential-gene region; and wherein the second nucleotide sequence comprises a Rep78-coding region and a second essential-gene region. In certain embodiments, the nucleic acid construct comprises a first nucleotide sequence and a separate second nucleotide sequence; wherein the first nucleotide sequence comprises a Rep52-coding region, a 2A sequence region, and a first essential-gene region; and wherein the second nucleotide sequence comprises a Rep78-coding region, a 2A sequence region, and a second essential-gene region. In certain embodiments, the nucleic acid construct comprises a first nucleotide sequence and a separate second nucleotide sequence; wherein the first nucleotide sequence comprises, in order, a Rep52-coding region, a 2A sequence region, and a first essential-gene region; and wherein the second nucleotide sequence comprises, in order, a Rep78-coding region, a 2A sequence region, and a second essential-gene region.

In certain embodiments, the essential baculoviral protein is a GP64 baculoviral envelope protein. In certain embodiments, the essential baculoviral protein is a VP39 baculoviral capsid protein.

In certain embodiments, a first nucleotide sequence comprises a Rep52-coding region, a Rep78-coding region, and an IRES sequence region. In certain embodiments, a first nucleotide sequence comprises an IRES sequence region located between a Rep52-coding region and a Rep78-coding region on the nucleotide sequence. In certain embodiments, a first nucleotide comprises, in order from the 5′-end to the 3′-end, a Rep52-coding region, an IRES sequence region, and a Rep78-coding region. In certain embodiments, a first nucleotide comprises, in order from the 5′-end to the 3′-end, a Rep78-coding region, an IRES sequence region, and a Rep52-coding region.

In certain embodiments, the first nucleotide sequence comprises a first open reading frame which comprises a Rep52-coding region, a second open reading frame which comprises a Rep78-coding region, and an IRES sequence region located between the first open reading frame and the second open reading frame. In certain embodiments, a first nucleotide sequence comprises, in order from the 5′-end to the 3′-end, a first open reading frame which comprises a Rep52-coding region, an IRES sequence region, and a second open reading frame which comprises a Rep78-coding region. In certain embodiments, a first nucleotide sequence comprises, in order from the 5′-end to the 3′-end, a first open reading frame which comprises a Rep78-coding region, an IRES sequence region, and a second open reading frame which comprises a Rep52-coding region.

In certain embodiments, a first nucleotide sequence comprises, in order from the 5′-end to the 3′-end: a first open reading frame which comprises a first start codon region, a Rep52-coding region, and a first stop codon region; an IRES sequence region; and a second open reading frame which comprises a second start codon region, a Rep78-coding region, and a second stop codon region. In certain embodiments, a first nucleotide sequence comprises, in order from the 5′-end to the 3′-end: a first open reading frame which comprises a first start codon region, a Rep78-coding region, and a first stop codon region; an IRES sequence region; and a second open reading frame which comprises a second start codon region, a Rep52-coding region, and a second stop codon region.

In certain embodiments of the present disclosure, Rep52 or Rep78 is transcribed from the baculoviral derived polyhedron promoter (polh). Rep52 or Rep78 can also be transcribed from a weaker promoter, for example a deletion mutant of the IE-1 promoter, the ΔIE-1 promoter, has about 20% of the transcriptional activity of that IE-1 promoter. A promoter substantially homologous to the ΔIE-1 promoter may be used. In respect to promoters, a homology of at least 50%, 60%, 70%, 80%, 90% or more, is considered to be a substantially homologous promoter.

### Expression Control

### Expression Control Regions

A viral expression construct (e.g. expressionBac) of the present disclosure can comprise one or more expression control region encoded by expression control sequences. In certain embodiments, the expression control sequences are for expression in a viral production cell, such as an insect cell. In certain embodiments, the expression control sequences are operably linked to a protein-coding nucleotide sequence. In certain embodiments, the expression control sequences are operably linked to a VP coding nucleotide sequence or a Rep coding nucleotide sequence.

Herein, the terms “coding nucleotide sequence”, “protein-encoding gene” or “protein-coding nucleotide sequence” refer to a nucleotide sequence that encodes or is translated into a protein product, such as VP proteins or Rep proteins. “Operably linked” means that the expression control sequence is positioned relative to the coding sequence such that it can promote the expression of the encoded gene product.

“Expression control sequence” refers to a nucleic acid sequence that regulates the expression of a nucleotide sequence to which it is operably linked. An expression control sequence is “operably linked” to a nucleotide sequence when the expression control sequence controls and regulates the transcription and/or the translation of the nucleotide sequence. Thus, an expression control sequence can comprise promoters, enhancers, untranslated regions (UTRs), internal ribosome entry sites (IRES), transcription terminators, a start codon in front of a protein-encoding gene, splicing signal for introns, and stop codons. The term “expression control sequence” is intended to comprise, at a minimum, a sequence whose presence are designed to influence expression, and can also comprise additional advantageous components. For example, leader sequences and fusion partner sequences are expression control sequences. The term can also comprise the design of the nucleic acid sequence such that undesirable, potential initiation codons in and out of frame, are removed from the sequence. It can also comprise the design of the nucleic acid sequence such that undesirable potential splice sites are removed. It comprises sequences or polyadenylation sequences (pA) which direct the addition of a polyA tail, i.e., a string of adenine residues at the 3′-end of an mRNA, sequences referred to as polyA sequences. It also can be designed to enhance mRNA stability. Expression control sequences which affect the transcription and translation stability, e.g., promoters, as well as sequences which effect the translation, e.g., Kozak sequences, are known in insect cells. Expression control sequences can be of such nature as to modulate the nucleotide sequence to which it is operably linked such that lower expression levels or higher expression levels are achieved.

In certain embodiments, the expression control sequence can comprise one or more promoters. Promoters can comprise, but are not limited to, baculovirus major late promoters, insect virus promoters, non-insect virus promoters, vertebrate virus promoters, nuclear gene promoters, chimeric promoters from one or more species comprising virus and non-virus elements, and/or synthetic promoters. In certain embodiments, a promoter can be Ctx, Op-EI, EI, ΔEI, EI-1, pH, PIO, polH (polyhedron), ΔpolH, Dmhsp70, Hr1, Hsp70, 4×Hsp27 EcRE+minimal Hsp70, IE, IE-1, ΔIE-1, ΔIE, p10, Δp10 (modified variations or derivatives of p10), p5, p19, p35, p40, p6.9, and variations or derivatives thereof. In certain embodiments, the promoter is a Ctx promoter. In certain embodiments, the promoter is a p10 promoter. In certain embodiments, the promoter is a polH promoter. In certain embodiments, a promoter can be selected from tissue-specific promoters, cell-type-specific promoters, cell-cycle-specific promoters, and variations or derivatives thereof. In certain embodiments, a promoter can be a CMV promoter, an alpha 1-antitrypsin (al-AT) promoter, a thyroid hormone-binding globulin promoter, a thyroxine-binding globulin (LPS) promoter, an HCR-ApoCII hybrid promoter, an HCR-hAAT hybrid promoter, an albumin promoter, an apolipoprotein E promoter, an α1-AT+EaIb promoter, a tumor-selective E2F promoter, a mononuclear blood IL-2 promoter, and variations or derivatives thereof. In certain embodiments, the promoter is a low-expression promoter sequence. In certain embodiments, the promoter is an enhanced-expression promoter sequence. In certain embodiments, the promoter can comprise Rep or Cap promoters as described in US Patent Application 20110136227, the content of which is incorporated herein by reference in its entirety as related to expression promoters, insofar as it does not conflict with the present disclosure.

In certain embodiments, a viral expression construct can comprise the same promoter in all nucleotide sequences. In certain embodiments, a viral expression construct can comprise the same promoter in two or more nucleotide sequences. In certain embodiments, a viral expression construct can comprise a different promoter in two or more nucleotide sequences. In certain embodiments, a viral expression construct can comprise a different promoter in all nucleotide sequences.

In certain embodiments the viral expression construct encodes elements to improve expression in certain cell types. In a further embodiment, the expression construct may comprise polh and/or ΔIE-1 insect transcriptional promoters, CMV mammalian transcriptional promoter, and/or p10 insect specific promoters for expression of a desired gene in a mammalian or insect cell.

More than one expression control sequence can be operably linked to a given nucleotide sequence. For example, a promoter sequence, a translation initiation sequence, and a stop codon can be operably linked to a nucleotide sequence.

In certain embodiments, the viral expression construct can comprise one or more expression control sequence between protein-coding nucleotide sequences. In certain embodiments, an expression control region can comprise an IRES sequence region which comprises an IRES nucleotide sequence encoding an internal ribosome entry sight (IRES). The internal ribosome entry sight (IRES) can be selected from the group consisting or: FMDV-IRES from Foot-and-Mouth-Disease virus, EMCV-IRES from Encephalomyocarditis virus, and combinations thereof.

In certain embodiments, the viral expression construct is as described in PCT/US2019/054600 and/or U.S. Provisional Patent Application No. 62/741,855 the contents of which are each incorporated by reference in their entireties.

In certain embodiments, the viral expression construct may contain a nucleotide sequence which comprises a start codon region, such as a sequence encoding AAV capsid proteins which comprise one or more start codon regions. In certain embodiments, the start codon region can be within an expression control sequence.

In certain embodiments, the viral expression construct may contain a nucleotide sequence which comprises a stop codon region, such as a sequence encoding AAV capsid proteins which comprise one or more stop codon regions. In certain embodiments, the stop codon region can be within an expression control sequence.

In certain embodiments, the viral expression construct comprises one or more start codon regions which include a start codon. In certain embodiments, the viral expression construct comprises one or more stop codon regions which include a stop codon. In certain embodiments, the viral expression construct comprises one or more start codon regions and one or more stop codon regions. In certain embodiments, the start codon region and/or stop codon region can be within an expression control sequence.

In certain embodiments, the viral expression construct comprises one or more expression control regions which comprise an expression control sequence. In certain embodiments, the expression control region comprises one or more promoter sequences. In certain embodiments, the expression control region comprises one or more promoter sequences selected from the group consisting of: baculovirus major late promoters, insect virus promoters, non-insect virus promoters, vertebrate virus promoters, nuclear gene promoters, chimeric promoters from one or more species including virus and non-virus elements, synthetic promoters, and variations or derivatives thereof. In certain embodiments, the expression control region comprises one or more promoter sequences selected from the group consisting of: Ctx promoter, polh insect transcriptional promoters, ΔIE-1 insect transcriptional promoters, p10 insect specific promoters, Δp10 insect specific promoters (variations or derivatives of p10), CMV mammalian transcriptional promoter, and variations or derivatives thereof. In certain embodiments, the expression control region comprises one or more low-expression promoter sequences. In certain embodiments, the expression control region comprises one or more enhanced-expression promoter sequences.

In certain embodiments, an expression control region can comprise a 2A sequence region which comprises a 2A nucleotide sequence encoding a viral 2A peptide. The sequence allows for co-translation of multiple polypeptides within a single open reading frame (ORF). As the ORF is translated, glycine and proline residues with the 2A sequence prevent the formation of a normal peptide bond, which results in ribosomal “skipping” and “self-cleavage” within the polypeptide chain. The viral 2A peptide can be selected from the group consisting of: F2A from Foot-and-Mouth-Disease virus, T2A from Thosea asigna virus, E2A from Equine rhinitis A virus, P2A from porcine teschovirus-1, BmCPV2A from cytoplasmic polyhedrosis virus, BmIFV 2A from B. mori flacherie virus, and combinations thereof.

In some embodiments, the first and/or second nucleotide sequence comprises a start codon and/or stop codon and/or internal ribosome entry site (IRES). In certain embodiments, the IRES nucleotide sequence encodes an internal ribosome entry site (IRES) selected from the group consisting of: FMDV-IRES from Foot-and-Mouth-Disease virus, EMCV-IRES from Encephalomyocarditis virus, and combinations thereof.

The method of the present disclosure is not limited by the use of specific expression control sequences. However, when a certain stoichiometry of VP products are achieved (close to 1:1:10 for VP1, VP2, and VP3, respectively) and also when the levels of Rep52 or Rep40 (also referred to as the p19 Reps) are significantly higher than Rep78 or Rep68 (also referred to as the p5 Reps), improved yields of AAV in production cells (such as insect cells) may be obtained. In certain embodiments, the p5/p19 ratio is below 0.6 more, below 0.4, or below 0.3, but always at least 0.03. These ratios can be measured at the level of the protein or can be implicated from the relative levels of specific mRNAs.

In certain embodiments, AAV particles are produced in viral production cells (such as mammalian or insect cells) wherein all three VP proteins are expressed at a stoichiometry approaching, about or which is 1:1:10 (VP1:VP2:VP3).

In certain embodiments, AAV particles are produced in viral production cells (such as mammalian or insect cells) wherein all three VP proteins are expressed at a stoichiometry approaching, about or which is 2:2:10 (VP1:VP2:VP3).

In certain embodiments, AAV particles are produced in viral production cells (such as mammalian or insect cells) wherein all three VP proteins are expressed at a stoichiometry approaching, about or which is 2:0:10 (VP1:VP2:VP3).

In certain embodiments, AAV particles are produced in viral production cells (such as mammalian or insect cells) wherein all three VP proteins are expressed at a stoichiometry approaching, about or which is 1-2:0-2:10 (VP1:VP2:VP3).

In certain embodiments, AAV particles are produced in viral production cells (such as mammalian or insect cells) wherein all three VP proteins are expressed at a stoichiometry approaching, about or which is 1-2:1-2:10 (VP1:VP2:VP3).

In certain embodiments, AAV particles are produced in viral production cells (such as mammalian or insect cells) wherein all three VP proteins are expressed at a stoichiometry approaching, about or which is 2-3:0-3:10 (VP1:VP2:VP3).

In certain embodiments, AAV particles are produced in viral production cells (such as mammalian or insect cells) wherein all three VP proteins are expressed at a stoichiometry approaching, about or which is 2-3:2-3:10 (VP1:VP2:VP3).

In certain embodiments, AAV particles are produced in viral production cells (such as mammalian or insect cells) wherein all three VP proteins are expressed at a stoichiometry approaching, about or which is 3:3:10 (VP1:VP2:VP3).

In certain embodiments, AAV particles are produced in viral production cells (such as mammalian or insect cells) wherein all three VP proteins are expressed at a stoichiometry approaching, about or which is 3-5:0-5:10 (VP1:VP2:VP3).

In certain embodiments, AAV particles are produced in viral production cells (such as mammalian or insect cells) wherein all three VP proteins are expressed at a stoichiometry approaching, about or which is 3-5:3-5:10 (VP1:VP2:VP3).

In certain embodiments, the expression control regions are engineered to produce a VP1:VP2:VP3 ratio selected from the group consisting of: about or exactly 1:0:10; about or exactly 1:1:10; about or exactly 2:1:10; about or exactly 2:1:10; about or exactly 2:2:10; about or exactly 3:0:10; about or exactly 3:1:10; about or exactly 3:2:10; about or exactly 3:3:10; about or exactly 4:0:10; about or exactly 4:1:10; about or exactly 4:2:10; about or exactly 4:3:10; about or exactly 4:4:10; about or exactly 5:5:10; about or exactly 1-2:0-2:10; about or exactly 1-2:1-2:10; about or exactly 1-3:0-3:10; about or exactly 1-3:1-3:10; about or exactly 1-4:0-4:10; about or exactly 1-4:1-4:10; about or exactly 1-5:1-5:10; about or exactly 2-3:0-3:10; about or exactly 2-3:2-3:10; about or exactly 2-4:2-4:10; about or exactly 2-5:2-5:10; about or exactly 3-4:3-4:10; about or exactly 3-5:3-5:10; and about or exactly 4-5:4-5:10.

### Viral Production Cells and Vectors

### Mammalian Cells

Viral production of the present disclosure disclosed herein describes processes and methods for producing AAV particles or viral vector that contacts a target cell to deliver a payload construct, e.g. a recombinant AAV particle or viral construct, which comprises a nucleotide encoding a payload molecule. The viral production cell may be selected from any biological organism, comprising prokaryotic (e.g., bacterial) cells, and eukaryotic cells, comprising, insect cells, yeast cells and mammalian cells.

In certain embodiments, the AAV particles of the present disclosure may be produced in a viral production cell that comprises a mammalian cell. Viral production cells may comprise mammalian cells such as A549, WEH1, 3T3, 10T1/2, BHK, MDCK, COS 1, COS 7, BSC 1, BSC 40, BMT 10, VERO. W138, HeLa, HEK293, HEK293T (293T), Saos, C2C12, L cells, HT1080, HepG2 and primary fibroblast, hepatocyte and myoblast cells derived from mammals. Viral production cells can comprise cells derived from mammalian species comprising, but not limited to, human, monkey, mouse, rat, rabbit, and hamster or cell type, comprising but not limited to fibroblast, hepatocyte, tumor cell, cell line transformed cell, etc.

AAV viral production cells commonly used for production of recombinant AAV particles comprise, but is not limited to HEK293 cells, COS cells, C127, 3T3, CHO, HeLa cells, KB cells, BHK, and other mammalian cell lines as described in U.S. Pat. Nos. 6,156,303, 5,387,484, 5,741,683, 5,691,176, 6,428,988 and 5,688,676; U.S. patent application 2002/0081721, and International Patent Publication Nos. WO 00/47757, WO 00/24916, and WO 96/17947, the contents of which are each incorporated herein by reference in their entireties, insofar as they do not conflict with the present disclosure. In certain embodiments, the AAV viral production cells are trans-complementing packaging cell lines that provide functions deleted from a replication-defective helper virus, e.g., HEK293 cells or other Ea trans-complementing cells.

In certain embodiments, the packaging cell line 293-10-3 (ATCC Accession No. PTA-2361) may be used to produce the AAV particles, as described in U.S. Pat. No. 6,281,010, the content of which is incorporated herein by reference in its entirety as related to the 293-10-3 packaging cell line and uses thereof, insofar as it does not conflict with the present disclosure.

In certain embodiments, of the present disclosure a cell line, such as a HeLA cell line, for trans-complementing E1 deleted adenoviral vectors, which encoding adenovirus E1a and adenovirus E1b under the control of a phosphoglycerate kinase (PGK) promoter can be used for AAV particle production as described in U.S. Pat. No. 6,365,394, the content of which is incorporated herein by reference in its entirety as related to the HeLA cell line and uses thereof, insofar as it does not conflict with the present disclosure.

In certain embodiments, AAV particles are produced in mammalian cells using a triple transfection method wherein a payload construct, parvoviral Rep and parvoviral Cap and a viral expression construct are comprised within three different constructs. The triple transfection method of the three components of AAV particle production may be utilized to produce small lots of virus for assays comprising transduction efficiency, target tissue (tropism) evaluation, and stability.

AAV particles to be formulated may be produced by triple transfection or baculovirus mediated virus production, or any other method known in the art. Any suitable permissive or packaging cell known in the art may be employed to produce the vectors. In certain embodiments, trans-complementing packaging cell lines are used that provide functions deleted from a replication-defective helper virus, e.g., 293 cells or other E1a trans-complementing cells.

The gene cassette may contain some or all of the parvovirus (e.g., AAV) cap and rep genes. In certain embodiments, some or all of the cap and rep functions are provided in trans by introducing a packaging vector(s) encoding the capsid and/or Rep proteins into the cell. In certain embodiments, the gene cassette does not encode the capsid or Rep proteins. Alternatively, a packaging cell line is used that is stably transformed to express the cap and/or rep genes.

Recombinant AAV virus particles are, in certain embodiments, produced and purified from culture supernatants according to the procedure as described in US2016/0032254, the content of which is incorporated herein by reference in its entirety as related to the production and processing of recombinant AAV virus particles, insofar as it does not conflict with the present disclosure. Production may also involve methods known in the art comprising those using 293T cells, triple transfection or any suitable production method.

In certain embodiments, mammalian viral production cells (e.g. 293T cells) can be in an adhesion/adherent state (e.g. with calcium phosphate) or a suspension state (e.g. with polyethylencimine (PEI)). The mammalian viral production cell is transfected with plasmids required for production of AAV, (i.e., AAV rep/cap construct, an adenoviral viral expression construct, and/or ITR flanked payload construct). In certain embodiments, the transfection process can comprise optional medium changes (e.g. medium changes for cells in adhesion form, no medium changes for cells in suspension form, medium changes for cells in suspension form if desired). In certain embodiments, the transfection process can comprise transfection mediums such as DMEM or F17. In certain embodiments, the transfection medium can comprise serum or can be serum-free (e.g. cells in adhesion state with calcium phosphate and with serum, cells in suspension state with PEI and without serum).

Cells can subsequently be collected by scraping (adherent form) and/or pelleting (suspension form and scraped adherent form) and transferred into a receptacle. Collection steps can be repeated as necessary for full collection of produced cells. Next, cell lysis can be achieved by consecutive freeze-thaw cycles (−80 C to 37 C), chemical lysis (such as adding detergent triton), mechanical lysis, or by allowing the cell culture to degrade after reaching ˜0% viability. Cellular debris is removed by centrifugation and/or depth filtration. The samples are quantified for AAV particles by DNase resistant genome titration by DNA qPCR.

AAV particle titers are measured according to genome copy number (genome particles per milliliter). Genome particle concentrations are based on DNA qPCR of the vector DNA as previously reported (Clark et al. (1999) Hum. Gene Ther., 10:1031-1039; Veldwijk et al. (2002) Mol. Ther., 6:272-278, the contents of which are each incorporated herein by reference in their entireties as related to the measurement of particle concentrations, insofar as they do not conflict with the present disclosure).

### Insect Cells

Viral production of the present disclosure comprises processes and methods for producing AAV particles or viral vectors that contact a target cell to deliver a payload construct, e.g. a recombinant viral construct, which comprises a nucleotide encoding a payload molecule. In certain embodiments, the AAV particles or viral vectors of the present disclosure may be produced in a viral production cell that comprises an insect cell.

Growing conditions for insect cells in culture, and production of heterologous products in insect cells in culture are well-known in the art, see U.S. Pat. No. 6,204,059, the content of which is incorporated herein by reference in its entirety as related to the growth and use of insect cells in viral production, insofar as it does not conflict with the present disclosure.

Any insect cell which allows for replication of parvovirus and which can be maintained in culture can be used in accordance with the present disclosure, AAV viral production cells commonly used for production of recombinant AAV particles comprise, but is not limited to, Spodoptera frugiperda, comprising, but not limited to the Sf9 or Sf21 cell lines, Drosophila cell lines, or mosquito cell lines, such as Aedes albopictus derived cell lines. Use of insect cells for expression of heterologous proteins is well documented, as are methods of introducing nucleic acids, such as vectors, e.g., insect-cell compatible vectors, into such cells and methods of maintaining such cells in culture. See, for example, Methods in Molecular Biology, ed. Richard, Humana Press, N J (1995); O'Reilly et al., Baculovirus Expression Vectors, A Laboratory Manual, Oxford Univ. Press (1994); Samulski et al., J. Vir. 63:3822-8 (1989); Kajigaya et al., Proc. Nat'l. Acad. Sci. USA 88: 4646-50 (1991); Ruffing et al., J. Vir. 66:6922-30 (1992); Kimbauer et al., Vir. 219:37-44 (1996); Zhao et al., Vir. 272:382-93 (2000); and Samulski et al., U.S. Pat. No. 6,204,059, the contents of which are each incorporated herein by reference in their entireties as related to the use of insect cells in viral production, insofar as they do not conflict with the present disclosure.

In one embodiment, the AAV particles are made using the methods described in WO2015/191508, the content of which is incorporated herein by reference in its entirety, insofar as it does not conflict with the present disclosure.

In certain embodiments, insect host cell systems, in combination with baculoviral systems (e.g., as described by Luckow et al., Bio/Technology 6: 47 (1988)) may be used. In certain embodiments, an expression system for preparing chimeric peptide is Trichoplusia ni, Tn 5B1-4 insect cells/baculoviral system, which can be used for high levels of proteins, as described in U.S. Pat. No. 6,660,521, the content of which is incorporated herein by reference in its entirety, insofar as it does not conflict with the present disclosure.

### Insect Cell Media

Expansion, culturing, transfection, infection and storage of insect cells can be carried out in any cell culture media, cell transfection media or storage media known in the art or presented in the present disclosure, including Hyclone SFX Insect Cell Culture Media, Expression System ESF AF Insect Cell Culture Medium, Basal IPL-41 Insect Cell Culture Media, ThermoFisher Sf900II media, ThermoFisher Sf900III media, ThermoFisher Grace's Insect Media, or modified media formulations thereof.

In certain embodiments, an insect cell culture medium of the present disclosure can comprise any of the formulation additives or elements described in the present disclosure, including (but not limited to) inorganic salts, acids, bases, buffers, surfactants (such as Poloxamer 188/Pluronic F-68), amino acid mixtures, nutrient mixtures, sugars (such as glucose), vitamins, lipids, hydrolysates (i.e. yeast extract), cholesterol, and other known culture media elements. Formulation ingredients and/or additives can be incorporated gradually, one or more boluses, or as “spikes” (incorporation of large volumes in a short time).

In certain embodiments, an insect cell culture medium of the present disclosure can comprise hydrolysates, such as yeast extract (e.g. yeast extract ultrafiltrates). In certain embodiments, the yeast extract can comprise one or more of: Bacto TC Yeastolate, Sigma Select Yeast Extract, BD Difco Yeast Extract UF, Sigma Yeast Autolysate, NuTek NTB3-UF, and NuTek NTB3-UF. In certain embodiments, the yeast extract can comprise BD Difco Yeast Extract UF. In certain embodiments, the yeast extract can comprise Sigma Yeast Autolysate. In certain embodiments, the insect cell culture medium comprises about 2.0 g/L, about 2.5 g/L, about 3.0 g/L, about 3.5 g/L, about 4.0 g/L, about 4.5 g/L, about 5.0 g/L, about 5.5 g/L, about 6.0 g/L, about 6.5 g/L, about 7.0 g/L, about 7.5 g/L, about 8.0 g/L, about 8.5 g/L, about 9.0 g/L, about 9.5 g/L, about 10.0 g/L, about 10.5 g/L, about 11.0 g/L, about 11.5 g/L, about 12.0 g/L, or about 12.5 g/L of hydrolysates (per 1 L of insect cell culture medium). In certain embodiments, the insect cell culture medium comprises about 6.0 g/L of hydrolysates, such as yeast extract. In certain embodiments, the insect cell culture medium comprises about 27.0 g/L of hydrolysates, such as yeast extract. In certain embodiments, the insect cell culture medium comprises about 54.0 g/L of hydrolysates, such as yeast extract.

In certain embodiments, an insect cell culture medium of the present disclosure comprises cholesterol. In certain embodiments, the insect cell culture medium comprises at least 2.0 mg/L, at least 2.5 mg/L, at least 3.0 mg/L, at least 3.5 mg/L, at least 4.0 mg/L, at least 4.5 mg/L, at least 5.0 mg/L, at least 5.5 mg/L, at least 6.0 mg/L, at least 6.5 mg/L, 7.0 mg/L, at least 7.5 mg/L, at least 8.0 mg/L, at least 8.5 mg/L, 9.0 mg/L, at least 9.5 mg/L, at least 10.0 mg/L, at least 10.5 mg/L, 11.0 mg/L, at least 11.5 mg/L, at least 12.0 mg/L, or at least 12.5 mg/L of cholesterol (per 1 L of insect cell culture medium). In certain embodiments, the insect cell culture medium comprises at least 2.0 mg/L of cholesterol. In certain embodiments, the insect cell culture medium comprises at least 4.0 mg/L of cholesterol. In certain embodiments, the insect cell culture medium comprises at least 6.0 mg/L of cholesterol. In certain embodiments, the insect cell culture medium comprises at least 8.0 mg/L of cholesterol. In certain embodiments, the insect cell culture medium comprises at least 10.0 mg/L of cholesterol. In certain embodiments, the insect cell culture medium comprises at least 12.0 mg/L of cholesterol. In certain embodiments, the insect cell culture medium comprises between 4.0-12.5 mg/L of cholesterol. In certain embodiments, the insect cell culture medium comprises between 4.0-8.0 mg/L of cholesterol. In certain embodiments, the insect cell culture medium comprises between 6.0-8.0 mg/L of cholesterol. In certain embodiments, the insect cell culture medium comprises between 6.0-12.5 mg/L of cholesterol. In certain embodiments, the insect cell culture medium comprises between 8.0-12.5 mg/L of cholesterol.

In certain embodiments, an insect cell culture medium of the present disclosure can comprise a lipid emulsion. In certain embodiments, the insect cell culture medium comprises at least 5.0 mL, at least 5.5 mL, at least 6.0 mL, at least 6.5 mL, 7.0 mL, at least 7.5 mL, at least 8.0 mL, at least 8.5 mL, at least 9.0 mL, at least 9.5 mL, at least 10.0 mL, at least 10.5 mL, at least 11.0 mL, at least 11.5 mL, at least 12.0 mL, at least 12.5 mL, at least 13.0 mL, at least 13.5 mL, at least 14.0 mL, at least 14.5 mL, 15.0 mL, at least 15.5 mL, at least 16.0 mL, at least 16.5 mL, at least 17.0 mL, at least 17.5 mL, at least 18.0 mL, at least 18.5 mL, at least 19.0 mL, at least 19.5 mL, at least 20.0 mL, at least 20.5 mL, at least 21.0 mL, at least 21.5 mL, at least 22.0 mL, or at least 22.5 mL of lipid emulsion per 1 L of insect cell culture medium.

In certain embodiments, the lipid emulsion can comprise one or more of: cod liver oil, Tween 80, alpha-tocopherol acetate, ethanol, 10% pluronic F-68, and water.

In certain embodiments, the lipid emulsion can comprise one or more of: arachidonic acid, dl-alpha-tocopherol acetate, ethanol 100%, linoleic acid, linolenic acid, myristic acid, oleic acid, palmitic acid, palmitoleic acid, pluronic f-68, stearic acid, and tween 80. In certain embodiments, the lipid emulsion comprises, per 500 mL: about 1.0 μL arachidonic acid, about 36.5 μL dl-alpha-tocopherol acetate, about 48.75 mL ethanol 100%, about 5.5 μL linoleic acid, about 5.5 μL linolenic acid, about 5 mg myristic acid, about 5.6 μL oleic acid, about 5 mg palmitic acid, about 5.6 μL palmitoleic acid, about 450 mL pluronic f-68, about 5 mg stearic acid, and about 1030 μL tween 80.

In certain embodiments, an insect cell culture medium of the present disclosure can comprise an amino acid mixture. In certain embodiments, the insect cell culture medium comprises at least at least 100 mL, at least 105 mL, at least 110 mL, at least 115 mL, at least 120 mL, at least 125 mL, at least 130 mL, at least 135 mL, at least 140 mL, at least 145 mL, 150 mL, at least 155 mL, at least 160 mL, at least 165 mL, at least 170 mL, at least 175 mL, at least 180 mL, at least 185 mL, at least 190 mL, at least 195 mL, at least 200 mL, at least 205 mL, at least 210 mL, at least 215 mL, at least 220 mL, at least 225 mL, at least 230 mL, at least 235 mL, at least 240 mL, at least 245 mL, at least 250 mL, or at least 255 mL of amino acid mixture per 1 L of insect cell culture medium.

In certain embodiments, the amino acid mixture can comprise one or more of: L-arginine, L-asparagine, L-aspartic acid, L-glutamic acid, L-glycine, L-histidine, L-isoleucine, L-leucine, L-lysine, L-methionine, L-phenylalanine, L-serine, L-threonine, L-tryptophan, L-valine, L-proline, L-cysteine.HCL.H2O.

In certain embodiments, the amino acid mixture comprises: about 54.9 mM L-arginine, about 39 mM L-asparagine, about 38.7 mM L-aspartic acid, about 118.4 mM L-glutamic acid, about 128.9 mM L-glycine, about 27.7 mM L-histidine, about 84.1 mM L-isoleucine, about 104 mM L-leucine, about 75.9 mM L-lysine, about 4.9 mM L-methionine, about 13.4 mM L-phenylalanine, about 247.7 mM L-serine, about 46.6 mM L-threonine, about 9.1 mM L-tryptophan, about 45.2 mM L-valine, about 82.2 mM L-proline, about 45 mM L-cysteine.HCL.H2O.

In certain embodiments, an insect cell culture medium of the present disclosure can comprise a nutrient mixture. In certain embodiments, the insect cell culture medium comprises at least 5.0 mL, at least 5.5 mL, at least 6.0 mL, at least 6.5 mL, 7.0 mL, at least 7.5 mL, at least 8.0 mL, at least 8.5 mL, at least 9.0 mL, at least 9.5 mL, at least 10.0 mL, at least 10.5 mL, at least 11.0 mL, at least 11.5 mL, at least 12.0 mL, at least 12.5 mL, at least 13.0 mL, at least 13.5 mL, at least 14.0 mL, at least 14.5 mL, 15.0 mL, at least 15.5 mL, at least 16.0 mL, at least 16.5 mL, at least 17.0 mL, at least 17.5 mL, at least 18.0 mL, at least 18.5 mL, at least 19.0 mL, at least 19.5 mL, at least 20.0 mL, at least 20.5 mL, at least 21.0 mL, at least 21.5 mL, at least 22.0 mL, or at least 22.5 mL nutrient mixture per 1 L of insect cell culture medium.

In certain embodiments, the nutrient mixture can comprise one or more of: Thiamine.HCL, Riboflavin, D-Calcium pantothenate, Pyridoxine HCl, Para-aminobenzoic acid, Nicotinic acid, i-Inositol, Biotin, Choline chloride, Vitamin B12, Folic Acid, Molybdic acid (ammonium salt), Cobalt chloride hexahydrate, Cupric chloride, Manganese chloride, Zinc chloride, Ferrous Sulfate, Aspartate. In certain embodiments, the nutrient mixture can comprise: Thiamine.HCL, Riboflavin, D-Calcium pantothenate, Pyridoxine HCl, Para-aminobenzoic acid, Nicotinic acid, i-Inositol, Biotin, Choline chloride, and Vitamin B12. In certain embodiments, the nutrient mixture can comprise Folic Acid. In certain embodiments, the nutrient mixture can comprise: Molybdic acid (ammonium salt), Cobalt chloride hexahydrate, Cupric chloride, Manganese chloride, and Zinc chloride. In certain embodiments, the nutrient mixture can comprise: Ferrous Sulfate and Aspartate. In certain embodiments, the nutrient mixture can comprise: Thiamine.HCL, Riboflavin, D-Calcium pantothenate, Pyridoxine HCl, Para-aminobenzoic acid, Nicotinic acid, i-Inositol, Biotin, Choline chloride, Vitamin B12, Folic Acid, Molybdic acid (ammonium salt), Cobalt chloride hexahydrate, Cupric chloride, Manganese chloride, Zinc chloride, Ferrous Sulfate and Aspartate.

In certain embodiments, the nutrient mixture can comprise: about 80 mg/L Thiamine.HCL, about 80 mg/L Riboflavin, about 86.25 mg/L D-Calcium pantothenate, about 400 mg/L Pyridoxine HCl, about 320 mg/L Para-aminobenzoic acid, about 160 mg/L Nicotinic acid, about 400 mg/L i-Inositol, about 160 mg/L Biotin, about 20 g/L Choline chloride, and about 240 mg/L Vitamin B12. In certain embodiments, the nutrient mixture can comprise about 80 mg/L Folic Acid. In certain embodiments, the nutrient mixture can comprise: about 6.86 mg/L Molybdic acid (ammonium salt), about 27.27 mg/L Cobalt chloride hexahydrate, about 19.95 mg/L Cupric chloride, about 20.58 mg/L Manganese chloride, and about 40 mg/L Zinc chloride. In certain embodiments, the nutrient mixture can comprise: about 550.48 mg/L Ferrous Sulfate and about 356 mg/L Aspartate. In certain embodiments, the nutrient mixture can comprise: about 80 mg/L Thiamine.HCL, about 80 mg/L Riboflavin, about 86.25 mg/L D-Calcium pantothenate, about 400 mg/L Pyridoxine HCl, about 320 mg/L Para-aminobenzoic acid, about 160 mg/L Nicotinic acid, about 400 mg/L i-Inositol, about 160 mg/L Biotin, about 20 g/L Choline chloride, about 240 mg/L Vitamin B12, about 80 mg/L Folic Acid, about 6.86 mg/L Molybdic acid (ammonium salt), about 27.27 mg/L Cobalt chloride hexahydrate, about 19.95 mg/L Cupric chloride, about 20.58 mg/L Manganese chloride, about 40 mg/L Zinc chloride, about 550.48 mg/L Ferrous Sulfate and about 356 mg/L Aspartate.

In certain embodiments, the insect cell culture medium is serum-free. In certain embodiments, the insect cell culture medium is free of proteins of animal origin. In certain embodiments, the insect cell culture medium comprises L-glutamate and/or L-glutamine. In certain embodiments, the insect cell culture medium comprises poloxamer 188 (e.g. 10% pluronic F-68).

In certain embodiments, the insect cell culture medium comprises: hydrolysates (such as yeast extract ultrafiltrate), L-glutamine, poloxamer 188 (e.g. 10% pluronic F-68), lipid emulsion and cholesterol. In certain embodiments, the insect cell culture medium is serum-free, and comprises per L of media: 6 g of hydrolysates (such as yeast extract ultrafiltrate), 8.5 mL of 200 mM L-glutamine, 2 mL of poloxamer 188 (e.g. 10% pluronic F-68), 8 mL of lipid emulsion, and 4.65 mL of cholesterol concentrate, with the remainder of volume comprising a base media such as Basal IPL-41 Insect Cell Culture Media or ESF AF Insect Cell Culture Medium.

In certain embodiments, the insect cell culture medium is ESF AF Insect Cell Culture Medium. In certain embodiments, the insect cell culture medium used in the production of AAV particles (e.g., ESF AF Insect Cell Culture Medium) increases titer at least 1.5-fold or at least 2-fold when compared to other insect cell culture medium. In certain embodiments, the insect cell culture medium is Basal IPL-41 Insect Cell Culture Media. In certain embodiments, the insect cell culture medium is a modified formulation of Basal IPL-41 Insect Cell Culture Media.

In certain embodiments, an insect cell culture medium of the present disclosure can include a media feed additive. In certain embodiments, the media feed additive is added to the insect cell culture medium in a single bolus. In certain embodiments, the media feed additive is added to the insect cell culture medium before BIIC infection. In certain embodiments, the media feed additive is added to the insect cell culture medium at BIIC infection. In certain embodiments, the media feed additive is added to the insect cell culture medium after BIIC infection.

In certain embodiments, the media feed additive is added to the insect cell culture medium in two boluses. In certain embodiments, the media feed additive is added to the insect cell culture medium before BIIC infection and at BIIC infection. In certain embodiments, the media feed additive is added to the insect cell culture medium before BIIC infection and after BIIC infection. In certain embodiments, the media feed additive is added to the insect cell culture medium at BIIC infection and after BIIC infection.

In certain embodiments, the media feed additive is added to the insect cell culture medium in three or more boluses. In certain embodiments, the media feed additive is added to the insect cell culture medium before BIIC infection, at BIIC infection, and after BIIC infection. In certain embodiments, the media feed additive is added to the insect cell culture medium in one or more boluses before BIIC infection, at BIIC infection, and in one or more boluses after BIIC infection. In certain embodiments, the media feed additive is added to the insect cell culture medium on a daily basis after BIIC infection.

In certain embodiments, the media feed additive is serum-free. In certain embodiments, the media feed additive is free of proteins of animal origin. In certain embodiments, the media feed additive comprises L-glutamate and/or L-glutamine. In certain embodiments, the media feed additive comprises poloxamer 188 (e.g. 101% pluronic F-68).

In certain embodiments, the media feed additive is serum-free, and comprises hydrolysates (such as yeast extract ultrafiltrate), a lipid emulsion, a nutrient mixture, an amino acid mixture, and glucose.

In certain embodiments, the media feed additive comprises hydrolysates (e.g. Yeastolate Ultrafiltrate), lipid emulsion, nutrient mixture, amino acid mixture, and glucose.

In certain embodiments, the media feed additive is serum-free, and comprises per 336 mL of feed additive: 9 g of hydrolysates (100 g/L in 90 mL), 18 mL of lipid emulsion, 8 mL of nutrient mixture, 200 mL of amino acid mixture, and 10.09 g of glucose (504 g/L in 20 mL).

In certain embodiments, the glucose in the media feed additive can be replaced with maltose. In certain embodiments, the glucose in the media feed additive can be replaced with sucrose. In certain embodiments, the glucose in the media feed additive can be replaced with trehelose. In certain embodiments, the glucose in the media feed additive can be replaced with a disaccharide comprising maltose and glucose. In certain embodiments, the glucose in the media feed additive can be replaced with a disaccharide comprising trehelose and glucose.

In certain embodiments, the media feed additive can include about 10 g to about 20 g of sugar. In certain embodiments, the media feed additive can include about 20 g to about 30 g of sugar. In certain embodiments, the media feed additive can include about 30 g to about 40 g of sugar.

### Baculovirus Production Systems

In certain embodiments, processes of the present disclosure can comprise production of AAV particles or viral vectors in a baculoviral system using a viral expression construct and a payload construct vector. In certain embodiments, the baculoviral system comprises Baculovirus expression vectors (BEVs) and/or baculovirus infected insect cells (BIICs). In certain embodiments, a viral expression construct or a payload construct of the present disclosure can be a bacmid, also known as a baculovirus plasmid or recombinant baculovirus genome. In certain embodiments, a viral expression construct or a payload construct of the present disclosure can be polynucleotide incorporated by homologous recombination (transposon donor/acceptor system) into a bacmid by standard molecular biology techniques known and performed by a person skilled in the art. Transfection of separate viral replication cell populations produces two or more groups (e.g. two, three) of baculoviruses (BEVs), one or more group which can comprise the viral expression construct (e.g., the baculovirus is an “Expression BEV” or “expressionBac”), and one or more group which can comprise the payload construct (e.g., the baculovirus is a “Payload BEV” or “payloadBac”). The baculoviruses may be used to infect a viral production cell for production of AAV particles or viral vector.

In certain embodiments, the process comprises transfection of a single viral replication cell population to produce a single baculovirus (BEV) group which comprises both the viral expression construct and the payload construct. These baculoviruses may be used to infect a viral production cell for production of AAV particles or viral vector.

In certain embodiments, BEVs are produced using a Bacmid Transfection agent, such as Promega FuGENE HD, WFI water, or ThermoFisher Cellfectin II Reagent. In certain embodiments, BEVs are produced and expanded in viral production cells, such as an insect cell.

In certain embodiments, the method utilizes seed cultures of viral production cells that comprise one or more BEVs, comprising baculovirus infected insect cells (BIICs). The seed BIICs have been transfected/transduced/infected with an Expression BEV which comprises a viral expression construct, and also a Payload BEV which comprises a payload construct. In certain embodiments, the seed cultures are harvested, divided into aliquots and frozen, and may be used at a later time to initiate transfection/transduction/infection of a naïve population of production cells. In certain embodiments, a bank of seed BIICs is stored at −80° C. or in LN₂ vapor.

Baculoviruses are made of several essential proteins which are essential for the function and replication of the Baculovirus, such as replication proteins, envelope proteins and capsid proteins. The Baculovirus genome thus comprises several essential-gene nucleotide sequences encoding the essential proteins. As a non-limiting example, the genome can comprise an essential-gene region which comprises an essential-gene nucleotide sequence encoding an essential protein for the Baculovirus construct. The essential protein can comprise: GP64 baculovirus envelope protein, VP39 baculovirus capsid protein, or other similar essential proteins for the Baculovirus construct.

Baculovirus expression vectors (BEV) for producing AAV particles in insect cells, comprising but not limited to Spodoptera frugiperda (Sf9) cells, provide high titers of viral vector product. Recombinant baculovirus encoding the viral expression construct and payload construct initiates a productive infection of viral vector replicating cells. Infectious baculovirus particles released from the primary infection secondarily infect additional cells in the culture, exponentially infecting the entire cell culture population in a number of infection cycles that is a function of the initial multiplicity of infection, see Urabe, M. et al. J Virol. 2006 February; 80(4):1874-85, the content of which is incorporated herein by reference in its entirety as related to the production and use of BEVs and viral particles, insofar as it does not conflict with the present disclosure.

In certain embodiments, the production system of the present disclosure addresses baculovirus instability over multiple passages by utilizing a titerless infected-cells preservation and scale-up system. Small scale seed cultures of viral producing cells are transfected with viral expression constructs encoding the structural and/or non-structural components of the AAV particles. Baculovirus-infected viral producing cells are harvested into aliquots that may be cryopreserved in liquid nitrogen; the aliquots retain viability and infectivity for infection of large scale viral producing cell culture Wasilko D J et al. Protein Expr Purif. 2009 June; 65(2):122-32, the content of which is incorporated herein by reference in its entirety as related to the production and use of BEVs and viral particles, insofar as it does not conflict with the present disclosure.

A genetically stable baculovirus may be used to produce a source of the one or more of the components for producing AAV particles in invertebrate cells. In certain embodiments, defective baculovirus expression vectors may be maintained episomally in insect cells. In such an embodiment the corresponding bacmid vector is engineered with replication control elements, comprising but not limited to promoters, enhancers, and/or cell-cycle regulated replication elements.

In certain embodiments, baculoviruses may be engineered with a marker for recombination into the chitinase/cathepsin locus. The chia/v-cath locus is non-essential for propagating baculovirus in tissue culture, and the V-cath (EC 3.4.22.50) is a cysteine endoprotease that is most active on Arg-Arg dipeptide containing substrates. The Arg-Arg dipeptide is present in densovirus and parvovirus capsid structural proteins but infrequently occurs in dependovirus VP1.

In certain embodiments, stable viral producing cells permissive for baculovirus infection are engineered with at least one stable integrated copy of any of the elements necessary for AAV replication and vector production comprising, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

In certain embodiments, the Baculovirus expression vectors (BEV) are based on the AcMNPV baculovirus or BmNPV baculovirus BmNPV. In certain embodiments, a bacmid of the present disclosure is based on (i.e. engineered variant of) an AcMNPV bacmid such as bmon14272, vAce25ko or vAclef11KO.

In certain embodiments, the Baculovirus expression vectors (BEV) is a BEV in which the baculoviral v-cath gene has been partially or fully deleted (“v-cath deleted BEV”) or mutated. In certain embodiments, the BEVs lack the v-cath gene or comprise a mutationally inactivated version of the v-cath gene. In certain embodiments, the BEVs lack the v-cath gene. In certain embodiments, the BEVs comprise a mutationally inactivated version of the v-cath gene.

Viral production bacmids of the present disclosure can comprise deletion of certain baculoviral genes or loci.

In certain embodiments, baculoviral inoculum banks can be produced using small-scale shake flasks, such as 3 L or 5 L shake flasks. However, this process is generally limited in the maximum cell density of the BIIC cells which can be produced, and thus requires centrifugation to concentrate resulting cells into a workable concentration. This correspondingly limits the volume (i.e. quantity) of the baculoviral inoculum bank (˜600 mL) which can be produced and stored using this method. This process also presents sterility concerns due to open operation.

In certain embodiments, baculoviral inoculum banks can be produced using bioreactors, such as 20-50 L bioreactors. However, this process is also generally limited in the maximum cell density of the BIIC cells which can be produced, and thus requires significant processing through Tangential Flow Filtration (TFF) and/or centrifugation to concentrate resulting cells into a workable concentration (with 3 L of culture material being required to produce about 600 mL of concentrated BIIC formulation, corresponding with a 15-25% yield). This correspondingly limits the volume (i.e. quantity) of the baculoviral inoculum bank (˜3000 mL) which can be produced and stored using this method. This process also presents sterility concerns due to open operation.

In certain embodiments, perfusion technology can be used in the production of baculoviral inoculum banks. Perfusion systems are fluid circulation systems which use combinations of pumps, filters and screens to retain cells inside a bioreactor while continually removing cell waste products and replacing media depleted of nutrients by cell metabolism. In certain embodiments, the perfusion system is an alternating tangential flow (ATF) perfusion system (e.g. XCell ATF system). In certain embodiments, a perfusion system can be used in coordination with bioreactors to manage and cycle cell culture media within a bioreactor during the production of Baculovirus Infected Insect Cells (BIICs). In certain embodiments, a perfusion system can be used to support the production of high quality BIIC banks having an unexpectedly high cell density at large-scale. In certain embodiments, a perfusion system can be used to provide an infection-cell-to-product-cell yield of greater than 70% (e.g. 75-80%, 80-85%, 85-90%, 90-95% or 95-100%). In certain embodiments, a perfusion system can be used to perform a media switch within the bioreactor, such as the replacements of a cell culture media with a cryopreservation media which allows for BIIC cells to be frozen and preserved.

The present disclosure presents methods for producing a baculovirus infected insect cell (BIIC), e.g., expression BIICs and/or payload BIICs. In certain embodiments, the present disclosure presents methods for producing a baculovirus infected insect cell (BIIC) which comprises the following steps: (a) introducing a volume of cell culture medium into a bioreactor; (b) introducing at least one viral production cell (VPC) into the bioreactor and expanding the number of VPCs in the bioreactor to a target VPC cell density; (c) introduction at least one Baculoviral Expression Vector (BEV) into the bioreactor, wherein the BEV comprises an AAV viral expression construct or an AAV payload construct; (d) incubating the mixture of VPCs and BEVs in the bioreactor under conditions which allow at least one BEV to infect at least one VPC to produce a baculovirus infected insect cell (BIIC); (e) incubating the bioreactor under conditions which allow the number of BIICs in the bioreactor to reach a target BIIC cell density; and (f) harvesting the BIICs from the bioreactor. In certain embodiments, the bioreactor has a volume of at least 5 L, 10 L, 20 L, 50 L, 100 L, or 200 L. In certain embodiments, the volume of cell culture medium (i.e. working volume) in the bioreactor is at least 5 L, 10 L, 20 L, 50 L, 100 L, or 200 L.

In certain embodiments, the VPC density at BEV introduction is 1.0×10⁵-2.5×10⁵, 2.5×10⁵-5.0×10⁵, 5.0×10⁵-7.5×10⁵, 7.5×10⁵-0.0×10⁶, 1.0×10⁶-5.0×10⁶, 1.0×10⁶-2.0×10⁶, 1.5×10⁶-2.5×10⁶, 2.0×10⁶-3.0×10⁶, 2.5×10⁶-3.5×10⁶, 3.0×10⁶-4.0×10⁶, 3.5×10⁶-4.5×10⁶, 4.0×10⁶-5.0×10⁶, 4.5×10⁶-5.5×10⁶, 5.0×10⁶-1.0×10⁷, 5.0×10⁶-6.0×10⁶, 5.5×10⁶-6.5×10⁶, 6.0×10⁶-7.0×10⁶, 6.5×10⁶-7.5×10⁶, 7.0×10⁶-8.0×10⁶, 7.5×10⁶-8.5×10⁶, 8.0×10⁶-9.0×10⁶, 8.5×10⁶-9.5×10⁶, 9.0×10⁶-10×10⁷, 9.5×10⁶-1.5×10⁷, 1.0×10⁷-5.0×10⁷, or 5.0×10⁷-1.0×10⁸ cells/mL. In certain embodiments, the VPC density at BEV introduction is 5.0×10⁵, 6.0×10⁵, 7.0×10⁵, 8.0×10⁵, 9.0×10⁵, 1.0×10⁶, 1.5×10⁶, 2.0×10⁶, 2.5×10⁶, 3.0×10⁶, 3.5×10⁶, 4.0×10⁶, 4.5×10⁶, 5.0×10⁶, 5.5×10⁶, 6.0×10⁶, 6.5×10⁶, 7.0×10⁶, 7.5×10⁶, 8.0×10⁶, 8.5×10⁶, 9.0×10⁶, 9.5×10⁶, 1.0×10⁷, 1.5×10⁷, 2.0×10⁷, 2.5×10⁷, 3.0×10⁷, 4.0×10⁷, 5.0×10⁷, 6.0×10⁷, 7.0×10⁷, 8.0×10⁷, or 9.0×10⁷ cells/mL.

In certain embodiments, the target VPC cell density at BEV introduction is 1.5-4.0×10⁶ cells/mL. In certain embodiments, the target VPC cell density at BEV introduction is 2.0-3.5×10⁶ cells/mL.

In certain embodiments, the BEVs are introduced into the bioreactor at a target Multiplicity of Infection (MOI) of BEVs to VPCs. In certain embodiments, the BEV MOI is 0.0005-0.003, or more specifically 0.001-0.002.

In certain embodiments, the bioreactor can comprise a perfusion system for managing the cell culture medium within the bioreactor. In certain embodiments, the perfusion system is used in a fed-batch AAV production process. In certain embodiments, the perfusion system is an alternating tangential flow (ATF) perfusion system (e.g. XCell ATF system). In certain embodiments, the perfusion system replaces at least a portion of the culture medium in the bioreactor while retaining at least 90% of the VPCs and BIICS within the bioreactor. In certain embodiments, the perfusion system removes cell waste products from the cell culture medium within the bioreactor. In certain embodiments, the perfusion system replaces cell culture media which has been depleted of nutrients by cellular metabolism. In certain embodiments, the perfusion system replaces the cell culture media with a cryopreservation media which allows for BIIC cells to be frozen and preserved. In certain embodiments, the perfusion system replaces the cell culture media with a cell culture media supplemented with growth or production boosting factors to increase the quality and quantity of the AAV product.

In certain embodiments, the BIICs are harvested from the bioreactor at a specific BIIC cell density. In certain embodiments, the BIICs harvested from the bioreactor have a specific BIIC cell density. In certain embodiments, the BIIC cell density at harvesting is 6.0-18.0×10⁶ cells/mL, 8.0-16.5×10⁶ cells/mL, 10.0-16.5×10⁶ cells/mL.

In some embodiments, BIICs (expression BIICs, payload BIICs) are used to transfect viral production cells, e.g., Sf9 cells. In some embodiments, baculoviruses comprising bacmids such as BEVs (expressionBacs, payloadBacs) are used to transfect viral production cells, e.g., Sf9 cells.

### Other

In certain embodiments expression hosts comprise, but are not limited to, bacterial species within the genera Escherichia, Bacillus, Pseudomonas, or Salmonella.

In certain embodiments, a host cell which comprises AAV rep and cap genes stably integrated within the cell's chromosomes, may be used for AAV particle production. In a non-limiting example, a host cell which has stably integrated in its chromosome at least two copies of an AAV rep gene and AAV cap gene may be used to produce the AAV particle according to the methods and constructs described in U.S. Pat. No. 7,238,526, the content of which is incorporated herein by reference in its entirety as related to the production of viral particles, insofar as it does not conflict with the present disclosure.

In certain embodiments, the AAV particle can be produced in a host cell stably transformed with a molecule comprising the nucleic acid sequences which permit the regulated expression of a rare restriction enzyme in the host cell, as described in US20030092161 and EP1183380, the contents of which are each incorporated herein by reference in their entireties as related to the production of viral particles, insofar as they do not conflict with the present disclosure.

In certain embodiments, production methods and cell lines to produce the AAV particle may comprise, but are not limited to those taught in PCT/US1996/010245, PCT/US1997/015716, PCT/US1997/015691, PCT/US1998/019479, PCT/US1998/019463, PCT/US2000/000415, PCT/US2000/040872, PCT/US2004/016614, PCT/US2007/010055, PCT/US1999/005870, PCT/US2000/004755, U.S. patent application Ser. Nos. 08/549,489, 08/462,014, 09/659,203, 10/246,447, 10/465,302, U.S. Pat. Nos. 6,281,010, 6,270,996, 6,261,551, 5,756,283 (Assigned to NIH), U.S. Pat. Nos. 6,428,988, 6,274,354, 6,943,019, 6,482,634, (Assigned to NIH: U.S. Pat. Nos. 7,238,526, 6,475,769), U.S. Pat. No. 6,365,394 (Assigned to NIH), U.S. Pat. Nos. 7,491,508, 7,291,498, 7,022,519, 6,485,966, 6,953,690, 6,258,595, EP2018421, EP1064393, EP1163354, EP835321, EP931158, EP950111, EP1015619, EP1183380, EP2018421, EP1226264, EP1636370, EP1163354, EP1064393, US20030032613, US20020102714, US20030073232, US20030040101 (Assigned to NIH), US20060003451, US20020090717, US20030092161, US20070231303, US20060211115, US20090275107, US2007004042, US20030119191, US20020019050, the contents of which are each incorporated herein by reference in their entireties, insofar as they do not conflict with the present disclosure.

### Viral Production Systems

### Large-Scale Production

In certain embodiments, AAV particle production may be modified to increase the scale of production. Large scale viral production methods according to the present disclosure may comprise any of the processes or processing steps taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference by reference in their entirety.

Methods of increasing AAV particle production scale typically comprise increasing the number of viral production cells. In certain embodiments, viral production cells comprise adherent cells. To increase the scale of AAV particle production by adherent viral production cells, larger cell culture surfaces are required. In certain embodiments, large-scale production methods comprise the use of roller bottles to increase cell culture surfaces. Other cell culture substrates with increased surface areas are known in the art. Examples of additional adherent cell culture products with increased surface areas comprise, but are not limited to iCELLis (Pall Corp, Port Washington, N.Y.), CELLSTACK®, CELLCUBE® (Corning Corp., Corning, NY) and NUNC™ CELL FACTORY™ (Thermo Scientific, Waltham, Mass.) In certain embodiments, large-scale adherent cell surfaces may comprise from about 1,000 cm² to about 100,000 cm².

In certain embodiments, large-scale viral production methods of the present disclosure may comprise the use of suspension cell cultures. Suspension cell culture can allow for significantly increased numbers of cells. Typically, the number of adherent cells that can be grown on about 10-50 cm² of surface area can be grown in about 1 cm³ volume in suspension.

In certain embodiments, large-scale cell cultures may comprise from about 10⁷ to about 10⁹ cells, from about 10⁸ to about 10¹⁰ cells, from about 10⁹ to about 10¹² cells or at least 10¹² cells. In certain embodiments, large-scale cultures may produce from about 10⁹ to about 10¹², from about 10¹⁰ to about 10¹³, from about 10¹¹ to about 10¹⁴, from about 10¹² to about 10¹⁵ or at least 10¹⁵ AAV particles.

Transfection of replication cells in large-scale culture formats may be carried out according to any methods known in the art. For large-scale adherent cell cultures, transfection methods may comprise, but are not limited to the use of inorganic compounds (e.g. calcium phosphate,) organic compounds (e.g. polyethyleneimine (PEI)) or the use of non-chemical methods (e.g. electroporation). With cells grown in suspension, transfection methods may comprise, but are not limited to the use of inorganic compounds (e.g. calcium phosphate,) organic compounds (e.g. polyethyleneimine (PEI)) or the use of non-chemical methods (e.g. electroporation). In certain embodiments, transfection of large-scale suspension cultures may be carried out according to the section entitled “Transfection Procedure” described in Feng, L. et al., 2008. Biotechnol Appl Biochem. 50:121-32, the contents of which are herein incorporated by reference in their entirety. According to such embodiments, PEI-DNA complexes may be formed for introduction of plasmids to be transfected. In certain embodiments, cells being transfected with PEI-DNA complexes may be ‘shocked’ prior to transfection. This comprises lowering cell culture temperatures to 4° C. for a period of about 1 hour. In certain embodiments, cell cultures may be shocked for a period of from about 10 minutes to about 5 hours. In certain embodiments, cell cultures may be shocked at a temperature of from about 0° C. to about 20° C.

In certain embodiments, transfections may comprise one or more vectors for expression of an RNA effector molecule to reduce expression of nucleic acids from one or more payload construct. Such methods may enhance the production of AAV particles by reducing cellular resources wasted on expressing payload constructs. In certain embodiments, such methods may be carried according to those taught in US Publication No. US2014/0099666, the contents of which are herein incorporated by reference in their entirety.

### Bioreactors

In certain embodiments, cell culture bioreactors may be used for large scale production of AAV particles. In certain embodiments, bioreactors comprise stirred tank reactors. Such reactors generally comprise a vessel, typically cylindrical in shape, with a stirrer (e.g. impeller.) In certain embodiments, such bioreactor vessels may be placed within a water jacket to control vessel temperature and/or to minimize effects from ambient temperature changes.

Bioreactor vessel volume may range in size from about 500 ml to about 2 L, from about 1 L to about 5 L, from about 2.5 L to about 20 L, from about 10 L to about 50 L, from about 25 L to about 100 L, from about 75 L to about 500 L, from about 250 L to about 2,000 L, from about 1,000 L to about 10,000 L, from about 5,000 L to about 50,000 L or at least 50,000 L. Vessel bottoms may be rounded or flat. In certain embodiments, animal cell cultures may be maintained in bioreactors with rounded vessel bottoms.

In certain embodiments, bioreactor vessels may be warmed through the use of a thermocirculator. Thermocirculators pump heated water around water jackets. In certain embodiments, heated water may be pumped through pipes (e.g. coiled pipes) that are present within bioreactor vessels. In certain embodiments, warm air may be circulated around bioreactors, comprising, but not limited to air space directly above culture medium. Additionally, pH and CO₂ levels may be maintained to optimize cell viability.

In certain embodiments, bioreactors may comprise hollow-fiber reactors. Hollow-fiber bioreactors may support the culture of both anchorage dependent and anchorage independent cells. Further bioreactors may comprise, but are not limited to, packed-bed or fixed-bed bioreactors. Such bioreactors may comprise vessels with glass beads for adherent cell attachment. Further packed-bed reactors may comprise ceramic beads.

In certain embodiments, viral particles are produced through the use of a disposable bioreactor. In certain embodiments, bioreactors may comprise GE WAVE bioreactor, a GE Xcellerex Bioreactor, a Sartorius Biostat Bioreactor, a ThermoFisher Hyclone Bioreactor, or a Pall Allegro Bioreactor.

In certain embodiments, AAV particle production in cell bioreactor cultures may be carried out according to the methods or systems taught in U.S. Pat. Nos. 5,064,764, 6,194,191, 6,566,118, 8,137,948 or US Patent Application No. US2011/0229971, the contents of each of which are herein incorporated by reference in their entirety.

In certain embodiments, perfusion technology can be used in the production of viral particles. Perfusion systems are fluid circulation systems which use filters and screens to retain cells inside a bioreactor while continually removing cell waste products and media depleted of nutrients by cell metabolism. In certain embodiments, the perfusion system is an alternating tangential flow (ATF) perfusion system (e.g. XCell ATF system). In certain embodiments, a perfusion system can be used in coordination with bioreactors to manage and cycle cell culture media within a bioreactor during the production of viral particles, such as AAV viral particles. In certain embodiments, a perfusion system can be used to support the production of high quality AAV viral particles having an unexpectedly high cell density at large-scale. In certain embodiments, a perfusion system can be used to perform a media switch within the bioreactor, such as the replacement of a cell culture media with media supplemented with growth or production boosting factors to increase the quality and quantity of the AAV product.

It is advantageous to produce large batches of AAV particles in single production campaigns for gene therapy clinical development activities, as the large batches of therapeutic materials ensure clinical study consistency and minimize the therapeutic and statistical variability resulting from multiple smaller manufacturing campaigns. It is advantageous to produce large batches of AAV particles in single production campaigns for commercial product development activities, as the large batches of therapeutic materials minimize the variability resulting from multiple smaller manufacturing campaigns and corresponding complications in quality control and product analysis associated with small-batch production.

### Expansion of Viral Production Cell (VPC) Mixtures

In certain embodiments, an AAV particle or viral vector of the present disclosure may be produced in a viral production cell (VPC), such as an insect cell. Production cells can be sourced from a Cell Bank (CB) and are often stored in frozen cell banks.

In certain embodiments, a viral production cell from a Cell Bank is provided in frozen form. The vial of frozen cells is thawed, typically until ice crystal dissipate. In certain embodiments, the frozen cells are thawed at a temperature between 10-50° C., 15-40° C., 20-30° C., 25-50° C., 30-45° C., 35-40° C., or 37-39° C. In certain embodiments, the frozen viral production cells are thawed using a heated water bath.

In certain embodiments, a thawed CB cell mixture will have a cell density of 1.0×10⁴-1.0×10⁹ cells/mL. In certain embodiments, the thawed CB cell mixture has a cell density of 1.0×10⁴-2.5×10⁴ cells/mL, 2.5×10⁴-5.0×10⁴ cells/mL, 5.0×10⁴-7.5×10⁴ cells/mL, 7.5×10⁴-1.0×10⁵ cells/mL, 1.0×10⁵-2.5×10⁵ cells/mL, 2.5×10⁵-5.0×10⁵ cells/mL, 5.0×10⁵-7.5×10⁵ cells/mL, 7.5×10⁵-1.0×10⁶ cells/mL, 1.0×10⁶-2.5×10⁶ cells/mL, 2.5×10⁶-5.0×10⁶ cells/mL, 5.0×10⁶-7.5×10⁶ cells/mL, 7.5×10⁶-1.0×10⁷ cells/mL, 1.0×10⁷-2.5×10⁷ cells/mL, 2.5×10⁷-5.0×10⁷ cells/mL, 5.0×10⁷-7.5×10⁷ cells/mL, 7.5×10⁷-1.0×10⁸ cells/mL, 1.0×10⁸-2.5×10⁸ cells/mL, 2.5×10⁸-5.0×10⁸ cells/mL, 5.0×10⁸-7.5×10⁸ cells/mL, or 7.5×10⁸-1.0×10⁹ cells/mL.

In certain embodiments, the volume of the CB cell mixture is expanded. This process is commonly referred to as a Seed Train, Seed Expansion, or CB Cellular Expansion. Cellular/Seed expansion can comprise successive steps of seeding and expanding a cell mixture through multiple expansion steps using successively larger working volumes. In certain embodiments, cellular expansion can comprise one, two, three, four, five, six, seven, or more than seven expansion steps. In certain embodiments, the working volume in the cellular expansion can comprise one or more of the following working volumes or working volume ranges: 5 mL, 10 mL, 20 mL, 5-20 mL, 25 mL, 30 mL, 40 mL, 50 mL, 20-50 mL, 75 mL, 100 mL, 125 mL, 150 mL, 175 mL, 200 mL, 50-200 mL, 250 mL, 300 mL, 400 mL, 500 mL, 750 mL, 1000 mL, 250-1000 mL, 1250 mL, 1500 mL, 1750 mL, 2000 mL, 1000-2000 mL, 2250 mL, 2500 mL, 2750 mL, 3000 mL, 2000-3000 mL, 3500 mL, 4000 mL, 4500 mL, 5000 mL, 3000-5000 mL, 5.5 L, 6.0 L, 7.0 L, 8.0 L, 9.0 L, 10.0 L, and 5.0-10.0 L.

In certain embodiments, a volume of cells from a first expanded cell mixture can be used to seed a second, separate Seed Train/Seed Expansion (instead of using thawed CB cell mixture). This process is commonly referred to as rolling inoculum. In certain embodiments, rolling inoculum is used in a series of two or more (e.g. two, three, four or five) separate Seed Trains/Seed Expansions.

In certain embodiments, large-volume cellular expansion can comprise the use of a bioreactor, such as a GE WAVE bioreactor, a GE Xcellerex Bioreactor, a Sartorius Biostat Bioreactor, a ThermoFisher Hyclone Bioreactor, or a Pall Allegro Bioreactor.

In certain embodiments, the cell density within a working volume is expanded to a target output cell density. In certain embodiments, the output cell density of an expansion step is 1.0×10⁵-5.0×10⁵, 5.0×10⁵-1.0×10⁶, 1.0×10⁶-5.0×10⁶, 5.0×10⁶-1.0×10⁷, 1.0×10⁷-5.0×10⁷, 5.0×10⁷-1.0×10⁸, 5.0×10⁵, 6.0×10⁵, 7.0×10⁵, 8.0×10⁵, 9.0×10⁵, 1.0×10⁶, 2.0×10⁶, 3.0×10⁶, 4.0×10⁶, 5.0×10⁶, 6.0×10⁶, 7.0×10⁶, 8.0×10⁶, 9.0×10⁶, 1.0×10⁷, 2.0×10⁷, 3.0×10⁷, 4.0×10⁷, 5.0×10⁷, 6.0×10⁷, 7.0×10⁷, 8.0×10⁷, or 9.0×10⁷ cells/mL.

In certain embodiments, the output cell density of a working volume provides a seeding cell density for a larger, successive working volume. In certain embodiments, the seeding cell density of an expansion step is 1.0×10⁵-5.0×10⁵, 5.0×10⁵-1.0×10⁶, 1.0×10⁶-5.0×10⁶, 5.0×10⁶-1.0×10⁷, 1.0×10⁷-5.0×10⁷, 5.0×10⁷-1.0×10⁸, 5.0×10⁵, 6.0×10, 7.0×10⁵, 8.0×10⁵, 9.0×10⁵, 1.0×10⁶, 2.0×10⁶, 3.0×10⁶, 4.0×10⁶, 5.0×10⁶, 6.0×10⁶, 7.0×10⁶, 8.0×10⁶, 9.0×10⁶, 1.0×10⁷, 2.0×10⁷, 3.0×10⁷, 4.0×10⁷, 5.0×10⁷, 6.0×10⁷, 7.0×10⁷, 8.0×10⁷, or 9.0×10⁷ cells/mL.

In certain embodiments, cellular expansion can last for 1-50 days. Each cellular expansion step or the total cellular expansion can last for 1-10 days, 1-5 days, 1-3 days, 2-3 days, 2-4 days, 2-5 days, 2-6 days, 3-4 days, 3-5 days, 3-6 days, 3-8 days, 4-5 days, 4-6 days, 4-8 days, 5-6 days, or 5-8 days. In certain embodiments, each cellular expansion step or the total cellular expansion can last for 1-100 generations, 1-1000 generations, 100-1000 generation, 100 generations or more, or 1000 generation or more.

In certain embodiments, infected or transfected production cells can be expanded in the same manner as CB cell mixtures, as set forth in the present disclosure.

### Infection of Viral Production Cells

In certain embodiments, AAV particles of the present disclosure are produced in a viral production cell (VPC), such as an insect cell, by infecting the VPC with a viral vector which comprises an AAV expression construct and/or a viral vector which comprises an AAV payload construct. In certain embodiments, the VPC is infected with an Expression BEV, which comprises an AAV expression construct and a Payload BEV which comprises an AAV payload construct.

In certain embodiments, AAV particles are produced by infecting a VPC with a viral vector which comprises both an AAV expression construct and an AAV payload construct. In certain embodiments, the VPC is infected with a single BEV which comprises both an AAV expression construct and an AAV payload construct.

In certain embodiments, VPCs (such as insect cells) are infected using Infection BIICs in an infection process which comprises the following steps: (i) A collection of VPCs are seeded into a Production Bioreactor; (ii) The seeded VPCs can optionally be expanded to a target working volume and cell density; (iii) Infection BIICs which comprise Expression BEVs and Infection BIICs which comprise Payload BEVs are injected into the Production Bioreactor, resulting in infected viral production cells; and (iv) incubation of the infected viral production cells to produce AAV particles within the viral production cells.

In certain embodiments, the VPC density at infection is 1.0×10⁵-2.5×10⁵, 2.5×10⁵-5.0×10⁵, 5.0×10⁵-7.5×10⁵, 7.5×10⁵-1.0×10⁶, 1.0×10⁶-5.0×10⁶, 1.0×10⁶-2.0×10⁶, 1.5×10⁶-2.5×10⁶, 2.0×10⁶-3.0×10⁶, 2.5×10⁶-3.5×10⁶, 3.0×10⁶-3.4×10⁶, 3.0×10⁶-4.0×10⁶, 3.5×10⁶-4.5×10⁶, 4.0×10⁶-5.0×10⁶, 4.5×10⁶-5.5×10⁶, 5.0×10⁶-1.0×10⁷, 5.0×10⁶-6.0×10⁶, 5.5×10⁶-6.5×10⁶, 6.0×10⁶-7.0×10⁶, 6.5×10⁶-7.5×10⁶, 7.0×10⁶-8.0×10⁶, 7.5×10⁶-8.5×10⁶, 8.0×10⁶-9.0×10⁶, 8.5×10⁶-9.5×10⁶, 9.0×10⁶-1.0×10⁷, 9.5×10⁶-1.5×10⁷, 1.0×10⁷-5.0×10⁷, or 5.0×10⁷-1.0×10⁸ cells/mL. In certain embodiments, the VPC density at infection is 5.0×10⁵, 6.0×10⁵, 7.0×10⁵, 8.0×10⁵, 9.0×10⁵, 1.0×10⁶, 1.5×10⁶, 2.0×10⁶, 2.5×10⁶, 3.0×10⁶, 3.1×10⁶, 3.2×10⁶, 3.3×10⁶, 3.4×10⁶, 3.5×10⁶, 4.0×10⁶, 4.5×10⁶, 5.0×10⁶, 5.5×10⁶, 6.0×10⁶, 6.5×10⁶, 7.0×10⁶, 7.5×10⁶, 8.0×10⁶, 8.5×10⁶, 9.0×10⁶, 9.5×10⁶, 1.0×10⁷, 1.5×10⁷, 2.0×10⁷, 2.5×10⁷, 3.0×10⁷, 4.0×10⁷, 5.0×10⁷, 6.0×10⁷, 7.0×10⁷, 8.0×10⁷, or 9.0×10⁷ cells/mL. In certain embodiments, the VPC density at infection is 2.0-3.5×10⁶ cells/mL. In certain embodiments, the VPC density at infection is 3.5-5.0×10⁶ cells/mL. In certain embodiments, the VPC density at infection is 5.0-7.5×10⁶ cells/mL. In certain embodiments, the VPC density at infection is 5.0-10.0×10⁶ cells/mL.

In certain embodiments, the VPC density at infection is 1.0×10⁵-2.5×10¹, 2.5×10⁵-5.0×10⁵, 5.0×10⁵-7.5×10⁵, 7.5×10⁵-1.0×10⁶, 1.0×10⁶-5.0×10⁶, 1.0×10⁶-2.0×10⁶, 1.5×10⁶-2.5×10⁶, 2.0×10⁶-3.0×10⁶, 2.5×10⁶-3.5×10⁶, 3.0×10⁶-3.4×10⁶, 3.0×10⁶-4.0×10⁶, 3.5×10⁶-4.5×10⁶, 4.0×10⁶-5.0×10⁶, 4.5×10⁶-5.5×10⁶, 5.0×10⁶-1.0×10⁷, 5.0×10⁶-6.0×10⁶, 5.5×10⁶-6.5×10⁶, 6.0×10⁶-7.0×10⁶, 6.5×10⁶-7.5×10⁶, 7.0×10⁶-8.0×10⁶, 7.5×10⁶-8.5×10⁶, 8.0×10⁶-9.0×10⁶, 8.5×10⁶-9.5×10⁶, 9.0×10⁶-1.0×10⁷, 9.5×10⁶-1.5×10⁷, 1.0×10⁷-5.0×10⁷, or 5.0×10⁷-1.0×10⁸ cells/mL. In certain embodiments, the VPC density at infection is 5.0×10⁵, 6.0×10, 7.0×10⁵, 8.0×10⁵, 9.0×10⁵, 1.0×10⁶, 1.5×10⁶, 2.0×10⁶, 2.5×10⁶, 3.0×10⁶, 3.1×10⁶, 3.2×10⁶, 3.3×10⁶, 3.4×10⁶, 3.5×10⁶, 4.0×10⁶, 4.5×10⁶, 5.0×10⁶, 5.5×10⁶, 6.0×10⁶, 6.5×10⁶, 7.0×10⁶, 7.5×10⁶, 8.0×10⁶, 8.5×10⁶, 9.0×10⁶, 9.5×10⁶, 1.0×10⁷, 1.5×10⁷, 2.0×10⁷, 2.5×10⁷, 3.0×10⁷, 4.0×10⁷, 5.0×10⁷, 6.0×10⁷, 7.0×10⁷, 8.0×10⁷, or 9.0×10⁷ cells/mL. In certain embodiments, the VPC density at infection is 2.0-3.5×10⁶ cells/mL. In certain embodiments, the VPC density at infection is 3.5-5.0×10⁶ cells/mL. In certain embodiments, the VPC density at infection is 5.0-7.5×10⁶ cells/mL. In certain embodiments, the VPC density at infection is 5.0-10.0×10⁶ cells/mL.

In certain embodiments, Infection BIICs are combined with the VPCs in target ratios of VPC-to-BIIC. In certain embodiments, the VPC-to-BIIC infection ratio (volume to volume) is between 1.0×10³-3.0×10³, 2.0×10³-4.0×10³, 3.0×10³-5.0×10³, 4.0×10³-6.0×10³, 5.0×10³-7.0×10³, 6.0×10³-8.0×10³, 7.0×10³-9.0×10³, 8.0×10³-1.0×10⁴, 9.0×10³-1.1×10⁴, 1.0×10³-5.0×10³, 5.0×10³-1.0×10⁴, 1.0×10⁴-3.0×10⁴, 2.0×10⁴-4.0×10⁴, 3.0×10⁴-5.0×10⁴, 4.0×10⁴-6.0×10⁴, 5.0×10⁴-7.0×10⁴, 6.0×10⁴-8.0×10⁴, 7.0×10⁴-9.0×10⁴, 8.0×10⁴-1 0.0×10⁵, 9.0×10⁴-1.1×10⁵, 1.0×10⁴-5.0×10⁴, 5.0×10⁴-1.0×10⁵, 1.0×10⁵-3.0×10⁵, 2.0×10⁵-4.0×10⁵, 3.0×10⁵-5.0×10⁵, 4.0×10⁵-6.0×10⁵, 5.0×10⁵-7.0×10⁵, 6.0×10⁵-8.0×10⁵, 7.0×10⁵-9.0×10⁵, 8.0×10⁵-1.0×10⁶, 9.0×10⁵-1.1×10⁶, 1.0×10⁵-5.0×10⁵, 5.0×10⁵-1.0×10⁶, 1.0×10⁶-3.0×10⁶, 2.0×10⁶-4.0×10⁶, 3.0×10⁶-5.0×10⁶, 4.0×10⁶-6.0×10⁶, 5.0×10⁶-7.0×10⁶, 6.0×10⁶-8.0×10⁶, 7.0×10⁶-9.0×10⁶, 8.0×10⁶-1.0×10⁷, 9.0×10⁶-1.1×10⁷, 1.0×10⁶-5.0×10⁶, or 5.0×10⁶-1.0×10⁷ (VPC volume to BIIC volume). In certain embodiments, the VPC-to-BIIC infection ratio (volume to volume) is about 1.0×10³, about 1.5×10³, about 2.0×10³, about 2.5×10³, about 3.0×10³, about 3.5×10³, about 4.0×10³, about 4.5×10³, about 5.0×10³, about 5.5×10³, about 6.0×10³, about 6.5×10³, about 7.0×10³, about 7.5×10³, about 8.0×10³, about 8.5×10³, about 9.0×10³, about 9.5×10³, about 1.0×10⁴, about 1.5×10⁴, about 2.0×10⁴, about 2.5×10⁴, about 3.0×10⁴, about 3.5×10⁴, about 4.0×10⁴, about 4.5×10⁴, about 5.0×10⁴, about 5.5×10⁴, about 6.0×10⁴, about 6.5×10⁴, about 7.0×10⁴, about 7.5×10⁴, about 8.0×10⁴, about 8.5×10⁴, about 9.0×10⁴, about 9.5×10⁴, about 1.0×10⁵, about 1.5×10⁵, about 2.0×10⁵, about 2.5×10⁵, about 3.0×10⁵, about 3.5×10⁵, about 4.0×10⁵, about 4.5×10⁵, about 5.0×10⁵, about 5.5×10⁵, about 6.0×10⁵, about 6.5×10⁵, about 7.0×10⁵, about 7.5×10⁵, about 8.0×10⁵, about 8.5×10⁵, about 9.0×10⁵, about 9.5×10⁵, about 1.0×10⁶, about 1.5×10⁶, about 2.0×10⁶, about 2.5×10⁶, about 3.0×10⁶, about 3.5×10⁶, about 4.0×10⁶, about 4.5×10⁶, about 5.0×10⁶, about 5.5×10⁶, about 6.0×10⁶, about 6.5×10⁶, about 7.0×10⁶, about 7.5×10⁶, about 8.0×10⁶, about 8.5×10⁶, about 9.0×10⁶, or about 9.5×10⁶ (VPC volume to BIIC volume). In certain embodiments, the VPC-to-BIIC infection ratio (cell to cell) is between 1.0×10³-3.0×10³, 2.0×10³-4.0×10³, 3.0×10³-5.0×10³, 4.0×10³-6.0×10³, 5.0×10³-7.0×10³, 6.0×10³-8.0×10³, 7.0×10³-9.0×10³, 8.0×10³-1.0×10⁴, 9.0×10³-1.1×10⁴, 1.0×10³-5.0×10³, 5.0×10³-1.0×10⁴, 1.0×10⁴-3.0×10⁴, 2.0×10⁴-4.0×10⁴, 3.0×10⁴-5.0×10⁴, 4.0×10⁴-6.0×10⁴, 5.0×10⁴-7.0×10⁴, 6.0×10⁴-8.0×10⁴, 7.0×10⁴-9.0×10⁴, 8.0×10⁴-1.0×10⁵, 9.0×10⁴-1.1×10⁵, 1.0×10⁴-5.0×10⁴, 5.0×10⁴-1.0×10⁵, 1.0×10⁵-3.0×10⁵, 2.0×10⁵-4.0×10⁵, 3.0×10⁵-5.0×10⁵, 4.0×10⁵-6.0×10⁵, 5.0×10⁵-7.0×10⁵, 6.0×10⁵-8.0×10⁵, 7.0×10⁵-9.0×10⁵, 8.0×10⁵-1.0×10⁶, 9.0×10⁵-1.1×10⁶, 1.0×10⁵-5.0×10⁵, 5.0×10⁵-1.0×10⁶, 1.0×10⁶-3.0×10⁶, 2.0×10⁶-4.0×10⁶, 3.0×10⁶-5.0×10⁶, 4.0×10⁶-6.0×10⁶, 5.0×10⁶-7.0×10⁶, 6.0×10⁶-8.0×10⁶, 7.0×10⁶-9.0×10⁶, 8.0×10⁶-1.0×10⁷, 9.0×10⁶-1.1×10⁷, 1.0×10⁶-5.0×10⁶, or 5.0×10⁶-1.0×10⁷ (VPC cells to BIIC cells). In certain embodiments, the VPC-to-BIIC infection ratio (cell to cell) is about 1.0×10³, about 1.5×10³, about 2.0×10³, about 2.5×10³, about 3.0×10³, about 3.5×10³, about 4.0×10³, about 4.5×10³, about 5.0×10³, about 5.5×10³, about 6.0×10³, about 6.5×10³, about 7.0×10³, about 7.5×10³, about 8.0×10³, about 8.5×10³, about 9.0×10³, about 9.5×10³, about 1.0×10⁴, about 1.5×10⁴, about 2.0×10⁴, about 2.5×10⁴, about 3.0×10⁴, about 3.5×10⁴, about 4.0×10⁴, about 4.5×10⁴, about 5.0×10⁴, about 5.5×10⁴, about 6.0×10⁴, about 6.5×10⁴, about 7.0×10⁴, about 7.5×10⁴, about 8.0×10⁴, about 8.5×10⁴, about 9.0×10⁴, about 9.5×10⁴, about 1.0×10⁵, about 1.5×10⁵, about 2.0×10⁵, about 2.5×10⁵, about 3.0×10⁵, about 3.5×10⁵, about 4.0×10⁵, about 4.5×10⁵, about 5.0×10⁵, about 5.5×10⁵, about 6.0×10⁵, about 6.5×10⁵, about 7.0×10⁵, about 7.5×10⁵, about 8.0×10⁵, about 8.5×10⁵, about 9.0×10⁵, about 9.5×10⁵, about 1.0×10⁶, about 1.5×10⁶, about 2.0×10⁶, about 2.5×10⁶, about 3.0×10⁶, about 3.5×10⁶, about 4.0×10⁶, about 4.5×10⁶, about 5.0×10⁶, about 5.5×10⁶, about 6.0×10⁶, about 6.5×10⁶, about 7.0×10⁶, about 7.5×10⁶, about 8.0×10⁶, about 8.5×10⁶, about 9.0×10⁶, or about 9.5×10⁶ (VPC cells to BIIC cells).

In certain embodiments, Infection BIICs which comprise Expression BEVs are combined with the VPCs in target ratios of VPC-to-expressionBIIC. In certain embodiments, the VPC-to-expressionBIIC infection ratio (volume to volume) is between 1.0×10³-3.0×10³, 2.0×10³-4.0×10³, 3.0×10³-5.0×10³, 4.0×10³-6.0×10³, 5.0×10³-7.0×10³, 6.0×10³-8.0×10³, 7.0×10³-9.0×10³, 8.0×10³-1.0×10⁴, 9.0×10³-1.1×10⁴, 1.0×10³-5.0×10³, 5.0×10³-1.0×10⁴, 1.0×10⁴-3.0×10⁴, 2.0×10⁴-4.0×10⁴, 3.0×10⁴-5.0×10⁴, 4.0×10⁴-6.0×10⁴, 5.0×10⁴-7.0×10⁴, 6.0×10⁴-8.0×10⁴, 7.0×10⁴-9.0×10⁴, 8.0×10⁴-1.0×10⁵, 9.0×10⁴-1.1×10⁵, 1.0×10⁴-5.0×10⁴, 5.0×10⁴-1.0×10⁵, 1.0×10⁵-3.0×10⁵, 2.0×10⁵-4.0×10⁵, 3.0×10⁵-5.0×10⁵, 4.0×10⁵-6.0×10⁵, 5.0×10⁵-7.0×10⁵, 6.0×10⁵-8.0×10⁵, 7.0×10⁵-9.0×10⁵, 8.0×10⁵-1.0×10⁶, 9.0×10⁵-1.1×10⁶, 1.0×10⁵-5.0×10⁵, 5.0×10⁵-1.0×10⁶, 1.0×10⁶-3.0×10⁶, 2.0×10⁶-4.0×10⁶, 3.0×10⁶-5.0×10⁶, 4.0×10⁶-6.0×10⁶, 5.0×10⁶-7.0×10⁶, 6.0×10⁶-8.0×10⁶, 7.0×10⁶-9.0×10⁶, 8.0×10⁶-1.0×10⁷, 9.0×10⁶-1.1×10⁷, 1.0×10⁶-5.0×10⁶, or 5.0×10⁶-1.0×10⁷ (VPC volume to expressionBIIC volume). In certain embodiments, the VPC-to-expressionBIIC infection ratio (volume to volume) is about 1.0×10³, about 1.5×10³, about 2.0×10³, about 2.5×10³, about 3.0×10³, about 3.5×10³, about 4.0×10³, about 4.5×10³, about 5.0×10³, about 5.5×10³, about 6.0×10³, about 6.5×10³, about 7.0×10³, about 7.5×10³, about 8.0×10³, about 8.5×10³, about 9.0×10³, about 9.5×10³, about 1.0×10⁴, about 1.5×10⁴, about 2.0×10⁴, about 2.5×10⁴, about 3.0×10⁴, about 3.5×10⁴, about 4.0×10⁴, about 4.5×10⁴, about 5.0×10⁴, about 5.5×10⁴, about 6.0×10⁴, about 6.5×10⁴, about 7.0×10⁴, about 7.5×10⁴, about 8.0×10⁴, about 8.5×10⁴, about 9.0×10⁴, about 9.5×10⁴, about 1.0×10⁵, about 1.5×10⁵, about 2.0×10⁵, about 2.5×10⁵, about 3.0×10⁵, about 3.5×10⁵, about 4.0×10⁵, about 4.5×10⁵, about 5.0×10⁵, about 5.5×10⁵, about 6.0×10⁵, about 6.5×10⁵, about 7.0×10⁵, about 7.5×10⁵, about 8.0×10⁵, about 8.5×10⁵, about 9.0×10⁵, about 9.5×10⁵, about 1.0×10⁶, about 1.5×10⁶, about 2.0×10⁶, about 2.5×10⁶, about 3.0×10⁶, about 3.5×10⁶, about 4.0×10⁶, about 4.5×10⁶, about 5.0×10⁶, about 5.5×10⁶, about 6.0×10⁶, about 6.5×10⁶, about 7.0×10⁶, about 7.5×10⁶, about 8.0×10⁶, about 8.5×10⁶, about 9.0×10⁶, or about 9.5×10⁶ (VPC volume to expressionBIIC volume). In certain embodiments, the VPC-to-expressionBIIC infection ratio (cell to cell) is between 1.0×10³-3.0×10³, 2.0×10³-4.0×10³, 3.0×10³-5.0×10³, 4.0×10³-6.0×10³, 5.0×10³-7.0×10³, 6.0×10³-8.0×10³, 7.0×10³-9.0×10³, 8.0×10³-1.0×10⁴, 9.0×10³-1.1×10⁴, 1.0×10³-5.0×10³, 5.0×10³-1.0×10⁴, 1.0×10⁴-3.0×10⁴, 2.0×10⁴-4.0×10⁴, 3.0×10⁴-5.0×10⁴, 4.0×10⁴-6.0×10⁴, 5.0×10⁴-7.0×10⁴, 6.0×10⁴-8.0×10⁴, 7.0×10⁴-9.0×10⁴, 8.0×10⁴-1.0×10⁵, 9.0×10⁴-1.1×10⁵, 1.0×10⁴-5.0×10⁴, 5.0×10⁴-1.0×10⁵, 1.0×10⁵-3.0×10⁵, 2.0×10⁵-4.0×10⁵, 3.0×10⁵-5.0×10⁵, 4.0×10⁵-6.0×10⁵, 5.0×10⁵-7.0×10⁵, 6.0×10⁵-8.0×10⁵, 7.0×10⁵-9.0×10⁵, 8.0×10⁵-1.0×10⁶, 9.0×10⁵-1.1×10⁶, 1.0×10⁵-5.0×10⁵, 5.0×10⁵-1.0×10⁶, 1.0×10⁶-3.0×10⁶, 2.0×10⁶-4.0×10⁶, 3.0×10⁶-5.0×10⁶, 4.0×10⁶-6.0×10⁶, 5.0×10⁶-7.0×10⁶, 6.0×10⁶-8.0×10⁶, 7.0×10⁶-9.0×10⁶, 8.0×10⁶-1.0×10⁷, 9.0×10⁶-1.1×10⁶, 1.0×10⁶-5.0×10⁶, or 5.0×10⁶-1.0×10⁷ (VPC cells to expressionBIIC cells). In certain embodiments, the VPC-to-expressionBIIC infection ratio (cell to cell) is about 1.0×10³, about 1.5×10³, about 2.0×10³, about 2.5×10³, about 3.0×10³, about 3.5×10³, about 4.0×10³, about 4.5×10³, about 5.0×10³, about 5.5×10³, about 6.0×10³, about 6.5×10³, about 7.0×10³, about 7.5×10³, about 8.0×10³, about 8.5×10³, about 9.0×10³, about 9.5×10³, about 1.0×10⁴, about 1.5×10⁴, about 2.0×10⁴, about 2.5×10⁴, about 3.0×10⁴, about 3.5×10⁴, about 4.0×10⁴, about 4.5×10⁴, about 5.0×10⁴, about 5.5×10⁴, about 6.0×10⁴, about 6.5×10⁴, about 7.0×10⁴, about 7.5×10⁴, about 8.0×10⁴, about 8.5×10⁴, about 9.0×10⁴, about 9.5×10⁴, about 1.0×10⁵, about 1.5×10⁵, about 2.0×10⁵, about 2.5×10⁵, about 3.0×10⁵, about 3.5×10⁵, about 4.0×10⁵, about 4.5×10⁵, about 5.0×10⁵, about 5.5×10⁵, about 6.0×10⁵, about 6.5×10⁵, about 7.0×10⁵, about 7.5×10⁵, about 8.0×10⁵, about 8.5×10⁵, about 9.0×10⁵, about 9.5×10⁵, about 1.0×10⁶, about 1.5×10⁶, about 2.0×10⁶, about 2.5×10⁶, about 3.0×10⁶, about 3.5×10⁶, about 4.0×10⁶, about 4.5×10⁶, about 5.0×10⁶, about 5.5×10⁶, about 6.0×10⁶, about 6.5×10⁶, about 7.0×10⁶, about 7.5×10⁶, about 8.0×10⁶, about 8.5×10⁶, about 9.0×10⁶, or about 9.5×10⁶ (VPC cells to expressionBIIC cells).

In certain embodiments, Infection BIICs which comprise Payload BEVs are combined with the VPCs in target ratios of VPC-to-payloadBIIC. In certain embodiments, the VPC-to-payloadBIIC infection ratio (volume to volume) is between 1.0×10³-3.0×10³, 2.0×10³-4.0×10³, 3.0×10³-5.0×10³, 4.0×10³-6.0×10³, 5.0×10³-7.0×10³, 6.0×10³-8.0×10³, 7.0×10³-9.0×10³, 8.0×10³-1.0×10⁴, 9.0×10³-1.1×10⁴, 1.0×10³-5.0×10³, 5.0×10³-1.0×10⁴, 1.0×10⁴-3.0×10⁴, 2.0×10⁴-4.0×10⁴, 3.0×10⁴-5.0×10⁴, 4.0×10⁴-6.0×10⁴, 5.0×10⁴-7.0×10⁴, 6.0×10⁴-8.0×10⁴, 7.0×10⁴-9.0×10⁴, 8.0×10⁴-1.0×10⁵, 9.0×10⁴-1.1×10⁵, 1.0×10⁴-5.0×10⁴, 5.0×10⁴-1.0×10⁵, 1.0×10⁵-3.0×10⁵, 2.0×10⁵-4.0×10⁵, 3.0×10⁵-5.0×10⁵, 4.0×10⁵-6.0×10⁵, 5.0×10⁵-7.0×10⁵, 6.0×10⁵-8.0×10⁵, 7.0×10⁵-9.0×10⁵, 8.0×10⁵-1.0×10⁶, 9.0×10⁵-1.1×10⁶, 1.0×10⁵-5.0×10⁵, 5.0×10⁵-1.0×10⁶, 1.0×10⁶-3.0×10⁶, 2.0×10⁶-4.0×10⁶, 3.0×10⁶-5.0×10⁶, 4.0×10⁶-6.0×10⁶, 5.0×10⁶-7.0×10⁶, 6.0×10⁶-8.0×10⁶, 7.0×10⁶-9.0×10⁶, 8.0×10⁶-1.0×10⁷, 9.0×10⁶-1.1×10⁷, 1.0×10⁶-5.0×10⁶, or 5.0×10⁶-1.0×10⁷ (VPC volume to payloadBIIC volume). In certain embodiments, the VPC-to-payloadBIIC infection ratio (volume to volume) is about 1.0×10³, about 1.5×10³, about 2.0×10³, about 2.5×10³, about 3.0×10³, about 3.5×10³, about 4.0×10³, about 4.5×10³, about 5.0×10³, about 5.5×10³, about 6.0×10³, about 6.5×10³, about 7.0×10³, about 7.5×10³, about 8.0×10³, about 8.5×10³, about 9.0×10³, about 9.5×10³, about 1.0×10⁴, about 1.5×10⁴, about 2.0×10⁴, about 2.5×10⁴, about 3.0×10⁴, about 3.5×10⁴, about 4.0×10⁴, about 4.5×10⁴, about 5.0×10⁴, about 5.5×10⁴, about 6.0×10⁴, about 6.5×10⁴, about 7.0×10⁴, about 7.5×10⁴, about 8.0×10⁴, about 8.5×10⁴, about 9.0×10⁴, about 9.5×10⁴, about 1.0×10⁵, about 1.5×10⁵, about 2.0×10⁵, about 2.5×10⁵, about 3.0×10⁵, about 3.5×10⁵, about 4.0×10⁵, about 4.5×10⁵, about 5.0×10⁵, about 5.5×10⁵, about 6.0×10⁵, about 6.5×10⁵, about 7.0×10⁵, about 7.5×10⁵, about 8.0×10⁵, about 8.5×10⁵, about 9.0×10⁵, about 9.5×10⁵, about 1.0×10⁶, about 1.5×10⁶, about 2.0×10⁶, about 2.5×10⁶, about 3.0×10⁶, about 3.5×10⁶, about 4.0×10⁶, about 4.5×10⁶, about 5.0×10⁶, about 5.5×10⁶, about 6.0×10⁶, about 6.5×10⁶, about 7.0×10⁶, about 7.5×10⁶, about 8.0×10⁶, about 8.5×10⁶, about 9.0×10⁶, or about 9.5×10⁶ (VPC volume to payloadBIIC volume). In certain embodiments, the VPC-to-payloadBIIC infection ratio (cell to cell) is between 1.0×10³-3.0×10³, 2.0×10³-4.0×10³, 3.0×10-5.0×10³, 4.0×10³-6.0×10³, 5.0×10³-7.0×10³, 6.0×10³-8.0×10³, 7.0×10³-9.0×10³, 8.0×10³-1.0×10⁴, 9.0×10³-1.1×10⁴, 1.1×10³-5.0×10³, 5.0×10³-1.0×10⁴, 1.0×10⁴-3.0×10⁴, 2.0×10⁴-4.0×10⁴, 3.0×10⁴-5.0×10⁴, 4.0×10⁴-6.0×10⁴, 5.0×10⁴-7.0×10⁴, 6.0×10⁴-8.0×10⁴, 7.0×10⁴-9.0×10⁴, 8.0×10⁴-1.0×10⁵, 9.0×10⁴-1.1×10⁵, 1.0×10⁴-5.0×10⁴, 5.0×10⁴-1.0×10⁵, 1.0×10⁵-3.0×10⁵, 2.0×10⁵-4.0×10⁵, 3.0×10⁵-5.0×10⁵, 4.0×10⁵-6.0×10⁵, 5.0×10⁵-7.0×10⁵, 6.0×10⁵-8.0×10⁵, 7.0×10⁵-9.0×10⁵, 8.0×10⁵-1.0×10⁶, 9.0×10⁵-1.1×10⁶, 1.0×10⁵-5.0×10⁵, 5.0×10-1.0×10⁶, 1.0×10⁶-3.0×10⁶, 2.0×10⁶-4.0×10⁶, 3.0×10⁶-5.0×10⁶, 4.0×10⁶-6.0×10⁶, 5.0×10⁶-7.0×10⁶, 6.0×10⁶-8.0×10⁶, 7.0×10⁶-9.0×10⁶, 8.0×10⁶-1.0×10⁷, 9.0×10⁶-1.1×10⁷, 1.0×10⁶-5.0×10⁶, or 5.0×10⁶-1.0×10⁷ (VPC cells to payloadBIIC cells). In certain embodiments, the VPC-to-payloadBIIC infection ratio (cell to cell) is about 1.0×10³, about 1.5×10³, about 2.0×10³, about 2.5×10³, about 3.0×10³, about 3.5×10³, about 4.0×10³, about 4.5×10³, about 5.0×10³, about 5.5×10³, about 6.0×10³, about 6.5×10³, about 7.0×10³, about 7.5×10³, about 8.0×10³, about 8.5×10³, about 9.0×10³, about 9.5×10³, about 1.0×10⁴, about 1.5×10⁴, about 2.0×10⁴, about 2.5×10⁴, about 3.0×10⁴, about 3.5×10⁴, about 4.0×10⁴, about 4.5×10⁴, about 5.0×10⁴, about 5.5×10⁴, about 6.0×10⁴, about 6.5×10⁴, about 7.0×10⁴, about 7.5×10⁴, about 8.0×10⁴, about 8.5×10⁴, about 9.0×10⁴, about 9.5×10⁴, about 1.0×10⁵, about 1.5×10⁵, about 2.0×10⁵, about 2.5×10⁵, about 3.0×10⁵, about 3.5×10⁵, about 4.0×10⁵, about 4.5×10⁵, about 5.0×10⁵, about 5.5×10⁵, about 6.0×10⁵, about 6.5×10⁵, about 7.0×10⁵, about 7.5×10⁵, about 8.0×10⁵, about 8.5×10⁵, about 9.0×10⁵, about 9.5×10⁵, about 1.0×10⁶, about 1.5×10⁶, about 2.0×10⁶, about 2.5×10⁶, about 3.0×10⁶, about 3.5×10⁶, about 4.0×10⁶, about 4.5×10⁶, about 5.0×10⁶, about 5.5×10⁶, about 6.0×10⁶, about 6.5×10⁶, about 7.0×10⁶, about 7.5×10⁶, about 8.0×10⁶, about 8.5×10⁶, about 9.0×10⁶, or about 9.5×10⁶ (VPC cells to payloadBIIC cells).

In certain embodiments, Infection BIICs which comprise Expression BEVs and Infection BIICs which comprise Payload BEVs are combined with the VPCs in target expressionBIIC-to-payloadBIIC ratios. In certain embodiments, the ratio of expressionBIICs to payloadBIICs is 10:1, 9:1, 8:1, 7:1, 6:1, 5:1, 4.5:1, 4:1, 3.5:1, 3:1, 2.5:1, 2:1, 1.5:1, 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:9, or 1:10. In certain embodiments, the ratio of expressionBIICs to payloadBIICs is between 6.5-7.5:1, 6-7:1, 5.5-6.5:1, 5-6:1, 4.5-5.5:1, 4-5:1, 3.5-4.5:1, 3-4:1, 2.5-3.5:1, 2-3:1, 1.5-2.5:1, 1-2:1, 1-1.5:1, 1:1-1.5, 1:1-2, 1:1.5-2.5, 1:2-3, 1:2.5-3.5, 1:3-4, 1:3.5-4.5, 1:4-5, 1:4.5-5.5, 1:5-6, 1:5.5-6.5, 1:6-7, or 1:6.5-7.5.

In certain embodiments, infected Viral Production Cells are incubated under a certain Dissolved Oxygen (DO) Content (DO %). In certain embodiments, infected Viral Production Cells are incubated under a DO % between 10%-50%, 20%-40%, 10%-20%, 15%-25%, 20%-30%, 25%-35%, 30%-40%, 35%-45%, 40%-50%, 10%-15%, 15%-20%, 20%-25%, 25%-30%, 30%-35%, 35%-40%, 40%-45%, or 45%-50%. In certain embodiments, infected Viral Production Cells are incubated under a DO % of about 10%, about 15%, about 20%, about 25%, about 30%, about 35%, about 40%, about 45%, about 50%. In certain embodiments, infected Viral Production Cells are incubated under a DO % between 20%-30% or about 25%. In certain embodiments, infected Viral Production Cells are incubated under a DO % between 25%-35% or about 30%. In certain embodiments, infected Viral Production Cells are incubated under a DO % between 30%-40% or about 35%. In certain embodiments, infected Viral Production Cells are incubated under a DO % between 35%-45% or about 40%.

### Cell Lysis

Cells of the present disclosure, comprising, but not limited to viral production cells, may be subjected to cell lysis according to any methods known in the art. Cell lysis may be carried out to obtain one or more agents (e.g. viral particles) present within any cells of the disclosure. In certain embodiments, a bulk harvest of AAV particles and viral production cells is subjected to cell lysis according to the present disclosure.

In certain embodiments, cell lysis may be carried out according to any of the methods or systems presented in U.S. Pat. Nos. 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935, 7,968,333, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety.

Cell lysis methods and systems may be chemical or mechanical. Chemical cell lysis typically comprises contacting one or more cells with one or more chemical lysis agent under chemical lysis conditions. Mechanical lysis typically comprises subjecting one or more cells to cell lysis carried out by mechanical force. Lysis can also be completed by allowing the cells to degrade after reaching ˜0% viability.

In certain embodiments, chemical lysis may be used to lyse cells. As used herein, the term “chemical lysis agent” refers to any agent that may aid in the disruption of a cell. In certain embodiments, lysis agents are introduced in solutions, termed lysis solutions or lysis buffers. As used herein, the term “chemical lysis solution” refers to a solution (typically aqueous) comprising one or more lysis agent. In addition to lysis agents, lysis solutions may comprise one or more buffering agents, solubilizing agents, surfactants, preservatives, cryoprotectants, enzymes, enzyme inhibitors and/or chelators. Lysis buffers are lysis solutions comprising one or more buffering agent. Additional components of lysis solutions may comprise one or more solubilizing agent. As used herein, the term “solubilizing agent” refers to a compound that enhances the solubility of one or more components of a solution and/or the solubility of one or more entities to which solutions are applied. In certain embodiments, solubilizing agents enhance protein solubility. In certain embodiments, solubilizing agents are selected based on their ability to enhance protein solubility while maintaining protein conformation and/or activity.

Exemplary lysis agents may comprise any of those described in U.S. Pat. Nos. 8,685,734, 7,901,921, 7,732,129, 7,223,585, 7,125,706, 8,236,495, 8,110,351, 7,419,956, 7,300,797, 6,699,706 and 6,143,567, the contents of each of which are herein incorporated by reference in their entirety. In certain embodiments, lysis agents may be selected from lysis salts, amphoteric agents, cationic agents, ionic detergents and non-ionic detergents. Lysis salts may comprise, but are not limited to, sodium chloride (NaCl) and potassium chloride (KCl.) Further lysis salts may comprise any of those described in U.S. Pat. Nos. 8,614,101, 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935 and 7,968,333, the contents of each of which are herein incorporated by reference in their entirety.

In certain embodiments, cell lysates agents include amino acids such as arginine, or acidified amino acid mixtures such as arginine HCl.

In certain embodiments, the cell lysate solution comprises a stabilizing additive. In certain embodiments, the stabilizing additive can comprise trehalose, glycine betaine, mannitol, potassium citrate, CuCl₂, proline, xylitol, NDSB 201, CTAB and K₂PO₄. In certain embodiments, the stabilizing additive can comprise amino acids such as arginine, or acidified amino acid mixtures such as arginine HCl. In certain embodiments, the stabilizing additive can comprise 0.1 M arginine or arginine HCL. In certain embodiments, the stabilizing additive can comprise 0.2 M arginine or arginine HCl. In certain embodiments, the stabilizing additive can comprise 0.25 M arginine or arginine HCl. In certain embodiments, the stabilizing additive can comprise 0.3 M arginine or arginine HCl. In certain embodiments, the stabilizing additive can comprise 0.4 M arginine or arginine HCl. In certain embodiments, the stabilizing additive can comprise 0.5 M arginine or arginine HCl. In certain embodiments, the stabilizing additive can comprise 0.6 M arginine or arginine HCl. In certain embodiments, the stabilizing additive can comprise 0.7 M arginine or arginine HCl. In certain embodiments, the stabilizing additive can comprise 0.8 M arginine or arginine HCl. In certain embodiments, the stabilizing additive can comprise 0.9 M arginine or arginine HCl. In certain embodiments, the stabilizing additive can comprise 1.0M arginine or arginine HCl.

Concentrations of salts may be increased or decreased to obtain an effective concentration for the rupture of cell membranes. Amphoteric agents, as referred to herein, are compounds capable of reacting as an acid or a base. Amphoteric agents may comprise, but are not limited to lysophosphatidylcholine, 3-((3-Cholamidopropyl) dimethylammonium)-1-propanesulfonate (CHAPS), ZWITTERGENT® and the like. Cationic agents may comprise, but are not limited to, cetyltrimethylammonium bromide (C (16) TAB) and Benzalkonium chloride. Lysis agents comprising detergents may comprise ionic detergents or non-ionic detergents.

Detergents may function to break apart or dissolve cell structures comprising, but not limited to cell membranes, cell walls, lipids, carbohydrates, lipoproteins and glycoproteins. Exemplary ionic detergents comprise any of those taught in U.S. Pat. Nos. 7,625,570 and 6,593,123 or US Publication No. US2014/0087361, the contents of each of which are herein incorporated by reference in their entirety. In certain embodiments, the lysis solution comprises one or more ionic detergents. Example of ionic detergents for use in a lysis solution comprise, but are not limited to, sodium dodecyl sulfate (SDS), cholate and deoxycholate. In certain embodiments, ionic detergents may be comprised in lysis solutions as a solubilizing agent. In certain embodiments, the lysis solution comprises one or more nonionic detergents. Non-ionic detergents for use in a lysis solution may comprise, but are not limited to, octylglucoside, digitonin, lubrol, C12E8, TWEEN®-20, TWEEN®-80, Triton X-100, Triton X-114, Brij-35, Brij-58, and Noniodet P-40. Non-ionic detergents are typically weaker lysis agents but may be comprised as solubilizing agents for solubilizing cellular and/or viral proteins. In certain embodiments, the lysis solution comprises one or more zwitterionic detergents. Zwitterionic detergents for use in a lysis solution may comprise, but are not limited to: Lauryl dimethylamine N-oxide (LDAO); N,N-Dimethyl-N-dodecylglycine betaine (Empigen® BB); 3-(N,N-Dimethylmyristylammonio) propanesulfonate (Zwittergent® 3-10); n-Dodecyl-N,N-dimethyl-3-ammonio-1-propanesulfonate (Zwittergent® 3-12); n-Tetradecyl-N,N-dimethyl-3-ammonio-1-propanesulfonate (Zwittergent® 3-14); 3-(N,N-Dimethyl palmitylammonio) propanesulfonate (Zwittergent® 3-16); 3-((3-cholamidopropyl) dimethylammonio)-1-propanesulfonate (CHAPS); and 3-([3-Cholamidopropyl] dimethylammonio)-2-hydroxy-1-propanesulfonate (CHAPSO).

In certain embodiments, the lysis solution comprises Triton X-100 (octyl phenol ethoxylate), such as 0.5% w/v of Triton X-100. In certain embodiments, the lysis solution comprises Lauryldimethylamine N-oxide (LDAO), such as 0.184% w/v (4×CMC) of LDAO. In certain embodiments, the lysis solution comprises a seed oil surfactant such as Ecosurf™ SA-9. In certain embodiments, the lysis solution comprises N,N-Dimethyl-N-dodecylglycine betaine (Empigen® BB). In certain embodiments, the lysis solution comprises a Zwittergent® detergent, such as Zwittergent® 3-12 (n-Dodecyl-N,N-dimethyl-3-ammonio-1-propanesulfonate), Zwittergent® 3-14 (n-Tetradecyl-N,N-dimethyl-3-ammonio-1-propanesulfonate), or Zwittergent® 3-16 (3-(N,N-Dimethyl palmitylammonio)propanesulfonate).

Further lysis agents may comprise enzymes and urea. In certain embodiments, one or more lysis agents may be combined in a lysis solution in order to enhance one or more of cell lysis and protein solubility. In certain embodiments, enzyme inhibitors may be comprised in lysis solutions in order to prevent proteolysis that may be triggered by cell membrane disruption.

In certain embodiments, the lysis solution comprises between 0.1-1.0% w/v, between 0.2-0.8% w/v, between 0.3-0.7% w/v, between 0.4-0.6% w/v, or about 0.5% w/v of a cell lysis agent (e.g. detergent). In certain embodiments, the lysis solution comprises between 0.3-0.35% w/v, between 0.35-0.4% w/v, between 0.4-0.45% w/v, between 0.45-0.5% w/v, between 0.5-0.55% w/v, between 0.55-0.6% w/v, between 0.6-0.65% w/v, or between 0.65-0.7% w/v of a cell lysis agent (e.g. detergent).

In certain embodiments, cell lysates generated from adherent cell cultures may be treated with one more nuclease, such as Benzonase nuclease (Grade 1, 99% pure) or c-LEcta Denarase nuclease (formerly Sartorius Denarase). In certain embodiments, nuclease is added to lower the viscosity of the lysates caused by liberated DNA.

In certain embodiments, chemical lysis uses a single chemical lysis mixture. In certain embodiments, chemical lysis uses several lysis agents added in series to provide a final chemical lysis mixture.

In certain embodiments, a chemical lysis mixture comprises an acidified amino acid mixture (such as arginine HCl), a non-ionic detergent (such as Triton X-100), and a nuclease (such as Benzonase nuclease). In certain embodiments, the chemical lysis mixture can comprise an acid or base to provide a target lysis pH.

In certain embodiments, the lysis solution comprises 0.5% w/v Triton X-100 (octyl phenol ethoxylate) and 200 mM arginine hydrochloride. In certain embodiments, the lysis solution comprises 0.5% w/v Triton X-100 (octyl phenol ethoxylate) and 200 mM arginine hydrochloride, and lacks detectable nuclease. In certain embodiments, the lysis solution consists of 0.5% w/v Triton X-100 (octyl phenol ethoxylate) and 200 mM arginine hydrochloride.

In certain embodiments, chemical lysis is conducted under chemical lysis conditions. As used herein, the term “chemical lysis conditions” refers to any combination of environmental conditions (e.g., temperature, pressure, pH, etc.) in which targets cells can be lysed by a chemical lysis agent.

In certain embodiments, the lysis pH is between 3.0-3.5, 3.5-4.0, 4.0-4.5, 4.5-5.0, 5.0-5.5, 5.5-6.0, 6.0-6.5, 6.5-7.0, 7.0-7.5, or 7.5-8.0. In certain embodiments, the lysis pH is between 6.0-7.0, 6.5-7.0, 6.5-7.5, or 7.0-7.5.

In certain embodiments, the lysis temperature is between 15-35° C., between 20-30° C., between 25-39° C., between 20-21° C., between 20-22° C., between 21-22° C., between 21-23° C., between 22-23° C., between 22-24° C., between 23-24° C., between 23-25° C., between 24-25° C., between 24-26° C., between 25-26° C., between 25-27° C., between 26-27° C., between 26-28° C., between 27-28° C. between 27-29° C., between 28-29° C., between 28-30° C., between 29-30° C., between 29-31° C., between 30-31° C., between 30-32° C., between 31-32° C., or between 31-33° C.

In certain embodiments, the lysis solution comprises 0.5% w/v Triton X-100 (octyl phenol ethoxylate) and 200 mM arginine hydrochloride, and lysis conditions comprise a duration of at least 4 hours (e.g., 4-6 hours, e.g., 4 hours) at 26° C.-28° C. (e.g., 27° C.). In certain embodiments, the lysis solution comprises 0.5% w/v Triton X-100 (octyl phenol ethoxylate) and 200 mM arginine hydrochloride, and lacks detectable nuclease, and lysis conditions comprise a duration of at least 4 hours (e.g., 4-6 hours, e.g., 4 hours) at 26° C.-28° C. (e.g., 27° C.). In certain embodiments, the lysis solution consists of 0.5% w/v Triton X-100 (octyl phenol ethoxylate) and 200 mM arginine hydrochloride, and lysis conditions comprise a duration of at least 4 hours (e.g., 4-6 hours, e.g., 4 hours) at 26° C.-28° C. (e.g., 27° C.).

In certain embodiments, mechanical cell lysis is carried out. Mechanical cell lysis methods may comprise the use of one or more lysis condition and/or one or more lysis force. As used herein, the term “lysis condition” refers to a state or circumstance that promotes cellular disruption. Lysis conditions may comprise certain temperatures, pressures, osmotic purity, salinity and the like. In certain embodiments, lysis conditions comprise increased or decreased temperatures. According to certain embodiments, lysis conditions comprise changes in temperature to promote cellular disruption. Cell lysis carried out according to such embodiments may comprise freeze-thaw lysis. As used herein, the term “freeze-thaw lysis” refers to cellular lysis in which a cell solution is subjected to one or more freeze-thaw cycle. According to freeze-thaw lysis methods, cells in solution are frozen to induce a mechanical disruption of cellular membranes caused by the formation and expansion of ice crystals. Cell solutions used according freeze-thaw lysis methods, may further comprise one or more lysis agents, solubilizing agents, buffering agents, cryoprotectants, surfactants, preservatives, enzymes, enzyme inhibitors and/or chelators. Once cell solutions subjected to freezing are thawed, such components may enhance the recovery of desired cellular products. In certain embodiments, one or more cryoprotectants are comprised in cell solutions undergoing freeze-thaw lysis. As used herein, the term “cryoprotectant” refers to an agent used to protect one or more substance from damage due to freezing. Cryoprotectants may comprise any of those taught in US Publication No. US2013/0323302 or U.S. Pat. Nos. 6,503,888, 6,180,613, 7,888,096, 7,091,030, the contents of each of which are herein incorporated by reference in their entirety. In certain embodiments, cryoprotectants may comprise, but are not limited to dimethyl sulfoxide, 1,2-propanediol, 2,3-butanediol, formamide, glycerol, ethylene glycol, 1,3-propanediol and n-dimethyl formamide, polyvinylpyrrolidone, hydroxyethyl starch, agarose, dextrans, inositol, glucose, hydroxyethylstarch, lactose, sorbitol, methyl glucose, sucrose and urea. In certain embodiments, freeze-thaw lysis may be carried out according to any of the methods described in U.S. Pat. No. 7,704,721, the contents of which are herein incorporated by reference in their entirety.

As used herein, the term “lysis force” refers to a physical activity used to disrupt a cell. Lysis forces may comprise, but are not limited to mechanical forces, sonic forces, gravitational forces, optical forces, electrical forces and the like. Cell lysis carried out by mechanical force is referred to herein as “mechanical lysis.” Mechanical forces that may be used according to mechanical lysis may comprise high shear fluid forces. According to such methods of mechanical lysis, a microfluidizer may be used. Microfluidizers typically comprise an inlet reservoir where cell solutions may be applied. Cell solutions may then be pumped into an interaction chamber via a pump (e.g. high-pressure pump) at high speed and/or pressure to produce shear fluid forces. Resulting lysates may then be collected in one or more output reservoir. Pump speed and/or pressure may be adjusted to modulate cell lysis and enhance recovery of products (e.g. viral particles.) Other mechanical lysis methods may comprise physical disruption of cells by scraping.

Cell lysis methods may be selected based on the cell culture format of cells to be lysed. For example, with adherent cell cultures, some chemical and mechanical lysis methods may be used. Such mechanical lysis methods may comprise freeze-thaw lysis or scraping. In another example, chemical lysis of adherent cell cultures may be carried out through incubation with lysis solutions comprising surfactant, such as Triton-X-100.

In certain embodiments, a method for harvesting AAV particles without lysis may be used for efficient and scalable AAV particle production. In a non-limiting example, AAV particles may be produced by culturing an AAV particle lacking a heparin binding site, thereby allowing the AAV particle to pass into the supernatant, in a cell culture, collecting supernatant from the culture; and isolating the AAV particle from the supernatant, as described in US Patent Application 20090275107, the contents of which are incorporated herein by reference in their entirety.

### Clarification and Purification: General

Cell lysates comprising viral particles may be subjected to clarification and purification. Clarification generally refers to the initial steps taken in the purification of viral particles from cell lysates and serves to prepare lysates for further purification by removing larger, insoluble debris from a bulk lysis harvest. Viral production can comprise clarification steps at any point in the viral production process. Clarification steps may comprise, but are not limited to, centrifugation and filtration. During clarification, centrifugation may be carried out at low speeds to remove larger debris only. Similarly, filtration may be carried out using filters with larger pore sizes so that only larger debris is removed.

Purification generally refers to the final steps taken in the purification and concentration of viral particles from cell lysates by removing smaller debris from a clarified lysis harvest in preparing a final Pooled Drug Substance. Viral production can comprise purification steps at any point in the viral production process. Purification steps may comprise, but are not limited to, filtration and chromatography. Filtration may be carried out using filters with smaller pore sizes to remove smaller debris from the product or with larger pore sizes to retain larger debris from the product. Filtration may be used to alter the concentration and/or contents of a viral production pool or stream. Chromatography may be carried out to selectively separate target particles from a pool of impurities.

Large-scale production of high-concentration AAV formulations is complicated by the tendency for high concentrations of AAV particles to aggregate or agglomerate. Small scale clarification and concentration systems, such as dialysis cassettes or spin centrifugation, are generally not sufficiently scalable for large-scale production. The present disclosure provides embodiments of a clarification, purification and concentration system for processing large volumes of high-concentration AAV production formulations. In certain embodiments, the large-volume clarification system comprises one or more of the following processing steps: Depth Filtration, Microfiltration (e.g. 0.2 μm Filtration), Affinity Chromatography, Ion Exchange Chromatography such as anion exchange chromatography (AEX) or cation exchange chromatography (CEX), a tangential flow filtration system (TFF), Nanofiltration (e.g. Virus Retentive Filtration (VRF)), Final Filtration (FF), and Fill Filtration.

Objectives of viral clarification and purification comprise high throughput processing of cell lysates and to optimize ultimate viral recovery. Advantages of comprising clarification and purification steps of the present disclosure comprise scalability for processing of larger volumes of lysate. In certain embodiments, clarification and purification may be carried out according to any of the methods or systems presented in U.S. Pat. Nos. 8,524,446, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498, 7,491,508, US Publication Nos. US2013/0045186, US2011/0263027, US2011/0151434, US2003/0138772, and International Publication Nos. WO2002012455, WO1996039530, WO1998010088, WO1999014354, WO1999015685, WO1999047691, WO2000055342, WO2000075353 and WO2001023597, the contents of each of which are herein incorporated by reference in their entirety.

In certain embodiments, the compositions comprising at least one AAV particle may be isolated or purified using the methods or systems described in U.S. Pat. Nos. 6,146,874, 6,660,514, 8,283,151 or U.S. Pat. No. 8,524,446, the contents of which are herein incorporated by reference in their entirety.

### Clarification and Purification: Centrifugation

According to certain embodiments, cell lysates may be clarified by one or more centrifugation steps. Centrifugation may be used to pellet insoluble particles in the lysate. During clarification, centrifugation strength (which can be expressed in terms of gravitational units (g), which represents multiples of standard gravitational force) may be lower than in subsequent purification steps. In certain embodiments, centrifugation may be carried out on cell lysates at a gravitation force from about 200 g to about 800 g, from about 500 g to about 1500 g, from about 1000 g to about 5000 g, from about 1200 g to about 10000 g or from about 8000 g to about 15000 g. In certain embodiments, cell lysate centrifugation is carried out at 8000 g for 15 minutes. In certain embodiments, density gradient centrifugation may be carried out in order to partition particulates in the cell lysate by sedimentation rate. Gradients used according to methods or systems of the present disclosure may comprise, but are not limited to, cesium chloride gradients and iodixanol step gradients. In certain embodiments, centrifugation uses a decanter centrifuge system. In certain embodiments, centrifugation uses a disc-stack centrifuge system. In certain embodiments, centrifugation comprises ultracentrifugation, such two-cycle CsCl gradient ultracentrifugation or iodixanol discontinuous density gradient ultracentrifugation.

### Clarification and Purification: Filtration

In certain embodiments, one or more microfiltration, nanofiltration and/or ultrafiltration steps may be used during clarification, purification and/or sterilization. The one or more microfiltration, nanofiltration or ultrafiltration steps can comprise the use of a filtration system such as EMD Millipore Express SHC XL10 0.5/0.2 μm filter, EMD Millipore Express SHCXL6000 0.5/0.2 μm filter, EMD Millipore Express SHCXL150 filter, EMD Millipore Millipak Gamma Gold 0.22 μm filter (dual-in-line sterilizing grade filters), a Pall Supor EKV, 0.2 μm sterilizing-grade filter, Asahi Planova 35N, Asahi Planova 20N, Asahi Planova 75N, Asahi Planova BioEx, Millipore Viresolve NFR or a Sartorius Sartopore 2XLG, 0.8/0.2 μm.

In certain embodiments, one or more microfiltration steps may be used during clarification, purification and/or sterilization. Microfiltration utilizes microfiltration membranes with pore sizes typically between 0.1 μm and 10 μm. Microfiltration is generally used for general clarification, sterilization, and removal of microparticulates. In certain embodiments, microfiltration is used to remove aggregated clumps of viral particles. In certain embodiments, a production process or system of the present disclosure comprises at least one microfiltration step. The one or more microfiltration steps can comprise a Depth Filtration step with a Depth Filtration system, such as EMD Millipore Millistak⁺ POD filter (DOHC media series), Millipore MC0SP23CL3 filter (C0SP media series), or Sartorius Sartopore filter series. Microfiltration systems of the present disclosure can be pre-rinsed, packed, equilibrated, flushed, processed, eluted, washed or cleaned with formulations known to those in the art, comprising AAV pharmaceutical, processing and storage formulations of the present disclosure. In certain embodiments, clarification comprises use of a C0SP media series filter. In some embodiments, the C0SP media series filter is effective to reduce or prevent 0.2-micron filter clogging.

In certain embodiments, one or more ultrafiltration steps may be used during clarification and purification. The ultrafiltration steps can be used for concentrating, formulating, desalting or dehydrating either processing and/or formulation solutions of the present disclosure. Ultrafiltration utilizes ultrafiltration membranes, with pore sizes typically between 0.001 and 0.1 μm. Ultrafiltration membranes can also be defined by their molecular weight cutoff (MWCO) and can have a range from 1 kD to 500 kD. Ultrafiltration is generally used for concentrating and formulating dissolved biomolecules such as proteins, peptides, plasmids, viral particles, nucleic acids, and carbohydrates. Ultrafiltration systems of the present disclosure can be pre-rinsed, packed, equilibrated, flushed, processed, eluted, washed or cleaned with formulations known to those in the art, comprising AAV pharmaceutical, processing and storage formulations of the present disclosure.

In certain embodiments, one or more nanofiltration steps may be used during clarification and purification. Nanofiltration utilizes nanofiltration membranes, with pore sizes typically less than 100 nm. Nanofiltration is generally used for removal of unwanted endogenous viral impurities (e.g. baculovirus). In certain embodiments, nanofiltration can comprise viral removal filtration (VRF). VRF filters can have a filtration size typically between 15 nm and 100 nm. Examples of VRF filters comprise (but are not limited to): Planova 15N, Planova 20N, and Planova 35N (Asahi-Kasei Corp, Tokyo, Japan); and Viresolve NFP and Viresolve NFR (Millipore Corp, Billerica, Mass., USA). Nanofiltration systems of the present disclosure can be pre-rinsed, packed, equilibrated, flushed, processed, eluted, washed or cleaned with formulations known to those in the art, comprising AAV pharmaceutical, processing and storage formulations of the present disclosure. In certain embodiments, nanofiltration is used to remove aggregated clumps of viral particles.

In certain embodiments, one or more tangential flow filtration (TFF) (also known as cross-flow filtration) steps may be used during clarification and purification. Tangential flow filtration is a form of membrane filtration in which a feed stream (which comprises the target agent/particle to be clarified and concentrated) flows from a feed tank into a filtration module or cartridge. Within the TFF filtration module, the feed stream passes parallel to a membrane surface, such that one portion of the stream passes through the membrane (permeate/filtrate) while the remainder of the stream (retentate) is recirculated back through the filtration system and into the feed tank.

In certain embodiments, the TFF filtration module can be a flat plate module (stacked planar cassette), a spiral wound module (spiral-wound membrane layers), or a hollow fiber module (bundle of membrane tubes). Examples of TFF systems for use in the present disclosure comprise, but are not limited to: Spectrum mPES Hollow Fiber TFF system (0.5 mm fiber ID, 100 kDA MWCO) or Millipore Ultracel PLCTK system with Pellicon-3 cassette (0.57 m², 30 kDA MWCO).

New buffer materials can be added to the TFF feed tank as the feed stream is circulated through the TFF filtration system. In certain embodiments, buffer materials can be fully replenished as the flow stream circulates through the TFF filtration system. In this embodiment, buffer material is added to the stream in equal amounts to the buffer material lost in the permeate, resulting in a constant concentration. In certain embodiments, buffer materials can be reduced as the flow stream circulates through the filtration system. In this embodiment, a reduced amount of buffer material is added to the stream relative to the buffer material lost in the permeate, resulting in an increased concentration. In certain embodiments, buffer materials can be replaced as the flow stream circulates through the filtration system. In this embodiment, the buffer added to stream is different from buffer materials lost in the permeate, resulting in an eventual replacement of buffer material in the stream. TFF systems of the present disclosure can be pre-rinsed, packed, equilibrated, flushed, processed, eluted, washed or cleaned with formulations known to those in the art, comprising AAV pharmaceutical, processing and storage formulations of the present disclosure.

In certain embodiments, a TFF load pool can be spiked with an excipient or diluent prior to filtration. In certain embodiments, a TFF load pool is spiked with a high-salt mixture (such as sodium chloride or potassium chloride) prior to filtration. In certain embodiments, a TFF load pool is spiked with a high-sugar mixture (such as 50% w/v sucrose) prior to filtration.

The effectiveness of TFF processing can depend on several factors, comprising (but not limited to): shear stress from flow design, cross-flow rate, filtrate flow control, transmembrane pressure (TMP), membrane conditioning, membrane composition (e.g. hollow fiber construction) and design (e.g. surface area), system flow design, reservoir design, and mixing strategy. In certain embodiment, the filtration membrane can be exposed to pre-TFF membrane conditioning.

In certain embodiments, TFF processing can comprise one or more microfiltration stages. In certain embodiments, TFF processing can comprise one or more ultrafiltration stages. In certain embodiments, TFF processing can comprise one or more nanofiltration stages.

In certain embodiments, TFF processing can comprise one or more concentration stages, such as an ultrafiltration (UF) or microfiltration (MF) concentration stage. In the concentration stage, a reduced amount of buffer material is replaced as the stream circulates through the filtration system (relative to the amount of buffer material lost as permeate). The failure to completely replace all of the buffer material lost in the permeate results in an increased concentration of viral particles within the filtration stream. In certain embodiments, an increased amount of buffer material is replaced as the stream circulates through the filtration system. The incorporation of excess buffer material relative to the amount of buffer material lost in the permeate results in a decreased concentration of viral particles within the filtration stream.

In certain embodiments, TFF processing can comprise one or more diafiltration (DF) stages. The diafiltration stage comprises replacement of a first buffer material (such as a high salt material) within a second buffer material (such a low-salt or zero-salt material). In this embodiment, a second buffer is added to flow stream which is different from a first buffer material lost in the permeate, resulting in an eventual replacement of buffer material in the stream.

In certain embodiments, TFF processing can comprise multiple stages in series. In certain embodiments, a TFF processing process can comprise an ultrafiltration (UF) concentration stage followed by a diafiltration stage (DF). In certain embodiments, TFF comprising UF followed by DF results in increased rAAV recovery relative to TFF comprising DF followed by UF. In some embodiments, TFF comprising UF followed by DF results in about 70-80% recovery of rAAV.

In certain embodiments, a TFF processing can comprise a diafiltration stage followed by an ultrafiltration concentration stage. In certain embodiments, a TFF processing can comprise a first diafiltration stage, followed by an ultrafiltration concentration stage, followed by a second diafiltration stage. In certain embodiments, a TFF processing can comprise a first diafiltration stage which incorporates a high-salt-low-sugar buffer material into the flow stream, followed by an ultrafiltration/concentration stage which results in a high concentration of the viral material in the flow stream, followed by a second diafiltration stage which incorporates a low-salt-high-sugar or zero-salt-high-sugar buffer material into the flow stream. In certain embodiments, the salt can be sodium chloride, sodium phosphate, potassium chloride, potassium phosphate, or a combination thereof. In certain embodiments, the sugar can be sucrose, such as a 5% w/v sucrose mixture or a 7% w/v sucrose mixture.

In certain embodiments, the one or more TFF steps can comprise a formulation diafiltration step in which at least a portion of the liquid media of the viral production pool is replaced with a high-sucrose formulation buffer. In certain embodiments, the high-sucrose formulation buffer comprises between 6-8% w/v of a sugar or sugar substitute and between 90-100 mM of an alkali chloride salt. In certain embodiments, the high-sucrose formulation buffer comprises 7% w/v of sucrose and between 90-100 mM sodium chloride. In certain embodiments, the high-sucrose formulation buffer comprises 7% w/v of sucrose, 10 mM Sodium Phosphate, between 95-100 mM sodium chloride, and 0.001% (w/v) Poloxamer 188. In certain embodiments, the formulation diafiltration step is the final diafiltration step in the one or more TFF steps. In certain embodiments, the formulation diafiltration step is the only diafiltration step in the one or more TFF steps.

In certain embodiments, TFF processing can comprise multiple stages which occur contemporaneously. As a non-limiting example, a TFF clarification process can comprise an ultrafiltration stage which occurs contemporaneously with a concentration stage.

Methods of cell lysate clarification and purification by filtration are well understood in the art and may be carried out according to a variety of available methods comprising, but not limited to passive filtration and flow filtration. Filters used may comprise a variety of materials and pore sizes. For example, cell lysate filters may comprise pore sizes of from about 1 μM to about 5 μM, from about 0.5 μM to about 2 μM, from about 0.1 μM to about 1 μM, from about 0.05 μM to about 0.05 μM and from about 0.001 μM to about 0.1 μM. Exemplary pore sizes for cell lysate filters may comprise, but are not limited to, 2.0, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.22, 0.21, 0.20, 0.19, 0.18, 0.17, 0.16, 0.15, 0.14, 0.13, 0.12, 0.11, 0.1, 0.09, 0.08, 0.07, 0.06, 0.05, 0.04, 0.03, 0.02, 0.01, 0.02, 0.019, 0.018, 0.017, 0.016, 0.015, 0.014, 0.013, 0.012, 0.011, 0.01, 0.009, 0.008, 0.007, 0.006, 0.005, 0.004, 0.003, 0.002, 0.001 and 0.001 μM. In certain embodiments, clarification may comprise filtration through a filter with 2.0 μM pore size to remove large debris, followed by passage through a filter with 0.45 μM pore size to remove intact cells.

Filter materials may be composed of a variety of materials. Such materials may comprise, but are not limited to, polymeric materials and metal materials (e.g. sintered metal and pored aluminum.) Exemplary materials may comprise, but are not limited to nylon, cellulose materials (e.g. cellulose acetate), polyvinylidene fluoride (PVDF), polyethersulfone, polyamic, polysulfone, polypropylene, and polyethylene terephthalate. In certain embodiments, filters useful for clarification of cell lysates may comprise, but are not limited to ULTIPLEAT PROFILE™ filters (Pall Corporation, Port Washington, N.Y.), SUPOR™ membrane filters (Pall Corporation, Port Washington, N.Y.).

In certain embodiments, flow filtration may be carried out to increase filtration speed and/or effectiveness. In certain embodiments, flow filtration may comprise vacuum filtration. According to such methods, a vacuum is created on the side of the filter opposite that of cell lysate to be filtered. In certain embodiments, cell lysates may be passed through filters by centrifugal forces. In certain embodiments, a pump is used to force cell lysate through clarification filters. Flow rate of cell lysate through one or more filters may be modulated by adjusting one of channel size and/or fluid pressure.

### Clarification and Purification: Chromatography

In certain embodiments, AAV particles in a formulation may be clarified and purified from cell lysates through one or more chromatography steps using one or more different methods of chromatography. Chromatography refers to any number of methods known in the art for selectively separating out one or more elements from a mixture. Such methods may comprise, but are not limited to, ion exchange chromatography (e.g. cation exchange chromatography and anion exchange chromatography), affinity chromatography (e.g. immunoaffinity chromatography, metal affinity chromatography, pseudo affinity chromatography such as Blue Sepharose resins), hydrophobic interaction chromatography (HIC), size-exclusion chromatography, and multimodal chromatography (MMC) (chromatographic methods that utilize more than one form of interaction between the stationary phase and analytes). In certain embodiments, methods or systems of viral chromatography may comprise any of those taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety.

Chromatography systems of the present disclosure can be pre-rinsed, packed, equilibrated, flushed, processed, eluted, washed or cleaned with formulations known to those in the art, comprising AAV pharmaceutical, processing and storage formulations of the present disclosure.

In certain embodiments, one or more ion exchange (IEX) chromatography steps may be used to isolate viral particles. The ion exchange step can comprise anion exchange (AEX) chromatography, cation exchange (CEX) chromatography, or a combination thereof. In certain embodiments, ion exchange chromatography is used in a bind/elute mode. Bind/elute IEX can be used by binding viral particles to a stationary phase based on charge-charge interactions between capsid proteins (or other charged components) of the viral particles and charged sites present on the stationary phase. This process can comprise the use of a column through which viral preparations (e.g. clarified lysates) are passed. After application of viral preparations to the charged stationary phase (e.g. column), bound viral particles may then be eluted from the stationary phase by applying an elution solution to disrupt the charge-charge interactions. Elution solutions may be optimized by adjusting salt concentration and/or pH to enhance recovery of bound viral particles. In certain embodiments, the elution solution can comprise a nuclease such as Benzonase nuclease. Depending on the charge of viral capsids being isolated, cation or anion exchange chromatography methods may be selected. In certain embodiments, ion exchange chromatography is used in a flow-through mode. Flow-through IEX can be used by binding non-viral impurities or unwanted viral particles to a stationary phase (based on charge-charge interactions) and allowing the target viral particles in the viral preparation to “flow through” the IEX system into a collection pool.

Methods or systems of ion exchange chromatography may comprise, but are not limited to any of those taught in U.S. Pat. Nos. 7,419,817, 6,143,548, 7,094,604, 6,593,123, 7,015,026 and 8,137,948, the contents of each of which are herein incorporated by reference in their entirety.

In certain embodiments, the IEX process uses an AEX chromatography system such as a Sartorius Sartobind Q membrane, a Sartorius Sartobind STIC membrane, a Millipore Fractogel TMAE HiCap(m) Flow-Through membrane, a GE Q Sepharose HP membrane, Poros XQ or Poros HQ. In certain embodiments, the IEX process uses a CEX system such as a Poros XS membrane. In certain embodiments, the AEX system comprises a stationary phase which comprises a trimethylammoniumethyl (TMAE) functional group. In certain embodiments, the IEX process uses a Multimodal Chromatography (MMC) system such as a Nuvia aPrime 4A membrane.

In certain embodiments, one or more affinity chromatography steps, such as immunoaffinity chromatography, may be used to isolate viral particles. Immunoaffinity chromatography is a form of chromatography that utilizes one or more immune compounds (e.g. antibodies or antibody-related structures) to retain viral particles. Immune compounds may bind specifically to one or more structures on viral particle surfaces, comprising, but not limited to one or more viral coat protein. In certain embodiments, immune compounds may be specific for a particular viral variant. In certain embodiments, immune compounds may bind to multiple viral variants. In certain embodiments, immune compounds may comprise recombinant single-chain antibodies. Such recombinant single chain antibodies may comprise those described in Smith, R. H. et al., 2009. Mol. Ther. 17(11):1888-96, the contents of which are herein incorporated by reference in their entirety. Such immune compounds (e.g., recombinant protein ligands) are capable of binding to several AAV capsid variants, comprising, but not limited to AAV1, AAV2, AAV3, AAV5, AAV6 and/or AAV8 or any of those taught herein. In some embodiments, such immune compounds (e.g., recombinant protein ligands) are capable of binding to at least AAV2. In certain embodiments, the AFC process uses a GE AVB Sepharose HP column resin, Poros CaptureSelect AAV8 resins (ThermoFisher), Poros CaptureSelect AAV9 resins (ThermoFisher) and Poros CaptureSelect AAVX resins (ThermoFisher).

In certain embodiments, one or more affinity chromatography steps precedes one or more anion exchange chromatography steps. In certain embodiments, one or more anion exchange chromatography steps precedes one or more affinity chromatography steps.

In certain embodiments, one or more size-exclusion chromatography (SEC) steps may be used to isolate viral particles. SEC may comprise the use of a gel to separate particles according to size. In viral particle purification, SEC filtration is sometimes referred to as “polishing.” In certain embodiments, SEC may be carried out to generate a final product that is near-homogenous. Such final products may in certain embodiments be used in pre-clinical studies and/or clinical studies (Kotin, R. M. 2011. Human Molecular Genetics. 20(1):R2-R6, the contents of which are herein incorporated by reference in their entirety.) In certain embodiments, SEC may be carried out according to any of the methods taught in U.S. Pat. Nos. 6,143,548, 7,015,026, 8,476,418, 6,410,300, 8,476,418, 7,419,817, 7,094,604, 6,593,123, and 8,137,948, the contents of each of which are herein incorporated by reference in their entirety.

In some embodiments, purification of recombinant AAV produces a total rAAV process yield of 30-50%.

### III. Compositions and Formulations

### General

Gene therapy drug products (such as rAAV particles) are challenging to incorporate into composition and formulations due to their limited stability in the liquid state and a high propensity for large-scale aggregation at low concentrations. Gene therapy drug products are often delivered directly to treatment areas (comprising CNS tissue); which requires that excipients and formulation parameters be compatible with tissue function, microenvironment, and volume restrictions.

According to the present disclosure, AAV particles may be prepared as, or comprised in, pharmaceutical compositions. It will be understood that such compositions necessarily comprise one or more active ingredients and, most often, one or more pharmaceutically acceptable excipients.

Relative amounts of the active ingredient (e.g. AAV particle), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, or at least 80% (w/w) active ingredient.

In certain embodiments, the AAV particle pharmaceutical compositions described herein may comprise at least one payload of the present disclosure. As a non-limiting example, the pharmaceutical compositions may contain an AAV particle with 1, 2, 3, 4 or 5 payloads.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated comprise, but are not limited to, humans and/or other primates; mammals, comprising commercially relevant mammals such as cattle, pigs, horses, sheep, cats, dogs, mice, rats, birds, comprising commercially relevant birds such as poultry, chickens, ducks, geese, and/or turkeys.

In certain embodiments, compositions are administered to humans, human patients or subjects.

Formulations of the present disclosure can comprise, without limitation, saline, liposomes, lipid nanoparticles, polymers, peptides, proteins, cells transfected with AAV particles (e.g., for transfer or transplantation into a subject) and combinations thereof.

Formulations of the pharmaceutical compositions described herein may be prepared by any method known or hereafter developed in the art of pharmacology. As used herein the term “pharmaceutical composition” refers to compositions comprising at least one active ingredient and optionally one or more pharmaceutically acceptable excipients.

In general, such preparatory methods comprise the step of associating the active ingredient with an excipient and/or one or more other accessory ingredients. As used herein, the phrase “active ingredient” generally refers either to an AAV particle carrying a payload region encoding the polynucleotide or polypeptides of the present disclosure or to the end product encoded by a viral genome of an AAV particle as described herein.

In certain embodiments, the formulations may comprise at least one inactive ingredient. As used herein, the term “inactive ingredient” refers to one or more inactive agents comprised in formulations. In certain embodiments, all, none or some of the inactive ingredients which may be used in the formulations of the present disclosure may be approved by the US Food and Drug Administration (FDA).

Formulations of the AAV particles and pharmaceutical compositions described herein may be prepared by any method known or hereafter developed in the art of pharmacology. In general, such preparatory methods comprise the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

In certain embodiments, formulations of the present disclosure are aqueous formulations (i.e. formulations which comprise water). In certain embodiments, formulations of the present disclosure comprise water, sanitized water, or Water-for-injection (WFI).

In certain embodiments, the AAV particles of the present disclosure may be formulated in PBS with 0.001%-0.1% (w/v) of Poloxamer 188 (e.g. Pluronic F-68) at a pH of about 7.0.

In certain embodiments, the AAV formulations described herein may contain sufficient AAV particles for expression of at least one expressed functional payload. As a non-limiting example, the AAV particles may contain viral genomes encoding 1, 2, 3, 4 or 5 functional payloads.

According to the present disclosure AAV particles may be formulated for CNS delivery. Agents that cross the brain blood barrier may be used. For example, some cell penetrating peptides that can target molecules to the brain blood barrier endothelium may be used for formulation (e.g., Mathupala, Expert Opin Ther Pat., 2009, 19, 137-140; the content of which is incorporated herein by reference in its entirety).

In certain embodiments, the AAV formulations described herein may comprise a buffering system which comprises phosphate, Tris, and/or Histidine. The buffering agents of phosphate, Tris, and/or Histidine may be independently used in the formulation in a range of 2-12 mM.

Formulations of the present disclosure can be used in any step of producing, processing, preparing, storing, expanding, or administering AAV particles and viral vectors of the present disclosure. In certain embodiments, pharmaceutical formulations and components can be use in AAV production, AAV processing, AAV clarification, AAV purification, and AAV finishing systems of the present disclosure, all of which can be pre-rinsed, packed, equilibrated, flushed, processed, eluted, washed or cleaned with formulations known to those in the art, comprising AAV pharmaceutical, processing and storage formulations of the present disclosure.

### Excipients and Diluents

The AAV particles of the present disclosure can be formulated into a pharmaceutical composition which comprises one or more excipients or diluents to (1) increase stability; (2) increase cell transfection or transduction; (3) permit the sustained or delayed release of the payload; (4) alter the biodistribution (e.g., target the viral particle to specific tissues or cell types); (5) increase the translation of encoded protein; (6) alter the release profile of encoded protein and/or (7) allow for regulatable expression of the payload of the present disclosure.

Relative amounts of the active ingredient (e.g. AAV particle), the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. In certain embodiments, the composition may comprise between 0.001% and 99% (w/w) of the active ingredient. By way of example, the composition may comprise between 0.001% and 100%, e.g., between 0.5 and 50%, between 1-30%, between 5-80%, or at least 80% (w/w) active ingredient. In certain embodiments, the composition may comprise between 0.001% and 99% (w/w) of the excipients and diluents. By way of example, the composition may comprise between 0.001% and 100%, e.g., between 0.5 and 50%, between 1-30%, between 5-80%, or at least 80% (w/w) excipients and diluents.

In certain embodiments, a pharmaceutically acceptable excipient may be at least 95%, at least 96%, at least 97%, at least 98%, at least 99%, or 100% pure. In certain embodiments, an excipient is approved for use for humans and for veterinary use. In certain embodiments, an excipient may be approved by United States Food and Drug Administration. In certain embodiments, an excipient may be of pharmaceutical grade. In certain embodiments, an excipient may meet the standards of the United States Pharmacopoeia (USP), the European Pharmacopoeia (EP), the British Pharmacopoeia, and/or the International Pharmacopoeia.

Excipients, as used herein, comprise, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary excipients and diluents which can be comprised in formulations of the present disclosure comprise, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary excipients and diluents which can be comprised in formulations of the present disclosure comprise, but are not limited to, 1,2,6-Hexanetriol; 1,2-Dimyristoyl-Sn-Glycero-3-(Phospho-S-(1-Glycerol)); 1,2-Dimyristoyl-Sn-Glycero-3-Phosphocholine; 1,2-Dioleoyl-Sn-Glycero-3-Phosphocholine; 1,2-Dipalmitoyl-Sn-Glycero-3-(Phospho-Rac-(1-Glycerol)); 1,2-Distearoyl-Sn-Glycero-3-(Phospho-Rac-(1-Glycerol)); 1,2-Distearoyl-Sn-Glycero-3-Phosphocholine; 1-O-Tolylbiguanide; 2-Ethyl-1,6-Hexanediol; Acetic Acid; Acetic Acid, Glacial; Acetic Anhydride; Acetone; Acetone Sodium Bisulfite; Acetylated Lanolin Alcohols; Acetylated Monoglycerides; Acetylcysteine; Acetyltryptophan, DL-; Acrylates Copolymer; Acrylic Acid-Isooctyl Acrylate Copolymer; Acrylic Adhesive 788; Activated Charcoal; Adcote 72A 103; Adhesive Tape; Adipic Acid; Aerotex Resin 3730; Alanine; Albumin Aggregated; Albumin Colloidal; Albumin Human; Alcohol; Alcohol, Dehydrated, Alcohol, Denatured; Alcohol, Diluted; Alfadex; Alginic Acid; Alkyl Ammonium Sulfonic Acid Betaine; Alkyl Aryl Sodium Sulfonate; Allantoin; Allyl .Alpha.-Ionone; Almond Oil; Alpha-Terpineol; Alpha-Tocopherol; Alpha-Tocopherol Acetate, Dl-; Alpha-Tocopherol, Dl-; Aluminum Acetate; Aluminum Chlorhydroxy Allantoinate; Aluminum Hydroxide; Aluminum Hydroxide—Sucrose, Hydrated; Aluminum Hydroxide Gel; Aluminum Hydroxide Gel F 500; Aluminum Hydroxide Gel F 5000; Aluminum Monostearate; Aluminum Oxide; Aluminum Polyester; Aluminum Silicate; Aluminum Starch Octenylsuccinate; Aluminum Stearate; Aluminum Subacetate; Aluminum Sulfate Anhydrous; Amerchol C; Amerchol-Cab; Aminomethylpropanol; Ammonia; Ammonia Solution; Ammonia Solution, Strong; Ammonium Acetate; Ammonium Hydroxide; Ammonium Lauryl Sulfate; Ammonium Nonoxynol-4 Sulfate; Ammonium Salt Of C-12-C-15 Linear Primary Alcohol Ethoxylate; Ammonium Sulfate; Ammonyx; Amphoteric-2; Amphoteric-9; Anethole; Anhydrous Citric Acid; Anhydrous Dextrose; Anhydrous Lactose; Anhydrous Trisodium Citrate; Aniseed Oil; Anoxid Sbn; Antifoam; Antipyrine; Apaflurane; Apricot Kernel Oil Peg-6 Esters; Aquaphor; Arginine; Arlacel; Ascorbic Acid; Ascorbyl Palmitate; Aspartic Acid; Balsam Peru; Barium Sulfate; Beeswax; Beeswax, Synthetic; Beheneth-10; Bentonite; Benzalkonium Chloride; Benzenesulfonic Acid; Benzethonium Chloride; Benzododecinium Bromide; Benzoic Acid; Benzyl Alcohol; Benzyl Benzoate; Benzyl Chloride; Betadex; Bibapcitide; Bismuth Subgallate; Boric Acid; Brocrinat; Butane; Butyl Alcohol; Butyl Ester Of Vinyl Methyl Ether/Maleic Anhydride Copolymer (125000 Mw); Butyl Stearate; Butylated Hydroxyanisole; Butylated Hydroxytoluene; Butylene Glycol; Butylparaben; Butyric Acid; C20-40 Pareth-24; Caffeine; Calcium; Calcium Carbonate; Calcium Chloride; Calcium Gluceptate; Calcium Hydroxide; Calcium Lactate; Calcobutrol; Caldiamide Sodium; Caloxetate Trisodium; Calteridol Calcium; Canada Balsam; Caprylic/Capric Triglyceride; Caprylic/Capric/Stearic Triglyceride; Captan; Captisol; Caramel; Carbomer 1342; Carbomer 1382; Carbomer 934; Carbomer 934p; Carbomer 940; Carbomer 941; Carbomer 980; Carbomer 981; Carbomer Homopolymer Type B (Allyl Pentaerythritol Crosslinked); Carbomer Homopolymer Type C (Allyl Pentaerythritol Crosslinked); Carbon Dioxide; Carboxy Vinyl Copolymer; Carboxymethylcellulose; Carboxymethylcellulose Sodium; Carboxypolymethylene; Carrageenan; Carrageenan Salt; Castor Oil; Cedar Leaf Oil; Cellulose; Cellulose, Microcrystalline; Cerasynt-Se; Ceresin; Ceteareth-12; Ceteareth-15; Ceteareth-30; Cetearyl Alcohol/Ceteareth-20; Cetearyl Ethylhexanoate; Ceteth-10; Ceteth-2; Ceteth-20; Ceteth-23; Cetostearyl Alcohol; Cetrimonium Chloride; Cetyl Alcohol; Cetyl Esters Wax; Cetyl Palmitate; Cetylpyridinium Chloride; Chlorobutanol; Chlorobutanol Hemihydrate; Chlorobutanol, Anhydrous; Chlorocresol; Chloroxylenol; Cholesterol; Choleth; Choleth-24; Citrate; Citric Acid; Citric Acid Monohydrate; Citric Acid, Hydrous; Cocamide Ether Sulfate; Cocamine Oxide; Coco Betaine; Coco Diethanolamide; Coco Monoethanolamide; Cocoa Butter; Coco-Glycerides; Coconut Oil; Coconut Oil, Hydrogenated; Coconut Oil/Palm Kernel Oil Glycerides, Hydrogenated; Cocoyl Caprylocaprate; Cola Nitida Seed Extract; Collagen; Coloring Suspension; Corn Oil; Cottonseed Oil; Cream Base; Creatine; Creatinine; Cresol; Croscarmellose Sodium; Crospovidone; Cupric Sulfate; Cupric Sulfate Anhydrous; Cyclomethicone; Cyclomethicone/Dimethicone Copolyol; Cysteine; Cysteine Hydrochloride; Cysteine Hydrochloride Anhydrous; Cysteine, D1-; D&C Red No. 28; D&C Red No. 33; D&C Red No. 36; D&C Red No. 39; D&C Yellow No. 10; Dalfampridine; Daubert 1-5 Pestr (Matte) 164z; Decyl Methyl Sulfoxide; Dehydag Wax Sx; Dehydroacetic Acid; Dehymuls E; Denatonium Benzoate; Deoxycholic Acid; Dextran; Dextran 40; Dextrin; Dextrose; Dextrose Monohydrate; Dextrose Solution; Diatrizoic Acid; Diazolidinyl Urea; Dichlorobenzyl Alcohol; Dichlorodifluoromethane; Dichlorotetrafluoroethane; Diethanolamine; Diethyl Pyrocarbonate; Diethyl Sebacate; Diethylene Glycol Monoethyl Ether; Diethylhexyl Phthalate; Dihydroxyaluminum Aminoacetate; Diisopropanolamine; Diisopropyl Adipate; Diisopropyl Dilinoleate; Dimethicone 350; Dimethicone Copolyol; Dimethicone Mdx4-4210; Dimethicone Medical Fluid 360; Dimethyl Isosorbide; Dimethyl Sulfoxide; Dimethylaminoethyl Methacrylate—Butyl Methacrylate—Methyl Methacrylate Copolymer; Dimethyldioctadecylammonium Bentonite; Dimethylsiloxane/Methylvinylsiloxane Copolymer; Dinoseb Ammonium Salt; Dipalmitoylphosphatidylglycerol, Dl-; Dipropylene Glycol; Disodium Cocoamphodiacetate; Disodium Laureth Sulfosuccinate; Disodium Lauryl Sulfosuccinate; Disodium Sulfosalicylate; Disofenin; Divinylbenzene Styrene Copolymer; Dmdm Hydantoin; Docosanol; Docusate Sodium; Duro-Tak 280-2516; Duro-Tak 387-2516; Duro-Tak 80-1196; Duro-Tak 87-2070; Duro-Tak 87-2194; Duro-Tak 87-2287; Duro-Tak 87-2296; Duro-Tak 87-2888; Duro-Tak 87-2979; Edetate Calcium Disodium; Edetate Disodium; Edetate Disodium Anhydrous; Edetate Sodium; Edetic Acid; Egg Phospholipids; Entsufon; Entsufon Sodium; Epilactose; Epitetracycline Hydrochloride; Essence Bouquet 9200; Ethanolamine Hydrochloride; Ethyl Acetate; Ethyl Oleate; Ethylcelluloses; Ethylene Glycol; Ethylene Vinyl Acetate Copolymer; Ethylenediamine; Ethylenediamine Dihydrochloride; Ethylene-Propylene Copolymer; Ethylene-Vinyl Acetate Copolymer (28% Vinyl Acetate); Ethylene-Vinyl Acetate Copolymer (9% Vinylacetate); Ethylhexyl Hydroxystearate; Ethylparaben; Eucalyptol; Exametazime; Fat, Edible; Fat, Hard; Fatty Acid Esters; Fatty Acid Pentaerythriol Ester; Fatty Acids; Fatty Alcohol Citrate; Fatty Alcohols; Fd&C Blue No. 1; Fd&C Green No. 3; Fd&C Red No. 4; Fd&C Red No. 40; Fd&C Yellow No. 10 (Delisted); Fd&C Yellow No. 5; Fd&C Yellow No. 6; Ferric Chloride; Ferric Oxide; Flavor 89-186; Flavor 89-259; Flavor Df-119; Flavor Df-1530; Flavor Enhancer; Flavor FIG. 827118; Flavor Raspberry Pfc-8407; Flavor Rhodia Pharmaceutical No. Rf 451; Fluorochlorohydrocarbons; Formaldehyde; Formaldehyde Solution; Fractionated Coconut Oil; Fragrance 3949-5; Fragrance 520a; Fragrance 6.007; Fragrance 91-122; Fragrance 9128-Y; Fragrance 93498g; Fragrance Balsam Pine No. 5124; Fragrance Bouquet 10328; Fragrance Chemoderm 6401-B; Fragrance Chemoderm 6411; Fragrance Cream No. 73457; Fragrance Cs-28197; Fragrance Felton 066m; Fragrance Firmenich 47373; Fragrance Givaudan Ess 9090/1c; Fragrance H-6540; Fragrance Herbal 10396; Fragrance Nj-1085; Fragrance P O Fl-147; Fragrance Pa 52805; Fragrance Pera Derm D; Fragrance Rbd-9819; Fragrance Shaw Mudge U-7776; Fragrance Tf 044078; Fragrance Ungerer Honeysuckle K 2771; Fragrance Ungerer N5195; Fructose; Gadolinium Oxide; Galactose; Gamma Cyclodextrin; Gelatin; Gelatin, Crosslinked; Gelfoam Sponge; Gellan Gum (Low Acyl); Gelva 737; Gentisic Acid; Gentisic Acid Ethanolamide; Gluceptate Sodium; Gluceptate Sodium Dihydrate; Gluconolactone; Glucuronic Acid; Glutamic Acid, Dl-; Glutathione; Glycerin; Glycerol Ester Of Hydrogenated Rosin; Glyceryl Citrate; Glyceryl Isostearate; Glyceryl Laurate; Glyceryl Monostearate; Glyceryl Oleate; Glyceryl Oleate/Propylene Glycol; Glyceryl Palmitate; Glyceryl Ricinoleate; Glyceryl Stearate; Glyceryl Stearate-Laureth-23; Glyceryl Stearate/Peg Stearate; Glyceryl Stearate/Peg-100 Stearate; Glyceryl Stearate/Peg-40 Stearate; Glyceryl Stearate-Stearamidoethyl Diethylamine; Glyceryl Trioleate; Glycine; Glycine Hydrochloride; Glycol Distearate; Glycol Stearate; Guanidine Hydrochloride; Guar Gum; Hair Conditioner (18n195-1m); Heptane; Hetastarch; Hexylene Glycol; High Density Polyethylene; Histidine; Human Albumin Microspheres; Hyaluronate Sodium; Hydrocarbon; Hydrocarbon Gel, Plasticized; Hydrochloric Acid; Hydrochloric Acid, Diluted; Hydrocortisone; Hydrogel Polymer; Hydrogen Peroxide; Hydrogenated Castor Oil; Hydrogenated Palm Oil; Hydrogenated Palm/Palm Kernel Oil Peg-6 Esters; Hydrogenated Polybutene 635-690; Hydroxide Ion; Hydroxyethyl Cellulose; Hydroxyethylpiperazine Ethane Sulfonic Acid; Hydroxymethyl Cellulose; Hydroxyoctacosanyl Hydroxystearate; Hydroxypropyl Cellulose; Hydroxypropyl Methylcellulose 2906; Hydroxypropyl-Beta-cyclodextrin; Hypromellose 2208 (15000 Mpa·S); Hypromellose 2910 (15000 Mpa·S); Hypromelloses; Imidurea; Iodine; Iodoxamic Acid; Iofetamine Hydrochloride; Irish Moss Extract; Isobutane; Isoceteth-20; Isoleucine; Isooctyl Acrylate; Isopropyl Alcohol; Isopropyl Isostearate; Isopropyl Myristate; Isopropyl Myristate—Myristyl Alcohol; Isopropyl Palmitate; Isopropyl Stearate; Isostearic Acid; Isostearyl Alcohol; Isotonic Sodium Chloride Solution; Jelene; Kaolin; Kathon Cg; Kathon Cg II; Lactate; Lactic Acid; Lactic Acid, Dl-; Lactic Acid, L-; Lactobionic Acid; Lactose; Lactose Monohydrate; Lactose, Hydrous; Laneth; Lanolin; Lanolin Alcohol—Mineral Oil; Lanolin Alcohols; Lanolin Anhydrous; Lanolin Cholesterols; Lanolin Nonionic Derivatives; Lanolin, Ethoxylated; Lanolin, Hydrogenated; Lauralkonium Chloride; Lauramine Oxide; Laurdimonium Hydrolyzed Animal Collagen; Laureth Sulfate; Laureth-2; Laureth-23; Laureth-4; Lauric Diethanolamide; Laurie Myristic Diethanolamide; Lauroyl Sarcosine; Lauryl Lactate; Lauryl Sulfate; Lavandula Angustifolia Flowering Top; Lecithin; Lecithin Unbleached; Lecithin, Egg; Lecithin, Hydrogenated; Lecithin, Hydrogenated Soy; Lecithin, Soybean; Lemon Oil; Leucine; Levulinic Acid; Lidofenin; Light Mineral Oil; Light Mineral Oil (85 Ssu); Limonene, (+/−)-; Lipocol Sc-15; Lysine; Lysine Acetate; Lysine Monohydrate; Magnesium Aluminum Silicate; Magnesium Aluminum Silicate Hydrate; Magnesium Chloride; Magnesium Nitrate; Magnesium Stearate; Maleic Acid; Mannitol; Maprofix; Mebrofenin; Medical Adhesive Modified S-15; Medical Antiform A-F Emulsion; Medronate Disodium; Medronic Acid; Meglumine; Menthol; Metacresol; Metaphosphoric Acid; Methanesulfonic Acid; Methionine; Methyl Alcohol; Methyl Gluceth-10; Methyl Gluceth-20; Methyl Gluceth-20 Sesquistearate; Methyl Glucose Sesquistearate; Methyl Laurate; Methyl Pyrrolidone; Methyl Salicylate; Methyl Stearate; Methylboronic Acid; Methylcellulose (4000 Mpa·S); Methylcelluloses; Methylchloroisothiazolinone; Methylene Blue; Methylisothiazolinone; Methylparaben; Microcrystalline Wax; Mineral Oil; Mono And Diglyceride; Monostearyl Citrate; Monothioglycerol; Multisterol Extract; Myristyl Alcohol; Myristyl Lactate; Myristyl-.Gamma.-Picolinium Chloride; N-(Carbamoyl-Methoxy Peg-40)-1,2-Distearoyl-Cephalin Sodium; N,N-Dimethylacetamide; Niacinamide; Nioxime; Nitric Acid; Nitrogen; Nonoxynol Iodine; Nonoxynol-15; Nonoxynol-9; Norflurane; Oatmeal; Octadecene-1/Maleic Acid Copolymer; Octanoic Acid; Octisalate, Octoxynol-1; Octoxynol-40; Octoxynol-9; Octyldodecanol; Octylphenol Polymethylene; Oleic Acid; Oleth-10/Oleth-5; Oleth-2; Oleth-20; Oleyl Alcohol; Oleyl Oleate; Olive Oil; Oxidronate Disodium; Oxyquinoline; Palm Kernel Oil; Palmitamine Oxide; Parabens; Paraffin; Paraffin, White Soft; Parfum Creme 45/3; Peanut Oil; Peanut Oil, Refined; Pectin; Peg 6-32 Stearate/Glycol Stearate; Peg Vegetable Oil; Peg-100 Stearate; Peg-12 Glyceryl Laurate; Peg-120 Glyceryl Stearate; Peg-120 Methyl Glucose Dioleate; Peg-15 Cocamine; Peg-150 Distearate; Peg-2 Stearate; Peg-20 Sorbitan Isostearate; Peg-22 Methyl Ether/Dodecyl Glycol Copolymer; Peg-25 Propylene Glycol Stearate; Peg-4 Dilaurate; Peg-4 Laurate; Peg-40 Castor Oil; Peg-40 Sorbitan Diisostearate; Peg-45/Dodecyl Glycol Copolymer; Peg-5 Oleate; Peg-50 Stearate; Peg-54 Hydrogenated Castor Oil; Peg-6 Isostearate; Peg-60 Castor Oil; Peg-60 Hydrogenated Castor Oil; Peg-7 Methyl Ether; Peg-75 Lanolin; Peg-8 Laurate; Peg-8 Stearate; Pegoxol 7 Stearate; Pentadecalactone; Pentaerythritol Cocoate; Pentasodium Pentetate; Pentetate Calcium Trisodium; Pentetic Acid; Peppermint Oil; Perflutren; Perfume 25677; Perfume Bouquet; Perfume E-1991; Perfume Gd 5604; Perfume Tana 90/42 Scba; Perfume W-1952-1; Petrolatum; Petrolatum, White; Petroleum Distillates; Phenol; Phenol, Liquefied; Phenonip; Phenoxyethanol; Phenylalanine; Phenylethyl Alcohol; Phenylmercuric Acetate; Phenylmercuric Nitrate; Phosphatidyl Glycerol, Egg; Phospholipid; Phospholipid, Egg; Phospholipon 90g; Phosphoric Acid; Pine Needle Oil (Pinus Sylvestris); Piperazine Hexahydrate; Plastibase-50w; Polacrilin; Polidronium Chloride; Poloxamer 124; Poloxamer 181; Poloxamer 182; Poloxamer 188; Poloxamer 237; Poloxamer 407; Poly(Bis(P-Carboxyphenoxy)Propane Anhydride):Sebacic Acid; Poly(Dimethylsiloxane/Methylvinylsiloxane/Methylhydrogensiloxane) Dimethylvinyl Or Dimethylhydroxy Or Trimethyl Endblocked; Poly(Dl-Lactic-Co-Glycolic Acid), (50:50; Poly(Dl-Lactic-Co-Glycolic Acid), Ethyl Ester Terminated, (50:50; Polyacrylic Acid (250000 Mw); Polybutene (1400 Mw); Polycarbophil; Polyester; Polyester Polyamine Copolymer; Polyester Rayon; Polyethylene Glycol 1000; Polyethylene Glycol 1450; Polyethylene Glycol 1500; Polyethylene Glycol 1540; Polyethylene Glycol 200; Polyethylene Glycol 300; Polyethylene Glycol 300-1600; Polyethylene Glycol 3350; Polyethylene Glycol 400; Polyethylene Glycol 4000; Polyethylene Glycol 540; Polyethylene Glycol 600; Polyethylene Glycol 6000; Polyethylene Glycol 8000; Polyethylene Glycol 900; Polyethylene High Density Containing Ferric Oxide Black (<1%); Polyethylene Low Density Containing Barium Sulfate (20-24%); Polyethylene T; Polyethylene Terephthalates; Polyglactin; Polyglyceryl-3 Oleate; Polyglyceryl-4 Oleate; Polyhydroxyethyl Methacrylate; Polyisobutylene; Polyisobutylene (1100000 Mw); Polyisobutylene (35000 Mw); Polyisobutylene 178-236; Polyisobutylene 241-294; Polyisobutylene 35-39; Polyisobutylene Low Molecular Weight; Polyisobutylene Medium Molecular Weight; Polyisobutylene/Polybutene Adhesive; Polylactide; Polyols; Polyoxyethylene—Polyoxypropylene 1800; Polyoxyethylene Alcohols; Polyoxyethylene Fatty Acid Esters; Polyoxyethylene Propylene; Polyoxyl 20 Cetostearyl Ether; Polyoxyl 35 Castor Oil; Polyoxyl 40 Hydrogenated Castor Oil; Polyoxyl 40 Stearate; Polyoxyl 400 Stearate; Polyoxyl 6 And Polyoxyl 32 Palmitostearate; Polyoxyl Distearate; Polyoxyl Glyceryl Stearate; Polyoxyl Lanolin; Polyoxyl Palmitate; Polyoxyl Stearate; Polypropylene; Polypropylene Glycol; Polyquaternium-10; Polyquaternium-7 (70/30 Acrylamide/Dadmac; Polysiloxane; Polysorbate 20; Polysorbate 40; Polysorbate 60; Polysorbate 65; Polysorbate 80; Polyurethane; Polyvinyl Acetate; Polyvinyl Alcohol; Polyvinyl Chloride; Polyvinyl Chloride-Polyvinyl Acetate Copolymer; Polyvinylpyridine; Poppy Seed Oil; Potash; Potassium Acetate; Potassium Alum; Potassium Bicarbonate; Potassium Bisulfite; Potassium Chloride; Potassium Citrate; Potassium Hydroxide; Potassium Metabisulfite; Potassium Phosphate, Dibasic; Potassium Phosphate, Monobasic; Potassium Soap; Potassium Sorbate; Povidone Acrylate Copolymer; Povidone Hydrogel; Povidone K17; Povidone K25; Povidone K29/32; Povidone K30; Povidone K90; Povidone K90f; Povidone/Eicosene Copolymer; Povidones; Ppg-12/Smdi Copolymer; Ppg-15 Stearyl Ether; Ppg-20 Methyl Glucose Ether Distearate; Ppg-26 Oleate; Product Wat; Proline; Promulgen D; Promulgen G; Propane; Propellant A-46; Propyl Gallate; Propylene Carbonate; Propylene Glycol; Propylene Glycol Diacetate; Propylene Glycol Dicaprylate; Propylene Glycol Monolaurate; Propylene Glycol Monopalmitostearate; Propylene Glycol Palmitostearate; Propylene Glycol Ricinoleate; Propylene Glycol/Diazolidinyl; Urea/Methylparaben/Propylparben; Propylparaben; Protamine Sulfate; Protein Hydrolysate; Pvm/Ma Copolymer; Quaternium-15; Quaternium-Cis-Form; Quaternium-52; Ra-2397; Ra-3011; Saccharin; Saccharin Sodium; Saccharin Sodium Anhydrous; Safflower Oil; Sd Alcohol 3a; Sd Alcohol 40; Sd Alcohol 40-2; Sd Alcohol 40b; Sepineo P 600; Serine; Sesame Oil; Shea Butter; Silastic Brand Medical Grade Tubing; Silastic Medical Adhesive, Silicone Type A; Silica, Dental; Silicon; Silicon Dioxide; Silicon Dioxide, Colloidal; Silicone; Silicone Adhesive 4102; Silicone Adhesive 4502; Silicone Adhesive Bio-Psa Q7-4201; Silicone Adhesive Bio-Psa Q7-4301; Silicone Emulsion; Silicone/Polyester Film Strip; Simethicone; Simethicone Emulsion; Sipon Ls 20np; Soda Ash; Sodium Acetate; Sodium Acetate Anhydrous; Sodium Alkyl Sulfate; Sodium Ascorbate; Sodium Benzoate; Sodium Bicarbonate; Sodium Bisulfate; Sodium Bisulfite; Sodium Borate; Sodium Borate Decahydrate; Sodium Carbonate; Sodium Carbonate Decahydrate; Sodium Carbonate Monohydrate; Sodium Cetostearyl Sulfate; Sodium Chlorate; Sodium Chloride; Sodium Chloride Injection; Sodium Chloride Injection, Bacteriostatic; Sodium Cholesteryl Sulfate; Sodium Citrate; Sodium Cocoyl Sarcosinate; Sodium Desoxycholate; Sodium Dithionite; Sodium Dodecylbenzenesulfonate; Sodium Formaldehyde Sulfoxylate; Sodium Gluconate; Sodium Hydroxide; Sodium Hypochlorite; Sodium Iodide; Sodium Lactate; Sodium Lactate, L-; Sodium Laureth-2 Sulfate; Sodium Laureth-3 Sulfate; Sodium Laureth-5 Sulfate; Sodium Lauroyl Sarcosinate; Sodium Lauryl Sulfate; Sodium Lauryl Sulfoacetate; Sodium Metabisulfite; Sodium Nitrate; Sodium Phosphate; Sodium Phosphate Dihydrate; Sodium Phosphate, Dibasic; Sodium Phosphate, Dibasic, Anhydrous; Sodium Phosphate, Dibasic, Dihydrate; Sodium Phosphate, Dibasic, Dodecahydrate; Sodium Phosphate, Dibasic, Heptahydrate; Sodium Phosphate, Monobasic; Sodium Phosphate, Monobasic, Anhydrous; Sodium Phosphate, Monobasic, Dihydrate; Sodium Phosphate, Monobasic, Monohydrate; Sodium Polyacrylate (2500000 Mw); Sodium Pyrophosphate; Sodium Pyrrolidone Carboxylate; Sodium Starch Glycolate; Sodium Succinate Hexahydrate; Sodium Sulfate; Sodium Sulfate Anhydrous; Sodium Sulfate Decahydrate; Sodium Sulfite; Sodium Sulfosuccinated Undecyclenic Monoalkylolamide; Sodium Tartrate; Sodium Thioglycolate; Sodium Thiomalate; Sodium Thiosulfate; Sodium Thiosulfate Anhydrous; Sodium Trimetaphosphate; Sodium Xylenesulfonate; Somay 44; Sorbic Acid; Sorbitan; Sorbitan Isostearate; Sorbitan Monolaurate; Sorbitan Monooleate; Sorbitan Monopalmitate; Sorbitan Monostearate; Sorbitan Sesquioleate; Sorbitan Trioleate; Sorbitan Tristearate; Sorbitol; Sorbitol Solution; Soybean Flour; Soybean Oil; Spearmint Oil; Spermaceti; Squalane; Stabilized Oxychloro Complex; Stannous 2-Ethylhexanoate; Stannous Chloride; Stannous Chloride Anhydrous; Stannous Fluoride; Stannous Tartrate; Starch; Starch 1500, Pregelatinized; Starch, Corn; Stearalkonium Chloride; Stearalkonium Hectorite/Propylene Carbonate; Stearamidoethyl Diethylamine; Steareth-10; Steareth-100; Steareth-2; Steareth-20; Steareth-21; Steareth-40; Stearic Acid; Stearic Diethanolamide; Stearoxytrimethylsilane; Steartrimonium Hydrolyzed Animal Collagen; Stearyl Alcohol; Sterile Water For Inhalation; Styrene/Isoprene/Styrene Block Copolymer; Succimer; Succinic Acid; Sucralose; Sucrose; Sucrose Distearate; Sucrose Polyesters; Sulfacetamide Sodium; Sulfobutylether .Beta.-Cyclodextrin; Sulfur Dioxide; Sulfuric Acid; Sulfurous Acid; Surfactol Qs; Tagatose, D-; Talc; Tall Oil; Tallow Glycerides; Tartaric Acid; Tartaric Acid, Dl-; Tenox; Tenox-2; Tert-Butyl Alcohol; Tert-Butyl Hydroperoxide; Tert-Butylhydroquinone; Tetrakis(2-Methoxyisobutylisocyanide)Copper(I) Tetrafluoroborate; Tetrapropyl Orthosilicate; Tetrofosmin; Theophylline; Thimerosal; Threonine; Thymol; Tin; Titanium Dioxide; Tocopherol; Tocophersolan; Total parenteral nutrition, lipid emulsion; Triacetin; Tricaprylin; Trichloromonofluoromethane; Trideceth-10; Triethanolamine Lauryl Sulfate; Trifluoroacetic Acid; Triglycerides, Medium Chain; Trihydroxystearin; Trilaneth-4 Phosphate; Trilaureth-4 Phosphate; Trisodium Citrate Dihydrate; Trisodium Hedta; Triton 720; Triton X-200; Trolamine; Tromantadine; Tromethamine (TRIS); Tryptophan; Tyloxapol; Tyrosine; Undecylenic Acid; Union 76 Amsco-Res 6038; Urea; Valine; Vegetable Oil; Vegetable Oil Glyceride, Hydrogenated; Vegetable Oil, Hydrogenated; Versetamide; Viscarin; Viscose/Cotton; Vitamin E; Wax, Emulsifying; Wecobee Fs; White Ceresin Wax; White Wax; Xanthan Gum; Zinc; Zinc Acetate; Zinc Carbonate; Zinc Chloride; and Zinc Oxide.

Pharmaceutical formulations of AAV particles disclosed herein may comprise cations or anions. In certain embodiments, the formulations comprise metal cations such as, but not limited to, Zn²⁺, Ca⁺², Cu²⁺, Mn²⁺, Mg⁺ and combinations thereof. As a non-limiting example, formulations may comprise polymers and complexes with a metal cation (See e.g., U.S. Pat. Nos. 6,265,389 and 6,555,525, each of which is herein incorporated by reference in its entirety).

Formulations of the present disclosure may also comprise one or more pharmaceutically acceptable salts. As used herein, “pharmaceutically acceptable salts” refers to derivatives of the disclosed compounds wherein the parent compound is modified by converting an existing acid or base moiety to its salt form (e.g., by reacting the free base group with a suitable organic acid).

In certain embodiments, additional excipients that may be used in formulating the pharmaceutical composition may comprise magnesium chloride (MgCl₂), arginine, sorbitol, and/or trehalose.

Formulations of the present disclosure may comprise at least one excipient and/or diluent in addition to the AAV particle. The formulation may comprise 1, 2, 3, 4, 5, 6, 7, 8, 9, 10, or more than 10 excipients and/or diluents in addition to the AAV particle.

In certain embodiments, the formulation may comprise, but is not limited to, phosphate-buffered saline (PBS). As a non-limiting example, the PBS may comprise sodium chloride, potassium chloride, disodium phosphate, monopotassium phosphate, and distilled water. In some instances, the PBS does not contain potassium or magnesium. In other instances, the PBS contains calcium and magnesium.

### Sodium Phosphate

In certain embodiments, at least one of the components in the formulation is sodium phosphate. The formulation may comprise monobasic, dibasic or a combination of both monobasic and dibasic sodium phosphate. In certain embodiments, the concentration of sodium phosphate in a formulation may be, but is not limited to, 0.1-15 mM (or any value or range therein). In certain embodiments, the formulation may include 0-10 mM of sodium phosphate. In certain embodiments, the formulation may comprise 2-12 mM of sodium phosphate. In certain embodiments, the formulation may comprise 2-3 mM of sodium phosphate. In certain embodiments, the formulation may comprise 9-10 mM of sodium phosphate. In certain embodiments, the formulation may comprise 10-11 mM of sodium phosphate. In certain embodiments, the formulation may comprise 2.7 mM of sodium phosphate. In certain embodiments, the formulation may comprise 10 mM of sodium phosphate.

### Potassium Phosphate

In certain embodiments, at least one of the components in the formulation is potassium phosphate. The formulation may comprise monobasic, dibasic or a combination of both monobasic and dibasic potassium phosphate. In certain embodiments, the concentration of potassium phosphate in a formulation may be, but is not limited to, 0.1-15 mM (or any value or range therein). In certain embodiments, the formulation may include 0-10 mM of potassium phosphate. In certain embodiments, the formulation may include 1-3 mM of potassium phosphate. In certain embodiments, the formulation may comprise 1-2 mM of potassium phosphate. In certain embodiments, the formulation may comprise 2-3 mM of potassium phosphate. In certain embodiments, the formulation may comprise 2-12 mM of potassium phosphate. In certain embodiments, the formulation may comprise 1.5 mM of potassium phosphate. As a non-limiting example, the formulation may comprise 1.54 mM of potassium phosphate. In certain embodiments, the formulation may comprise 2 mM of potassium phosphate.

### Sodium Chloride

In certain embodiments, at least one of the components in the formulation is sodium chloride. In certain embodiments, the concentration of sodium chloride in a formulation may be, but is not limited to, 75-220 mM (or any value or range therein). In certain embodiments, the formulation may include 80-220 mM of sodium chloride. In certain embodiments, the formulation may include 80-150 mM of sodium chloride. In certain embodiments, the formulation may include 75 mM of sodium chloride. In certain embodiments, the formulation may comprise 80-220 mM of sodium chloride. In certain embodiments, the formulation may comprise 83 mM of sodium chloride. In certain embodiments, the formulation may comprise 92 mM of sodium chloride. In certain embodiments, the formulation may comprise 95 mM of sodium chloride. In certain embodiments, the formulation may comprise 98 mM of sodium chloride. In certain embodiments, the formulation may comprise 100 mM of sodium chloride. In certain embodiments, the formulation may comprise 107 mM of sodium chloride. In certain embodiments, the formulation may comprise 109 mM of sodium chloride. In certain embodiments, the formulation may comprise 118 mM of sodium chloride. In certain embodiments, the formulation may comprise 125 mM of sodium chloride. In certain embodiments, the formulation may comprise 127 mM of sodium chloride. In certain embodiments, the formulation may comprise 133 mM of sodium chloride. In certain embodiments, the formulation may comprise 142 mM of sodium chloride. In certain embodiments, the formulation may comprise 150 mM of sodium chloride. In certain embodiments, the formulation may comprise 155 mM of sodium chloride. In certain embodiments, the formulation may comprise 180 mM of sodium chloride. In certain embodiments, the formulation may comprise 192 mM of sodium chloride. In certain embodiments, the formulation may comprise 210 mM of sodium chloride.

### Potassium Chloride

In certain embodiments, at least one of the components in the formulation is potassium chloride. In certain embodiments, the concentration of potassium chloride in a formulation may be, but is not limited to, 0.1-15 mM (or any value or range therein). In certain embodiments, the formulation may include 0-10 mM of potassium chloride. In certain embodiments, the formulation may include 1-3 mM of potassium chloride. In certain embodiments, the formulation may comprise 1-2 mM of potassium chloride. In certain embodiments, the formulation may comprise 2-3 mM of potassium chloride. In certain embodiments, the formulation may comprise 1.5 mM of potassium chloride. In certain embodiments, the formulation may comprise 2.7 mM of potassium chloride.

### Magnesium Chloride

In certain embodiments, at least one of the components in the formulation is magnesium chloride. In certain embodiments, the concentration of magnesium chloride may be, but is not limited to, 1-100 mM (or any value or range therein). In certain embodiments, the formulation may include 0-75 mM of magnesium chloride. In certain embodiments, the formulation may comprise 0-75 mM of magnesium chloride. In certain embodiments, the formulation may comprise 0-5 mM of magnesium chloride. In certain embodiments, the formulation may comprise 50-100 mM of magnesium chloride. In certain embodiments, the formulation may comprise 2 mM of magnesium chloride. In certain embodiments, the formulation may comprise 75 mM of magnesium chloride.

### Tris

In certain embodiments, at least one of the components in the formulation is Tris (also called tris(hydroxymethyl)aminomethane, tromethamine or THAM). In certain embodiments, the concentration of Tris in a formulation may be, but is not limited to, 0.1-15 mM. In certain embodiments, the formulation may include 0-10 mM of Tris. In certain embodiments, the formulation may include 2-12 mM of Tris. In certain embodiments, the formulation may include 10 mM of Tris. In certain embodiments, the formulation may comprise 10 mM of Tris.

### Histidine

In certain embodiments, at least one of the components in the formulation is Histidine.

In certain embodiments, the concentration of Histidine. In certain embodiments, the formulation may include 2-12 mM of Histidine. In certain embodiments, the formulation may comprise 0-10 mM of Histidine. In certain embodiments, the formulation may comprise 2-12 mM of Histidine. In certain embodiments, the formulation may comprise 10 mM of Histidine.

### Arginine

In certain embodiments, at least one of the components in the formulation is arginine.

In certain embodiments, the concentration of arginine may be, but is not limited to, 1-100 mM. In certain embodiments, the formulation may include 0-75 mM of arginine. In certain embodiments, the formulation may include 50-100 mM. In certain embodiments, the formulation may comprise 0-75 mM of arginine. In certain embodiments, the formulation may comprise 75 mM of arginine.

### Hydrochloric Acid

In certain embodiments, at least one of the components in the formulation is hydrochloric acid. In certain embodiments, the concentration of hydrochloric acid in a formulation may be, but is not limited to, 0.1-15 mM. In certain embodiments, the formulation may include 0-10 mM of hydrochloric acid. In certain embodiments, the formulation may comprise 6.2-6.3 mM of hydrochloric acid. In certain embodiments, the formulation may comprise 8.9-9 mM of hydrochloric acid. In certain embodiments, the formulation may comprise 6.2 mM of hydrochloric acid. In certain embodiments, the formulation may comprise 6.3 mM of hydrochloric acid. In certain embodiments, the formulation may comprise 8.9 mM of hydrochloric acid. In certain embodiments, the formulation may comprise 9 mM of hydrochloric acid.

### Sugar

In certain embodiments, the formulation may include at least one sugar and/or sugar substitute. In certain embodiments, the formulation may include at least one sugar and/or sugar substitute to increase the stability of the formulation. In certain embodiments, the formulation may include a sugar and/or sugar substitute at 0.1-10% w/v (or any value or range therein). In certain embodiments, the formulation may include a sugar and/or sugar substitute in a range of 0.1-10% w/v (or any value or range therein). In certain embodiments, the formulation may include 0-10% w/v of a sugar and/or sugar substitute. In certain embodiments, the formulation may include 0.1-1%, 1-2%, 2-3%, 3-4%, 4-5%, 5-6%, 6-7%, 7-8%, 8-9%, or 9-10% w/v of a sugar and/or sugar substitute.

In certain embodiments, the formulation may include at least one sugar which is sucrose. In certain embodiments, the formulation may include sucrose at 0.1-10% w/v (or any value or range therein). In certain embodiments, the formulation may include 0.1-1%, 1-2%, 2-3%, 3-4%, 4-5%, 5-6%, 6-7%, 7-8%, 8-9%, or 9-10% w/v of sucrose.

In certain embodiments, the formulation may include at least one sugar which is trehalose. In certain embodiments, the formulation may include trehalose at 0.1-10% w/v (or any value or range therein). In certain embodiments, the formulation may include 0.1-1%, 1-2%, 2-3%, 3-4%, 4-5%, 5-6%, 6-7%, 7-8%, 8-9%, or 9-10% w/v of trehalose.

In certain embodiments, the formulation may include at least one sugar substitute which is sorbitol. In certain embodiments, the formulation may include sorbitol at 0.1-10% w/v (or any value or range therein). In certain embodiments, the formulation may include 0.1-1%, 1-2%, 2-3%, 34%, 4-5%, 5-6%, 6-7%, 7-8%, 8-9%, or 9-10% w/v of sorbitol.

### Surfactant

In certain embodiments, formulations of pharmaceutical compositions described herein may comprise a surfactant. Surfactants may help control shear forces in suspension cultures. Surfactants used herein may be anionic, zwitterionic, or non-ionic surfactants and may comprise those known in the art that are suitable for use in pharmaceutical formulations.

Examples of anionic surfactants comprise, but are not limited to, sulfate, sulfonate, phosphate esters, and carboxylates.

Examples of nonionic surfactants comprise, but are not limited to, ethoxylates, fatty alcohol ethoxylates, alkylphenol ethoxylates (e.g., nonoxynols, Triton X-100), fatty acid ethoxylates, ethoxylated amines and/or fatty acid amides (e.g., polyethoxylated tallow amine, cocamide monoethanolamine, cocamide diethanolamine), ethylene oxide/propylene oxide copolymer (e.g., Poloxamers such as Pluronic® F-68 or F-127), esters of fatty acids and polyhydric alcohols, fatty acid alkanolamides, ethoxylated aliphatic acids, ethoxylated aliphatic alcohols, ethoxylated sorbitol fatty acid esters, ethoxylated glycerides, ethoxylated block copolymers with EDTA (ethylene diaminetetraacetic acid), ethoxylated cyclic ether adducts, ethoxylated amide and imidazoline adducts, ethoxylated amine adducts, ethoxylated mercaptan adducts, ethoxylated condensates with alkyl phenols, ethoxylated nitrogen-based hydrophobes, ethoxylated polyoxypropylenes, polymeric silicones, fluorinated surfactants, and polymerizable surfactants.

Examples of zwitterionic surfactants comprise, but are not limited to, alkylamido betaines and amine oxides thereof, alkyl betaines and amine oxides thereof, sulfo betaines, hydroxy sulfo betaines, amphoglycinates, amphopropionates, balanced amphopoly-carboxyglycinates, and alkyl polyaminoglycinates. Proteins have the ability of being charged or uncharged depending on the pH; thus, at the right pH, a protein, preferably with a pI of about 8 to 9, such as modified Bovine Serum Albumin or chymotrypsinogen, could function as a zwitterionic surfactant. Various mixtures of surfactants can be used if desired.

### Copolymers

In certain embodiments, at least one of the components in the formulation is copolymer.

In certain embodiments, the formulation may comprise at least one copolymer at a concentration of 0.00001%, 0.0001%, 0.001%, 0.01%, 0.1%, or 1% w/v.

In certain embodiments, the formulation may comprise at least one copolymer in a range of 0.00001%-0.0001%, 0.00001%-0.001%, 0.00001%-0.01%, 0.00001%-0.1%, 0.00001%-1%, 0.0001%-0.001%, 0.0001%-0.01%, 0.0001%-0.1%, 0.0001%-1%, 0.001%-0.01%, 0.001%-0.1%, 0.001%-1%, 0.01%-0.1%, 0.01%-01%, or 0.1-1% w/v.

In certain embodiments, the formulation may comprise 0.001% w/v copolymer.

In certain embodiments, the copolymer is an ethylene oxide/propylene oxide copolymer.

In certain embodiments, the formulation may comprise at least one ethylene oxide/propylene oxide copolymer at a concentration of 0.00001%, 0.0001%, 0.001%, 0.01%, 0.1%, or 1% w/v.

In certain embodiments, the formulation may comprise at least one ethylene oxide/propylene oxide copolymer in a range of 0.00001%-0.0001%, 0.00001%-0.001%, 0.00001%-0.01%, 000001%-0.1%, 000001%-1%, 0.0001%-0.001%, 0.0001%-0.01%, 0.0001%-0.1%, 0.0001%-1%, 0.001%-0.01%, 0.001%-0.1%, 0.001%-1%, 0.01%-0.1%, 0.01%-1%, or 0.1-1% w/v.

In certain embodiments, the formulation may comprise 0.001% w/v ethylene oxide/propylene oxide copolymer.

In certain embodiments, the formulation may comprise at least one ethylene oxide/propylene copolymer which is a Poloxamer. In certain embodiments, the formulation may comprise Poloxamer at a concentration of 0.00001%, 0.0001%, 0.001%, 0.01%, 0.1%, or 1% w/v.

In certain embodiments, the formulation may comprise Poloxamer in a range of 0.00001%-0.0001%, 0.00001%-0.001%, 0.00001%-0.01%, 0.00001%-0.1%, 0.00001%-1%, 0.0001%-0.001%, 0.0001%-0.01%, 0.0001%-0.1%, 0.0001%-1%, 0.001%-0.0%, 0.001%-0.1%, 0.001%-1%, 0.01%-0.1%, 0.01%-1%, or 0.1-1% w/v.

In certain embodiments, the formulation may comprise 0.001% w/v Poloxamer.

In certain embodiments, the formulation may comprise at least one ethylene oxide/propylene copolymer which is Poloxamer 188. In certain embodiments, the formulation may comprise Poloxamer 188 at a concentration of 0.00001%, 0.0001%, 0.001%, 0.01%, 0.1%, or 1% w/v.

In certain embodiments, the formulation may comprise Poloxamer 188 in a range of 0.00001%-0.0001%, 0.00001%-0.001%, 0.00001%-0.01%, 0.00001%-0.1%, 0.00001%-1%, 0.0001%-0.001%, 0.0001%0.01%, 0.0001%-0.1%, 0.0001%-1%, 0.001%-0.01%, 0.001%-0.1%, 0.001%-1%, 0.01%-0.1%, 0.01%-1%, or 0.1-1% w/v.

In certain embodiments, the formulation may comprise 0.001%-0.1 w/v Poloxamer 188.

In certain embodiments, the formulation may comprise at least one ethylene oxide/propylene copolymer which is Pluronic® F-68. In certain embodiments, the formulation may comprise Pluronic® F-68 at a concentration of 0.00001%, 0.0001%, 0.001%, 0.01%, 0.1%, or 1% w/v.

In certain embodiments, the formulation may comprise Pluronic® F-68 in a range of 0.00001%-0.0001%, 0.00001%-0.001%, 0.00001%-0.01%, 0.00001-0.1%, 0.00001%-1%, 0.0001%-0.001%, 0.0001%-0.01%, 0.0001%-0.1%, 0.0001%-1%, 0.001%-0.01%, 0.001%-0.1%, 0.001%-1%, 0.01%-0.1%, 0.01%-1%, or 0.1-1% w/v.

In certain embodiments, the formulation may comprise 0.001%-0.1% w/v Pluronic® F-68. In certain embodiments, the formulation may comprise 0.001% w/v Pluronic® F-68.

### Formulation Properties

In certain embodiments, the formulation has been optimized to have a specific pH, osmolality, concentration, concentration of AAV particle, and/or total dose of AAV particle.

pH

In certain embodiments, the formulation may be optimized for a specific pH. In certain embodiments, the formulation may comprise a pH buffering agent (also referred to herein as “buffering agent”) which is a weak acid or base that, when used in the formulation, maintains the pH of the formulation near a chosen value even after another acid or base is added to the formulation. The pH of the formulation may be, but is not limited, to 0, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, and 14.

In certain embodiments, the formulation may be optimized for a specific pH range. The pH range may be, but is not limited to, 0-4, 1-5, 2-6, 3-7, 4-8, 5-9, 6-10, 7-11, 8-12, 9-13, 10-14, 0-1.5, 1-2.5, 2-3.5, 3-4.5, 4-5.5, 5-6.5, 6-7.5, 7-8.5, 8-9.5, 9-10.5, 10-11.5, 11-12.5, 12-13.5, 0-1, 1-2, 2-3, 3-4, 4-5, 5-6, 6-7, 7-8, 8-9, 9-10, 10-11, 11-12, 12-13, 13-14, 0-0.5, 0.5-1, 1-1.5, 1.5-2, 2-2.5, 2.5-3, 3-3.5, 3.5-4, 4-4.5, 4.5-5, 5-5.5, 5.5-6, 6-6.5, 6.5-7, 7-7.5, 7.2-8.2, 7.2-7.6, 7.3-7.7, 7.5-8, 7.8-8.2, 8-8.5, 8.5-9, 9-9.5, 9.5-10, 10-10.5, 10.5-11, 11-11.5, 11.5-12, 12-12.5, 12.5-13, 13-13.5, or 13.5-14.

In certain embodiments, the pH of the formulation is between 6 and 8.5. In certain embodiments, the pH of the formulation is between 7 and 8.5. In certain embodiments, the pH of the formulation is between 7 and 7.6. In certain embodiments, the pH of the formulation is 7. In certain embodiments, the pH of the formulation is 7.1. In certain embodiments, the pH of the formulation is 7.2. In certain embodiments, the pH of the formulation is 7.3. In certain embodiments, the pH of the formulation is 7.4. In certain embodiments, the pH of the formulation is 7.5. In certain embodiments, the pH of the formulation is 7.6. In certain embodiments, the pH of the formulation is 7.7. In certain embodiments, the pH of the formulation is 7.8. In certain embodiments, the pH of the formulation is 7.9. In certain embodiments, the pH of the formulation is 8. In certain embodiments, the pH of the formulation is 8.1. In certain embodiments, the pH of the formulation is 8.2. In certain embodiments, the pH of the formulation is 8.3. In certain embodiments, the pH of the formulation is 8.4. In certain embodiments, the pH of the formulation is 8.5. In certain embodiments, the pH is determined when the formulation is at 5° C. In certain embodiments, the pH is determined when the formulation is at 25° C.

Suitable buffering agents may comprise, but not limited to, Tris HCl, Tris base, sodium phosphate (monosodium phosphate and/or disodium phosphate), potassium phosphate (monopotassium phosphate and/or dipotassium phosphate), histidine, boric acid, citric acid, glycine, HEPES (4-(2-hydroxyethyl)-1-piperazineethanesulfonic acid), and MOPS (3-(N-morpholino)propanesulfonic acid).

Concentration of buffering agents in the formulation may be between 1-50 mM, between 1-25 mM, between 5-30 mM, between 5-20 mM, between 5-15 mM, between 10-40 mM, or between 15-30 mM. Concentration of buffering agents in the formulation may be about 1 mM, 5 mM, 7.5 mM, 10 mM, 12.5 mM, 15 mM, 20 mM, 25 mM, 30 mM, 35 mM, 40 mM, or 50 mM.

In certain embodiments, the formulation may comprise, but is not limited to, phosphate-buffered saline (PBS). As a non-limiting example, the PBS may comprise sodium chloride, potassium chloride, disodium phosphate, monopotassium phosphate, and distilled water. In some instances, the PBS does not contain potassium or magnesium. In other instances, the PBS contains calcium and magnesium.

In certain embodiments, buffering agents used in the formulations of pharmaceutical compositions described herein may comprise sodium phosphate (monosodium phosphate and/or disodium phosphate). As a non-limiting example, sodium phosphate may be adjusted to a pH (at 5° C.) within the range of 7.4±0.2. In certain embodiments, buffering agents used in the formulations of pharmaceutical compositions described herein may comprise Tris base. Tris base may be adjusted with hydrochloric acid to any pH within the range of 7.1 and 9.1. As a non-limiting example, Tris base used in the formulations described herein may be adjusted to 8.0±0.2. As a non-limiting example. Tris base used in the formulations described herein may be adjusted to 7.5±0.2.

### Osmolality

In certain embodiments, the formulation may be optimized for a specific osmolality. The osmolality of the formulation may be, but is not limited to, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404,405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494,495, 496, 497, 498, 499, or 500 mOsm/kg (milliosmoles/kg).

In certain embodiments, the formulation may be optimized for a specific range of osmolality. The range may be, but is not limited to, 350-360, 360-370, 370-380, 380-390, 390-400, 400-410, 410-420, 420-430, 430-440, 440-450, 450-460, 460-470, 470-480, 480-490, 490-500, 350-370, 360-380, 370-390, 380-400, 390-410, 400-420, 410-430, 420-440, 430-450, 440-460, 450-470, 460-480, 470-490, 480-500, 350-375, 375-400, 400-425, 425-450, 450-475, 475-500, 350-380, 360-390, 370-400, 380-410, 390-420, 400-430, 410-440, 420-450, 430-460, 440-470, 450-480, 460-490, 470-500, 350-390, 360-400, 370-410, 380-420, 390-430, 400-440, 410-450, 420-460, 430-470, 440-480, 450-490, 460-500, 350-400, 360-410, 370-420, 380-430, 390-440, 400-450, 410-460, 420-470, 430-480, 440-490, 450-500, 350-410, 360-420, 370-430, 380-440, 390-450, 400-460, 410-470, 420-480, 430-490, 440-500, 350-420, 360-430, 370-440, 380-450, 390-460, 400-470, 410-480, 420-490, 430-500, 350-430, 360-440, 370-450, 380-460, 390-470, 400-480, 410-490, 420-500, 350-440, 360-450, 370-460, 380-470, 390-480, 400-490, 410-500, 350-450, 360-460, 370-470, 380-480, 390-490, 400-500, 350-460, 360-470, 370-480, 380-490, 390-500, 350-470, 360-480, 370-490, 380-500, 350-480, 360-490, 370-500, 350-490, 360-500, or 350-500 mOsm/kg.

In certain embodiments, the osmolality of the formulation is between 350-500 mOsm/kg. In certain embodiments, the osmolality of the formulation is between 400-500 mOsm/kg. In certain embodiments, the osmolality of the formulation is between 400480 mOsm/kg. In certain embodiments, the osmolality is 395 mOsm/kg. In certain embodiments, the osmolality is 413 mOsm/kg. In certain embodiments, the osmolality is 420 mOsm/kg. In certain embodiments, the osmolality is 432 mOsm/kg. In certain embodiments, the osmolality is 447 mOsm/kg. In certain embodiments, the osmolality is 450 mOsm/kg. In certain embodiments, the osmolality is 452 mOsm/kg. In certain embodiments, the osmolality is 459 mOsm/kg. In certain embodiments, the osmolality is 472 mOsm/kg. In certain embodiments, the osmolality is 490 mOsm/kg. In certain embodiments, the osmolality is 496 mOsm/kg.

### Concentration of AAV Particle

In certain embodiments, the concentration of AAV particle in the formulation may be between about 1×10⁶ VG/ml and about 1×10¹⁶ VG/ml. As used herein, “VG/ml” represents vector genomes (VG) per milliliter (ml). VG/ml also may describe genome copy per milliliter or DNase resistant particle per milliliter.

In certain embodiments, the formulation may comprise an AAV particle concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 7.1×10¹², 7.2×10¹², 7.3×10¹², 7.4×10¹², 7.5×10¹², 7.6×10¹², 7.7×10¹², 7.8×10¹², 7.9×10¹², 8×10¹², 8.1×10¹², 8.2×10¹², 8.3×10¹², 8.4×10¹², 8.5×10¹², 8.6×10¹², 8.7×10¹², 8.8×10¹², 8.9×10¹², 9×10¹², 1×10¹³, 1.1×10¹³, 1.2×10¹³, 1.3×10¹³, 1.4×10¹³, 1.5×10¹³, 1.6×10¹³, 1.7×10¹³, 1.8×10¹³, 1.9×10¹³, 2×10¹³, 2.1×10¹³, 2.2×10¹³, 2.3×10¹³, 2.4×10¹³, 2.5×10¹³, 2.6×10¹³, 2.7×10¹³, 2.8×10¹³, 2.9×10¹³, 3×10¹³, 3.1×10¹³, 3.2×10¹³, 3.3×10¹³, 3.4×10¹³, 3.5×10¹³, 3.6×10¹³, 3.7×10¹³, 3.8×10¹³, 3.9×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/ml.

In certain embodiments, the concentration of AAV particle in the formulation is between 1×10¹¹ and 5×10¹³, between 1×10¹² and 5×10¹², between 2×10¹² and 1×10¹³, between 5×10¹² and 1×10¹³, between 1×10¹³ and 2×10¹³, between 2×10¹³ and 3×10¹³, between 2×10¹³ and 2.5×10¹³, between 2.5×10¹³ and 3×10¹³, or no more than 5×10¹³ VG/ml.

In certain embodiments, the concentration of AAV particle in the formulation is 2.7×10¹¹ VG/ml. In certain embodiments, the concentration of AAV particle in the formulation is 9×10¹¹ VG/ml. In certain embodiments, the concentration of AAV particle in the formulation is 1.2×10¹² VG/ml. In certain embodiments, the concentration of AAV particle in the formulation is 2.7×10¹² VG/ml. In certain embodiments, the concentration of AAV particle in the formulation is 4×10¹² VG/ml. In certain embodiments, the concentration of AAV particle in the formulation is 6×10²¹ VG/ml. In certain embodiments, the concentration of AAV particle in the formulation is 7.9×10¹² VG/ml. In certain embodiments, the concentration of AAV particle in the formulation is 8×10¹² VG/ml. In certain embodiments, the concentration of AAV particle in the formulation is 1×10¹³ VG/ml. In certain embodiments, the concentration of AAV particle in the formulation is 1.8×10¹³ VG/ml. In certain embodiments, the concentration of AAV particle in the formulation is 2.2×10¹³ VG/ml. In certain embodiments, the concentration of AAV particle in the formulation is 2.7×10¹³ VG/ml. In certain embodiments, the concentration of AAV particle in the formulation is 3.5×10¹³ VG/ml. In certain embodiments, the concentration of AAV particle in the formulation is 2.7-3.5×10¹³ VG/ml. In certain embodiments, the concentration of AAV particle in the formulation is 7.0×10¹³ VG/ml. In certain embodiments, the concentration of AAV particle in the formulation is 5.0×10¹² VG/ml.

In certain embodiments, the concentration of AAV particle in the formulation may be between about 1×10⁶ total capsid/mL and about 1×10¹⁶ total capsid/ml. In certain embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 3.1×10¹², 3.2×10¹², 3.3×10¹², 3.4×10¹², 3.5×10¹², 3.6×10¹², 3.7×10¹², 3.8×10¹², 3.9×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 9×10¹², 1×10¹³, 2×10¹³, 2.1×10¹³, 2.2×10¹³, 2.3×10¹³, 2.4×10¹³, 2.5×10¹³, 2.6×10¹³, 2.7×10¹³, 2.8×10¹³, 2.9×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ total capsid/ml.

Total Dose of AAV particle

In certain embodiments, the total dose of the AAV particle in the formulation may be between about 1×10⁶ VG and about 1×10¹⁶ VG. In certain embodiments, the formulation may comprise a total dose of AAV particle of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷ 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 7.1×10¹², 7.2×10¹², 7.3×10¹², 7.4×10¹², 7.5×10¹², 7.6×10¹², 7.7×10¹², 7.8×10¹², 7.9×10¹², 8×10¹², 8.1×10¹², 8.2×10¹², 8.3×10¹², 8.4×10¹², 8.5×10¹², 8.6×10¹², 8.7×10¹², 8.8×10¹², 8.9×10¹², 9×10¹², 1×10¹³, 1.1×10¹³, 1.2×10¹³, 1.3×10¹³, 1.4×10¹³, 1.5×10¹³, 1.6×10¹³, 1.7×10¹³, 1.8×10¹³, 1.9×10¹³, 2×10¹³, 2.1×10¹³, 2.2×10¹³, 2.3×10¹³, 2.4×10¹³, 2.5×10¹³, 2.6×10¹³, 2.7×10¹³, 2.8×10¹³, 2.9×10¹³, 3×10¹³, 3.1×10¹³, 3.2×10¹³, 3.3×10¹³, 3.4×10¹³, 3.5×10¹³, 3.6×10¹³, 3.7×10¹³, 3.8×10¹³, 3.9×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG.

In certain embodiments, the total dose of AAV particle in the formulation is between 1×10¹¹ and 5×10¹³ VG. In certain embodiments, the total dose of AAV particle in the formulation is between 1×10¹¹ and 2×10¹⁴ VG.

In certain embodiments, the total dose of AAV particle in the formulation is 1.4×10¹¹ VG. In certain embodiments, the total dose of AAV particle in the formulation is 4.5×10¹¹ VG. In certain embodiments, the total dose of AAV particle in the formulation is 6.8×10¹¹ VG. In certain embodiments, the total dose of AAV particle in the formulation is 1.4×10¹² VG. In certain embodiments, the total dose of AAV particle in the formulation is 2.2×10¹² VG. In certain embodiments, the total dose of AAV particle in the formulation is 4.6×10¹¹ VG. In certain embodiments, the total dose of AAV particle in the formulation is 9.2×10¹² VG. In certain embodiments, the total dose of AAV particle in the formulation is 1.0×10¹³ VG. In certain embodiments, the total dose of AAV particle in the formulation is 2.3×10¹³ VG.

### Measurement and Analysis

Expression of payloads or the downregulating effect of such payloads from viral genomes may be determined using various methods known in the art such as, but not limited to immunochemistry (e.g., IHC), in situ hybridization (ISH), enzyme-linked immunosorbent assay (ELISA), affinity ELISA, ELISPOT, flow cytometry, immunocytology, surface plasmon resonance analysis, kinetic exclusion assay, liquid chromatography-mass spectrometry (LCMS), high-performance liquid chromatography (HPLC), BCA assay, immunoelectrophoresis, Western blot, SDS-PAGE, protein immunoprecipitation, and/or PCR.

### Bioavailability

In certain embodiments, AAV particles formulated into a composition with a delivery agent as described herein can exhibit an increase in bioavailability as compared to a composition lacking a delivery agent as described herein. As used herein, the term “bioavailability” refers to the systemic availability of a given amount of AAV particle or expressed payload administered to a mammal. Bioavailability can be assessed by measuring the area under the curve (AUC) or the maximum serum or plasma concentration (Cₘₐₓ) of the composition following. AUC is a determination of the area under the curve plotting the serum or plasma concentration of a compound (e.g., AAV particles or expressed payloads) along the ordinate (Y-axis) against time along the abscissa (X-axis). Generally, the AUC for a particular compound can be calculated using methods known to those of ordinary skill in the art and as described in G. S. Banker, Modern Pharmaceutics, Drugs and the Pharmaceutical Sciences, v. 72, Marcel Dekker, New York, Inc., 1996, the contents of which are herein incorporated by reference in its entirety.

The Cₘₐₓ value is the maximum concentration of the AAV particle or expressed payload achieved in the serum or plasma of a mammal following administration of the AAV particle to the mammal. The Cₘₐₓ value of can be measured using methods known to those of ordinary skill in the art. The phrases “increasing bioavailability” or “improving the pharmacokinetics,” as used herein mean that the systemic availability of a first AAV particle or expressed payload, measured as AUC, Cₘₐₓ, or Cₘᵢₙ in a mammal is greater, when co-administered with a delivery agent as described herein, than when such co-administration does not take place. In certain embodiments, the bioavailability can increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

### Therapeutic Window

As used herein “therapeutic window” refers to the range of plasma concentrations, or the range of levels of therapeutically active substance at the site of action, with a high probability of eliciting a therapeutic effect. In certain embodiments, the therapeutic window of the AAV particle formulations as described herein can increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

### Volume of Distribution

As used herein, the term “volume of distribution” refers to the fluid volume that would be required to contain the total amount of the drug in the body at the same concentration as in the blood or plasma: V_{dist} equals the amount of drug in the body/concentration of drug in blood or plasma. For example, for a 10 mg dose and a plasma concentration of 10 mg/L, the volume of distribution would be 1 liter. The volume of distribution reflects the extent to which the drug is present in the extravascular tissue. A large volume of distribution reflects the tendency of a compound to bind to the tissue components compared with plasma protein binding. In a clinical setting, V_{dist} can be used to determine a loading dose to achieve a steady state concentration. In certain embodiments, the volume of distribution of the AAV particle formulations as described herein can decrease at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%.

### Biological Effect

In certain embodiments, the biological effect of the AAV particle formulations delivered to the animals may be categorized by analyzing the payload expression in the animals. The payload expression may be determined from analyzing a biological sample collected from a mammal administered the AAV particle formulations of the present disclosure. For example, a protein expression of 50-200 pg/ml for the protein encoded by the AAV particles delivered to the mammal may be seen as a therapeutically effective amount of protein in the mammal.

### IV. Definitions

At various places in the present disclosure, substituents or properties of compounds of the present disclosure are disclosed in groups or in ranges. It is specifically intended that the present disclosure comprise each and every individual or sub-combination of the members of such groups and ranges.

Unless stated otherwise, the following terms and phrases have the meanings described below. The definitions are not meant to be limiting in nature and serve to provide a clearer understanding of certain aspects of the present disclosure.

Adeno-associated virus: The term “adeno-associated virus” or “AAV” as used herein refers to members of the dependovirus genus comprising any particle, sequence, gene, protein, or component derived therefrom.

AAV Particle: As used herein, an “AAV particle” is a virus which comprises a capsid and a viral genome with at least one payload region and at least one ITR region, AAV particle may be derived from any serotype, described herein or known in the art, comprising combinations of serotypes (i.e., “pseudotyped” AAV) or from various genomes (e.g., single stranded or self-complementary). In addition, the AAV particle may be replication defective and/or targeted.

Activity: As used herein, the term “activity” refers to the condition in which things are happening or being done. Compositions of the present disclosure may have activity and this activity may involve one or more biological events.

Administering: As used herein, the term “administering” refers to providing a pharmaceutical agent or composition to a subject.

Administered in combination: As used herein, the term “administered in combination” or “combined administration” means that two or more agents are administered to a subject at the same time or within an interval such that there may be an overlap of an effect of each agent on the patient. In certain embodiments, they are administered within about 60, 30, 15, 10, 5, or 1 minute of one another. In certain embodiments, the administrations of the agents are spaced sufficiently closely together such that a combinatorial (e.g., a synergistic) effect is achieved.

Amelioration: As used herein, the term “amelioration” or “ameliorating” refers to a lessening of severity of at least one indicator of a condition or disease. For example, in the context of neurodegeneration disorder, amelioration comprises the reduction of neuron loss.

Animal: As used herein, the term “animal” refers to any member of the animal kingdom. In certain embodiments, “animal” refers to humans at any stage of development. In certain embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In certain embodiments, animals comprise, but are not limited to, mammals, birds, reptiles, amphibians, fish, and worms. In certain embodiments, the animal is a transgenic animal, genetically-engineered animal, or a clone.

Antisense strand: As used herein, the term “the antisense strand” or “the first strand” or “the guide strand” of a siRNA molecule refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process.

Approximately: As used herein, the term “approximately” or “about,” as applied to one or more values of interest, refers to a value that is similar to a stated reference value. As used herein, the term “about” means+/−10% of the recited value. In certain embodiments, the term “approximately” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization-based connectivity sufficiently stable such that the “associated” entities remain physically associated.

Baculoviral expression vector (BEV): As used herein a BEV is a baculoviral expression vector, i.e., a polynucleotide vector of baculoviral origin. A baculovirus expression vector (BEV) is a recombinant baculovirus that has been genetically modified to lead the expression of a foreign gene. Systems using BEVs are known as baculoviral expression vector systems (BEVSs).

mBEV or modified BEV: As used herein, a modified BEV is an expression vector of baculoviral origin which has been altered from a starting BEV (whether wild type or artificial) by the addition and/or deletion and/or duplication and/or inversion of one or more: genes; gene fragments; cleavage sites; restriction sites; sequence regions; sequence(s) encoding a payload or gene of interest; or combinations of the foregoing.

Bifunctional: As used herein, the term “bifunctional” refers to any substance, molecule or moiety which is capable of or maintains at least two functions. The functions may affect the same outcome or a different outcome. The structure that produces the function may be the same or different.

BIIC: As used herein a BIIC is a baculoviral infected insect cell.

Biocompatible: As used herein, the term “biocompatible” means compatible with living cells, tissues, organs or systems posing little to no risk of injury, toxicity or rejection by the immune system.

Biodegradable: As used herein, the term “biodegradable” means capable of being broken down into innocuous products by the action of living things.

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, an AAV particle of the present disclosure may be considered biologically active if even a portion of the encoded payload is biologically active or mimics an activity considered biologically relevant.

Capsid: As used herein, the term “capsid” refers to the protein shell of a virus particle.

Codon optimized: As used herein, the terms “codon optimized” or “codon optimization” refers to a modified nucleic acid sequence which encodes the same amino acid sequence as a parent/reference sequence, but which has been altered such that the codons of the modified nucleic acid sequence are optimized or improved for expression in a particular system (such as a particular species or group of species). As a non-limiting example, a nucleic acid sequence which comprises an AAV capsid protein can be codon optimized for expression in insect cells or in a particular insect cell such Spodoptera frugiperda cells. Codon optimization can be completed using methods and databases known to those in the art.

Complementary and substantially complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present disclosure, the ability to substitute a T is implied, unless otherwise stated. Perfect complementarity or 100% complementarity refers to the situation in which each nucleotide unit of one polynucleotide strand can form hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form hydrogen bond with each other, the polynucleotide strands exhibit 10% complementarity. In the same example, if 18 base pairs on each strand can form hydrogen bonds with each other, the polynucleotide strands exhibit 90% complementarity.

Compound: Compounds of the present disclosure comprise all of the isotopes of the atoms occurring in the intermediate or final compounds. “Isotopes” refers to atoms having the same atomic number but different mass numbers resulting from a different number of neutrons in the nuclei. For example, isotopes of hydrogen comprise tritium and deuterium.

The compounds and salts of the present disclosure can be prepared in combination with solvent or water molecules to form solvates and hydrates by routine methods.

Conditionally active: As used herein, the term “conditionally active” refers to a mutant or variant of a wild-type polypeptide, wherein the mutant or variant is more or less active at physiological conditions than the parent polypeptide. Further, the conditionally active polypeptide may have increased or decreased activity at aberrant conditions as compared to the parent polypeptide. A conditionally active polypeptide may be reversibly or irreversibly inactivated at normal physiological conditions or aberrant conditions.

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In certain embodiments, two or more sequences are said to be “completely conserved” if they are 100% identical to one another. In certain embodiments, two or more sequences are said to be “highly conserved” if they are at least 70% identical, at least 80% identical, at least 90% identical, or at least 95% identical to one another. In certain embodiments, two or more sequences are said to be “highly conserved” if they are about 70% identical, about 80% identical, about 90% identical, about 95%, about 98%, or about 99% identical to one another. In certain embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In certain embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an polynucleotide or polypeptide or may apply to a portion, region or feature thereof.

Control Elements: As used herein, “control elements”, “regulatory control elements” or “regulatory sequences” refers to promoter regions, polyadenylation signals, transcription termination sequences, upstream regulatory domains, origins of replication, internal ribosome entry sites (“IRES”), enhancers, and the like, which provide for the replication, transcription and translation of a coding sequence in a recipient cell. Not all of these control elements need always be present as long as the selected coding sequence is capable of being replicated, transcribed and/or translated in an appropriate host cell.

Controlled Release: As used herein, the term “controlled release” refers to a pharmaceutical composition or compound release profile that conforms to a particular pattern of release to affect a therapeutic outcome.

Cytostatic: As used herein, “cytostatic” refers to inhibiting, reducing, suppressing the growth, division, or multiplication of a cell (e.g., a mammalian cell (e.g., a human cell)), bacterium, virus, fungus, protozoan, parasite, prion, or a combination thereof.

Cytotoxic: As used herein, “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

Delivery: As used herein, “delivery” refers to the act or manner of delivering an AAV particle, a compound, substance, entity, moiety, cargo or payload.

Delivery Agent: As used herein, “delivery agent” refers to any substance which facilitates, at least in part, the in vivo delivery of an AAV particle to targeted cells.

Destabilized: As used herein, the term “destabilize,” or “destabilizing region” means a region or molecule that is less stable than a starting, wild-type or native form of the same region or molecule.

Detectable label: As used herein, “detectable label” refers to one or more markers, signals, or moieties which are attached, incorporated or associated with another entity that is readily detected by methods known in the art comprising radiography, fluorescence, chemiluminescence, enzymatic activity, absorbance and the like. Detectable labels comprise radioisotopes, fluorophores, chromophores, enzymes, dyes, metal ions, ligands such as biotin, avidin, streptavidin and haptens, quantum dots, and the like. Detectable labels may be located at any position in the peptides or proteins disclosed herein. They may be within the amino acids, the peptides, or proteins, or located at the N- or C-termini.

Digest: As used herein, the term “digest” means to break apart into smaller pieces or components. When referring to polypeptides or proteins, digestion results in the production of peptides.

Distal: As used herein, the term “distal” means situated away from the center or away from a point or region of interest.

Dosing regimen: As used herein, a “dosing regimen” is a schedule of administration or physician determined regimen of treatment, prophylaxis, or palliative care.

Encapsulate: As used herein, the term “encapsulate” means to enclose, surround or encase.

Engineered: As used herein, embodiments of the present disclosure are “engineered” when they are designed to have a feature or property, whether structural or chemical, that varies from a starting point, wild type or native molecule.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

ExpressionBac: As used herein, “expressionBac” or “rep/cap bac” refers to a baculovirus comprising an adeno-associated virus (AAV) viral expression construct and/or region. In some embodiments, the viral expression construct of the expressionBac comprises one or more polynucleotides encoding capsid and/or replication genes for an AAV, such as but not limited to AAV2. For example, the one or more polynucleotides encoding capsid and/or replication genes for an AAV may encode VP1, VP2, VP3, Rep52, and/or Rep78, and these polynucleotides may be present in the construct in one or more open reading frames, e.g., in two open reading frames.

Expression BIIC: As used herein, “expression BIIC” or “rep/cap BIIC” refers to an insect cell comprising one or more baculoviruses (e.g. expressionBac) which comprise bacmids comprising a viral expression construct. In some embodiments, the expression construct comprises one or more polynucleotides encoding capsid and/or replication genes for an AAV, such as but not limited to AAV2. For example, the one or more polynucleotides encoding capsid and/or replication genes for an AAV may encode VP1, VP2, VP3, Rep52, and/or Rep78, and these polynucleotides may be present in the construct in one or more open reading frames, e.g., in two open reading frames. In some embodiments, the insect cell is an Sf9 cell.

Feature: As used herein, a “feature” refers to a characteristic, a property, or a distinctive element.

Formulation: As used herein, a “formulation” comprises at least one AAV particle and a delivery agent or excipient.

Fragment: A “fragment,” as used herein, refers to a portion. For example, fragments of proteins may comprise polypeptides obtained by digesting full-length protein isolated from cultured cells.

Functional: As used herein, a “functional” biological molecule is a biological molecule in a form in which it exhibits a property and/or activity by which it is characterized.

Gene expression: The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

Homology: As used herein, the term “homology” refers to the overall relatedness between polymeric molecules, e.g. between polynucleotide molecules (e.g. DNA molecules and/or RNA molecules) and/or between polypeptide molecules. In certain embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the present disclosure, two polynucleotide sequences are considered to be homologous if the polypeptides they encode are at least about 50%, 60%, 70%, 80%, 90%, 95%, or even 99% for at least one stretch of at least about 20 amino acids. In certain embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the present disclosure, two protein sequences are considered to be homologous if the proteins are at least about 50%, 60%, 70%, 80%, or 90% identical for at least one stretch of at least about 20 amino acids.

Heterologous Region: As used herein the term “heterologous region” refers to a region which would not be considered a homologous region.

Homologous Region: As used herein the term “homologous region” refers to a region which is similar in position, structure, evolution origin, character, form or function.

Identity: As used herein, the term “identity” refers to the overall relatedness between polymeric molecules, e.g., between polynucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; the contents of which are each incorporated herein by reference in their entireties, insofar as they do not conflict with the present disclosure. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences comprise, but are not limited to those disclosed in Carillo, H., and Lipman, D., SIAM J Applied Math., 48:1073 (1988); the contents of which are each incorporated herein by reference in their entireties, insofar as they does not conflict with the present disclosure. Techniques for determining identity are codified in publicly available computer programs. Exemplary computer software to determine homology between two sequences comprise, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically, a reduction in the level of an mRNA results in a reduction in the level of a polypeptide translated therefrom. The level of expression may be determined using standard techniques for measuring mRNA or protein.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In certain embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. As used herein, the term “substantially isolated” is meant that a substance is substantially separated from the environment in which it was formed or detected. Partial separation can comprise, for example, a composition enriched in the substance or AAV particles of the present disclosure. Substantial separation can comprise compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

Linker: As used herein “linker” refers to a molecule or group of molecules which connects two molecules. A linker may be a nucleic acid sequence connecting two nucleic acid sequences encoding two different polypeptides. The linker may or may not be translated. The linker may be a cleavable linker.

MicroRNA (miRNA) binding site: As used herein, a microRNA (miRNA) binding site represents a nucleotide location or region of a nucleic acid transcript to which at least the “seed” region of a miRNA binds.

Modified: As used herein “modified” refers to a changed state or structure of a molecule of the present disclosure. Molecules may be modified in many ways comprising chemically, structurally, and functionally. As used herein, embodiments of the disclosure are “modified” when they have or possess a feature or property, whether structural or chemical, that varies from a starting point, wild type or native molecule.

Mutation: As used herein, the term “mutation” refers to any changing of the structure of a gene, resulting in a variant (also called “mutant”) form that may be transmitted to subsequent generations. Mutations in a gene may be caused by the alternation of single base in DNA, or the deletion, insertion, or rearrangement of larger sections of genes or chromosomes.

Naturally Occurring: As used herein, “naturally occurring” or “wild-type” means existing in nature without artificial aid, or involvement of the hand of man.

Non-human vertebrate: As used herein, a “non-human vertebrate” comprises all vertebrates except Homo sapiens, comprising wild and domesticated species. Examples of non-human vertebrates comprise, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Nucleic Acid: As used herein, the term “nucleic acid”, “polynucleotide” and “oligonucleotide” refer to any nucleic acid polymers composed of either polydeoxyribonucleotides (containing 2-deoxy-D-ribose), or polyribonucleotides (containing D-ribose), or any other type of polynucleotide which is an N glycoside of a purine or pyrimidine base, or modified purine or pyrimidine bases. There is no intended distinction in length between the term “nucleic acid”, “polynucleotide” and “oligonucleotide”, and these terms will be used interchangeably. These terms refer only to the primary structure of the molecule. Thus, these terms comprise double- and single-stranded DNA, as well as double- and single stranded RNA.

Off-target: As used herein, “off target” refers to any unintended effect on any one or more target, gene, or cellular transcript.

Open reading frame: As used herein, “open reading frame” or “ORF” refers to a sequence which does not contain a stop codon within the given reading frame, other than at the end of the reading frame.

Operably linked: As used herein, the phrase “operably linked” refers to a functional connection between two or more molecules, constructs, transcripts, entities, moieties or the like. As a non-limiting example, a promoter is “operably linked” to a nucleotide sequence when the promoter sequence controls and/or regulates the transcription of the nucleotide sequence.

Patient: As used herein, “patient” refers to a subject who may seek or need treatment, requires treatment, is receiving treatment, will receive treatment, or a subject who is under care by a trained professional for a particular disease or condition.

Payload: As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide or multi-polypeptide, or a modulatory nucleic acid or regulatory nucleic acid.

PayloadBac: As used herein, “payloadBac” refers to a baculovirus comprising a payload construct and/or region. In some embodiments, the payload construct and/or region of the payloadBac comprises a polynucleotide encoding the payload.

Payload BIIC: As used herein, “payloadBIIC” refers to an insect cell comprising one or more baculovirus (e.g. payloadBac) comprising a payload construct and/or region. In some embodiments, the payload construct and/or region comprises a polynucleotide encoding the payload. In some embodiments, the insect cell is an Sf9 cell.

Payload construct: As used herein, “payload construct” is one or more vector construct which comprises a polynucleotide region encoding or comprising a payload that is flanked on one or both sides by an inverted terminal repeat (ITR) sequence. The payload construct may present a template that is replicated in a viral production cell to produce a therapeutic viral genome.

Payload construct vector: As used herein, “payload construct vector” is a vector encoding or comprising a payload construct, and regulatory regions for replication and expression of the payload construct in bacterial cells.

Payload construct expression vector: As used herein, a “payload construct expression vector” is a vector encoding or comprising a payload construct and which further comprises one or more polynucleotide regions encoding or comprising components for viral expression in a viral replication cell.

Peptide: As used herein, “peptide” is less than or equal to 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may comprise, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients comprise, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (coin), stearic acid, sucrose, talc, titanium dioxide, vitamin A, vitamin E, vitamin C, and xylitol.

Pharmaceutically acceptable salts: The present disclosure also comprises pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts comprise, but are not limited to, mineral or organic acid salts of basic residues such as amines; alkali or organic salts of acidic residues such as carboxylic acids; and the like. Representative acid addition salts comprise acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts comprise sodium, lithium, potassium, calcium, magnesium, and the like, as well as nontoxic ammonium, quaternary ammonium, and amine cations, comprising, but not limited to ammonium, tetramethylammonium, tetraethylammonium, methylamine, dimethylamine, trimethylamine, triethylamine, ethylamine, and the like. The pharmaceutically acceptable salts of the present disclosure comprise the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile can be used. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), the contents of which are each incorporated herein by reference in their entireties, insofar as they do not conflict with the present disclosure.

Pharmaceutically acceptable solvate: The term “pharmaceutically acceptable solvate,” as used herein, means a compound of the present disclosure wherein molecules of a suitable solvent are incorporated in the crystal lattice. A suitable solvent is physiologically tolerable at the dosage administered. For example, solvates may be prepared by crystallization, recrystallization, or precipitation from a solution that comprises organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Pharmacokinetic: As used herein, “pharmacokinetic” refers to any one or more properties of a molecule or compound as it relates to the determination of the fate of substances administered to a living organism. Pharmacokinetics is divided into several areas comprising the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Polypeptide: As used herein, “polypeptide” refers to a polymer of amino acid residues (natural or unnatural) linked together most often by peptide bonds. The term, as used herein, refers to proteins, polypeptides, and peptides of any size, structure, or function. In some instances, the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The term polypeptide may also apply to amino acid polymers in which one or more amino acid residues are an artificial chemical analogue of a corresponding naturally occurring amino acid.

Physicochemical: As used herein, “physicochemical” means of or relating to a physical and/or chemical property.

Preventing: As used herein, the term “preventing” or “prevention” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Proliferate: As used herein, the term “proliferate” means to grow, expand or increase or cause to grow, expand or increase rapidly. “Proliferative” means having the ability to proliferate. “Anti-proliferative” means having properties counter to or inapposite to proliferative properties.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease.

Protein of interest: As used herein, the terms “proteins of interest” or “desired proteins” comprise those provided herein and fragments, mutants, variants, and alterations thereof.

Proximal: As used herein, the term “proximal” means situated nearer to the center or to a point or region of interest.

Purified: As used herein, “purify,” “purified,” “purification” means to make substantially pure or clear from unwanted components, material defilement, admixture or imperfection. “Purified” refers to the state of being pure. “Purification” refers to the process of making pure.

Region: As used herein, the term “region” refers to a zone or general area. In certain embodiments, when referring to a protein or protein module, a region may comprise a linear sequence of amino acids along the protein or protein module or may comprise a three-dimensional area, an epitope and/or a cluster of epitopes. In certain embodiments, regions comprise terminal regions. As used herein, the term “terminal region” refers to regions located at the ends or termini of a given agent. When referring to proteins, terminal regions may comprise N- and/or C-termini. N-termini refer to the end of a protein comprising an amino acid with a free amino group. C-termini refer to the end of a protein comprising an amino acid with a free carboxyl group. N- and/or C-terminal regions may there for comprise the N- and/or C-termini as well as surrounding amino acids. In certain embodiments, N- and/or C-terminal regions comprise from about 3 amino acid to about 30 amino acids, from about 5 amino acids to about 40 amino acids, from about 10 amino acids to about 50 amino acids, from about 20 amino acids to about 100 amino acids and/or at least 100 amino acids. In certain embodiments, N-terminal regions may comprise any length of amino acids that comprises the N-terminus but does not comprise the C-terminus. In certain embodiments, C-terminal regions may comprise any length of amino acids, which comprise the C-terminus, but do not comprise the N-terminus.

In certain embodiments, when referring to a polynucleotide, a region may comprise a linear sequence of nucleic acids along the polynucleotide or may comprise a three-dimensional area, secondary structure, or tertiary structure. In certain embodiments, regions comprise terminal regions. As used herein, the term “terminal region” refers to regions located at the ends or termini of a given agent. When referring to polynucleotides, terminal regions may comprise 5′ and 3′ termini. 5′ termini refer to the end of a polynucleotide comprising a nucleic acid with a free phosphate group. 3′ termini refer to the end of a polynucleotide comprising a nucleic acid with a free hydroxyl group. 5′ and 3′ regions may there for comprise the 5′ and 3′ termini as well as surrounding nucleic acids. In certain embodiments, 5′ and 3′ terminal regions comprise from about 9 nucleic acids to about 90 nucleic acids, from about 15 nucleic acids to about 120 nucleic acids, from about 30 nucleic acids to about 150 nucleic acids, from about 60 nucleic acids to about 300 nucleic acids and/or at least 300 nucleic acids. In certain embodiments, 5′ regions may comprise any length of nucleic acids that comprises the 5′ terminus but does not comprise the 3′ terminus. In certain embodiments, 3′ regions may comprise any length of nucleic acids, which comprise the 3′ terminus, but does not comprise the 5′ terminus.

RNA or RNA molecule: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

RNA interfering or RNAi: As used herein, the term “RNA interfering” or “RNAi” refers to a sequence specific regulatory mechanism mediated by RNA molecules which results in the inhibition or interfering or “silencing” of the expression of a corresponding protein-coding gene. RNAi has been observed in many types of organisms, comprising plants, animals and fungi. RNAi occurs in cells naturally to remove foreign RNAs (e.g., viral RNAs). Natural RNAi proceeds via fragments cleaved from free dsRNA which direct the degradative mechanism to other similar RNA sequences. RNAi is controlled by the RNA-induced silencing complex (RISC) and is initiated by short/small dsRNA molecules in cell cytoplasm, where they interact with the catalytic RISC component argonaute. The dsRNA molecules can be introduced into cells exogenously. Exogenous dsRNA initiates RNAi by activating the ribonuclease protein Dicer, which binds and cleaves dsRNAs to produce double-stranded fragments of 21-25 base pairs with a few unpaired overhang bases on each end. These short double stranded fragments are called small interfering RNAs (siRNAs).

Sample: As used herein, the term “sample” or “biological sample” refers to a subset of its tissues, cells or component parts (e.g. body fluids, comprising but not limited to blood, mucus, lymphatic fluid, synovial fluid, cerebrospinal fluid, saliva, amniotic fluid, amniotic cord blood, urine, vaginal fluid and semen). A sample further may comprise a homogenate, lysate or extract prepared from a whole organism or a subset of its tissues, cells or component parts, or a fraction or portion thereof, comprising but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

Self-complementary viral particle: As used herein, a “self-complementary viral particle” is a particle comprised of at least two components, a protein capsid and a polynucleotide sequence encoding a self-complementary genome enclosed within the capsid.

Sense Strand: As used herein, the term “the sense strand” or “the second strand” or “the passenger strand” of a siRNA molecule refers to a strand that is complementary to the antisense strand or first strand. The antisense and sense strands of a siRNA molecule are hybridized to form a duplex structure. As used herein, a “siRNA duplex” comprises a siRNA strand having sufficient complementarity to a section of about 10-50 nucleotides of the mRNA of the gene targeted for silencing and a siRNA strand having sufficient complementarity to form a duplex with the other siRNA strand.

Short interfering RNA or siRNA: As used herein, the terms “short interfering RNA,” “small interfering RNA” or “siRNA” refer to an RNA molecule (or RNA analog) comprising between about 5-60 nucleotides (or nucleotide analogs) which is capable of directing or mediating RNAi. In certain embodiments, a siRNA molecule comprises between about 15-30 nucleotides or nucleotide analogs, such as between about 16-25 nucleotides (or nucleotide analogs), between about 18-23 nucleotides (or nucleotide analogs), between about 19-22 nucleotides (or nucleotide analogs) (e.g., 19, 20, 21 or 22 nucleotides or nucleotide analogs), between about 19-25 nucleotides (or nucleotide analogs), and between about 19-24 nucleotides (or nucleotide analogs). The term “short” siRNA refers to a siRNA comprising 5-23 nucleotides, such as 21 nucleotides (or nucleotide analogs), for example, 19, 20, 21 or 22 nucleotides. The term “long” siRNA refers to a siRNA comprising 24-60 nucleotides, such as about 24-25 nucleotides, for example, 23, 24, 25 or 26 nucleotides. Short siRNAs may, in some instances, comprise fewer than 19 nucleotides, e.g., 16, 17 or 18 nucleotides, or as few as 5 nucleotides, provided that the shorter siRNA retains the ability to mediate RNAi. Likewise, long siRNAs may, in some instances, comprise more than 26 nucleotides, e.g., 27, 28, 29, 30, 35, 40, 45, 50, 55, or even 60 nucleotides, provided that the longer siRNA retains the ability to mediate RNAi or translational repression absent further processing, e.g., enzymatic processing, to a short siRNA. siRNAs can be single stranded RNA molecules (ss-siRNAs) or double stranded RNA molecules (ds-siRNAs) comprising a sense strand and an antisense strand which hybridized to form a duplex structure called siRNA duplex.

Signal Sequences: As used herein, the phrase “signal sequences” refers to a sequence which can direct the transport or localization of a protein.

Single unit dose: As used herein, a “single unit dose” is a dose of any therapeutic administered in one dose/at one time/single route/single point of contact, i.e., single administration event. In certain embodiments, a single unit dose is provided as a discrete dosage form (e.g., a tablet, capsule, patch, loaded syringe, vial, etc.).

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Split dose: As used herein, a “split dose” is the division of single unit dose or total daily dose into two or more doses.

Stable: As used herein “stable” refers to a compound that is sufficiently robust to survive isolation to a useful degree of purity from a reaction mixture, and in certain embodiments, capable of formulation into an efficacious therapeutic agent.

Stabilized: As used herein, the term “stabilize”, “stabilized,” “stabilized region” means to make or become stable.

Subject: As used herein, the term “subject” or “patient” refers to any organism to which a composition in accordance with the present disclosure may be administered, e.g., for experimental, diagnostic, prophylactic, and/or therapeutic purposes. Typical subjects comprise animals (e.g., mammals such as mice, rats, rabbits, non-human primates, and humans) and/or plants. The subject or patient may seek or need treatment, require treatment, is receiving treatment, will receive treatment, or is under care by a trained professional for a particular disease or condition.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term means within 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In certain embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In certain embodiments, an individual who is susceptible to a disease, disorder, and/or condition will develop the disease, disorder, and/or condition. In certain embodiments, an individual who is susceptible to a disease, disorder, and/or condition will not develop the disease, disorder, and/or condition.

Sustained release: As used herein, the term “sustained release” refers to a pharmaceutical composition or compound release profile that conforms to a release rate over a specific period of time.

Synthetic: The term “synthetic” means produced, prepared, and/or manufactured by the hand of man. Synthesis of polynucleotides or polypeptides or other molecules of the present disclosure may be chemical or enzymatic.

Targeting: As used herein, “targeting” means the process of design and selection of nucleic acid sequence that will hybridize to a target nucleic acid and induce a desired effect.

Targeted Cells: As used herein, “targeted cells” refers to any one or more cells of interest. The cells may be found in vitro, in vivo, in situ or in the tissue or organ of an organism. The organism may be an animal, such as a mammal, a human, or a human patient.

Terminal region: As used herein, the term “terminal region” refers to a region on the 5′ or 3′ end of a region of linked nucleosides or amino acids (polynucleotide or polypeptide, respectively).

Terminally optimized: The term “terminally optimized” when referring to nucleic acids means the terminal regions of the nucleic acid are improved in some way, e.g., codon optimized, over the native or wild type terminal regions.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In certain embodiments, a therapeutically effective amount is provided in a single dose. In certain embodiments, a therapeutically effective amount is administered in a dosage regimen comprising a plurality of doses. Those skilled in the art will appreciate that in certain embodiments, a unit dosage form may be considered to comprise a therapeutically effective amount of a particular agent or entity if it comprises an amount that is effective when administered as part of such a dosage regimen.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in 24-hour period. It may be administered as a single unit dose.

Transfection: As used herein, the term “transfection” refers to methods to introduce exogenous nucleic acids into a cell. Methods of transfection comprise, but are not limited to, chemical methods, physical treatments and cationic lipids or mixtures.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Vector: As used herein, a “vector” is any molecule or moiety which transports, transduces or otherwise acts as a carrier of a heterologous molecule. Vectors of the present disclosure may be produced recombinantly and may be based on and/or may comprise adeno-associated virus (AAV) parent or reference sequence. Such parent or reference AAV sequences may serve as an original, second, third or subsequent sequence for engineering vectors. In non-limiting examples, such parent or reference AAV sequences may comprise any one or more of the following sequences: a polynucleotide sequence encoding a polypeptide or multi-polypeptide, which sequence may be wild-type or modified from wild-type and which sequence may encode full-length or partial sequence of a protein, protein domain, or one or more subunits of a protein; a polynucleotide comprising a modulatory or regulatory nucleic acid which sequence may be wild-type or modified from wild-type; and a transgene that may or may not be modified from wild-type sequence. These AAV sequences may serve as either the “donor” sequence of one or more codons (at the nucleic acid level) or amino acids (at the polypeptide level) or “acceptor” sequences of one or more codons (at the nucleic acid level) or amino acids (at the polypeptide level).

Viral genome: As used herein, a “viral genome” or “vector genome” refers to the nucleic acid sequence(s) encapsulated in an AAV particle.

### V. Equivalents and Scope

Those skilled in the art will recognize or be able to ascertain using no more than routine experimentation, many equivalents to the specific embodiments in accordance with the present disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that comprise “or” between one or more members of a group are considered satisfied if one, more than one, or all of the group members are present in, employed in, or otherwise relevant to a given product or process unless indicated to the contrary or otherwise evident from the context. The present disclosure comprises embodiments in which exactly one member of the group is present in, employed in, or otherwise relevant to a given product or process. The present disclosure comprises embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are comprised. Furthermore, it is to be understood that unless otherwise indicated or otherwise evident from the context and understanding of one of ordinary skill in the art, values that are expressed as ranges can assume any specific value or subrange within the stated ranges in different embodiments of the present disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the present disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the present disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the present disclosure.

All publications, patent applications, patents, and other references mentioned herein are incorporated by reference in their entirety. In case of conflict, the present specification, comprising definitions, will control. In addition, section headings, the materials, methods, and examples are illustrative only and not intended to be limiting.

### EXAMPLES

### Example 1. Production of Source Rep/Cap BIICs (A)

### CP BEV Pool

One vial of the Sf9 CB was thawed in a 125 mL shaker flask (37° C. using waterbath, 1-5 minutes until ice crystals dissipate), and then diluted into 19-20 mL working volume of Hyclone SFX Insect Cell Culture Media. The shaker flask was incubated at 27° C. (135 rpm shaking, 0% v/v of CO₂) in a first expansion (P0, 3-4 days) until the cell density of the Sf9 cell mixture was expanded to 4.0-6.0×10⁶ cells/mL.

The cell mixture was then seeded and expanded through multiple additional expansion steps using larger shaker flasks, with a target output density of 4.0-6.0×10⁶ cells/mL for each expansion step to allow for a consistent seeding density of 0.5-3.0×10⁶ cells/mL in subsequent expansion steps. Expansions were completed at 27° C. for 3-5 days (0% v/v of CO₂) with 135 rpm shaking (<2 L working volume) or 90 rpm shaking (>2 L working volume). The following additional expansions were completed: (i) expansion up to 200 mL working volume in a 1.0 L flask; and (ii) expansion up to 1000 mL working volume in a 3 L flask.

A Rep/Cap Transfection Mixture was prepared by combining 5 μg of Rep/Cap Bacmid material with 375 μL of WFI water. The diluted Bacmid mixture was combined with 30 μL of Promega FuGENE HD (Transfection Agent) and an additional 345 μL of WFI water, and then incubated at 27° C. for 15 minutes to provide a Transfection Cocktail.

25 mL of expanded Sf9 cell mixture was seeded into a 125 mL flask (1.0×10⁶ cells/mL seeding concentration) and expanded to a target infection density of 2.5-4.0×10⁶ cells/mL. The Transfection Cocktail was added to the 125 mL flask and incubated at 27° C. for 5-7 days (0% v/v of CO₂, 135 rpm agitation). The resulting mixture was centrifuged in 50 mL conical tubes for 5 minutes, and the supernatant containing P1 BEVs was collected and pooled with other P1 BEV supernatants. The P1 BEV pool was stored at 5° C.

Expanded Sf9 cell mixture was seeded into a Cellstar 6-well Cell Polystyrene Culture Plate (2 mL per well, 0.5-1.0×10⁶ cells/mL seeding concentration) with gentle rocking to evenly distribute cells, followed by incubation at 27° C. for 90 minutes (0% v/v of CO₂, 0 rpm agitation). P1 BEVs were serial diluted with Hyclone SFX Insect Cell Culture Media to a target dilution of 1.0×10⁷ BEVs/mL, and then 1 mL of diluted P1 BEV mixture was added to each well with gentle rocking to evenly distribute P1 BEVs. The infection mixture was incubated at 27° C. for 90 minutes (0% v/v of CO₂, 0 rpm agitation).

Agarose gel was prepared by combining 4% w/v agarose 1:3 with Life Technologies Sf-900 Medium overlay (melt agarose at 70° C., cool to 37° C. for combination). 2 mL of Agarose Overlay was then added to each well, and the plates were maintained at room temperature for 15-20 minutes for the agarose gel to harden. Overlaid plates were then incubated at 27° C. for 5-14 days (0% v/v of CO₂, 0 rpm agitation) until plaque formation was observed. Plaques in each well were processed through testing and Plaque Picking to provide a single Plaque for Clonal Plaque Purification (i.e. Single Plaque Expansion). The Single Plaque was expanded using Sf9 cell mixture and incubated at 27° C. for 3-5 days (0% v/v of CO₂, 0 rpm agitation). The resulting CP1 BEVs were harvested using centrifugation in 50 mL conical tubes for 5 minutes and collection of supernatant containing CP1 BEVs into a CP1 BEV pool.

### BEV Infection/BIIC Production

Sf9 cell mixture was seeded and expanded through multiple expansion steps using larger shaker flasks, with a target output density of 4.0-6.0×10⁶ cells/mL for each expansion step to allow for a consistent seeding density of 0.5-2.0×10⁶ cells/mL in subsequent expansion steps. Expansions were completed at 27° C. for 3-5 days (0% v/v of CO₂) with 135 rpm shaking (≤2 L working volume) or 90 rpm shaking (>2 L working volume). The following expansions were completed: (i) expansion up to 200 mL working volume in a 1.0 L flask; (ii) expansion up to 1000 mL working volume in a 3 L flask; and (iii) expansion up to 2500 mL working volume in a 5 L flask, with a final output density of 2.0-4.0×10⁶ cells/mL.

200 mL of expanded Sf9 cell mixture was seeded into a 1.0 L flask (1.0×10⁶ cells/mL seeding density) and expanded to a viable infection cell density of ≥2.0×10⁶ cells/mL, and then infected with 0.01 MOI of CP1 BEV. Infected cells were then incubated and expanded at 27° C. for 48-80 hours (0% v/v of CO₂, 135 rpm) until cells reach ≥2.0×10⁶ cells/mL (VCD), ≥16.5 μm cell diameter and ≥75% cell viability. The infected cells were harvested by spinning down (polypropylene centrifuge tubes, 5 min) and resuspending the cell pellet at 4.0×10⁷ cells/mL in Hyclone SFX Insect Cell Culture Media, followed by the addition of 300 mM Trehalose, 14% v/v of DMSO, and additional SFX Culture Media to provide target VCD of 2.0×10⁶ cells/mL. Rep/Cap Source BIICs were aliquoted into 2 mL or 5 mL cryovials and frozen down to ≤−65° C. using control rate freezer, and then stored at −80° C. or in LN₂ vapor.

### Example 2. Production of Source Transgene BIICs (A)

Transgene Source BIICs were produced according to Example 1, with Transgene Bacmid material used for P1 BEV production instead of Rep/Cap Bacmid material.

### Example 3. Production Source Rep/Cap BIICs (B)

### CP BEV Pool

One vial of the Sf9 CB was thawed in a 125 mL shaker flask (37° C. using waterbath, 1-5 minutes until ice crystals dissipate), and then diluted into 40 mL working volume of Hyclone SFX Insect Cell Culture Media. The shaker flask was incubated for a first expansion (P0, 3-4 days) until the cell density of the Sf9 cell mixture was expanded to 4.0-6.0×10⁶ cells/mL.

The culture was then seeded and expanded through multiple additional expansion steps using larger shaker flasks, with a target output density of 4.0-6.0×10⁶ cells/mL for each expansion step to allow for a consistent seeding density of 0.5-3.0×10⁶ cells/mL in subsequent expansion steps. Expansions were completed at 27° C. for 3-5 days and comprised: (i) expansion up to 200 mL working volume in a 1.0 L flask (P1); and (ii) expansion up to 1000 mL working volume in a 3 L flask (P2).

A Rep/Cap Transfection Mixture was prepared by combining 30 μg of Rep/Cap Bacmid material with 0.6 mL of ThermoFisher Grace's Insect Media (Transfection Media). The diluted Bacmid mixture was combined with 30 μL of ThermoFisher Cellfectin II Reagent (Transfection Agent) and an additional 0.6 mL of Transfection Media, followed by incubation at 18-25° C. for 25-35 minutes, and then further dilution with 4.8 mL of Transfection Media to provide a Transfection Cocktail.

60 mL of expanded Sf9 cell mixture was seeded into a 125 mL flask and expanded up to 1.0×10⁶ cells/mL seeding concentration. The Sf9 cell mixture was then seeded into a 6-well Cell Culture Plate (2 mL per well, 1.0×10⁶ cells/mL seeding concentration). 1 mL of Transfection Cocktail was added to each well, and the plate was incubated at 27° C. for 4-5 hours. 2 mL of Hyclone SFX Insect Cell Culture Media was added to each well, and the plates were then incubated at 27° C. for 3-4 days. The resulting mixtures were centrifuged in 50 mL conical tubes for 5 minutes, and supernatant containing P1 BEVs was collected and pooled with other P1 BEV supernatants. The P1 BEV pool was stored at 4-8° C.

Expanded Sf9 cell mixture was seeded into a 6-well Cell Culture Plate (2 mL per well, 0.5-1.0 cells/mL seeding concentration) with gentle rocking to evenly distribute cells, followed by incubation at 27° C. for 90 minutes. P1 BEVs were serial diluted with Hyclone SFX Insect Cell Culture Media to a target dilution of 1.0-5.0×10⁷ BEVs, and then 1 mL of diluted P1 BEV mixture was added to each well with gentle rocking to evenly distribute P1 BEVs. The infection mixture was incubated at 27° C. for 90 minutes.

Agarose gel was prepared by combining 4% w/v agarose 1:3 with Life Technologies Sf-900 Medium overlay. Agarose Overlay was added to each well, and the plates were maintained at room temperature for 15-20 minutes for the agarose gel to harden. Overlaid plates were then incubated at 27° C. for 10 days until plaque formation was observed. Plaques in each well were processed through testing and Plaque Picking to provide a single Plaque for Clonal Plaque Purification (i.e. Single Plaque Expansion). The Single Plaque was expanded using Sf9 cell mixture of 120 mL pools in 500 mL flask, with incubation at 27° C. for 4 days. The resulting CP2 BEVs were harvested using centrifugation in 50 mL conical tubes for 5 minutes and collection of supernatant containing CP2 BEVs into a CP2 BEV pool.

### BEV Infection/BIIC Production

Sf9 cell mixture was seeded and expanded through multiple expansion steps up to 3000 mL working volume in a 5 L flask, with a final infection density of 1.0×10⁶ cells/mL. The expanded Sf9 cell mixture was then infected with 0.01 MOI of CP2 BEV. Infected cells were incubated and expanded at 27° C. for 48-36 hours, then harvested by spinning down (polypropylene centrifuge tubes, 5 min) and resuspending the cell pellet at 2.0×10⁷ cells/mL in Hyclone SFX Insect Cell Culture Media, followed by the addition of 300 mM Trehalose, 14% v/v of DMSO, and additional SFX Culture Media to provide target VCD of 2.0×10⁶ cells/mL. Rep/Cap Source BIICs were aliquoted into 2 mL or 5 mL cryovials and frozen down to ≤−65° C. using control rate freezer, and then stored at −80° C. or in LN₂ vapor.

### Example 4. Production of Source Transgene BIICs (B)

Transgene Source BIICs were produced according to Example 3, with Transgene Bacmid material used for P1 BEV production instead of Rep/Cap Bacmid material.

### Example 5. Production of Infection BIICs from Source BIICs

One vial of Sf9 9f4 CB was thawed in a 125 mL shaker flask (37° C. using waterbath, 1-5 minutes until ice crystals dissipate), and then diluted into 20 mL working volume of ESF-AF culture medium. The shaker flask was incubated at 27° C. (100 rpm shaking, 2-inch orbital diameter) in a non-humidified, ambient air, temperature regulated incubator in a first expansion until the cell density was expanded to between 5.0-8.0×10⁶ cells/mL.

The culture was then seeded and expanded through multiple additional expansion steps using larger shaker flasks, with a target output density of 5.0-8.0×10⁶ cells/mL for each expansion step to allow for a consistent seeding density of 1.0-4.0×10⁶ cells/mL in subsequent expansion steps. Expansions were completed at 27° C. for 3-5 days with 100 rpm shaking (≤2 L working volume) or 80 rpm shaking (>2 L working volume).

The following additional expansions were completed: (i) expansion up to 100 mL working volume in a 500 mL flask; (ii) expansion up to 400 mL working volume in a 1.0 L flask; (iii) expansion up to 1500 mL working volume in a 3 L flask; and (iv) expansion up to 2500 mL working volume in each of two 5 L Production Flasks (Rep/Cap Production Flask and Transgene Production Flask).

The Rep/Cap Production Flask was incubated until cell concentration was expanded to 1.8-2.5×10⁶ cells/mL and was then infected with Rep/Cap Source BIIC (Sf9:BIIC Infection Ratio of 1.0×10⁴ cell-to-cell (c/c), equivalent to 1.0×10⁵ (v/v) infection ratio). The infected cells were incubated for 72 hours (target cell diameter of ≥19.0 μm, cell culture density target of ≥3.0×10⁶ cells/mL), and then harvested by spinning down (polypropylene centrifuge tubes, 5 min at 4.0° C.) and resuspending the cell pellet at 2.0×10⁷ cells/mL in 50% 2× Freezing media (858 mL/L of ESF-AF Media, 140 mL/L of Dimethyl Sulfoxide, 113 mL/L of Trehalose, dihydrate) and 50% ESF-AF Media. The resuspended culture of Rep/Cap Infection BIICs was aliquoted into 2 mL or 5 mL cryovials and stored in LN₂ vapor.

The Transgene Production Flask was incubated until cell concentration was expanded to 1.8-2.5×10⁶ cells/mL and was then infected with Transgene Source BIIC (Sf9:BIIC Infection Ratio of 1.0×10⁴ cell-to-cell (c/c), equivalent to 1.0×10⁵ v/v infection ratio). The infected cells were incubated for 96-100 hours (target cell diameter of ≥19.0 μm, cell culture density target of ≥3.0×10⁶ cells/mL), and then harvested by spinning down (polypropylene centrifuge tubes, 5 min at 4.0° C.) and resuspending the cell pellet at 2.0×10⁷ cells/mL in 50% 2× Freezing media (858 mL/L of ESF-AF Media, 140 mL/L of Dimethyl Sulfoxide, 113 mL/L of Trehalose, dihydrate) and 50% ESF-AF Media. The resuspended culture of Transgene Infection BIICs was aliquoted into 2 mL or 5 mL cryovials and stored in LN₂ vapor.

### Example 6. High Cell Density Perfusion—BIIC Production

A collection of studies was completed to investigate the impact of using alternating perfusion technology in the production of baculoviral inoculum banks, such as banks of Baculovirus Infected Insect Cells (BIIC). Perfusion systems were used in coordination with bioreactors to manage and cycle cell culture media within a bioreactor during BIIC production. A High Cell Density ATF Perfusion system supported the production of high quality BIIC banks having an unexpectedly high cell density at large-scale.

### A. Evaluation of ATF Unit

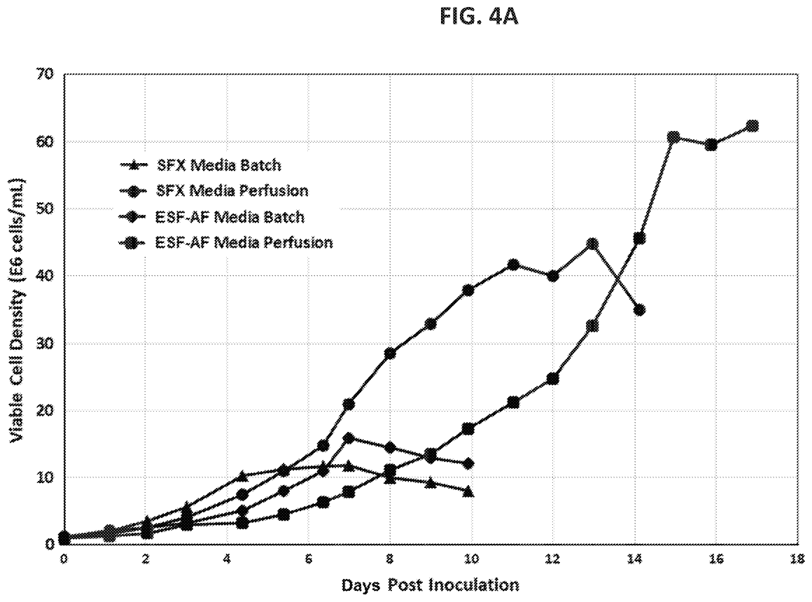

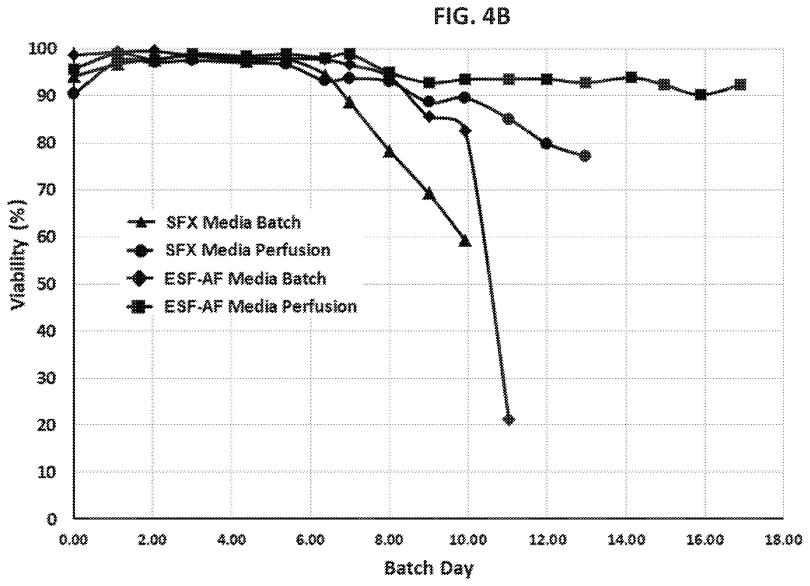

Viral Production Cells were expanded in 3 L flasks (1.5 L working volume) according to the general procedures in Example 2, using an XCell ATF system with certain batches (0.5 sLPM target exchange rate, 1 Vessel volume exchanged per day starting at 2.5-3.0×10⁶ cells/mL). Parameters and results are shown in Table 1 (VCD—Viable Cell Density (×10⁶ cells/mL); CVB—Cell Viability (%); ACD—Average Cell Diameter (μm)) as well as FIG. 4A and FIG. 4B.

TABLE 1
Perfusion vs Batch Analysis

| | | VCD | | |
|---|---|---|---|---|
| Bioreactor | Days Post | (×10⁶ | CVB | ACD |
| Conditions | Infection | cells/mL) | (%) | (μm) |
| SFX Media | 0 | 0.913 | 94 | 14.4 |
| Batch | 1 | 1.97 | 96.65 | 13.85 |
| | 2 | 3.52 | 97.6 | 13.5 |
| | 3 | 5.71 | 98.3 | 13.85 |
| | 4 | 10.265 | 97.25 | 14.2 |
| | 5 | 11.25 | 97.6 | 14.8 |
| | 6 | 11.7 | 94.4 | 15.4 |
| | 7 | 11.9 | 88.6 | 15.8 |
| | 8 | 10.05 | 78.3 | 16.3 |
| | 9 | 9.31 | 69.2 | 16.9 |
| | 10 | 8.1 | 59.3 | 17.4 |
| SFX Media | 0 | 1.13 | 90.3 | 14.6 |
| ATF Perfusion | 1 | 2.105 | 97.4 | 13.7 |
| | 2 | 2.56 | 97.1 | 14 |
| | 3 | 4.15 | 97.55 | 13.95 |
| | 4 | 7.47 | 97.05 | 14.2 |
| | 5 | 11.05 | 96.6 | 14.35 |
| | 6 | 14.75 | 93.2 | 14.6 |
| | 7 | 21 | 93.7 | 14.8 |
| | 8 | 28.5 | 93 | 15.2 |
| | 9 | 32.9 | 88.6 | 15 |
| | 10 | 37.9 | 89.5 | 15.3 |
| | 11 | 41.7 | 85 | 15.9 |
| | 12 | 40 | 79.7 | 16.2 |
| | 13 | 44.8 | 77 | 16.8 |
| | 14 | 35 | 63 | 17.6 |
| ESF-AF Media | 0 | 1.31 | 98.7 | 15.1 |
| Batch | 1 | 1.675 | 99.35 | 14.8 |
| | 2 | 2.56 | 99.5 | 14.3 |
| | 3 | 3.29 | 98.65 | 14.7 |
| | 4 | 5.16 | 98.05 | 14.9 |
| | 5 | 8.1 | 98 | 14.95 |
| | 6 | 11.05 | 97.7 | 15.05 |
| | 7 | 15.9 | 96.6 | 15.8 |
| | 8 | 14.5 | 94.1 | 17.2 |
| | 9 | 12.9 | 85.5 | 17.1 |
| | 10 | 12.1 | 82.6 | 16.8 |
| | 11 | 2.55 | 21.2 | 17.7 |
| ESF-AF Media | 0 | 1.04 | 95.7 | 14.8 |
| ATF Perfusion | 1 | 1.275 | 98.95 | 14.75 |
| | 2 | 1.73 | 97.6 | 14.4 |
| | 3 | 2.98 | 98.95 | 14.25 |
| | 4 | 3.285 | 98.45 | 14.4 |
| | 5 | 4.515 | 98.85 | 14.7 |
| | 6 | 6.395 | 98.1 | 14.9 |
| | 7 | 7.9 | 98.8 | 15 |
| | 8 | 11.2 | 94.9 | 15.5 |
| | 9 | 13.5 | 92.7 | 16.1 |
| | 10 | 17.25 | 93.4 | 16 |
| | 11 | 21.3 | 93.5 | 16.2 |
| | 12 | 24.8 | 93.5 | 16.6 |
| | 13 | 32.6 | 92.7 | 16.6 |
| | 14 | 45.7 | 93.8 | 16.7 |
| | 15 | 60.7 | 92.3 | 16.7 |
| | 16 | 59.5 | 90.1 | 16.8 |
| | 17 | 62.4 | 92.3 | 17 |

The results from the ATF Evaluation study showed that batch units (no ATF perfusion) were limited to peak VCDs of about 15×10⁶ cells/mL, while the SFX Media system with ATF perfusion was able to reach about 45×10⁶ cells/mL and the ESF-AF Media system with ATF perfusion was able to reach above 60×10⁶ cells/mL (see FIG. 4A).

Results also showed that batch units (no ATF perfusion) had significant decreases in cell viability after 6 days (SFX Media batch system) and 10 days (SFX Media batch system), while the SFX Media system with ATF perfusion was able to maintain a cell viability above 75% for 13 days and the ESF-AF Media system with ATF perfusion was able to maintain a cell viability above 90% for 17 days (see FIG. 4B).

### B. Infection Density and Scale Analysis

BIICs were produced in 500 mL Shake Flasks (200 mL working volume) and 3 L glass bioreactors (1.0-1.5 L working volume) according to the general procedures in Example 2 (using SFX Media), with varying target Infection Cell Densities and a target Infection MOI of 0.001. XCell ATF system was used with certain batches (1 Vessel volume exchanged per day starting at 2.5-3.0×10⁶ cells/mL. Resulting BIICs were banked and frozen at 2×10⁷ viable cells/mL at a volume of 1 m. For systems which included ATF perfusion, the cryopreservation banking media was introduced directly into the bioreactor using the ATF perfusion system.

Parameters and results are shown in Table 2 (VCD—Viable Cell Density (×10⁶ cells/mL); CVB—Cell Viability (%); ACD—Average Cell Diameter (μm)). No significant impact was observed in cell doubling time between shake flask and bioreactor. Infection kinetics were consistent across an infection density range of 2.0-6.0×10⁶ cells/mL.

TABLE 2
Infection Density and Scale Analysis

| | Infection Cell | | VCD | | |
|---|---|---|---|---|---|
| Bioreactor | Density | Days Post | (×10⁶ | CVB | ACD |
| Conditions | (cells/mL) | Infection | cells/mL) | (%) | (μm) |
| 500 mL | 2 × 10⁶ | −1 | 1 | 94.1 | 13.66 |
| Flask | | 0 | 1.82 | 94.6 | 14.6 |
| | | 1 | 3.1 | 94 | 14 |
| | | 2 | 4.8 | 95.8 | 16.2 |
| | | 3 | 5.45 | 93.1 | 17.8 |
| | | 4 | 3.17 | 67.1 | 18 |
| | | 5 | 1.53 | 37.7 | 18.1 |
| 3 L | 2 × 10⁶ | −1 | 0.955 | 98 | 14.3 |
| Bioreactor | | 0 | 1.47 | 98.6 | 13.9 |
| No Perfusion | | 1 | 2.72 | 99.2 | 13.7 |
| | | 2 | 4.56 | 98.9 | 14.5 |
| | | 3 | 5.01 | 97.2 | 16.8 |
| | 4 × 10⁶ | −1 | 1.98 | 99.6 | 14.1 |
| | | 0 | 3.05 | 99.6 | 13.8 |
| | | 1 | 5.24 | 99 | 14.2 |
| | | 2 | 6.76 | 98.9 | 14.7 |
| | | 3 | 9.45 | 95.6 | 16.6 |
| | 8 × 10⁶ | −1 | 3.82 | 99.4 | 13.9 |
| | | 0 | 6.02 | 99 | 14 |
| | | 1 | 9.09 | 99.4 | 14.7 |
| | | 2 | 10.6 | 98.1 | 15.3 |
| | | 3 | 10.9 | 90.8 | 16.8 |
| 3 L | 2 × 10⁶ | −1 | 1.19 | 97.6 | 13.4 |
| Bioreactor | | 0 | 1.66 | 98.5 | 13.7 |
| Perfusion | | 1 | 2.77 | 99 | 13.6 |
| | | 2 | 3.32 | 95.9 | 15.2 |
| | | 3 | 3.81 | 96.4 | 17 |
| | 10 × 10⁶ | −5 | 1.87 | 98.2 | 14 |
| | Batch 1 | −4 | 2.12 | 96.9 | 14.1 |
| | | −3 | 3.31 | 97.4 | 14.3 |
| | | −2 | 5.93 | 96 | 14.3 |
| | | −1 | 9.32 | 95.6 | 14.8 |
| | | 0 | 11.9 | 93.8 | 14.7 |
| | | 1 | 16.2 | 87.6 | 14.8 |
| | | 2 | 16 | 78.8 | 15.2 |
| | | 3 | 16.3 | 79.3 | 16.3 |
| | 10 × 10⁶ | −4 | 2.27 | 98.4 | 13.8 |
| | Batch 2 | −3 | 3.42 | 95.3 | 13.7 |
| | | −2 | 5.52 | 95.2 | 13.7 |
| | | −1 | 8.43 | 95 | 14.2 |
| | | 0 | 9.05 | 84.6 | 14.9 |
| | | 1 | 11.8 | 80.8 | 16.1 |
| | | 2 | 14.2 | 87.9 | 16.4 |
| | | 3 | 15.2 | 81.4 | 17 |

The results from the Infection Density and Scale analysis showed that ATF perfusion systems were able to sustain a VCD up to more than 15.0×10⁶ cells/mL for an infection density or 10×10⁶ cells/mL.

### C. Gas Sparger Analysis

BIICs were produced in a 500 mL Shake Flasks (200 mL working volume) and 2 L glass bioreactors (1.3 L working volume, one bioreactor using a ring-type macro sparger (R), one bioreactor using a micro sparger (M)) according to the general procedures in Example 2 (using SFX Media), with varying Infection Cell Density and a target Infection MOI of 0.001. XCell ATF system was used with certain batches (1 Vessel volume exchanged per day starting at 2.5-3.0×10⁶ cells/mL.

Parameters and results are shown in Table 3 (VCD—Viable Cell Density (×10⁶ cells/mL); CVB—Cell Viability (%); ACD—Average Cell Diameter (μm)).

No impact on infection kinetics was observed across different type of spargers, and the cell growth trends were generally shown to be consistent across sparger types (even with higher infection density). There was a lower viability at harvest for micro sparger conditions, but sparger selection (Macro vs. Micro) had a generally minimal impact on cell growth parameters.

TABLE 3
Gas Sparger Analysis

| | Infection Cell | | VCD | | |
|---|---|---|---|---|---|
| Bioreactor | Density | Days Post | (×10⁶ | CVB | ACD |
| Conditions | (cells/mL) | Infection | cells/mL) | (%) | (μm) |
| 500 mL | 2 × 10⁶ | −1 | 1 | 94.1 | 13.66 |
| Flask | | 0 | 1.82 | 94.6 | 14.6 |
| | | 1 | 3.1 | 94 | 14 |
| | | 2 | 4.8 | 95.8 | 16.2 |
| | | 3 | 5.45 | 93.1 | 17.8 |
| | | 4 | 3.17 | 67.1 | 18 |
| | | 5 | 1.53 | 37.7 | 18.1 |
| 2 L Bioreactor | 10 × 10⁶ | −5 | 0.95 | 99.2 | 13.6 |
| Perfusion | | −4 | 1.29 | 98.7 | 14 |
| Ring Macro | | −3 | 2.4 | 99.5 | 14.3 |
| Sparger | | −2 | 3.56 | 99.2 | 13.5 |
| | | −1 | 6 | 98.3 | 13.8 |
| | | 0 | 9.75 | 96.7 | 14 |
| | | 1 | 12.7 | 97.3 | 14.2 |
| | | 2 | 16.8 | 96.4 | 14.8 |
| | | 3 | 19 | 91.7 | 17.5 |
| 2 L Bioreactor | 20 × 10⁶ | −5 | 2.6 | 99 | 13.6 |
| Perfusion | | −4 | 4.44 | 99.7 | 13.9 |
| Micro Sparger | | −3 | 7.08 | 99 | 13.9 |
| | | −2 | 12 | 97.3 | 14.3 |
| | | −1 | 16 | 91.8 | 15 |
| | | 0 | 20.9 | 94.1 | 15.2 |
| | | 1 | 23.5 | 90.2 | 15.9 |
| | | 2 | 26.3 | 85.1 | 16.15 |
| | | 3 | 31.9 | 72.7 | 16.5 |

### D. BIIC-Type Analysis

BIICs were produced in 500 mL Shake Flasks (200 mL working volume) and 2 L glass bioreactors (1.0-1.5 L working volume, 3 runs) according to the general procedures in Example 2 (using SFX Media). BIICs were also produced in 2 L glass bioreactors (1.0-1.5 L working volume, 3 runs) according to the general procedures in Example 1 (using SFX Media). Samples were produced using varying target Infection Cell Densities and a target Infection MOI of 0.001. XCell ATF system was used with certain batches (1 Vessel volume exchanged per day starting at 2.5-3.0×10⁶ cells/mL. Resulting BIICs were banked and frozen at 2×10⁷ viable cells/mL at a volume of 1 m. For systems which included ATF perfusion, the cryopreservation banking media was introduced directly into the bioreactor using the ATF perfusion system.

Parameters and results are shown in Table 4 (VCD—Viable Cell Density (×10⁶ cells/mL); CVB—Cell Viability (%); ACD—Average Cell Diameter (μm)).

No significant impact on infection kinetics was observed across different type of BIICs and multiple runs. Cell growth trends were consistent across BIIC type.

TABLE 4
BIIC-Type Analysis

| | Infection Cell | | VCD | | |
|---|---|---|---|---|---|
| Bioreactor | Density | Days Post | (×10⁶ | CVB | ACD |
| Conditions | (cells/mL) | Infection | cells/mL) | (%) | (μm) |
| 500 mL | 2 × 10⁶ | −1 | 1 | 94.1 | 13.66 |
| Flask | | 0 | 1.82 | 94.6 | 14.6 |
| | | 1 | 3.1 | 94 | 14 |
| | | 2 | 4.8 | 95.8 | 16.2 |
| | | 3 | 5.45 | 93.1 | 17.8 |
| | | 4 | 3.17 | 67.1 | 18 |
| | | 5 | 1.53 | 37.7 | 18.1 |
| 2 L Bioreactor | Run 1 | −5 | 1.87 | 98.2 | 14 |
| Perfusion | 2 × 10⁶ | −4 | 2.12 | 96.9 | 14.1 |
| Transgene | | −3 | 3.31 | 97.4 | 14.3 |
| BIIC | | −2 | 5.93 | 96 | 14.3 |
| | | −1 | 9.32 | 95.6 | 14.8 |
| | | 0 | 11.9 | 93.8 | 14.7 |
| | | 1 | 16.2 | 87.6 | 14.8 |
| | | 2 | 16 | 78.8 | 15.2 |
| | | 3 | 16.3 | 79.3 | 16.3 |
| | Run 2 | −4 | 2.27 | 98.4 | 13.8 |
| | 10 × 10⁶ | −3 | 3.42 | 95.3 | 13.7 |
| | | −2 | 5.52 | 95.2 | 13.7 |
| | | −1 | 8.43 | 95 | 14.2 |
| | | 0 | 9.05 | 84.6 | 14.9 |
| | | 1 | 11.8 | 80.8 | 16.1 |
| | | 2 | 14.2 | 87.9 | 16.4 |
| | | 3 | 15.2 | 81.4 | 17 |
| | Run 3 | −5 | 1.66 | 94.7 | 14.2 |
| | 2 × 10⁶ | −4 | 2.49 | 96.4 | 14.1 |
| | | −3 | 4.34 | 99.1 | 14.2 |
| | | −2 | — | — | — |
| | | −1 | 8.37 | 99.4 | 13.2 |
| | | 0 | 13.7 | 95.4 | 14.5 |
| | | 1 | — | — | — |
| | | 2 | 30.7 | 95.2 | 15.4 |
| | | 3 | 26.4 | 93.2 | 17.2 |
| 2 L Bioreactor | Run 1 | −5 | 2.01 | 96.4 | 14.3 |
| Perfusion | 2 × 10⁶ | −4 | 2.89 | 96.9 | 14.21 |
| Rep/Cap | | −3 | 5.76 | 99 | 14 |
| BIIC | | −2 | — | — | — |
| | | −1 | 9.26 | 97.7 | 13.6 |
| | | 0 | 12.9 | 92.7 | 14.5 |
| | | 1 | — | — | — |
| | | 2 | 28.3 | 91.9 | 15.3 |
| | | 3 | 27 | 90.3 | 16.1 |
| | | 4 | 23.7 | 84.5 | 17.2 |
| | Run 2 | −4 | 1.6 | 98.6 | 14.02 |
| | 2 × 10⁶ | −3 | 3.38 | 97.7 | 13.93 |
| | | −2 | 4.55 | 97 | 13.7 |
| | | −1 | 7.65 | 96.3 | 13.9 |
| | | 0 | 14.1 | 97.5 | 14 |
| | | 1 | 17.4 | 92.3 | 14.6 |
| | | 2 | 26.2 | 90.8 | 16 |
| | | 3 | 19.3 | 80.1 | 17.7 |
| | Run 3 | −4 | 1.72 | 98.9 | 14.08 |
| | 2 × 10⁶ | −3 | 3.2 | 98.1 | 13.93 |
| | | −2 | 4.7 | 97.5 | 13.8 |
| | | −1 | 8.35 | 96.5 | 13.9 |
| | | 0 | 14.2 | 98.1 | 14.2 |
| | | 1 | 20.2 | 92.8 | 14.8 |
| | | 2 | 29 | 92.5 | 15.6 |
| | | 3 | 24.5 | 85.2 | 16.6 |

### Example 7. Upstream—Production of Bulk Particle Pool (A)

One vial of the Sf9 9f4 CB was thawed in a 125 mL shaker flask (37° C. using waterbath, 1-5 minutes until ice crystals dissipate), and then diluted into 20 mL working volume of ESF-AF culture medium. The shaker flask was incubated at 27° C. (130-150 rpm shaking, 25 mm orbital diameter) for about 48 hours in a non-humidified, ambient air, temperature regulated incubator in a first expansion until the cell density was expanded to between 5.0-8.0×10⁶ cells/mL.

The culture was then seeded and expanded through multiple additional expansion steps using larger shaker flasks, with a target output density of 5.0-8.0×10⁶ cells/mL for each expansion step to allow for a consistent target seeding density of 1.0-4.0×10⁶ cells/mL in subsequent expansion steps. Expansions were completed at 27° C. for 3-5 days with 130-150 rpm shaking (≤400 mL working volume) or 100-120 rpm shaking (>400 mL working volume).

The following additional expansions were completed: (i) expansion up to 100 mL working volume in a 250 or 500 mL flask; (ii) expansion up to 400 mL working volume in a 1.0 L flask; (iii) expansion up to 1500 mL working volume in a 3 L flask; and (iv) expansion up to 2500 mL working volume in each of two 5 L flasks (5000 mL total working volume).

The expanded culture mixture was transferred to a 50 L GE WAVE bioreactor (0.25 mL/min fixed air sparge, oxygen on demand up to 40% dissolved O₂, 20 rpm rocking, 9° rocking angle, 250 mL/min air inlet) for an additional expansion (3-5 days at 27° C.) up to a 25 L working volume with a target output density of 5.0-8.0×10⁶ cells/mL. The culture medium was then seeded into a stirred-tank GE Xcellerex Bioreactor (68 rpm agitation, 0.5 mL/min fixed air sparge, cascading oxygen on demand up to 40% dissolved O₂, 0.5 mL/min headspace flow rate), and expanded (N-1 Bioreactor step, 2-3 days at 27° C.) up to a 125 L working volume with a target output density of 1.0-5.0×10⁶ cells/mL.

The culture mixture was seeded into a single-use Production Bioreactor with a seeding density of 0.8-1.5×10⁶ cells/mL and 200 L working volume. The culture medium was further expanded in the Bioreactor (6 W/m³ impeller, 0.8 mL/min fixed air sparge, cascading oxygen on demand up to 40% dissolved O₂, 0.8 mL/min headspace flow rate) up to 3.0-3.2×10⁶ cells/mL in a 200 L working volume.

The cells in the Bioreactor were then co-infected with Rep/Cap Infection BIICs (1:250k v/v) and Transgene Infection BIICs (1:80k v/v). Infected cells were incubated for 144 hours (6 days) and the bulk harvest was collected for lysis and processing through Downstream processing.

Samples were taken through each of the expansion and bioreaction steps to monitor cell density and viability throughout the upstream process.

In one alternative, the expansion Bioreactor was a Pall 200 L Allegro Bioreactor maintained as 35 rpm agitation, 1.3 mL/min fixed air sparge, cascading oxygen on demand up to 40% dissolved O₂, and 0.8 mL/min headspace flow rate.

In one alternative, the processing parameters for the Production Bioreactor were 41 agitation rpm (pre-infection), 51 agitation rpm (post-infection), 2.5 mL/min fixed air sparge, cascading oxygen on demand up to 40% dissolved O₂, and 1.2 mL/min headspace flow rate, with a target expansion up to 3.2-3.4×10⁶ cells/mL in a 200 L working volume.

In one alternative, the expanded culture mixture was transferred to a Pall Allegro XRS 25 L Bioreactor for an additional expansion (25 cpm agitation, cascading oxygen on demand up to 40% dissolved O₂, 0.3 mL/min fixed air sparge, 0.5 mL/min headspace flow rate 3-5 days at 27° C.) up to a 10 L working volume with a target output density of 5.0×10⁶-1.0×10⁷ cells/mL.

In one alternative, the N-1 bioreactor was a Pall 125 L Allegro Bioreactor (45 rpm agitation, cascading oxygen on demand up to 40% dissolved O₂, 0.8 L/min air overlay, 27° C. vessel temp, 1.5 L/min O₂ flow rate) with a target output density of 5.0×10⁶-1.0×10⁷ cells/mL.

In one alternative, the N production bioreactor was a PD 200 L Allegro Bioreactor (60 rpm agitation, cascading oxygen on demand up to 40% dissolved O₂, 1.2 L/min air overlay, 27±1° C. vessel temp, 2.5 L/min O₂ flow rate). The culture mixture was seeded into the Production Bioreactor with a target seeding density of about 1.0×10⁶ cells/mL and 200 L working volume. The culture medium was further expanded in the Bioreactor up to about 3.2×10⁶ cells/mL in a 200 L working volume. The cells in the Bioreactor were then co-infected with Rep/Cap Infection BIICs (1:300k v/v) and Transgene Infection BIICs (1:100k v/v). Infected cells were incubated for 168 hours (7 days). Post-infection, the bioreactor conditions were adjusted as follows: 70 rpm agitation, cascading oxygen on demand up to 40% dissolved O₂, 1.2 L/min air overlay, 27° C. vessel temp, 3.0 L/min O₂ flow rate. The bulk harvest was collected for lysis and processing through Downstream processing.

### Example 8. Upstream—Production of Bulk Particle Pool (B)

One vial of the Sf9 9f4 CB was thawed in a 125 mL shaker flask (37° C. using waterbath, 1-5 minutes until ice crystals dissipate), and then diluted into 20 mL working volume of ESF-AF culture medium. The shaker flask was incubated at 27° C. (100 rpm shaking, 2-inch orbital diameter) in a non-humidified, ambient air, temperature regulated incubator in a first expansion until the cell density was expanded to between 5.0-8.0×10⁶ cells/mL.

The culture was then seeded and expanded through multiple additional expansion steps using larger shaker flasks, with a target output density of 4.0-8.0×10⁶ cells/mL for each expansion step to allow for a consistent seeding density of 0.5-3.0×10⁶ cells/mL in subsequent expansion steps. Expansions were completed at 27° C. for 3-5 days with 100 rpm shaking (≤2 L working volume) or 80 rpm shaking (>2 L working volume).

The following additional expansions were completed: (i) expansion up to 200 mL working volume in a 1 L flask; (ii) expansion up to 1000 mL working volume in a 3 L flask; and (iii) expansion up to 3000 mL working volume in 5 L flasks.

5 L of expanded culture mixture was spiked with 10% w/v Pluronic F-68 (2.045 v/v spike), which was then transferred to a 50 L GE WAVE bioreactor (0.25 mL/min fixed air sparge, oxygen on demand up to 40% dissolved O₂, 20 rpm rocking up to 9° angle) for an additional expansion (3-5 days at 27° C.) up to a 25 L working volume with a target output density of 2.0-6.0×10⁶ cells/mL.

The culture medium was spiked again with 10% w/v Pluronic F-68 (2.045 v/v spike) and then seeded into a GE 250 L Xcellerex Bioreactor with a seeding density of 0.8×10⁶ cells/mL and 125 L working volume (Hyclone SFX Insect Cell Culture Media). The culture medium was expanded in the Bioreactor for 2-4 days (cascading oxygen on demand up to 40% dissolved O₂, 1 L/min air overlay, 27° C. vessel temp, 60° C. vent heater temp, downward mixer direction of 80 rpm) up to 3.0×10⁶ cells/mL in a 200 L working volume.

The cells in the Bioreactor were then co-infected with Rep/Cap Infection BIICs and Transgene Infection BIICs (1:1 BIIC ratio, 5.0×10³ SF9:BIIC ratio). Infected cells were incubated for 5-7 days and the bulk harvest was collected for lysis and processing through Downstream processing.

Samples were taken through each of the expansion and bioreaction steps to monitor cell density and viability throughout the upstream process.

### Example 9. Upstream—Production of Bulk Particle Pool (C)

One vial of the Sf9 9f4 CB was thawed in a 125 mL shaker flask (37° C. using waterbath, 1-5 minutes until ice crystals dissipate), and then diluted into 20 mL working volume of ESF-AF culture medium. The shaker flask was incubated at 27° C. (100 rpm shaking, 2-inch orbital diameter) in a non-humidified, ambient air, temperature regulated incubator in a first expansion until the cell density was expanded to between 5.0-8.0×10⁶ cells/mL.

The culture was then seeded and expanded through multiple additional expansion steps using larger shaker flasks, with a target output density of 3.0-6.0×10⁶ cells/mL for each expansion step to allow for a consistent seeding density of 0.5-4.0×10⁶ cells/mL in subsequent expansion steps. Expansions were completed at 27° C. for 3-5 days with 100 rpm shaking (≤2 L working volume) or 80 rpm shaking (>2 L working volume).

The following additional expansions were completed: (i) expansion up to 200 mL working volume in a 1 L flask; (ii) expansion up to 1000 mL working volume in a 3 L flask; and (iii) expansion up to 3000 mL working volume in 5 L flasks.

5 L of expanded culture mixture was spiked with 10% w/v Pluronic F-68 (2.045 v/v spike), which was then transferred to a 50 L GE WAVE bioreactor (0.25 mL/min fixed air sparge, oxygen on demand up to 40% dissolved O₂, 20 rpm rocking up to 9° angle) for an additional expansion (3-5 days at 27° C.) up to a 25 L working volume with a target output density of 2.0-5.0×10⁶ cells/mL.

The culture medium was spiked again with 10% w/v Pluronic F-68 (2.5 v/v spike) and then seeded into a ThermoFisher 250 L HyPerforma Bioreactor with a seeding density of 1.0-2.0×10⁶ cells/mL and 125 L working volume (Hyclone SFX Insect Cell Culture Media). The culture medium was expanded in the Bioreactor for 2-3 days (cascading oxygen on demand up to 40% dissolved O₂, 0.25 L/min air sparge, 7 L/min air overlay, 27° C. vessel temp, 65° C. vent heater temp, 60 rpm mixer) up to 2.5-2.75×10⁶ cells/mL in a 200 L working volume.

The cells in the Bioreactor were then co-infected with Rep/Cap Infection BIICs (1:250k v/v) and Transgene Infection BIICs (1:50k v/v). Infected cells were incubated for 1-2 days and the bulk harvest was collected for lysis and processing through Downstream processing.

Samples were taken through each of the expansion and bioreaction steps to monitor cell density and viability throughout the upstream process.

### Example 10. Evaluation of Perfusion BIIC Banks

A study was completed to investigate the impact of using ATF Perfusion systems in the production and cryo-preservations of BIICs for use in AAV production, AAV particles were produced according to the general procedures in Example 9, using BIICs produced and analyzed in Example 6D. A first Control sample used a combination of Control Transgene BIICs and Control Rep/Cap BIICs; a second set of samples used the Perfusion Transgene BIICs from Example 6D (Run 1, Run 2 and Run 3) in combination with Control Rep/Cap BIICs; and a third set of samples used Control Transgene BIICs in combination with the Perfusion Rep/Cap BIICs from Example 6D (Run 1, Run 2 and Run 3).

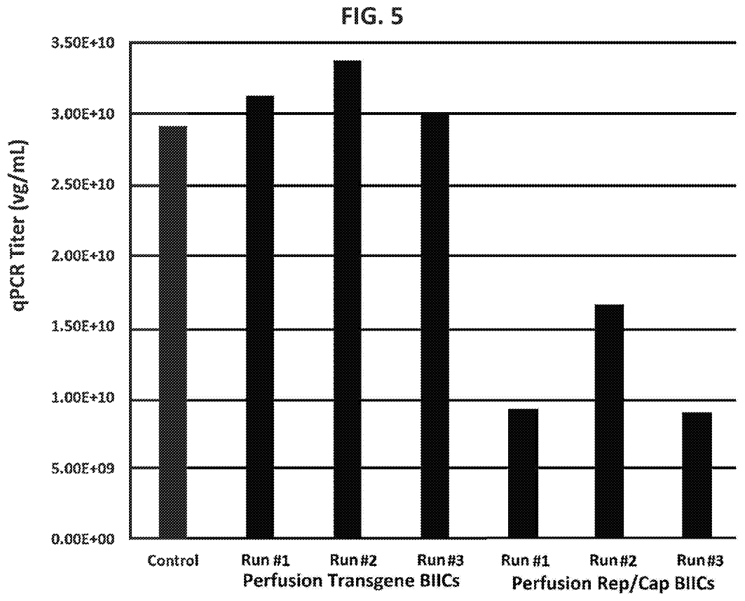

Resulting clarified lysate pools were analyzed for AAV particles titer (qPCR). The results are shown in FIG. 5.

The results of the evaluation of ATF Perfusion BIIC Banks showed that the Perfusion Transgene BIICs from Example 6D provided AAV titers which were equal or higher than the Control sample; while Perfusion Rep/Cap BIICs from Example 6D provided AAV titers which were lower than the Control sample. All BIIC sample provided AAV titers of about 1.0×10¹⁰ VG/mL or higher.

### Example 11. Fill and Finish

A Drug Substance was transferred to a Biosafety Cabinet (BSC) and filtered through a 0.22 μm filter (dual-in-line sterilizing grade filters). The filtered Drug Substance pool was then aseptically filled into 2 ml Cryovials utilizing a programmable Peristaltic dispensing pump within the BSC. Product vials were stoppered, seal capped, 100% visually inspected and labeled (at 25° C.), and then stored at <−65° C.

We claim:

1. A method for producing a baculovirus infected insect cell (BIIC), comprising:
(a) introducing a volume of cell culture medium into a bioreactor;
(b) introducing at least one viral production cell (VPC) into the bioreactor and expanding the number of VPCs in the bioreactor to a target VPC cell density;
(c) introduction at least one Baculoviral Expression Vector (BEV) into the bioreactor, wherein the at least one BEV comprises an AAV viral expression construct or a payload construct;
(d) incubating the mixture of VPCs and BEVs in the bioreactor under conditions which allow at least one BEV to infect at least one VPC to produce a baculovirus infected insect cell (BIIC);
(e) incubating the bioreactor under conditions which allow the number of BIICs in the bioreactor to reach a target BIIC cell density; and
(f) optionally harvesting the BIICs from the bioreactor.

2. The method of claim 1, wherein the bioreactor comprises a perfusion system for managing the cell culture medium within the bioreactor.

3. The method of claim 2, wherein the perfusion system is an alternating tangential flow (ATF) perfusion system.

4. The method of claim 2 or claim 3, wherein the perfusion system replaces at least a portion of the culture medium in the bioreactor while retaining at least 90% of the VPCs and BIICs within the bioreactor.

5. The method of claim 4, wherein the perfusion system removes cell waste products from the cell culture medium within the bioreactor.

6. The method of claim 4 or claim 5, wherein the perfusion system replaces cell culture media which has been depleted of nutrients by cellular metabolism.

7. The method of any one of claims 1-6, wherein the perfusion system replaces cell culture media with a cryopreservation media after the bioreactor reaches the target BIIC cell density, which allows for BIIC cells to be frozen and preserved before or after being harvested from the bioreactor.

8. The method of any one of claims 1-7, wherein the volume of cell culture medium in the bioreactor is at least 5 L, 10 L, 20 L, 50 L, 100 L, or 200 L.

9. The method of any one of claims 1-8, wherein the VPCs are insect cells.

10. The method of claim 9, wherein the VPCs are Sf9 cells.

11. The method of any one of claims 1-10, wherein the target VPC cell density at BEV infection is 1.5-4.0×10⁶ cells/mL.

12. The method of any one of claims 1-10, wherein the target VPC cell density at BEV infection is 2.0-3.5×10⁶ cells/mL.

13. The method of any one of claims 1-12, wherein BEVs are introduced into the bioreactor at a target Multiplicity of Infection (MOI) of BEVs to VPCs.

14. The method of claim 13, wherein BEV MOI is 0.0005-0.003.

15. The method of claim 13, wherein BEV MOI is 0.001-0.002.

16. The method of any one of claims 1-15, wherein BIIC cell density at harvesting is 6.0-18.0×10⁶ cells/mL.

17. The method of any one of claims 1-15, wherein BIIC cell density at harvesting is 8.0-16.5×10⁶ cells/mL.

18. The method of any one of claims 1-15, wherein BIIC cell density at harvesting is 10.0-16.5×10⁶ cells/mL.

19. The method of any one of claims 1-18, wherein the at least one BEV comprises at least one baculovirus (expressionBac) which comprises an AAV viral expression construct.

20. The method of any one of claims 1-18, wherein the at least one BEV comprises at least one baculovirus (payloadBac) which comprises a payload construct comprising a polynucleotide encoding a payload.

21. A baculovirus infected insect cell (BIIC) produced by the method of any one of claims 1-20.

22. A method for producing an adeno-associated virus (AAV) comprising a polynucleotide encoding a payload, said method comprising:
(a) culturing viral production cells (VPCs) in a bioreactor to a target cell density, wherein the bioreactor comprises a cell culture medium;
(b) introducing into the bioreactor at least one baculovirus infected insect cell (BIIC), wherein the at least one BIIC introduced into the bioreactor comprises at least one expressionBIIC which comprises at least one expressionBac, wherein the at least one BIIC introduced into the bioreactor comprises at least one payloadBIIC which comprises at least one payloadBac, and wherein the at least one BIIC introduced into the bioreactor comprises at least one BIIC of claim 21;
(c) incubating the VPCs in the bioreactor under conditions that result in the production of one or more AAVs within one or more VPCs, wherein one or more of the AAVs comprise the polynucleotide encoding the payload; and
(d) harvesting a viral production pool from the bioreactor, wherein the viral production pool comprises one or more VPCs comprising one or more AAVs.

23. The method of claim 22, wherein the VPCs are insect cells.

24. The method of claim 23, wherein the VPCs are Sf9 cells.

25. The method of any one of claims 22-24, wherein the target cell density of the VPCs of step (a) is 3.0×10⁶-3.4×10⁶ cells/mL (e.g., 3.2×10⁶-3.4×10⁶ cells/mL; e.g., 3.2×10⁶ cells/mL).

26. An adeno-associated virus (AAV) produced by the method of any one of claims 22-25.

27. A pharmaceutical composition comprising the AAV of claim 26 and at least one pharmaceutically acceptable excipient.

28. The pharmaceutical composition of claim 27, for use in treating and/or preventing a disease.

29. A method of treating a disease comprising administering an effective amount of the pharmaceutical composition of claim 27.

30. Use of the pharmaceutical composition of claim 27 in the manufacture of a medicament for treating and/or preventing a disease.
